(19) United States
(12) Patent Application Publication
(10) Pub. No.: US 2024/0101563 A1
(43) Pub. Date: Mar. 28, 2024

(54) NOVEL COMPOUNDS

(71) Applicant: AC Immune SA, Lausanne (CH)

(72) Inventor: Jérôme MOLETTE, Lausanne (CH)

(73) Assignee: AC Immune SA, Lausanne (CH)

(21) Appl. No.: 18/227,103

(22) Filed: Jul. 27, 2023

(30) Foreign Application Priority Data
Jul. 28, 2022 (EP) 22 187 634.5
Dec. 23, 2022 (EP) 22 216 498.0

(51) Int. Cl. C07D 487/04; C07D 471/04; C07D 519/00

(52) U.S. Cl. CPC C07D 487/04; C07D 471/04; C07D 519/00

(57) ABSTRACT

The present invention relates to novel compounds for the treatment, alleviation or prevention of a group of diseases, disorders and abnormalities which are responsive to the modulation or inhibition of the activation of a component of the NLRP3 inflammasome pathway. In particular, the component of the inflammasome pathway is a NOD-like receptor (NLR) family, pyrin domain-containing protein 3 (NLRP3) inflammasome. More particularly, the compounds of the present invention have the capability to modulate the NLRP3 inflammasome pathway. Further, the compounds of the present invention are suitable for the treatment, alleviation or prevention of a group of diseases, disorders and abnormalities which are responsive to the modulation, in particular decrease of IL-1 beta and/or IL-18 levels.

### FIELD OF THE INVENTION

The present invention relates to novel compounds that are useful for the treatment, alleviation or prevention of a disease, disorder or abnormality which is responsive to the modulation, in particular inhibition of the activation, of a component of the NLRP3 inflammasome pathway. In particular, the component of the inflammasome pathway is NOD-like receptor (NLR) family, pyrin domain-containing protein 3 (NLRP3) inflammasome. More particularly, the compounds of the present invention have the capability to modulate, e.g., inhibit the activation of, the NLRP3 inflammasome pathway. Further, the compounds of the present invention have the capability to modulate, in particular decrease, IL-1 beta and/or IL-18 levels. The present invention relates to novel compounds for the treatment, alleviation or prevention of a disease, disorder or abnormality which is responsive to the inhibition of the activation of the NLRP3 inflammasome pathway. The present invention relates to novel compounds for the treatment, alleviation or prevention of a disease, disorder or abnormality which is responsive to the modulation of IL-1 beta and/or IL-18 levels. The present invention relates to pharmaceutical compositions comprising said compounds, methods of using said compounds in the treatment of various diseases, disorders or abnormalities which is responsive to the above-mentioned modulation, medicaments containing them and their uses thereof.

### BACKGROUND OF THE INVENTION

Inflammasome protein complexes are the key components of inflammatory signalling. These complexes assemble in response to various danger signals such as molecules from infectious agents (pathogen-associated molecular patterns, PAMPs) as well as altered host molecules, products of sterile tissue damage and environmental factors (danger associated molecular patterns, DAMPs). The inflammasome family consists of NALP1-14, IPAF, and NAIP 1-6, with each family member providing specificity towards different PAMPs/DAMPs including nucleic acids, bacterial proteins, metabolites, protein aggregates and the activity of toxins (Sharma, D. & Kanneganti, T. D. The cell biology of inflammasomes: mechanisms of inflammasome activation and regulation. J. Cell Bio. 213, 617-629 (2016)). Inflammasomes are typically composed of a sensor (a cytosolic pattern-recognition receptor, PRR) and an adaptor protein called apoptosis associated speck-like protein containing a caspase-recruitment domain (CARD) (ASC), and an effector such as the protease caspase-1 (Broz, P.; Dixit, V. M. Inflammasomes: Mechanism of Assembly, Regulation and Signalling. Nat. Rev. Immunol. 2016, 16, 407-420).

NLRP3 (NOD-like receptor (NLR) family, pyrin domain-containing protein 3 inflammasome is one of the best-described family members. It is a tripartite protein of the NLR family and contains an amino-terminal PYRIN (PYD) domain, a nucleotide-binding NACHT domain and a carboxy-terminal leucine-rich repeat (LRR) domain. In response to various agents including aggregated proteins, crystals and altered cellular ion homeostasis, the NLRP3 sensor molecule assembles into a multi-molecular complex with apoptosis-associated speck-like protein containing a caspase activation and recruitment domain (ASC aka PYCARD) adaptor protein. ASC protein polymerization into a large complex (ASC speck) leads to activation of caspase-1 effector protein and subsequent cleavage of pro-IL-1 beta (β) and pro-IL18 into their active secreted forms and mediates pyroptosis (Heneka et al., 2018 Nat Rev Neurosci). IL-1 beta (p) acts through IL-1 beta (13) receptors, insertion andy pro-inflammatory signals including IL-6 and TNF alpha secretion, and attracts and activates cells of adaptive immune system at the sites of infection. NLRP3/ASC complexes seems to be released into the extracellular environment where they can propagate inflammation.

Multiple genetic and pharmacological evidence highlight the importance of NLRP3 inflammasome in human disease. NLRP3 gain-of-function mutations lead to the inherited cryopyrin-associated periodic syndromes (CAPS) including Muckle-Wells syndrome (MWS), familial cold auto-inflammatory syndrome (FCAS) and neonatal-onset multisystem inflammatory disease (NOMID).

Accumulation of tissue damage products associated with ageing results in activation of NLRP3 inflammasome in multiple diseases including metabolic disorders, Alzheimer's disease, Parkinson's disease, amyotrophic lateral sclerosis, atherosclerosis, obesity, lung diseases, liver diseases and gout.

Vast experimental evidence from animal models points out the detrimental role of excessive NLRP3 activation in a wide spectrum of diseases. NLRP3-inflammasome genetic or pharmacological downregulation showed protection in models of Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 and type 2 diabetes, rheumatoid arthritis, myelodysplastic syndrome, among others (Heneka et al., Nat. Rev. Neurosci. 2018 October; 19(10):610-621; Mangan et al., Nat. Rev. Drug Discov. 2018 August; 17(8):588-606).

For the reasons described above modulation of NLRP3 inflammasome pathway activity represents a promising therapeutic approach.

Current treatments for NLRP3-related diseases include biologics targeting IL-1. These are the recombinant IL-1 receptor antagonist anakinra, the neutralizing IL-1 beta (β) antibody canakinumab and the soluble decoy IL-1 receptor rilonacept. However, their activity is limited to downstream effectors of inflammasome and their bioavailability for central nervous system (CNS) applications is limited.

Several small molecules have been shown to inhibit the NLRP3 inflammasome pathway (Baldwin, A. G., Brough, D. & Freeman, S. Inhibiting the NLRP3 inflammasome pathway: a chemical perspective. J. Med. Chem. 59, 1691-1710 (2016); reviewed in Mangan et al., Nat Rev Drug Discov. 2018 August; 17(8):588-606). These include various chemical classes such as sulfonylurea-based compounds (glyburide, CP-456,773 (aka CRID3 and MCC950) and its derivatives); fenamate classes of non-steroidal anti-inflammatory drugs; hydroxysulfonamide analogue JC-171; novel boron compound series; benzimidazole-containing structure Fc11a-2; polyketide spirodalesol; acrylate and acrylamide derivatives; 3,4-methylenedioxy-β-nitrostyrene; β-sulfonyl nitrile molecule OLT1177; CY-09; BOT-4-one; and Michael acceptors. Most of these compounds have a promiscuous mode of action and limited potency.

WO2016131098, WO2017/140778 and WO2018215818 refer to sulfonylurea and related compounds and their use in treating or identifying a disease or condition responsive to inhibition of NLRP3 or inhibition of the activation of NLRP3 or related components of the inflammatory process.

WO2019008025, WO2019008029, WO2019034686, WO2019034688, WO2019034690, WO2019034692, WO2019034693, WO2019034696, WO2019034697, WO2019068772, WO2019092170, WO2019092171 and WO2019092172 refer to novel compounds (e.g. sulfonylureas, sulfonylthioureas, sulfoximine ureas and sulfoximine thioureas), useful in the treatment and prevention of medical disorders and diseases, most especially by NLRP3 inhibition.

WO2017184604, WO2017184623, WO2017184624, WO2019023145, WO2019023147 and WO2019079119 refer to chemical entities that are useful for treating a condition, disease, or disorder in which a decrease or increase in NLRP3 activity contributes to the pathology and/or symptoms and/or progression of the condition, disease, or disorder in a subject.

WO2019211463, WO2020021447, and WO2021043966, WO2021239885, WO2021219784, WO2021214284, WO2021209552, WO2021209539 disclose compounds for inhibiting NLRP3 and/or NLRP3 inflammasome pathway.

WO2018136890 refers to sulfonylurea and sulfonyl thiourea compounds and their use in treating a disease or condition responsive to modulation of cytokines such as IL-1 beta (p) and IL-18, modulation of NLRP3 or inhibition of the activation of NLRP3 or related components of the inflammatory process.

WO2018225018 and WO2019043610 refer to NLRP3 modulators as well as to the use of the novel inhibitor compounds in the treatment of diseases or conditions as well as treatment of disease states mediated by NLRP3 as well as treatment of diseases or conditions in which interleukin 1 beta (@) activity and interleukin-18 (IL-18) are implicated.

WO2018015445 refers to sulfonylurea compounds which possess inflammasome inhibitory activity and are accordingly useful in methods of treatment of the human or animal body.

WO2020018975 discloses sulfonimidamide derivatives defined as inhibitors of interleukin-1 activity and NLRP3 modulators in connection with inflammatory processes.

WO9832733 refers to aryl and heteroaryl substituted sulfonyl ureas that are inhibitors of interleukin-1 alpha (a) and interleukin-1 beta (p) processing and release.

WO2020018970 discloses sulfonylureas defined as inhibitors of interleukin-1 activity.

WO2020/234715 discloses pyridazine-3-yl phenol compounds defined as inhibitors of NOD-like receptor protein 3 (NLRP3) inflammasome activity.

WO2021/193897 refers to substituted pyridazine compounds that are described as having suppressive action on NLRP3 inflammasome activity.

The crosstalk between the NLRP3 inflammasome pathway and Tau pathology has been recently deciphered. Ising et al. (Nature 2019 November; 575(7784):669-673) investigated the important role of microglia and NLRP3 inflammasome pathway activation in the pathogenesis of tauopathies in the Tau22 mouse model of FrontoTemporal Dementia (FTD). Genetic ablation of components of the NLRP3 inflammasome pathway in Tau22 mice reduced Tau aggregation/phosphorylation as well as improved cognition. Stancu et al. (Acta Neuropathol. 2019; 137(4): 599-617) investigated the role of inflammasome activation in prion-like or templated seeding of Tau pathology. Significant inhibition of exogenously seeded Tau pathology was found in ASC deficient—PS19 Tau transgenic mice. Furthermore, it was demonstrated that chronic intracerebral administration of the NLRP3 inhibitor, MCC950, inhibits exogenously seeded Tau pathology. Finally, ASC deficiency also decreased non-exogenously seeded Tau pathology in PS19 mice.

There is a need to identify and develop specific NLRP3 inflammasome pathway inhibitors and/or modulators of interleukin activity with beneficial pharmacological and/or physiological and/or physicochemical properties.

The present invention provides compound of formula (I) which have surprisingly been found to be capable of modulating a component of the NLRP3 inflammasome pathway, in particular inhibiting the activation, of a component of the NLRP3 inflammasome pathway, such as NLRP3 inflammasome. Thus, such compounds are beneficial in the treatment of a disease, disorder, or abnormality which is responsive to the modulation of a component of the NLRP3 inflammasome pathway and/or which is responsive to the modulation of IL-1 beta and/or IL-18 levels that commonly lead to pathological inflammation.

### SUMMARY OF THE INVENTION

Accordingly, the present invention provides compounds that can be employed in the treatment, alleviation or prevention of a disease, disorder or an abnormality which is responsive to the modulation, in particular inhibition, of a component of the NLRP3 inflammasome pathway, or which is responsive to the modulation, in particular decrease, of IL-1 beta and/or IL-18 levels.

Various embodiments of the invention are described herein.

Within one aspect of the invention, provided herein is a compound of formula (I′)

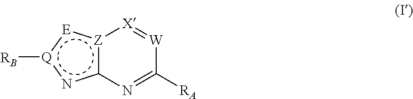

or a stereoisomer, a racemic mixture, a tautomer, a polymorph, a pharmaceutically acceptable salt, a prodrug, a hydrate, or a solvate thereof;
wherein
X′ is selected from CH or N;
W is selected from N, CH or CR_{c};
Q is selected from N and C;
E is selected from NRₐ and CRₐ;
Z is selected from N and C;
wherein at least one of Q and Z is C, and/or E is CRₐ;
R_{c} is selected from the group consisting of —C₁-C₄alkyl, —O—C₁-C₄alkyl, —C₁-C₄alkyl-OH, -halo or —C₁-C₄alkyl-Hal;
Rₐ is selected from the group consisting of —H and —C₁-C₃alkyl;
R_{A} and R_{B} are each selected from

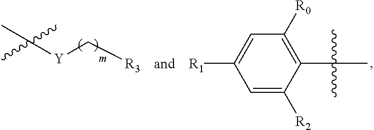

wherein one of R_{A} and R_{B} is

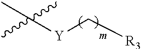

and the other of R_{A} and R_{B} is

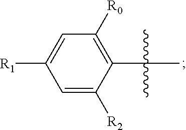

R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH, NR_{d}, O or is a bond;
R_{d} is selected from the group consisting of —C₁-C₄alkyl, —C₁-C₄alkyl-OH, or —C₁-C₄alkyl-Hal;
R₃ is selected from the group consisting of
4-, 5- or 6-membered heterocycloalkyl containing one or two heteroatoms, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH; —NR₅R₆ and halo; or
8-, 9- or 10-membered bicyclic fused heterocycloalkyl containing one to three heteroatoms, wherein said heteroatom(s) is/are independently selected from N and O, optionally substituted at any available position with one or two substituents independently selected from the group consisting of C₁-C₄alkyl, haloC₁-C₄alkyl, hydroxyC₁-C₄alkyl, —OH, —NR₅R₆ and halo; or
C₃-C₆cycloalkyl optionally substituted with one or two substituents independently selected from the group consisting of C₁-C₄alkyl, haloC₁-C₄alkyl, hydroxyC₁-C₄alkyl, —OH and halo; or
C₁-C₆alkyl optionally substituted with one or two substituents independently selected from the group consisting of —OH, halo, haloC₁-C₄alkyl, C₁-C₄alkoxy and —NR₅R₆;
R₅ and R₆ are independently selected from H and C₁-C₃alkyl; and
m is 0, 1 or 2.

Within one aspect of the invention, provided herein is a compound having the formula (II′)

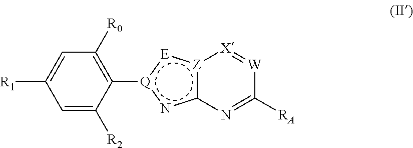

or a stereoisomer, a racemic mixture, a tautomer, a polymorph, a pharmaceutically acceptable salt, a prodrug, a hydrate, or a solvate thereof;
wherein
X′ is selected from CH or N;
W is selected from N, CH or CR_{c};
Q is selected from N and C;
E is selected from NRₐ and CRₐ;
Z is selected from N and C;
wherein at least one of Q and Z is C, and/or E is CRₐ;
R_{c} is selected from the group consisting of C₁-C₄alkyl, —O—C₁-C₄alkyl, C₁-C₄alkyl-OH, halo or haloC₁-C₄alkyl;
Rₐ is selected from the group consisting of —H and —C₁-C₃alkyl;
R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
R_{A} is

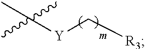

Y is selected from NH, NR_{d}, O or is a bond;
R_{d} is selected from the group consisting of —C₁-C₄alkyl, —C₁-C₄alkyl-OH or —C₁-C₄alkyl-Hal;
R₃ is selected from the group consisting of
4-, 5- or 6-membered heterocycloalkyl containing one or two heteroatoms, preferably one heteroatom, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH; —NR₅R₆ and halo; or
8-, 9- or 10-membered bicyclic fused heterocycloalkyl containing one to three heteroatoms, wherein said heteroatom(s) is/are independently selected from N and O, optionally substituted at any available position with one or two substituents independently selected from the group consisting of C₁-C₄alkyl, haloC₁-C₄alkyl, hydroxyC₁-C₄alkyl, —OH, —NR₅R₆ and halo; or C₃-C₆cycloalkyl optionally substituted with one or two substituents independently selected from the group consisting of C₁-C₄alkyl, haloC₁-C₄alkyl, hydroxyC₁-C₄alkyl, —OH and halo; or
C₁-C₆alkyl optionally substituted with one or two substituents independently selected from the group consisting of —OH, halo, haloC₁-C₄alkyl, C₁-C₄alkoxy and —NR₅R₆;
R₅ and R₆ are independently selected from H and C₁-C₃alkyl; and
m is 0, 1 or 2.

Within one aspect, provided herein is a compound of formula (I)

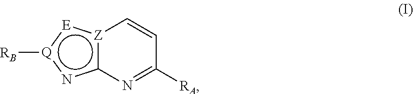

or a stereoisomer, a racemic mixture, a tautomer, a polymorph, a pharmaceutically acceptable salt, a prodrug, a hydrate, or a solvate thereof;
wherein
Q is selected from N and C;
E is selected from NRₐ and CRₐ;
Z is selected from N and C;
wherein at least one of Q and Z is C, and/or E is CRₐ;
Rₐ is selected from the group consisting of —H and —C₁-C₃alkyl;
R_{A} and R_{B} are each selected from

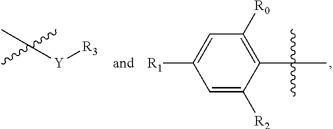

wherein one of R_{A} and R_{B} is

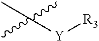

and the other of R_{A} and R_{B} is

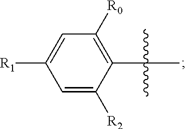

R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH and O; and
R₃ is selected from the group consisting of
4-, 5- or 6-membered heterocycloalkyl containing one or two heteroatoms, preferably one heteroatom, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH and halo;
C₃-C₆cycloalkyl optionally substituted with one or two substituents independently selected from the group consisting of C₁-C₄alkyl, haloC₁-C₄alkyl, hydroxyC₁-C₄alkyl, —OH and halo; and
C₁-C₆alkyl optionally substituted with one or two substituents independently selected from the group consisting of —OH, halo, haloC₁-C₄alkyl, C₁-C₄alkoxy and —NR₅R₆, wherein R₅ and R₆ are independently selected from H and C₁-C₃alkyl.

Within another aspect, there is provided a compound of formula (II)

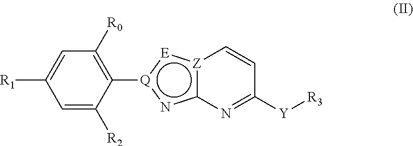

or a stereoisomer, a racemic mixture, a tautomer, a polymorph, a pharmaceutically acceptable salt, a prodrug, a hydrate, or a solvate thereof;
wherein
Q is selected from N and C;
E is selected from NRₐ and CRₐ;
Z is selected from N and C;
wherein at least one of Q and Z is C, and/or E is CRₐ;
Rₐ is selected from the group consisting of —H and —C₁-C₃alkyl;
R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH and O; and
R₃ is selected from the group consisting of
4-, 5- or 6-membered heterocycloalkyl containing one or two heteroatoms, preferably one heteroatom, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH and halo;
C₃-C₆cycloalkyl optionally substituted with one or two substituents independently selected from the group consisting of C₁-C₄alkyl, haloC₁-C₄alkyl, hydroxyC₁-C₄alkyl, —OH and halo; and
C₁-C₆alkyl optionally substituted with one or two substituents independently selected from the group consisting of —OH, halo, haloC₁-C₄alkyl, C₁-C₄alkoxy and —NR₅R₆, wherein R₅ and R₆ are independently selected from H and C₁-C₃alkyl.

In another aspect there is provided a compound of formula (III)

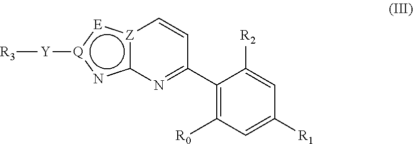

or a stereoisomer, a racemic mixture, a tautomer, a polymorph, a pharmaceutically acceptable salt, a prodrug, a hydrate, or a solvate thereof;
wherein
Q is selected from N and C;
E is selected from NRₐ and CRₐ;
Z is selected from N and C;
wherein at least one of Q and Z is C, and/or E is CRₐ;
Rₐ is selected from the group consisting of —H and —C₁-C₃alkyl;
R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH and O; and
R₃ is selected from the group consisting of
4-, 5- or 6-membered heterocycloalkyl containing one or two heteroatoms, preferably one heteroatom, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH and halo;
C₃-C₆cycloalkyl optionally substituted with one or two substituents independently selected from the group consisting of C₁-C₄alkyl, haloC₁-C₄alkyl, hydroxyC₁-C₄alkyl, —OH and halo; and C₁-C₄alkyl optionally substituted with one or two substituents independently selected from the group consisting of —OH, halo, haloC₁-C₄alkyl, C₁-C₄alkoxy and —NR₅R₄, wherein R₅ and R₆ are independently selected from H and C₁-C₃alkyl.

In another aspect there is provided a compound of formula (II′a)

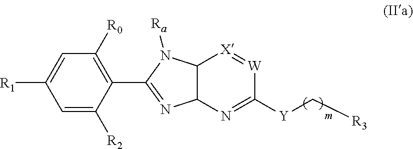

or a stereoisomer, a racemic mixture, a tautomer, a polymorph, a pharmaceutically acceptable salt, a prodrug, a hydrate, or a solvate thereof;
wherein
X′ is selected from CH or N;
W is selected from N, CH or CR_{c};
R_{c} is selected from the group consisting of C₁-C₄alkyl, —O—C₁-C₄alkyl, C₁-C₄alkyl-OH, halo or haloC₁-C₄alkyl;
Rₐ is selected from the group consisting of —H and —C₁-C₃alkyl;
R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH, NR_{d}, O or is a bond;
R_{d} is selected from the group consisting of —C₁-C₄alkyl, —C₁-C₄alkyl-OH, or —C₁-C₄alkyl-Hal;
R₃ is selected from the group consisting of
4-, 5- or 6-membered heterocycloalkyl containing one or two heteroatoms, preferably one heteroatom, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH; —NR₅R₆ and halo; or
8-, 9- or 10-membered bicyclic fused heterocycloalkyl containing one to three heteroatoms, wherein said heteroatom(s) is/are independently selected from N and O, optionally substituted at any available position with one or two substituents independently selected from the group consisting of C₁-C₄alkyl, haloC₁-C₄alkyl, hydroxyC₁-C₄alkyl, —OH, —NR₆R₆ and halo; or
C₃-C₆cycloalkyl optionally substituted with one or two substituents independently selected from the group consisting of C₁-C₄alkyl, haloC₁-C₄alkyl, hydroxyC₁-C₄alkyl, —OH and halo; or
C₁-C₆alkyl optionally substituted with one or two substituents independently selected from the group consisting of —OH, halo, haloC₁-C₄alkyl, C₁-C₄alkoxy and —NR₅R₆;
R₅ and R₆ are independently selected from H and C₁-C₃alkyl; and
m is 0, 1 or 2.

In another aspect there is provided a compound of formula (IIa)

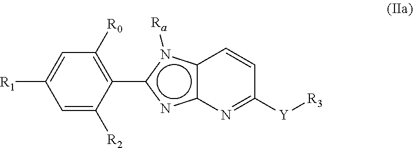

or a stereoisomer, a racemic mixture, a tautomer, a polymorph, a pharmaceutically acceptable salt, a prodrug, a hydrate, or a solvate thereof;
wherein
Rₐ is selected from the group consisting of —H and —C₁-C₃alkyl;
R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH and O; and
R₃ is selected from the group consisting of
4-, 5- or 6-membered heterocycloalkyl containing one or two heteroatoms, preferably one heteroatom, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH and halo;
C₃-C₆cycloalkyl optionally substituted with one or two substituents independently selected from the group consisting of C₁-C₄alkyl, haloC₁-C₄alkyl, hydroxyC₁-C₄alkyl, —OH and halo; and
C₁-C₆alkyl optionally substituted with one or two substituents independently selected from the group consisting of —OH, halo, haloC₁-C₄alkyl, C₁-C₄alkoxy and —NR₅R₆, wherein R₅ and R₆ are independently selected from H and C₁-C₃alkyl.

In another aspect there is provided a compound of formula (II′b)

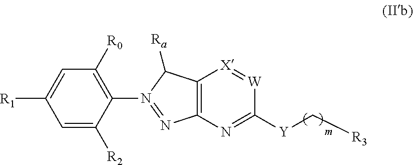

or a stereoisomer, a racemic mixture, a tautomer, a polymorph, a pharmaceutically acceptable salt, a prodrug, a hydrate, or a solvate thereof;
wherein
X′ is selected from CH or N;
W is selected from N, CH or CR_{c};
R_{c} is selected from the group consisting of —C₁-C₄alkyl, —O—C₁-C₄alkyl, —C₁-C₄alkyl-OH, -halo or C₁-C₄alkyl-Hal;
Rₐ is selected from the group consisting of —H and —C₁-C₃alkyl;
R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH, NR_{d}, O or is a bond;
R_{d} is selected from the group consisting of —C₁-C₄alkyl, —C₁-C₄alkyl-OH, or —C₁-C₄alkyl-Hal;
R₃ is selected from the group consisting of
4-, 5- or 6-membered heterocycloalkyl containing one or two heteroatoms, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH; —NR₅R₆ and halo; or
8-, 9- or 10-membered bicyclic fused heterocycloalkyl containing one to three heteroatoms, wherein said heteroatom(s) is/are independently selected from N and O, optionally substituted at any available position with one or two substituents independently selected from the group consisting of C₁-C₄alkyl, haloC₁-C₄alkyl, hydroxyC₁-C₄alkyl, —OH, —NR₅R₆ and halo; or
C₃-C₆cycloalkyl optionally substituted with one or two substituents independently selected from the group consisting of C₁-C₄alkyl, haloC₁-C₄alkyl, hydroxyC₁-C₄alkyl, —OH and halo; or
C₁-C₆alkyl optionally substituted with one or two substituents independently selected from the group consisting of —OH, halo, haloC₁-C₄alkyl, C₁-C₄alkoxy and —NR₅R₆;
R₅ and R₆ are independently selected from H and C₁-C₃alkyl;
m is 0, 1 or 2.

In another aspect there is provided a compound of formula (IIb)

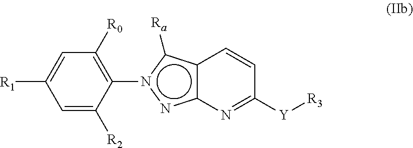

or a stereoisomer, a racemic mixture, a tautomer, a polymorph, a pharmaceutically acceptable salt, a prodrug, a hydrate, or a solvate thereof;
wherein
Rₐ is selected from the group consisting of —H and —C₁-C₃alkyl;
R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH and O; and
R₃ is selected from the group consisting of
4-, 5- or 6-membered heterocycloalkyl containing one or two heteroatoms, preferably one heteroatom, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH and halo;
C₃-C₆cycloalkyl optionally substituted with one or two substituents independently selected from the group consisting of C₁-C₄alkyl, haloC₁-C₄alkyl, hydroxyC₁-C₄alkyl, —OH and halo; and
C₁-C₆alkyl optionally substituted with one or two substituents independently selected from the group consisting of —OH, halo, haloC₁-C₄alkyl, C₁-C₄alkoxy and —NR₅R₆, wherein R₅ and R₆ are independently selected from H and C₁-C₃alkyl.

In another aspect there is provided a compound of formula (IV)

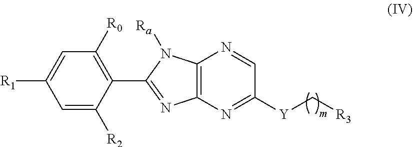

or a stereoisomer, a racemic mixture, a tautomer, a polymorph, a pharmaceutically acceptable salt, a prodrug, a hydrate, or a solvate thereof; wherein
Rₐ is selected from the group consisting of —H and —C₁-C₃alkyl;
R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH, NR_{d}, O or is a bond;
R_{d} is selected from the group consisting of —C₁-C₄alkyl, —C₁-C₄alkyl-OH, or —C₁-C₄alkyl-Hal;
R₃ is selected from the group consisting of
4-, 5- or 6-membered heterocycloalkyl containing one or two heteroatoms, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH; —NR₅R₆ and halo; or
8-, 9- or 10-membered bicyclic fused heterocycloalkyl containing one to three heteroatoms, wherein said heteroatom(s) is/are independently selected from N and O, optionally substituted at any available position with one or two substituents independently selected from the group consisting of C₁-C₄alkyl, haloC₁-C₄alkyl, hydroxyC₁-C₄alkyl, —OH, —NR₅R₆ and halo; or
C₃-C₆cycloalkyl optionally substituted with one or two substituents independently selected from the group consisting of C₁-C₄alkyl, haloC₁-C₄alkyl, hydroxyC₁-C₄alkyl, —OH and halo; or
C₁-C₆alkyl optionally substituted with one or two substituents independently selected from the group consisting of —OH, halo, haloC₁-C₄alkyl, C₁-C₄alkoxy and —NR₅R₆;
R₅ and R₆ are independently selected from H and C₁-C₃alkyl;
m is 0, 1 or 2.

In another aspect there is provided a compound of formula (V)

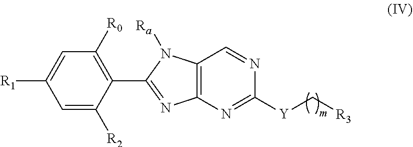

or a stereoisomer, a racemic mixture, a tautomer, a polymorph, a pharmaceutically acceptable salt, a prodrug, a hydrate, or a solvate thereof;
wherein
Rₐ is selected from the group consisting of —H and —C₁-C₃alkyl;
R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH, NR_{d}, O or is a bond;
R_{d} is selected from the group consisting of —C₁-C₄alkyl, —C₁-C₄alkyl-OH, or —C₁-C₄alkyl-Hal;
Rₐ is selected from the group consisting of
4-, 5- or 6-membered heterocycloalkyl containing one or two heteroatoms, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH; —NR₅R₆ and halo; or
8-, 9- or 10-membered bicyclic fused heterocycloalkyl containing one to three heteroatoms, wherein said heteroatom(s) is/are independently selected from N and O, optionally substituted at any available position with one or two substituents independently selected from the group consisting of C₁-C₄alkyl, haloC₁-C₄alkyl, hydroxyC₁-C₄alkyl, —OH, —NR₅R₆ and halo; or
C₃-C₆cycloalkyl optionally substituted with one or two substituents independently selected from the group consisting of C₁-C₄alkyl, haloC₁-C₄alkyl, hydroxyC₁-C₄alkyl, —OH and halo; or
C₁-C₄alkyl optionally substituted with one or two substituents independently selected from the group consisting of —OH, halo, haloC₁-C₄alkyl, C₁-C₄alkoxy and —NR₅R₆;
R₅ and R₆ are independently selected from H and C₁-C₃alkyl;
m is 0, 1 or 2.

Within the present invention any reference to the compounds of formula (I′), (I), (II′), (II), (II′a), (II′b), (IIa), (IIb), (III′), (III), (IV) or (V), or the preferred embodiments thereof is intended to also refer to the stereoisomers, or racemic mixtures, or tautomers, or polymorphs, or pharmaceutically acceptable salts, or prodrugs, or hydrates, or solvates thereof.

Compounds of formula (I′), (I), (II′), (II), (II′a), (II′b), (IIa), (IIb), (III′), (III), (IV) or (V), or a stereoisomer, a racemic mixture, a tautomer, a polymorph, a pharmaceutically acceptable salt, a prodrug, a hydrate, or a solvate thereof, are suitable for the treatment, alleviation or prevention of a disease, disorder or an abnormality which is responsive to the modulation, in particular inhibition, of a component of the NLRP3 inflammasome pathway, or which is responsive to the modulation, in particular decrease, of IL-1 beta and/or IL-18 levels. In particular, the component of the inflammasome pathway is the NLRP3 inflammasome. Activation of the NLRP3 inflammasome pathway can trigger the formation of ASC specks, cleavage and activation of Caspase-1 and Caspase-8 and subsequent activation and release IL-1 beta, IL-18, gasdermin D cleavage and pore formation, pyroptosis, and release of IL-1alpha, IL-33, IL-17 and High-Mobility Group Box (HMGB) protein. The compounds of formula (I′), (I), (II′), (II), (II′a), (II′b), (IIa), (IIb), (III′), (III), (IV) or (V), or a stereoisomer, a racemic mixture, a tautomer, a polymorph, a pharmaceutically acceptable salt, a prodrug, a hydrate, or a solvate thereof, have the capability to modulate, in particular decrease, IL-1 beta and/or IL-18 levels.

The compounds of formula (I′), (I), (II′), (II), (II′a), (II′b), (IIa), (IIb), (III′), (III), (IV) or (V), or a stereoisomer, a racemic mixture. a tautomer, a polymorph, a pharmaceutically acceptable salt, a prodrug, a hydrate, or a solvate thereof, display high capability in modulating and, in particular inhibiting the activation of, a component of the NLRP3 inflammasome pathway, in particular wherein the component of the inflammasome pathway is the NLRP3 inflammasome. Due to their unique design features, these compounds display properties such as modulating or inhibiting the activation of the NLRP3 inflammasome pathway allowing them to be a successful medicament for the treatment, alleviation or prevention of diseases, disorders and abnormalities responsive to the modulation or inhibition of a component of the NLRP3 inflammasome pathway such as, for example, Alzheimer's disease, Parkinson's disease, CAPS, non-alcoholic fatty liver disease (NAFLD), non-alcoholic steatohepatitis (NASH) and gout.

In a further embodiment, the invention relates to a pharmaceutical composition comprising a compound of formula (I′), (I), (II′), (II), (II′a), (II′b), (IIa), (IIb), (III′), (III), (IV) or (V), or a stereoisomer, a racemic mixture, a tautomer, a polymorph, a pharmaceutically acceptable salt, a prodrug, a hydrate, or a solvate thereof, and optionally comprising at least one pharmaceutically acceptable carrier, diluent, adjuvant or excipient.

In another embodiment, the present invention refers to a compound of formula (I′), (I), (II′), (II), (II′a), (II′b), (IIa), (IIb), (III′), (III), (IV) or (V), or a stereoisomer, a racemic mixture, a tautomer, a polymorph, a pharmaceutically acceptable salt, a prodrug, a hydrate, or a solvate thereof, for use as a medicament.

Yet another embodiment, the present invention refers to a compound of formula (I′), (I), (II′), (II), (II′a), (II′b), (IIa), (IIb), (III′), (III), (IV) or (V), or a stereoisomer, a racemic mixture, a tautomer, a polymorph, a pharmaceutically acceptable salt, a prodrug, a hydrate, or a solvate thereof, for use in the treatment, alleviation or prevention of a disease, disorder, or abnormality which is responsive to the modulation, in particular inhibition of activation, of a component of the NLRP3 inflammasome pathway and/or which is responsive to the modulation, in particular decrease, of IL-1 beta and/or IL-18 levels.

A further embodiment is concerned with the use of the compound of formula (I′), (I), (II′), (II), (II′a), (II′b), (IIa), (IIb), (III′), (III), (IV) or (V), or a stereoisomer, a racemic mixture, a tautomer, a polymorph, a pharmaceutically acceptable salt, a prodrug, a hydrate, or a solvate thereof, for the manufacture of a medicament for treating, alleviating or preventing a disease, disorder or abnormality which is responsive to the modulation, in particular inhibition of activation, of a component of the NLRP3 inflammasome pathway and/or which is responsive to the modulation, in particular decrease, of IL-1 beta and/or IL-18 levels.

In yet another embodiment, the present invention is directed to a method of treating, alleviating or preventing a disease, disorder or abnormality which is responsive to the modulation, in particular inhibition of activation, of a component of the NLRP3 inflammasome pathway or which is responsive to the modulation, in particular decrease, of IL-1 beta and/or IL-18 levels, the method comprising administering a therapeutically effective amount of a compound of formula (I′), (I), (II′), (II), (II′a), (II′b), (IIa), (IIb), (III′), (III), (IV) or (V), or a stereoisomer, a racemic mixture, a tautomer, a polymorph, a pharmaceutically acceptable salt, a prodrug, a hydrate, or a solvate thereof, to a subject in need thereof (e.g. a patient).

A pharmaceutical composition comprising a combination of a compound of formula (I′), (I), (II′), (II), (II′a), (II′b), (IIa), (IIb), (III′), (III), (IV) or (V), or a stereoisomer, a racemic mixture, a tautomer, a polymorph, a pharmaceutically acceptable salt, a prodrug, a hydrate, or a solvate thereof, and at least one further biologically active compound differing from the compound of formula (I′), (I), (II′), (II), (II′a), (II′b), (IIa), (IIb), (III′), (III), (IV) or (V), or a stereoisomer, a racemic mixture, a tautomer, a polymorph, a pharmaceutically acceptable salt, a prodrug, a hydrate, or a solvate thereof, and optionally comprising at least one pharmaceutically acceptable carrier, diluent, adjuvant or excipient, is also the subject-matter of the present invention.

In particular, the further biologically active compound can be one which is used for the treatment of a disease, disorder, or abnormality associated with a disease targeting different pathomechanism, e.g. an anti-amyloid beta antibody, anti-Tau antibody, amyloid beta small molecule inhibitor, Tau aggregation small molecule inhibitor, anti-alpha synuclein antibody or alpha-synuclein aggregation small molecule inhibitor, anti-TDP-43 antibody or anti-TDP-43 aggregation small molecule inhibitor, among others. When a compound of the invention is used in combination with a further biologically active compound, the dose of each compound may differ from the dose if the compound is used as monotherapy.

An additional embodiment relates to the use of the compound of formula (I′), (I), (II′), (II), (II′a), (II′b), (IIa), (IIb), (III′), (III), (IV) or (V), or a stereoisomer, a racemic mixture, a tautomer, a polymorph, a pharmaceutically acceptable salt, a prodrug, a hydrate, or a solvate thereof, as an analytical reference or an in vitro screening tool.

The present invention is described hereinafter.

### BRIEF DESCRIPTION OF THE FIGURES

FIG. 1: Inhibition of IL-1beta release by a compound of the invention (Example compound 53) in LPS/ATP induced acute peritonitis mouse model.

FIG. 2: Inhibition of IL-1beta release by a compound of the invention (compound 18) in LPS/ATP induced acute peritonitis mouse model.

### DETAILED DESCRIPTION OF THE INVENTION

The present invention relates to compounds of formula (I′), and to compounds of formula (I), (II′), (II), (II′a), (II′b), (IIa), (IIb), (III′), (III), IV) or (V) including stereoisomers, racemic mixtures, tautomers, polymorphs, pharmaceutically acceptable salts, prodrugs, hydrates, or solvates thereof.

Any of the definitions of R_{A}, R_{B}, R₀, R₁, R₂, R₃, R₄, R₅, R₆, Rₐ, R_{c}, R_{d}, E, Q, W, X, X′, Z, Y, m, n, and/or a which are given with respect to the compounds of formula (I′) and the subformulae thereof apply analogously to compounds of formula (I), (II′), (II), (II′a), (II′b), (IIa), (IIb), (III′), (III), (IV) or (V), wherever R_{A}, R_{B}, R₀, R₁, R₂, R₃, R₄, R₅, R₆, Rₐ, R_{c}, R_{d}, E, Q, W, X, X′, Z, Y, m, n, and/or a are used.

Within one aspect of the invention, provided herein is a compound of formula (I′)

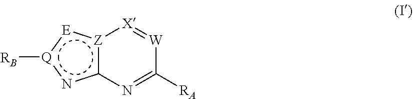

or a stereoisomer, a racemic mixture, a tautomer, a polymorph, a pharmaceutically acceptable salt, a prodrug, a hydrate, or a solvate thereof;
wherein
X′ is selected from CH or N;
W is selected from N, CH or CR_{c};
Q is selected from N and C;
E is selected from NRₐ and CRₐ;
Z is selected from N and C;
wherein at least one of Q and Z is C, and/or E is CRₐ;
R_{c} is selected from the group consisting of —C₁-C₄alkyl, —O—C₁-C₄alkyl, —C₁-C₄alkyl-OH, -halo or C₁-C₄alkyl-Hal;
Rₐ is selected from the group consisting of —H and —C₁-C₃alkyl;
R_{A} and R_{B} are each selected from

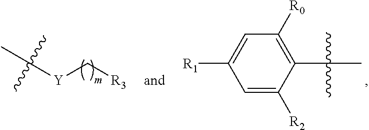

wherein one of R_{A} and R_{B} is

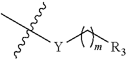

and the other of R_{A} and R_{B} is

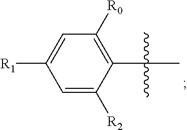

R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH, NR_{d}, O or is a bond;
R_{d} is selected from the group consisting of —C₁-C₄alkyl, —C₁-C₄alkyl-OH, or —C₁-C₄alkyl-Hal;
R₃ is selected from the group consisting of
4-, 5- or 6-membered heterocycloalkyl containing one or two heteroatoms, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH; —NR₅R₆ and halo; or
8-, 9- or 10-membered bicyclic fused heterocycloalkyl containing one to three heteroatoms, wherein said heteroatom(s) is/are independently selected from N and O, optionally substituted at any available position with one or two substituents independently selected from the group consisting of C₁-C₄alkyl, haloC₁-C₄alkyl, hydroxyC₁-C₄alkyl, —OH, —NR₅R₆ and halo; or
C₃-C₆cycloalkyl optionally substituted with one or two substituents independently selected from the group consisting of C₁-C₄alkyl, haloC₁-C₄alkyl, hydroxyC₁-C₄alkyl, —OH and halo; or
C₁-C₆alkyl optionally substituted with one or two substituents independently selected from the group consisting of —OH, halo, haloC₁-C₄alkyl, C₁-C₄alkoxy and —NR₅R₆;
R₅ and R₆ are independently selected from H and C₁-C₃alkyl; and
m is 0, 1 or 2.

The present invention also relates to compounds of formula (I) as defined below

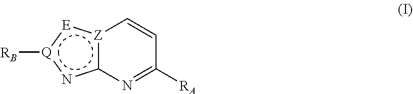

or a stereoisomer, a racemic mixture, a tautomer, a polymorph, a pharmaceutically acceptable salt, a prodrug, a hydrate, or a solvate thereof;
wherein
Q is selected from N and C;
E is selected from NRₐ and CRₐ;
Z is selected from N and C;
wherein at least one of Q and Z is C, and/or E is CRₐ;
Rₐ is selected from the group consisting of —H and —C₁-C₃alkyl;
R_{A} and R_{B} are each selected from

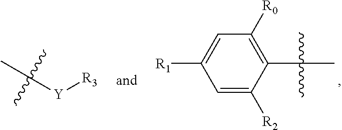

wherein one of R_{A} and R_{B} is

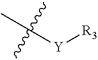

and the other of R_{A} and R_{B} is

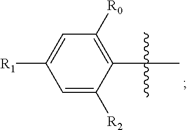

R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH and O; and
R₃ is selected from the group consisting of
4-, 5- or 6-membered heterocycloalkyl containing one or two heteroatoms, preferably one heteroatom, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH and halo;
C₃-C₆cycloalkyl optionally substituted with one or two substituents independently selected from the group consisting of C₁-C₄alkyl, haloC₁-C₄alkyl, hydroxyC₁-C₄alkyl, —OH and halo; and
C₁-C₆alkyl optionally substituted with one or two substituents independently selected from the group consisting of —OH, halo, haloC₁-C₄alkyl, C₁-C₄alkoxy and —NR₅R₆, wherein R₅ and R₆ are independently selected from H and C₁-C₃alkyl.

In a preferred embodiment R₀ is selected from —H and C₁-C₃alkyl, more preferably —H or methyl. In one preferred embodiment R₀ is —H. In another preferred embodiment R₀ is C₁-C₃alkyl, preferably methyl or ethyl, more preferably methyl.

In a preferred embodiment, R₁ is selected from —CF₃, —OCF₃ and —OCHF₂. In one preferred embodiment R₁ is —CF₃. In another preferred embodiment R₁ is —OCF₃. In another preferred embodiment R₁ is —OCHF₂.

In another preferred embodiment R₁ is halo. Where R₁ is halo, the halogen (halo) is preferably chloro.

In one preferred embodiment R₂ is —OH or H, more preferably —OH. In another embodiment R₂ is —H. In another embodiment R₂ is —CF₂.

In one preferred embodiment R₀ is —H, R₁ is selected from —CF₃, —OCF₃, —OCHF₂ and —Cl, and R₂ is —OH or —H. In one preferred embodiment R₀ is —H, R₁ is —CF₃, and R₂ is —OH.

In another preferred embodiment R₀ is —H or methyl, R₁ is selected from —CF₃, or —Cl, and R₂ is —OH.

In more preferred embodiments
R₀ is —H, R₁ is —CF₃, and R₂ is —OH; or
R₀ is —H, R₁ is —Cl, and R₂ is —OH; or
R₀ is —CH₃, R₁ is —Cl, and R₂ is —OH.
R₀ is —CH₃, R₁ is —CF₃, and R₂ is —OH.

In one embodiment R₃ is selected from the group consisting of
4-, 5- or 6-membered heterocycloalkyl containing one or two heteroatoms, wherein said heteroatom(s) is/are independently selected from N and/or O, optionally substituted with one or two substituents independently selected from the group consisting of —NR₅R₆, C₁-C₄alkyl or OH;
8-, 9- or 10-membered bicyclic fused heterocycloalkyl containing one to three heteroatoms, wherein said heteroatom(s) is/are independently selected from N and O, optionally substituted at any available position with one or two substituents independently selected from the group consisting of C₁-C₄alkyl, haloC₁-C₄alkyl, hydroxyC₁-C₄alkyl, —OH, —NR₅R₆ and halo;
C₃-C₆cycloalkyl optionally substituted with one or two substituents independently selected from the group consisting of C₁-C₄alkyl, —OH and halo;
hydroxyC₁-C₆alkyl;
haloC₁-C₄alkyl, preferably —CF₃, or
C₁-C₆alkyl optionally substituted with —NR₅R₆;
wherein R₅ and R₆ are independently selected from H and C₁-C₃alkyl; and
m is 0 or 1.

In another particular embodiment R₃ is selected from the group consisting of
4-, 5- or 6-membered heterocycloalkyl containing one heteroatom, wherein said heteroatom is N or O, wherein the 4-, 5- or 6-membered heterocycloalkyl is optionally substituted with one or two substituents independently selected from the group consisting of C₁-C₄alkyl, haloC₁-C₄alkyl, hydroxyC₁-C₄alkyl, —OH and halo, preferably optionally substituted with one or two substituents independently selected from the group consisting of C₁-C₄alkyl;
C₃-C₆cycloalkyl optionally substituted with one or two substituents independently selected from the group consisting of C₁-C₄alkyl, —OH and halo;
hydroxyC₁-C₄alkyl; and
C₁-C₆alkyl optionally substituted with —NR₅R₆, wherein R₅ and R₆ are independently selected from H and C₁-C₃alkyl.

In one preferred embodiment R₃ is selected from a 4-, 5- or 6-membered heterocycloalkyl containing one or two heteroatoms, preferably one heteroatom, wherein said heteroatom(s) is/are N or O. The 4-, 5- or 6-membered heterocycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the heterocyclic group.

In one preferred embodiment R₃ is selected from the group consisting of

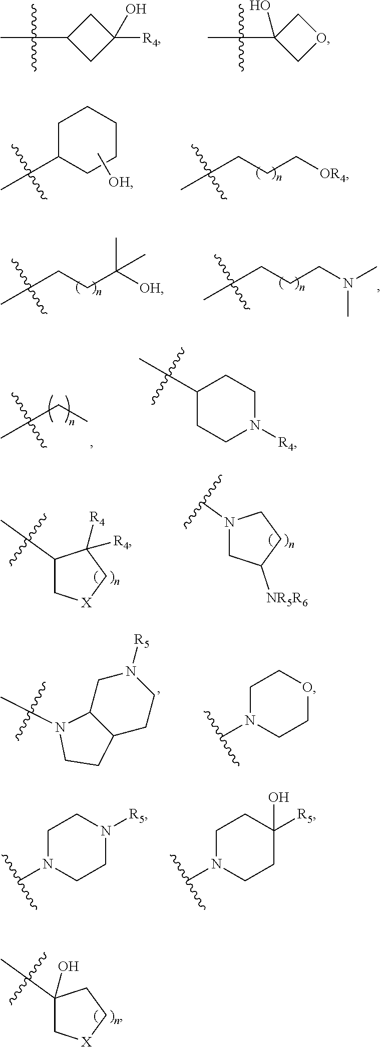

### Me, and CF₃;

wherein
X is selected from O and NR₄;
R₄ is independently selected from H, halo or C₁-C₃alkyl;
R₅ is independently selected from H or C₁-C₃alkyl, preferably H or Me;
R₆ is selected from the group consisting of C₁-C₄alkyl; and
n is selected from 0, 1 or 2.

In another preferred embodiment, R₃ is selected from the group consisting of

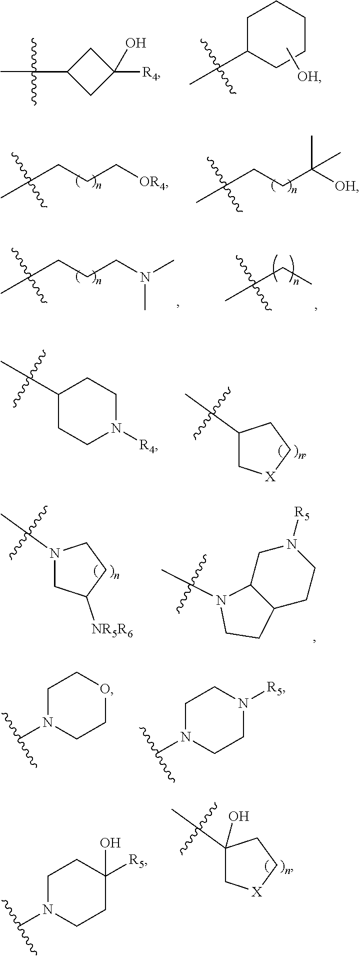

### Me, and CF₃;

wherein
X is selected from O and NR₄;
R₄ is independently selected from H, halo or C₁-C₃alkyl;
R₅ is independently selected from —H or -Me;
R₆ is selected from the group consisting of C₁-C₄alkyl; and
n is selected from 0, 1 or 2.

In one preferred embodiment R₃ is selected from the group consisting of

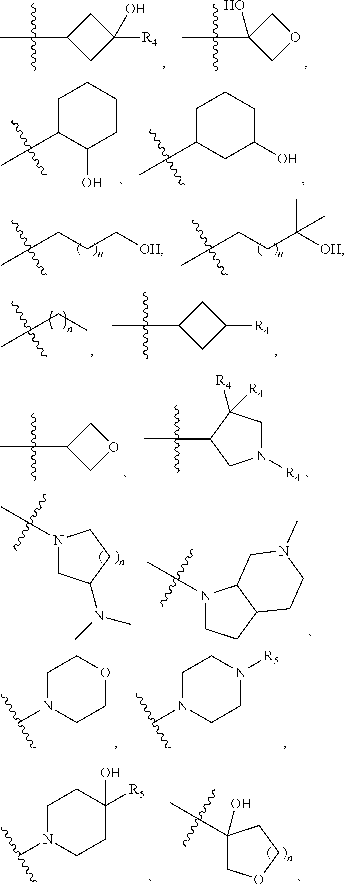

### Me, and CF₃;

wherein
R₄ is independently selected from H, F, or C₁-C₃alkyl; and
R₅ is methyl; and
n is selected from 0, 1 or 2.

In one more preferred embodiment R₃ is selected from the group consisting of

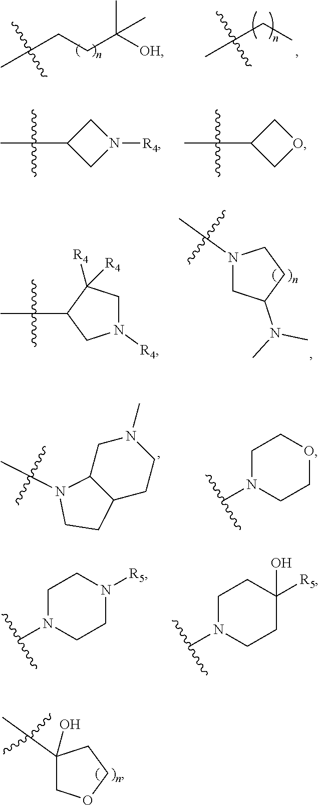

### Me, and CF₃;

wherein R₄ is independently selected from —H, —F, or —C₁-C₃alkyl;
R₅ is methyl; and
n is selected from 0, 1 or 2.

In one preferred embodiment, R₃ is selected from the group consisting of

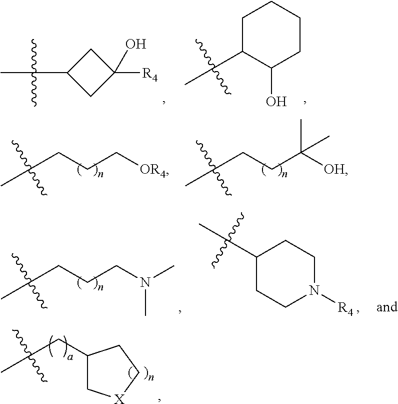

wherein
X is selected from O and NR₄;
R₄ is independently selected from —H or —C₁-C₃alkyl;
a is selected from 0 and 1; and
n is selected from 0, 1 or 2.

In a further preferred embodiment, R₃ is selected from the group consisting of

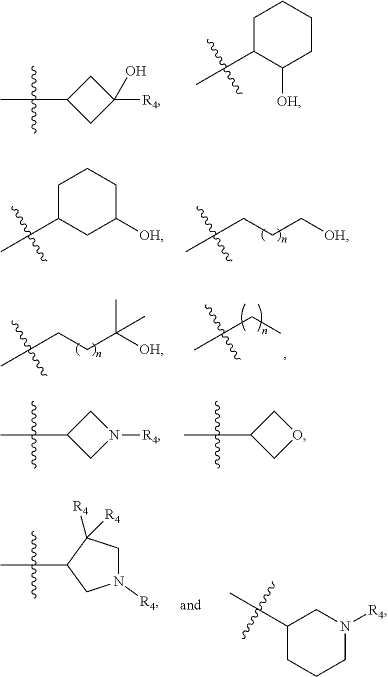

wherein R₄ is independently selected from —H or —C₁-C₃alkyl; and
n is selected from 0, 1 or 2.

In a further preferred embodiment, R₃ is selected from the group consisting of

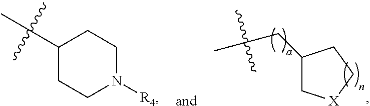

wherein R₄ is independently selected from —H or —C₁-C₃alkyl; and
n is selected from 0, 1 or 2.

In one preferred embodiment R₃ is a 4-, 5- or 6-membered heterocycloalkyl containing one heteroatom, wherein said heteroatom is N. The 4-, 5- or 6-membered heterocycloalkyl can be substituted with one or two substituents independently selected from the group consisting of C₁-C₄alkyl, haloC₁-C₄alkyl, hydroxyC₁-C₄alkyl, —OH and halo (preferably C₁-C₄alkyl). The substituents can be at any available position on the heterocyclic group.

In another embodiment R₃ is a 4-, 5- or 6-membered heterocycloalkyl containing one heteroatom, wherein said heteroatom is O. The 4-, 5- or 6-membered heterocycloalkyl can be substituted with one or two substituents independently selected from the group consisting of C₁-C₄alkyl, haloC₁-C₄alkyl, hydroxyC₁-C₄alkyl, —OH and halo (preferably C₁-C₄alkyl). The substituents can be at any available position on the heterocyclic group.

In another preferred embodiment R₃ is C₁-C₄alkyl.

In one preferred embodiment R₃ is

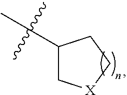

wherein X is selected from O and NR₄, preferably
X is NR₄;
R₄ is independently selected from —H or —C₁-C₃alkyl;
a is selected from 0 and 1 (preferably a is 0); and
n is selected from 0, 1 or 2. In one preferred embodiment n is 2. In one preferred embodiment n is 1. In another preferred embodiment n is 0.

In one preferred embodiment R₃ is

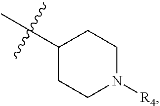

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In another preferred embodiment R₃ is

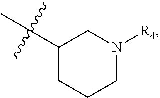

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In another embodiment R₃ is

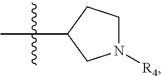

wherein R₄ is independently selected from —H or C₁-C₃alkyl. In a preferred embodiment R₄ is H. In another embodiment R₄ is C₁-C₃alkyl, for instance methyl, ethyl or propyl. In one preferred embodiment R₄ is methyl, whereby R₃ is

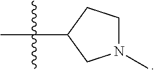 [SMILES: *C1CCN(C)C1]

In another embodiment R₃ is

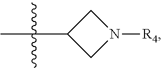

wherein R₄ is independently selected from —H or C₁-C₃alkyl. In a preferred embodiment R₄ is H. In another embodiment R₄ is C₁-C₃alkyl, for instance methyl, ethyl or propyl. In a one embodiment R₄ is methyl, whereby R₃ is

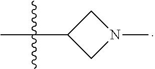 [SMILES: *C1CN(C)C1]

In another embodiment R₃ is

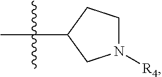

wherein R₄ is independently selected from —H or C₁-C₃alkyl. In a preferred embodiment R₄ is H. In another embodiment R₄ is C₁-C₃alkyl, for instance methyl, ethyl or propyl. In one preferred embodiment R₄ is methyl, whereby R₃ is

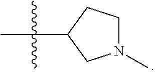 [SMILES: *C1CCN(C)C1]

In another preferred embodiment R₃ is a 4, 5 or 6-membered heterocycloalkyl containing one heteroatom, wherein said heteroatom is O. In one preferred embodiment R₃ is

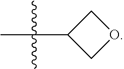 [SMILES: *C1COC1]

In another embodiment R₃ is a C₃-C₆cycloalkyl, preferably cyclohexyl, cyclopropyl or cyclobutyl, more preferably cyclopropyl or cyclobutyl. The C₃-C₆cycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the cycloalkyl group.

In one preferred embodiment R₃ is

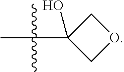 [SMILES: *C1(O)COC1]

In one preferred embodiment R₃ is

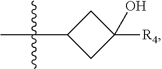

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl. In one preferred embodiment R₃ is

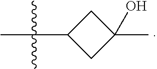 [SMILES: *C1CC(C)(O)C1]

In another preferred embodiment R₃ is

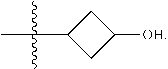 [SMILES: *C1CC(O)C1]

In another preferred embodiment R₃ is

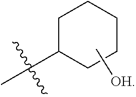

In one preferred embodiment R₃ is

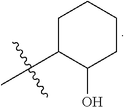 [SMILES: *C1CCCCC1O]

In another preferred embodiment R₃ is

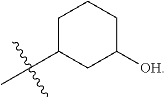 [SMILES: *C1CCCC(O)C1]

In another preferred embodiment R₃ is C₁-C₄alkyl, for example C₁-C₃alkyl, for example methyl, ethyl or propyl. In one preferred embodiment R₃ is methyl. In another preferred embodiment the alkyl can be substituted with one or two substituents independently selected from the group consisting of —OH, halo, haloC₁-C₄alkyl, C₁-C₄alkoxy and —NR₅R₆, wherein R₅ and R₆ are independently selected from H and C₁-C₃alkyl.

In one preferred embodiment R₃ is hydroxyC₁-C₄alkyl (i.e. C₁-C₆alkyl substituted with —OH), more preferably hydroxyC₁-C₃alkyl, preferable hydroxyethyl.

In one preferred embodiment R₃ is

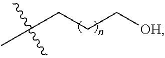

wherein n is 0, 1 or 2. In another preferred embodiment R₃ is

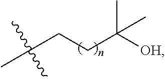

wherein n is 0, 1 or 2.

In another embodiment R₃ is C₁-C₄alkoxyC₁-C₆alkyl (i.e. C₁-C₄alkyl substituted with C₁-C₄alkoxy), preferably C₁-C₃alkoxyC₁-C₄alkyl.

In one embodiment R₃ is

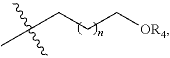

wherein R₄ is —C₁-C₃alkyl; and n is selected from 0, 1 or 2.

In another preferred embodiment, R₃ is C₁-C₄alkyl substituted with —NR₅R₆, wherein R₅ and R₆ are independently selected from H and C₁-C₃alkyl. In a preferred embodiment R₅ and R₆ are both C₁-C₃alkyl, e.g. methyl, ethyl or propyl, more preferably R₅ and R₆ are both methyl. In another embodiment R₅ and R₆ are both H.

In one embodiment R₃ is

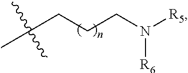

wherein R₅ and R₆ are independently selected from H and C₁-C₃alkyl and n is 0, 1 or 2. In a preferred embodiment R₅ and R₆ are both C₁-C₃alkyl, e.g. methyl, ethyl or propyl, more preferably R₅ and R₆ are both methyl. In another embodiment R₅ and R₆ are both H.

In one preferred embodiment R₃ is

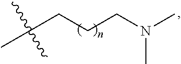

wherein n is 0, 1 or 2.

In another preferred embodiment, R₃ is

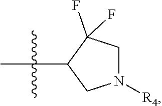

wherein R₄ is independently selected from —H or C₁-C₃alkyl. In a preferred embodiment R₄ is C₁-C₃alkyl, for instance methyl, ethyl or propyl, preferably methyl.

In another preferred embodiment, R₃ is

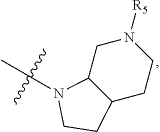

wherein R₅ is H and C₁-C₃alkyl.

In one preferred embodiment Z is C. In another embodiment Z is N.

In one preferred embodiment Q is C. In another embodiment Q is N.

In some embodiments one of Q and Z is C and the other is N. In one embodiment Q is N and Z is C. In another embodiment one of Q is C and Z is N. In one preferred embodiment Q and Z are both C.

In some embodiments E is NRₐ, wherein Rₐ is selected from H and —C₁-C₃alkyl. In one preferred embodiment Rₐ is H. In another preferred embodiment Rₐ is —C₁-C₃alkyl. In a preferred embodiment Rₐ is methyl, In another preferred embodiment Rₐ is ethyl. In another preferred embodiment Rₐ is propyl or isopropyl. In another embodiment E is CRₐ, wherein Rₐ is selected from H and —C₁-C₃alkyl. In one preferred embodiment Rₐ is H. In another preferred embodiment Rₐ is —C₁-C₃alkyl, preferably Rₐ is methyl.

In an embodiment Q and Z are both C and E is CRₐ, wherein Rₐ is as defined above. In one preferred embodiment Q and Z are both C and E is NRₐ, wherein Rₐ is as defined above. In another preferred embodiment Q is N, Z is C and E is CRₐ, wherein Rₐ is as defined above.

In a preferred embodiment X′ is N. In another preferred embodiment X′ is CH.

In a preferred embodiment W is N. In another preferred embodiment W is CH. In another preferred embodiment W is CR_{c} with R_{c} being —CH₂—OH, —O—CH₃ or F.

In one preferred embodiment Y is NH. In another preferred embodiment Y is O. In another embodiment Y is N—CH₃. In another preferred embodiment Y is a bond.

In a preferred embodiment, R_{c} is selected from the group consisting of —O—C₁-C₄alkyl, —C₁-C₄alkyl-OH, and -halo. In a further preferred embodiment, R_{c} is selected from the group consisting of —O—C₁-C₂alkyl, —C₁-C₄alkyl-OH, and —F, such as —C₁-C₄alkyl-OH, —OCH₃, and —F.

In a preferred embodiment, R_{d} is selected from the group consisting of C₁-C₄alkyl.

In one preferred embodiment there is provided a compound having the formula (II′)

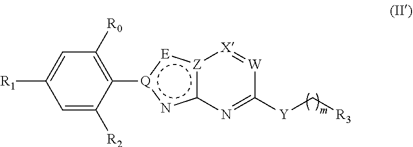

or a stereoisomer, a racemic mixture, a tautomer, a polymorph, a pharmaceutically acceptable salt, a prodrug, a hydrate, or a solvate thereof;
wherein Q, E, Z, X′, W, R₀, R₁, R₂, R₃, m and Y are as defined in any of the above embodiments.

In one preferred embodiment there is provided a compound having the formula (II)

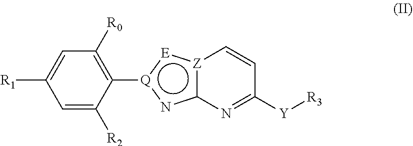

or a stereoisomer, a racemic mixture, a tautomer, a polymorph, a pharmaceutically acceptable salt, a prodrug, a hydrate, or a solvate thereof;
wherein
Q, E, Z, R₀, R₁, R₂, R₃, and Y are as defined in any of the above embodiments.

The following paragraphs apply to formulas (II′) and/or (II).

In a preferred embodiment R₀ is selected from —H and C₁-C₃alkyl, more preferably —H or methyl. In one preferred embodiment R₀ is —H. In another preferred embodiment R₀ is C₁-C₃alkyl, preferably methyl or ethyl, more preferably methyl.

In a preferred embodiment, R₁ is selected from —CF₃, —OCF₃ and —OCHF₂. In one preferred embodiment R₁ is —CF₃. In another preferred embodiment R₁ is —OCF₃. In another preferred embodiment R₁ is —OCHF₂.

In another embodiment R₁ is halo. Where R₁ is halo, the halogen (halo) is preferably chloro.

In one preferred embodiment R₂ is —OH or H, more preferably —OH. In another embodiment R₂ is —H.

In another embodiment R₂ is —CF₃.

In one preferred embodiment R₀ is —H, R₁ is selected from —CF₃, —OCF₃, —OCHF₂ and —Cl, and R₂ is —OH or —H. In one preferred embodiment R₀ is —H, R₁ is —CF₃, and R₂ is —OH.

In one embodiment R₃ is selected from the group consisting of
4-, 5- or 6-membered heterocycloalkyl containing one or two heteroatoms, wherein said heteroatom(s) is/are independently selected from N and/or O, optionally substituted with one or two substituents independently selected from the group consisting of —NR₅R₆, C₁-C₄alkyl or OH;
8-, 9- or 10-membered bicyclic fused heterocycloalkyl containing one to three heteroatoms, wherein said heteroatom(s) is/are independently selected from N and O, optionally substituted at any available position with one or two substituents independently selected from the group consisting of C₁-C₄alkyl, haloC₁-C₄alkyl, hydroxyC₁-C₄alkyl, —OH, —NR₅R₆ and halo; or
C₃-C₆cycloalkyl optionally substituted with one or two substituents independently selected from the group consisting of C₁-C₄alkyl, —OH and halo; or
hydroxyC₁-C₆alkyl; or
haloC₁-C₄alkyl, preferably —CF₃, or
C₁-C₆alkyl optionally substituted with —NR₅R₆,
wherein R₅ and R₆ are independently selected from H and C₁-C₃alkyl; and m is 0 or 1.

In one preferred embodiment R₃ is selected from the group consisting of
4-, 5- or 6-membered heterocycloalkyl containing one heteroatom, wherein said heteroatom is N or O, optionally substituted with one or two substituents independently selected from the group consisting of C₁-C₄alkyl, haloC₁-C₄alkyl, hydroxyC₁-C₄alkyl, —OH and halo, preferably optionally substituted with one or two substituents independently selected from the group consisting of C₁-C₄alkyl;
C₃-C₆cycloalkyl optionally substituted with one or two substituents independently selected from the group consisting of C₁-C₄alkyl, —OH and halo;
hydroxyC₁-C₆alkyl; and
C₁-C₆alkyl optionally substituted with —NR₅R₆, wherein R₅ and R₆ are independently selected from H and C₁-C₃alkyl.

In one preferred embodiment R₃ is selected from a 4-, 5- or 6-membered heterocycloalkyl containing one or two heteroatoms, preferably one heteroatom, wherein said heteroatom(s) is/are N or O. The 4-, 5- or 6-membered heterocycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the heterocyclic group.

In one preferred embodiment R₃ is selected from the group consisting of

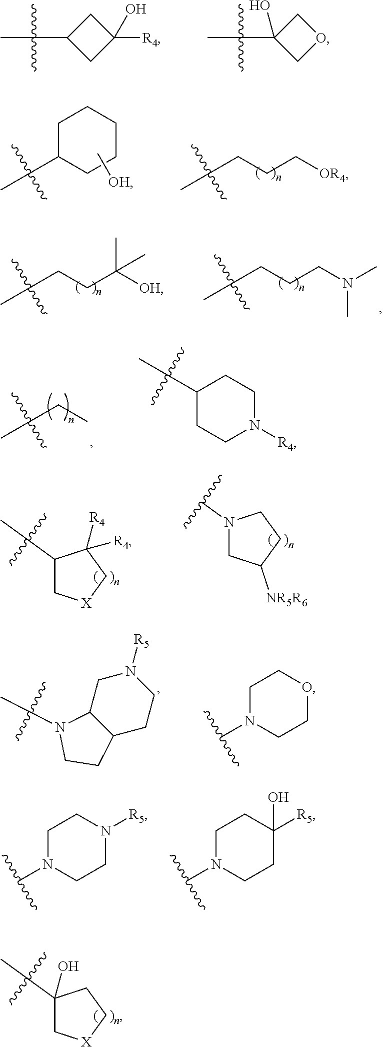

### Me, and CF₃;

wherein
X is selected from O and NR₄;
R₄ is independently selected from H, halo or C₁-C₃alkyl;
R₅ is independently selected from H or C₁-C₃alkyl, preferably H or Me;
R₆ is selected from the group consisting of C₁-C₄alkyl; and
n is selected from 0, 1 or 2.

In one preferred embodiment, R₃ is selected from the group consisting of

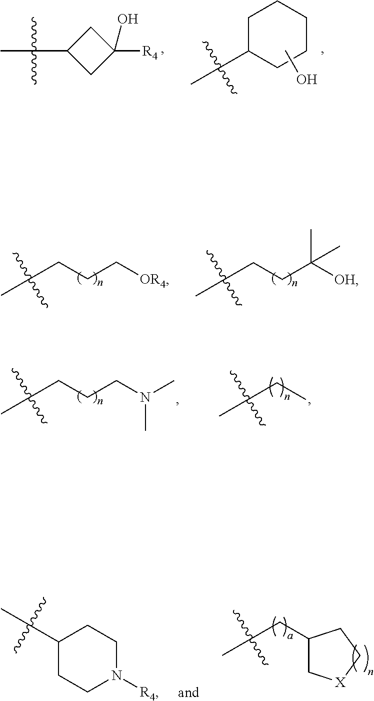

wherein X is selected from O and NR₄;
R₄ is independently selected from —H or —C₁-C₃alkyl;
n is selected from 0, 1 or 2; and a is selected from 0 or 1.

In one preferred embodiment R₃ is selected from the group consisting of

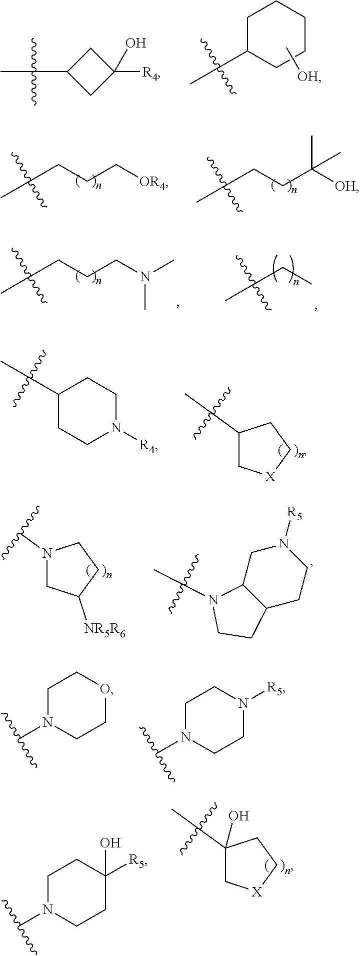

### Me, and CF₃;

wherein
X is selected from O and NR₄;
R₄ is independently selected from H, halo or C₁-C₃alkyl;
R₅ is independently selected from —H or -Me;
R₆ is selected from the group consisting of C₁-C₄alkyl; and
n is selected from 0, 1 or 2.

In one more preferred embodiment R₃ is selected from the group consisting of

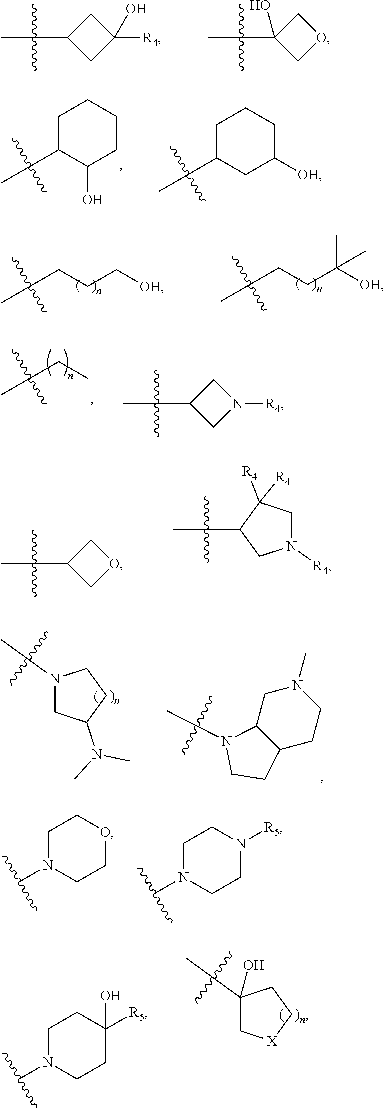

### Me, and CF₃;

wherein
R₄ is independently selected from H, F, or C₁-C₃alkyl; and
R₅ is methyl; and
n is selected from 0, 1 or 2.

In one preferred embodiment, R₃ is selected from the group consisting of

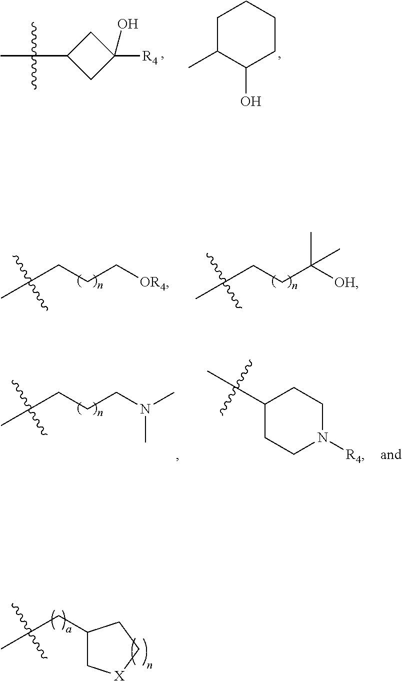

wherein X is selected from O and NR₄;
R₄ is independently selected from —H or —C₁-C₃alkyl;
a is selected from 0 and 1; and
n is selected from 0, 1 or 2.

In a further preferred embodiment, R₃ is selected from the group consisting of

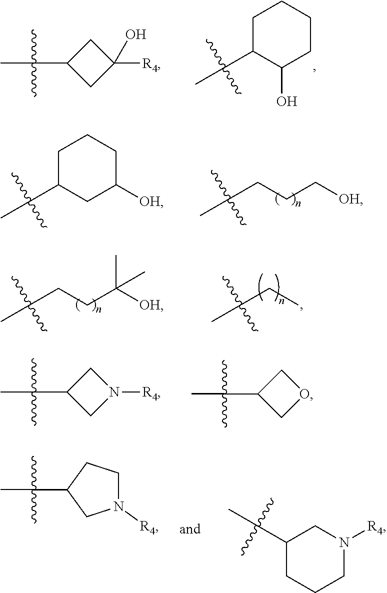

wherein R₄ is independently selected from —H or —C₁-C₃alkyl; and
n is selected from 0, 1 or 2.

In a further preferred embodiment, R₃ is selected from the group consisting of

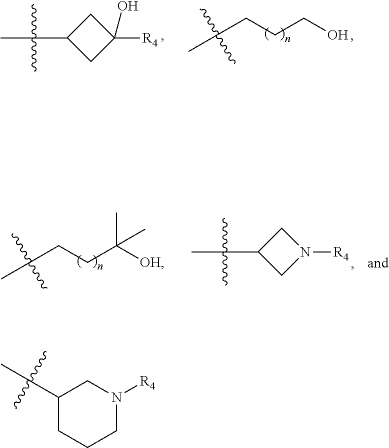

wherein R₄ is independently selected from —H or —C₁-C₃alkyl; and
n is selected from 0, 1 or 2.

In one preferred embodiment R₃ is a 4-, 5- or 6-membered heterocycloalkyl containing one heteroatom, wherein said heteroatom is N. The 4-, 5- or 6-membered heterocycloalkyl can be substituted with one or two substituents independently selected from the group consisting of C₁-C₄alkyl, haloC₁-C₄alkyl, hydroxyC₁-C₄alkyl, —OH and halo (preferably C₁-C₄alkyl). The substituents can be at any available position on the heterocyclic group.

In another embodiment R₃ is a 4-, 5- or 6-membered heterocycloalkyl containing one heteroatom, wherein said heteroatom is O. The 4-, 5- or 6-membered heterocycloalkyl can be substituted with one or two substituents independently selected from the group consisting of C₁-C₄alkyl, haloC₁-C₄alkyl, hydroxyC₁-C₄alkyl, —OH and halo (preferably C₁-C₄alkyl). The substituents can be at any available position on the heterocyclic group.

In one preferred embodiment R₃ is

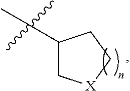

wherein X is selected from O and NR₄, preferably X is NR₄;
R₄ is independently selected from —H or —C₁-C₃alkyl;
a is selected from 0 and 1 (preferably a is 0); and
n is selected from 0, 1 or 2. In one preferred embodiment n is 2. In one preferred embodiment n is 1. In another preferred embodiment n is 0.

In one preferred embodiment R₃ is

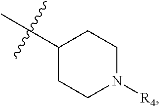

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In another preferred embodiment R₃ is

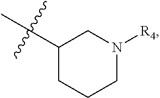

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In another embodiment R₃ is

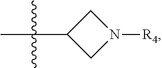

wherein R₄ is independently selected from —H or C₁-C₃alkyl. In a preferred embodiment R₄ is H. In another embodiment R₄ is C₁-C₃alkyl, for instance methyl, ethyl or propyl. In a one embodiment R₄ is methyl, whereby R₃ is

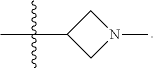 [SMILES: *C1CN(C)C1]

In another embodiment R₃ is

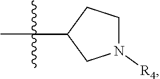

wherein R₄ is independently selected from —H or C₁-C₃alkyl. In a preferred embodiment R₄ is H. In another embodiment R₄ is C₁-C₃alkyl, for instance methyl, ethyl or propyl. In one preferred embodiment R₄ is methyl, whereby R₃ is

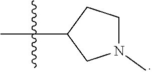 [SMILES: *C1CCN(C)C1]

In another preferred embodiment R₃ is a 4, 5 or 6-membered heterocycloalkyl containing one heteroatom, wherein said heteroatom is O. In one preferred embodiment R₃ is

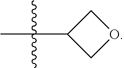 [SMILES: *C1COC1]

In another embodiment R₃ is a C₃-C₆cycloalkyl, preferably cyclohexyl, cyclopropyl or cyclobutyl, more preferably cyclopropyl or cyclobutyl. The C₃-C₆cycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the cycloalkyl group.

In one preferred embodiment R₃ is

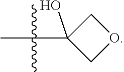 [SMILES: *C1(O)COC1]

In one preferred embodiment R₃ is

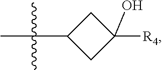

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl. In one preferred embodiment R₃ is

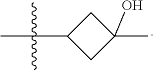 [SMILES: *C1CC(C)(O)C1]

In another preferred embodiment R₃ is

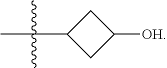 [SMILES: *C1CC(O)C1]

In another preferred embodiment R₃ is

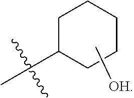

In one preferred embodiment R₃ is

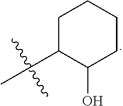 [SMILES: *C1CCCCC1O]

In another preferred embodiment R₃ is

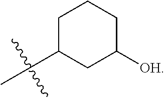 [SMILES: *C1CCCC(O)C1]

In another preferred embodiment R₃ is C₁-C₆alkyl, for example C₁-C₃alkyl, for example methyl, ethyl or propyl. In one preferred embodiment R₃ is methyl. In another preferred embodiment the alkyl can be substituted with one or two substituents independently selected from the group consisting of —OH, halo, haloC₁-C₄alkyl, C₁-C₄alkoxy and —NR₅R₆, wherein R₅ and R₆ are independently selected from H and C₁-C₃alkyl. In one preferred embodiment R₃ is hydroxyC₁-C₆alkyl (i.e. C₁-C₄alkyl substituted with —OH), more preferably hydroxyC₁-C₃alkyl, preferable hydroxyethyl.

In one preferred embodiment R₃ is

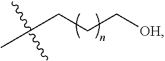

wherein n is 0, 1 or 2. In another preferred embodiment R₃ is

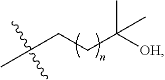

wherein n is 0, 1 or 2.

In another embodiment R₃ is C₁-C₄alkoxyC₁-C₆alkyl (i.e. C₁-C₆alkyl substituted with C₁-C₄alkoxy), preferably C₁-C₃alkoxyC₁-C₄alkyl.

In one embodiment R₃ is

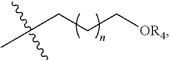

wherein R₄ is —C₁-C₃alkyl; and n is selected from 0, 1 or 2.

In another preferred embodiment, R₃ is C₁-C₄alkyl substituted with —NR₅R₆, wherein R₅ and R₆ are independently selected from H and C₁-C₃alkyl. In a preferred embodiment R₅ and R₆ are both C₁-C₃alkyl, e.g. methyl, ethyl or propyl, more preferably R₅ and R₆ are both methyl. In another embodiment R₅ and R₆ are both H.

In one embodiment R₃ is

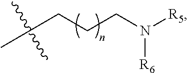

wherein R₅ and R₆ are independently selected from H and C₁-C₃alkyl and n is 0, 1 or 2. In a preferred embodiment R₅ and R₆ are both C₁-C₃alkyl, e.g. methyl, ethyl or propyl, more preferably R₅ and R₆ are both methyl. In another embodiment R₅ and R₆ are both H.

In one preferred embodiment R₃ is

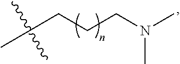

wherein n is 0, 1 or 2.

In another preferred embodiment, R₃ is

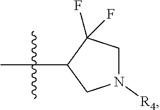

wherein R₄ is independently selected from —H or C₁-C₃alkyl. In a preferred embodiment R₄ is C₁-C₃alkyl, for instance methyl, ethyl or propyl, preferably methyl.

In another preferred embodiment, R₃ is

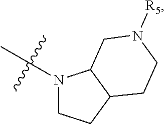

wherein R₅ is H and C₁-C₃alkyl.

In another preferred embodiment R₃ is C₁-C₆alkyl.

In one preferred embodiment Z is C. In another embodiment Z is N. In one preferred embodiment Q is C. In another embodiment Q is N.

In some embodiments one of Q and Z is C and the other is N. In one embodiment Q is N and Z is C. In another embodiment one of Q is C and Z is N. In one preferred embodiment Q and Z are both C.

In some embodiments E is NRₐ, wherein Rₐ is selected from H and —C₁-C₃alkyl. In one preferred embodiment Rₐ is H. In another preferred embodiment Rₐ is —C₁-C₃alkyl, preferably Rₐ is methyl. In other embodiment E is CRₐ, wherein Rₐ is selected from H and —C₁-C₃alkyl. In one preferred embodiment Rₐ is H. In another preferred embodiment Rₐ is —C₁-C₃alkyl, preferably Rₐ is methyl.

In one preferred embodiment Q and Z are both C and E is NRₐ, wherein Rₐ is as defined above. In another embodiment Q and Z are both C and E is CRₐ, wherein Rₐ is as defined above. In another preferred embodiment Q is N, Z is C and E is CR_{c}, wherein Rₐ is as defined above.

In formula (II′), in a preferred embodiment X′ is N. In another preferred embodiment X′ is CH.

In a preferred embodiment W is N. In another preferred embodiment W is CH. In another preferred embodiment W is CR_{c} with R_{c} being —CH₂—OH, —O—CH₃ or F.

In one preferred embodiment Y is NH. In another preferred embodiment Y is O. In another embodiment Y is N—CH₃. In another preferred embodiment Y is a bond.

In a preferred embodiment, R_{c} is selected from the group consisting of —O—C₁-C₄alkyl, C₁-C₄alkyl-OH, and halo. In a further preferred embodiment, R_{c} is selected from the group consisting of —O—C₁-C₂alkyl, C₁-C₄alkyl-OH, and F, such as C₁-C₄alkyl-OH, OCH₃, and F.

In a preferred embodiment, R_{d} is selected from the group consisting of C₁-C₄alkyl.

In another embodiment there is provided a compound having the formula (III′)

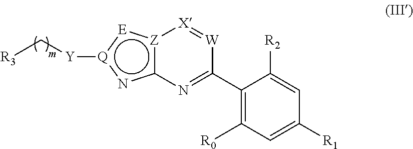

or a stereoisomer, a racemic mixture, a tautomer, a polymorph, a pharmaceutically acceptable salt, a prodrug, a hydrate, or a solvate thereof;
wherein Q, E, Z, X′, W, R₀, R₁, R₂, R₃, m and Y are as defined in any of the above embodiments.

In another embodiment there is provided a compound having the formula (III)

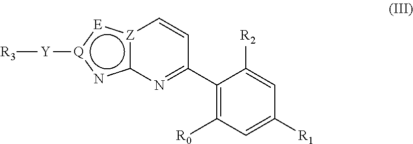

or a stereoisomer, a racemic mixture, a tautomer, a polymorph, a pharmaceutically acceptable salt, a prodrug, a hydrate, or a solvate thereof;
wherein Q, E, Z, R₀, R₁, R₂, R₃, and Y are as defined in any of the above embodiments.

The following paragraphs apply to formulas (III′) and/or (III).

In a preferred embodiment R₀ is selected from —H and C₁-C₃alkyl, more preferably —H or methyl. In one preferred embodiment R₀ is —H. In another preferred embodiment R₀ is C₁-C₃alkyl, preferably methyl or ethyl, more preferably methyl.

In a preferred embodiment, R₁ is selected from —CF₃, —OCF₃ and —OCHF₂. In one preferred embodiment R₁ is —CF₃. In another preferred embodiment R₁ is —OCF₃. In another preferred embodiment R₁ is —OCHF₂.

In another embodiment R₁ is halo Where R₁ is halo, the halogen (halo) is preferably chloro.

In one preferred embodiment R₂ is —OH or H, more preferably —OH. In another embodiment R₂ is —H.

In another embodiment R₂ is —CF₃.

In one preferred embodiment R₀ is —H, R₁ is selected from —CF₃, —OCF₃, —OCHF₂ and —Cl, and R₂ is —OH or —H. In one preferred embodiment R₀ is —H, R₁ is —CF₃, and R₂ is —OH.

In one embodiment R₃ is selected from the group consisting of
4-, 5- or 6-membered heterocycloalkyl containing one or two heteroatoms, wherein said heteroatom(s) is/are independently selected from N and/or O, optionally substituted with one or two substituents independently selected from the group consisting of —NR₅R₆, C₁-C₄alkyl or OH; or
8-, 9- or 10-membered bicyclic fused heterocycloalkyl containing one to three heteroatoms, wherein said heteroatom(s) is/are independently selected from N and O, optionally substituted at any available position with one or two substituents independently selected from the group consisting of C₁-C₄alkyl, haloC₁-C₄alkyl, hydroxyC₁-C₄alkyl, —OH, —NR₅R₆ and halo; or
C₃-C₆cycloalkyl optionally substituted with one or two substituents independently selected from the group consisting of C₁-C₄alkyl, —OH and halo; or
hydroxyC₁-C₆alkyl; or
haloC₁-C₄alkyl, preferably —CF₃; or
C₁-C₆alkyl optionally substituted with —NR₅R₆,
wherein R₅ and R₆ are independently selected from H and C₁-C₃alkyl; and
m is 0 or 1.

In one preferred embodiment R₃ is selected from the group consisting of
4-, 5- or 6-membered heterocycloalkyl containing one heteroatom, wherein said heteroatom is N or O, optionally substituted with one or two substituents independently selected from the group consisting of C₁-C₄alkyl, haloC₁-C₄alkyl, hydroxyC₁-C₄alkyl, —OH and halo, preferably optionally substituted with one or two substituents independently selected from the group consisting of C₁-C₄alkyl;
C₃-C₆cycloalkyl optionally substituted with one or two substituents independently selected from the group consisting of C₁-C₄alkyl, —OH and halo;
hydroxyC₁-C₄alkyl; and
C₁-C₆alkyl optionally substituted with —NR₅R₆, wherein R₅ and R₆ are independently selected from H and C₁-C₃alkyl.

In one preferred embodiment R₃ is selected from a 4-, 5- or 6-membered heterocycloalkyl containing one or two heteroatoms, preferably one heteroatom, wherein said heteroatom(s) is/are N or O. The 4-, 5- or 6-membered heterocycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the heterocyclic group.

In one preferred embodiment R₃ is selected from the group consisting of

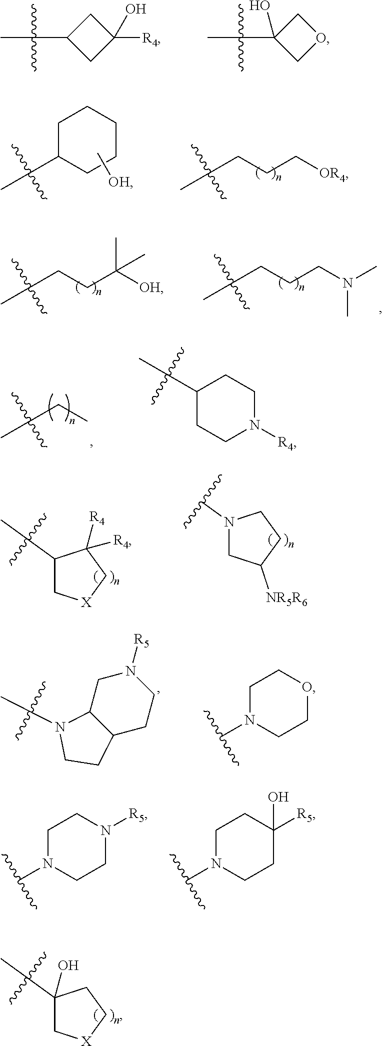

### Me, and CF₃;

wherein
X is selected from O and NR₄;
R₄ is independently selected from H, halo or C₁-C₃alkyl;
R₅ is independently selected from H or C₁-C₃alkyl, preferably H or Me;
R₆ is selected from the group consisting of C₁-C₄alkyl; and
n is selected from 0, 1 or 2.

In one preferred embodiment, R₃ is selected from the group consisting of

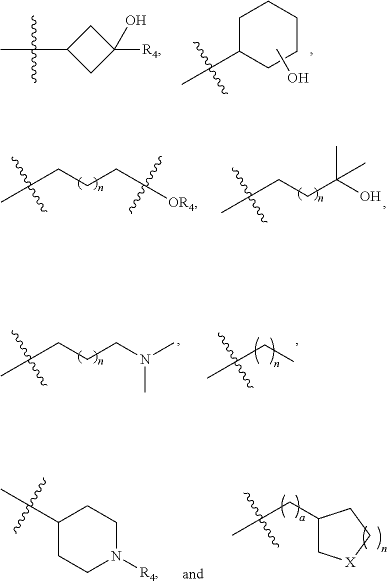

wherein X is selected from O and NR₄;
R₄ is independently selected from —H or —C₁-C₃alkyl;
a is selected from 0 and 1; and
n is selected from 0, 1 or 2.

In one preferred embodiment R₃ is selected from the group consisting of

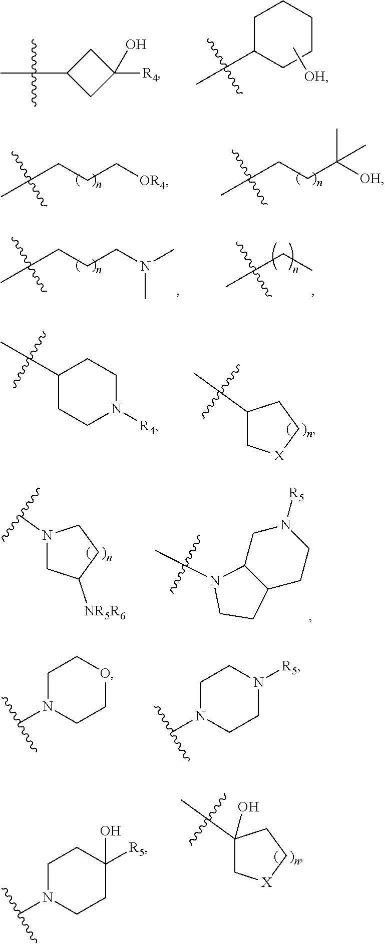

### Me, and CF₃;

wherein
X is selected from O and NR₄;
R₄ is independently selected from H, halo or C₁-C₃alkyl;
R₅ is independently selected from —H or -Me;
R₆ is selected from the group consisting of C₁-C₄alkyl; and
n is selected from 0, 1 or 2.

In one preferred embodiment, R₃ is selected from the group consisting of

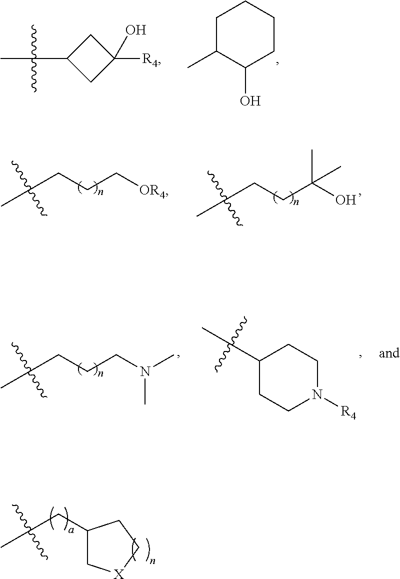

wherein X is selected from O and NR₄;
R₄ is independently selected from —H or —C₁-C₃alkyl;
a is selected from 0 and 1; and
n is selected from 0, 1 or 2.

In a furthermore preferred embodiment, R₃ is selected from the group consisting of

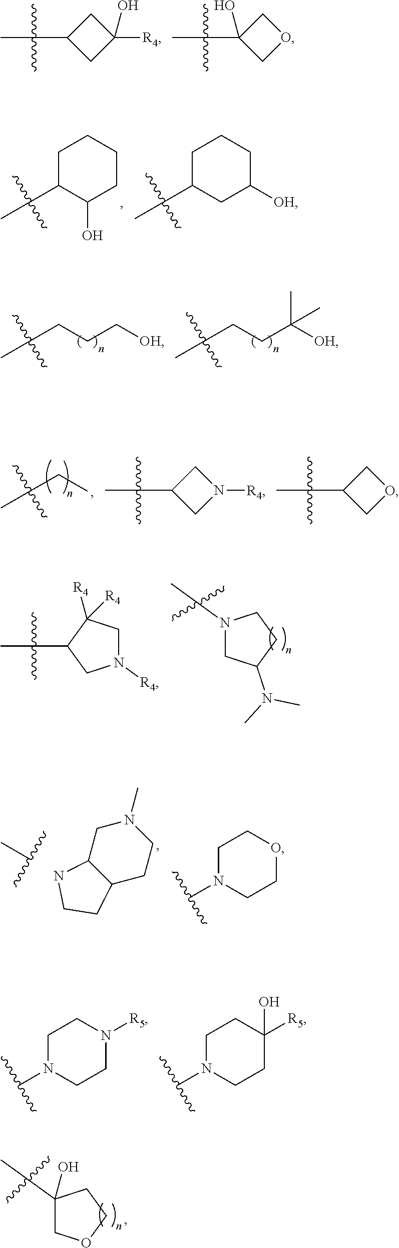

### Me, and CF₃;

wherein
R₄ is independently selected from H, F, or C₁-C₃alkyl; and
R₅ is methyl; and
n is selected from 0, 1 or 2.

In a further preferred embodiment, R₃ is selected from the group consisting of

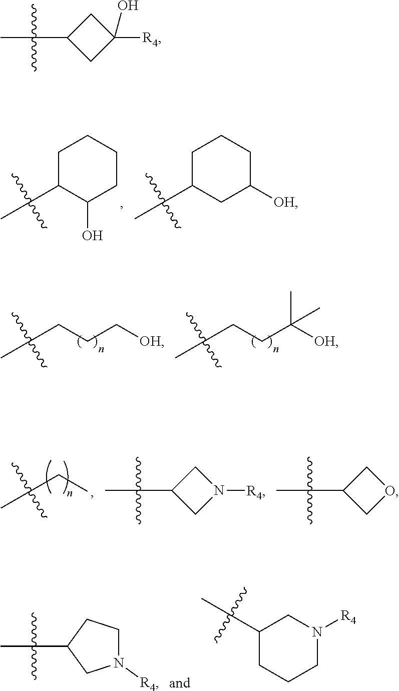

wherein R₄ is independently selected from —H or —C₁-C₃alkyl; and
n is selected from 0, 1 or 2.

In a further embodiment, R₃ is selected from the group consisting of

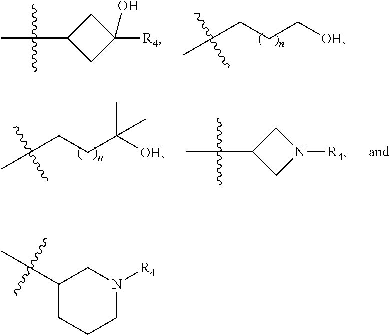

wherein R₄ is independently selected from —H or —C₁-C₃alkyl; and
n is selected from 0, 1 or 2.

In one preferred embodiment R₃ is a 4-, 5- or 6-membered heterocycloalkyl containing one heteroatom, wherein said heteroatom is N. The 4-, 5- or 6-membered heterocycloalkyl can be substituted with one or two substituents independently selected from the group consisting of C₁-C₄alkyl, haloC₁-C₄alkyl, hydroxyC₁-C₄alkyl, —OH and halo (preferably C₁-C₄alkyl). The substituents can be at any available position on the heterocyclic group.

In one preferred embodiment R₃ is

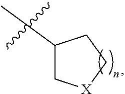

wherein X is selected from O and NR₄, preferably
X is NR₄;
R₄ is independently selected from —H or —C₁-C₃alkyl;
a is selected from 0 and 1 (preferably a is 0); and
n is selected from 0, 1 or 2. In one preferred embodiment n is 2. In one preferred embodiment n is 1.

In another preferred embodiment n is 0.

In one preferred embodiment R₃ is

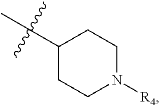

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In another preferred embodiment R₃ is

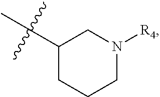

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In another embodiment R₃ is

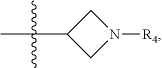

wherein R₄ is independently selected from —H or C₁-C₃alkyl. In a preferred embodiment R₄ is H. In another embodiment R₄ is C₁-C₃alkyl, for instance methyl, ethyl or propyl. In a one embodiment R₄ is methyl, whereby R₃ is

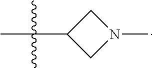 [SMILES: *C1CN(C)C1]

In another embodiment R₃ is

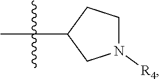

wherein R₄ is independently selected from —H or C₁-C₃alkyl. In a preferred embodiment R₄ is H. In another embodiment R₄ is C₁-C₃alkyl, for instance methyl, ethyl or propyl. In one preferred embodiment R₄ is methyl, whereby R₃ is

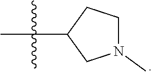 [SMILES: *C1CCN(C)C1]

In another preferred embodiment R₃ is a 4, 5 or 6-membered heterocycloalkyl containing one heteroatom, wherein said heteroatom is O. In one preferred embodiment R₃ is

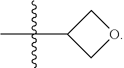 [SMILES: *C1COC1]

In another embodiment R₃ is a C₃-C₆cycloalkyl, preferably cyclohexyl, cyclopropyl or cyclobutyl, more preferably cyclopropyl or cyclobutyl. The C₃-C₆cycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the cycloalkyl group.

In one preferred embodiment R₃ is

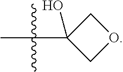 [SMILES: *C1(O)COC1]

In one preferred embodiment R₃ is

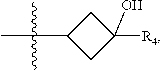

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl. In one preferred embodiment R₃ is

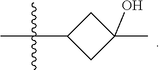 [SMILES: *C1CC(C)(O)C1]

In another preferred embodiment R₃ is

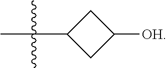 [SMILES: *C1CC(O)C1]

In another preferred embodiment R₃ is

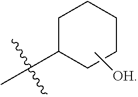

In one preferred embodiment R₃ is

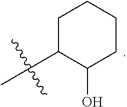 [SMILES: *C1CCCCC1O]

In another preferred embodiment R₃ is

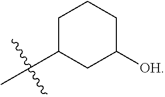 [SMILES: *C1CCCC(O)C1]

In another preferred embodiment R₃ is C₁-C₄alkyl, for example C₁-C₃alkyl, for example methyl, ethyl or propyl. In one preferred embodiment R₃ is methyl. In another preferred embodiment the alkyl can be substituted with one or two substituents independently selected from the group consisting of —OH, halo, haloC₁-C₄alkyl, C₁-C₄alkoxy and —NR₅R₆, wherein R₅ and R₆ are independently selected from H and C₁-C₃alkyl. In one preferred embodiment R₃ is hydroxyC₁-C₄alkyl (i.e. C₁-C₄alkyl substituted with —OH), more preferably hydroxyC₁-C₃alkyl, preferable hydroxyethyl.

In one preferred embodiment R₃ is

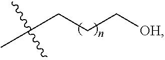

wherein n is 0, 1 or 2. In another preferred embodiment R₃ is

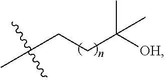

wherein n is 0, 1 or 2.

In another embodiment R₃ is C₁-C₄alkoxyC₁-C₆alkyl (i.e. C₁-C₆alkyl substituted with C₁-C₄alkoxy), preferably C₁-C₃alkoxyC₁-C₄alkyl.

In one embodiment R₃ is

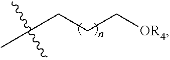

wherein R₄ is —C₁-C₃alkyl; and n is selected from 0, 1 or 2.

In another preferred embodiment, R₃ is C₁-C₆alkyl substituted with —NR₅R₆, wherein R₅ and R₆ are independently selected from H and C₁-C₃alkyl. In a preferred embodiment R₅ and R₆ are both C₁-C₃alkyl, e.g. methyl, ethyl or propyl, more preferably R₅ and R₆ are both methyl. In another embodiment R₅ and R₆ are both H.

In one embodiment R₃ is

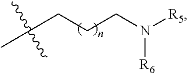

wherein R₅ and R₆ are independently selected from H and C₁-C₃alkyl and wherein n is 0, 1 or 2. In a preferred embodiment R₅ and R₆ are both C₁-C₃alkyl, e.g. methyl, ethyl or propyl, more preferably R₅ and R₆ are both methyl. In another embodiment R₅ and R₆ are both H.

In one preferred embodiment R₃ is

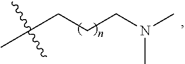

wherein n is 0, 1 or 2.

In one preferred embodiment Z is C. In another embodiment Z is N. In one preferred embodiment Q is C. In another embodiment Q is N.

In some embodiments one of Q and Z is C and the other is N. In one embodiment Q is N and Z is C. In another embodiment one of Q is C and Z is N.

In one preferred embodiment Q and Z are both C.

In some embodiments E is NRₐ, wherein Rₐ is selected from H and —C₁-C₃alkyl. In one preferred embodiment Rₐ is H. In another preferred embodiment Rₐ is —C₁-C₃alkyl, preferably Rₐ is methyl. In other embodiment E is CRₐ, wherein Rₐ is selected from H and —C₁-C₃alkyl. In one preferred embodiment Rₐ is H. In another preferred embodiment Rₐ is —C₁-C₃alkyl, preferably Rₐ is methyl.

In one preferred embodiment Q and Z are both C and E is NRₐ, wherein Rₐ is as defined above. In another embodiment Q and Z are both C and E is CRₐ, wherein Rₐ is as defined above. In another preferred embodiment Q is N, Z is C and E is CRₐ, wherein Rₐ is as defined above.

In a preferred embodiment X′ is N. In another preferred embodiment X′ is CH.

In a preferred embodiment W is N. In another preferred embodiment W is CH. In another preferred embodiment W is CR_{c} with R_{c} being —CH₂—OH, —O—CH₃ or F.

In one preferred embodiment Y is NH. In another preferred embodiment Y is O. In another embodiment Y is N—CH₃. In another preferred embodiment Y is a bond.

In a preferred embodiment, R_{c} is selected from the group consisting of —O—C₁-C₄alkyl, C₁-C₄alkyl-OH, and halo. In a further preferred embodiment, R_{c} is selected from the group consisting of —O—C₁-C₂alkyl, C₁-C₄alkyl-OH, and F, such as C₁-C₄alkyl-OH, OCH₃, and F.

In a preferred embodiment, R_{d} is selected from the group consisting of C₁-C₄alkyl.

In another preferred embodiment, R₃ is

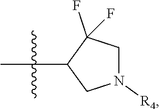

wherein R₄ is independently selected from —H or C₁-C₃alkyl. In a preferred embodiment R₄ is C₁-C₃alkyl, for instance methyl, ethyl or propyl, preferably methyl.

In another preferred embodiment, R₃ is

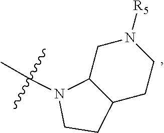

wherein R₅ is H and C₁-C₃alkyl.

In an embodiment R₃ is OH.

In another preferred embodiment R₃ is C₁-C₆alkyl.

In another preferred embodiment there is provided a compound of formula (II′), having the formula (II′a)

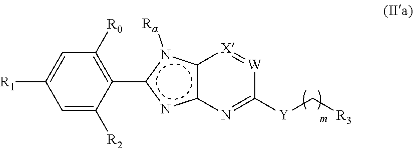

or a stereoisomer, a racemic mixture, a tautomer, a polymorph, a pharmaceutically acceptable salt, a prodrug, a hydrate, or a solvate thereof;
wherein X′, W, R₀, R₁, R₂, R₃, Rₐ, m and Y are as defined in any of the above embodiments.

In another preferred embodiment there is provided a compound of formula (II), having the formula (IIa)

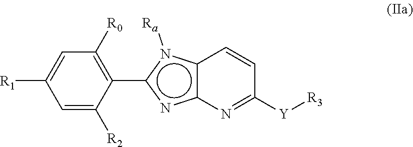

or a stereoisomer, a racemic mixture, a tautomer, a polymorph, a pharmaceutically acceptable salt, a prodrug, a hydrate, or a solvate thereof;
wherein R₀, R₁, R₂, R₃, Rₐ and Y are as defined in any of the above embodiments.

The following paragraphs apply to formulas (IIa′) and/or (IIa).

In a preferred embodiment R₀ is selected from —H and C₁-C₃alkyl, more preferably —H or methyl. In one preferred embodiment R₀ is —H. In another preferred embodiment R₀ is C₁-C₃alkyl, preferably or ethyl, more preferably methyl.

In a preferred embodiment, R₁ is selected from —CF₃, —OCF₃ and —OCHF₂. In one preferred embodiment R₁ is —CF₃. In another preferred embodiment R₁ is —OCF₃. In another preferred embodiment R₁ is —OCHF₂.

In another embodiment R₁ is halo. Where R₁ is halo, the halogen (halo) is preferably chloro.

In one preferred embodiment R₂ is —OH or H, more preferably —OH. In another embodiment R₂ is —H. In another embodiment R₂ is —CF₃.

In one preferred embodiment R₀ is —H, R₁ is selected from —CF₃, —OCF₃, —OCHF₂ and —Cl, and R₂ is —OH or —H. In one preferred embodiment R₀ is —H, R₁ is —CF₃, and R₂ is —OH.

In one embodiment R₃ is selected from the group consisting of
4-, 5- or 6-membered heterocycloalkyl containing one or two heteroatoms, wherein said heteroatom(s) is/are independently selected from N and/or O, optionally substituted with one or two substituents independently selected from the group consisting of —NR₅R₆, C₁-C₄alkyl or OH;
8-, 9- or 10-membered bicyclic fused heterocycloalkyl containing one to three heteroatoms, wherein said heteroatom(s) is/are independently selected from N and O, optionally substituted at any available position with one or two substituents independently selected from the group consisting of C₁-C₄alkyl, haloC₁-C₄alkyl, hydroxyC₁-C₄alkyl, —OH, —NR₅R₆ and halo; or
C₃-C₆cycloalkyl optionally substituted with one or two substituents independently selected from the group consisting of C₁-C₄alkyl, —OH and halo; or hydroxyC₁-C₆alkyl; or
haloC₁-C₄alkyl, preferably —CF₃; or
C₁-C₆alkyl optionally substituted with —NR₅R₆, wherein R₅ and R₆ are independently selected from H and C₁-C₃alkyl; and
m is 0 or 1.

In another embodiment R₃ is selected from the group consisting of
4-, 5- or 6-membered heterocycloalkyl containing one heteroatom, wherein said heteroatom is N or O, optionally substituted with one or two substituents independently selected from the group consisting of C₁-C₄alkyl, haloC₁-C₄alkyl, hydroxyC₁-C₄alkyl, —OH and halo, preferably optionally substituted with one or two substituents independently selected from the group consisting of C₁-C₄alkyl;
C₃-C₆cycloalkyl optionally substituted with one or two substituents independently selected from the group consisting of C₁-C₄alkyl, —OH and halo;
hydroxyC₁-C₆alkyl; and
C₁-C₆alkyl optionally substituted with —NR₅R₆, wherein R₅ and R₆ are independently selected from H and C₁-C₃alkyl.

In one preferred embodiment R₃ is selected from a 4-, 5- or 6-membered heterocycloalkyl containing one or two heteroatoms, preferably one heteroatom, wherein said heteroatom(s) is/are N or O. The 4-, 5- or 6-membered heterocycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the heterocyclic group.

In one preferred embodiment R₃ is selected from the group consisting of

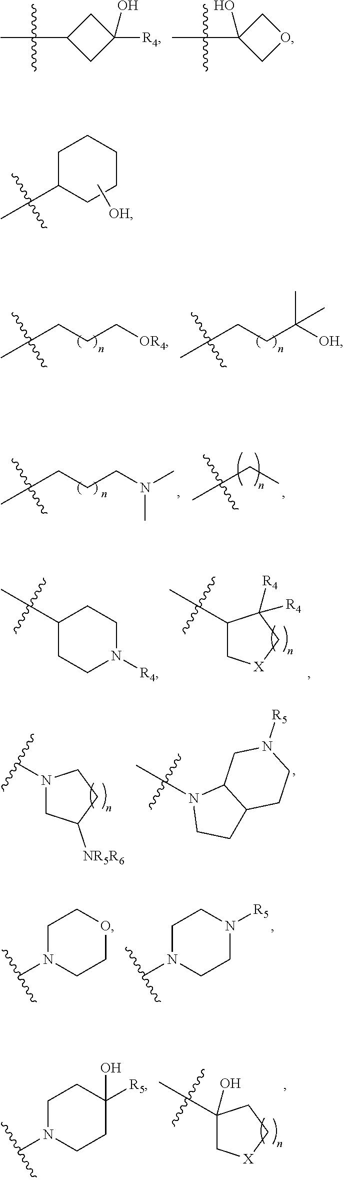

### Me, and CF₃;

wherein
X is selected from O and NR₄;
R₄ is independently selected from H, halo or C₁-C₃alkyl;
R₅ is independently selected from H or C₁-C₃alkyl, preferably H or Me;
R₆ is selected from the group consisting of C₁-C₄alkyl; and
n is selected from 0, 1 or 2.

In one preferred embodiment, R₃ is selected from the group consisting of

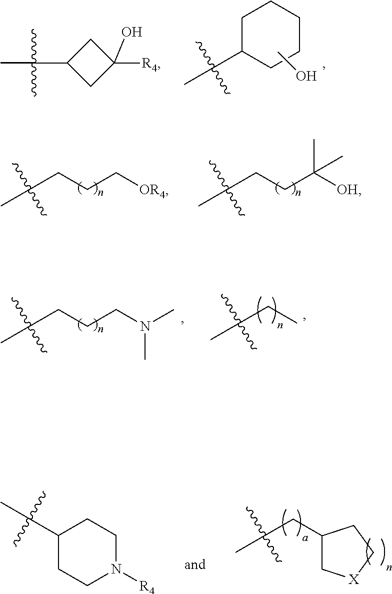

wherein X is selected from O and NR₄;
R₄ is independently selected from —H or —C₁-C₃alkyl;
a is selected from 0 and 1; and
n is selected from 0, 1 or 2.

In one preferred embodiment R₃ is selected from the group consisting of

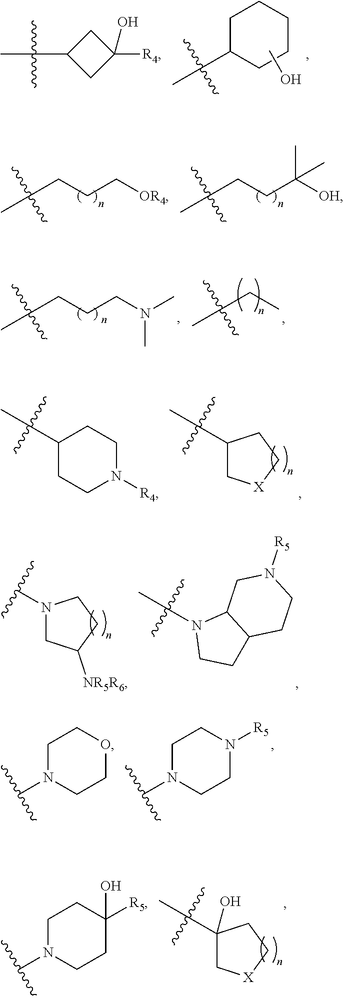

### Me, and CF₃;

wherein
X is selected from O and NR₄;
R₄ is independently selected from H, halo or C₁-C₃alkyl;
R₅ is independently selected from —H or -Me;
R₆ is selected from the group consisting of C₁-C₄alkyl; and
n is selected from 0, 1 or 2.

In one preferred embodiment, R₃ is selected from the group consisting of

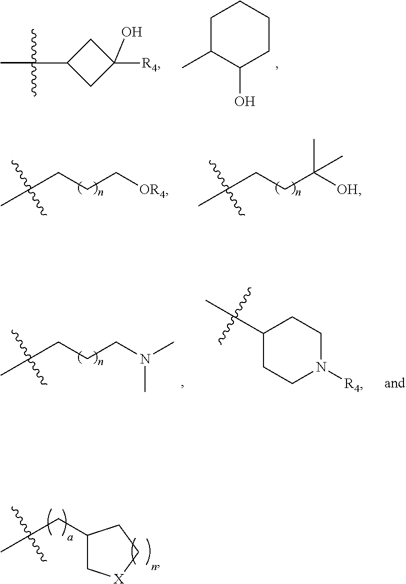

wherein X is selected from O and NR₄;
R₄ is independently selected from —H or —C₁-C₃alkyl;
a is selected from 0 and 1; and
n is selected from 0, 1 or 2.

In a further preferred embodiment, R₃ is selected from the group consisting of

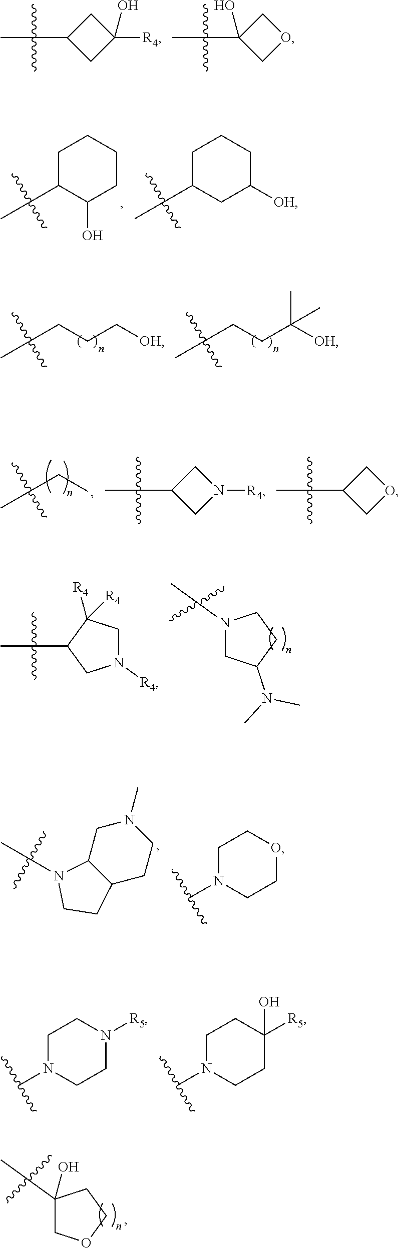

### Me, and CF₃;

wherein
R₄ is independently selected from H, F, or C₁-C₃alkyl; and
R₅ is methyl; and
n is selected from 0, 1 or 2.

In a further preferred embodiment, R₃ is selected from the group consisting of

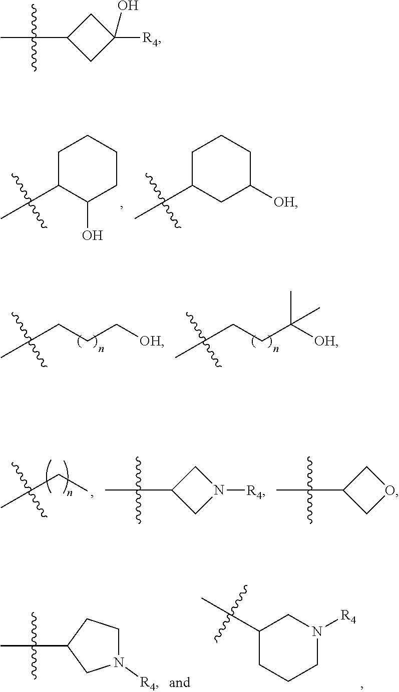

wherein R₄ is independently selected from —H or —C₁-C₃alkyl; and
n is selected from 0, 1 or 2.

In a further preferred embodiment, R₃ is selected from the group consisting of

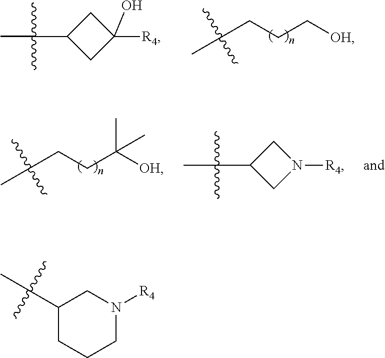

wherein R₄ is independently selected from —H or —C₁-C₃alkyl; and
n is selected from 0, 1 or 2.

In one preferred embodiment R₃ is a 4-, 5- or 6-membered heterocycloalkyl containing one heteroatom, wherein said heteroatom is N. The 4-, 5- or 6-membered heterocycloalkyl can be substituted with one or two substituents independently selected from the group consisting of C₁-C₄alkyl, haloC₁-C₄alkyl, hydroxyC₁-C₄alkyl, —OH and halo (preferably C₁-C₄alkyl). The substituents can be at any available position on the heterocyclic group.

In one preferred embodiment R₃ is

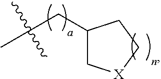

wherein X is selected from O and NR₄, preferably X is NR₄;
R₄ is independently selected from —H or —C₁-C₃alkyl;
a is selected from 0 and 1 (preferably a is 0); and
n is selected from 0, 1 or 2. In one preferred embodiment n is 2. In one preferred embodiment n is 1. In another preferred embodiment n is 0.

In one preferred embodiment R₃ is

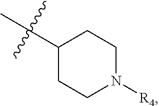

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In another preferred embodiment R₃ is

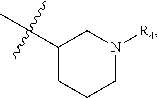

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In another embodiment R₃ is

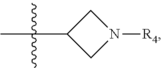

wherein R₄ is independently selected from —H or C₁-C₃alkyl. In a preferred embodiment R₄ is H. In another embodiment R₄ is C₁-C₃alkyl, for instance methyl, ethyl or propyl. In a one embodiment R₄ is methyl, whereby R₃ is

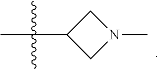 [SMILES: *C1CN(C)C1]

In another embodiment R₃ is

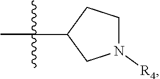

wherein R₄ is independently selected from —H or C₁-C₃alkyl. In a preferred embodiment R₄ is H. In another embodiment R₄ is C₁-C₃alkyl, for instance methyl, ethyl or propyl. In one preferred embodiment R₄ is methyl, whereby R₃ is

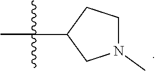 [SMILES: *C1CCN(C)C1]

In another preferred embodiment R₃ is a 4, 5 or 6-membered heterocycloalkyl containing one heteroatom, wherein said heteroatom is O. In one preferred embodiment R₃ is

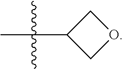 [SMILES: *C1COC1]

In another embodiment R₃ is a C₃-C₆cycloalkyl, preferably cyclohexyl, cyclopropyl or cyclobutyl, more preferably cyclopropyl or cyclobutyl. The C₃-C₆cycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the cycloalkyl group.

In one preferred embodiment R₃ is

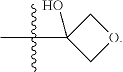 [SMILES: *C1(O)COC1]

In one preferred embodiment R₃ is

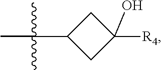

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl. In one preferred embodiment R₃ is

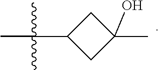 [SMILES: *C1CC(C)(O)C1]

In another preferred embodiment R₃ is

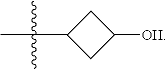 [SMILES: *C1CC(O)C1]

In another preferred embodiment R₃ is

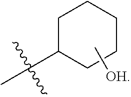

In one preferred embodiment R₃ is

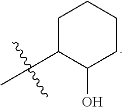 [SMILES: *C1CCCCC1O]

In another preferred embodiment R₃ is

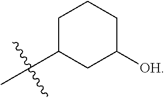 [SMILES: *C1CCCC(O)C1]

In another preferred embodiment R₃ is C₁-C₆alkyl, for example C₁-C₃alkyl, for example methyl, ethyl or propyl. In one preferred embodiment R₃ is methyl. In another preferred embodiment the alkyl can be substituted with one or two substituents independently selected from the group consisting of —OH, halo, haloC₁-C₄alkyl, C₁-C₄alkoxy and —NR₅R₆, wherein R₅ and R₆ are independently selected from H and C₁-C₃alkyl. In one preferred embodiment R₃ is hydroxyC₁-C₆alkyl (i.e. C₁-C₆alkyl substituted with —OH), more preferably hydroxyC₁-C₃alkyl, preferable hydroxyethyl.

In one preferred embodiment R₃ is

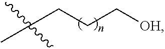

wherein n is 0, 1 or 2. In another preferred embodiment R₃ is

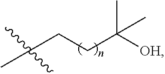

wherein n is 0, 1 or 2.

In another embodiment R₃ is C₁-C₄alkoxyC₁-C₆alkyl (i.e. C₁-C₆alkyl substituted with C₁-C₄alkoxy), preferably C₁-C₃alkoxyC₁-C₄alkyl.

In one embodiment R₃ is

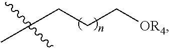

wherein R₄ is —C₁-C₃alkyl; and n is selected from 0, 1 or 2.

In another preferred embodiment, R₃ is C₁-C₆alkyl substituted with —NR₅R₆, wherein R₅ and R₆ are independently selected from H and C₁-C₃alkyl. In a preferred embodiment R₅ and R₆ are both C₁-C₃alkyl, e.g. methyl, ethyl or propyl, more preferably R₅ and R₆ are both methyl. In another embodiment R₅ and R₆ are both H.

In one embodiment R₃ is

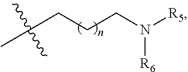

wherein R₅ and R₆ are independently selected from H and C₁-C₃alkyl and wherein n is 0, 1 or 2. In a preferred embodiment R₅ and R₆ are both C₁-C₃alkyl, e.g. methyl, ethyl or propyl, more preferably R₅ and R₆ are both methyl. In another embodiment R₅ and R₆ are both H.

In one preferred embodiment R₃ is

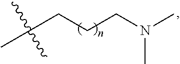

wherein n is 0, 1 or 2.

Rₐ is selected from the group consisting of —H and —C₁-C₃alkyl. In some embodiments Rₐ is H. In other embodiments Rₐ is —C₁-C₃alkyl, for example methyl, ethyl, or propyl, preferably methyl.

In a preferred embodiment X′ is N. In another preferred embodiment X′ is CH.

In a preferred embodiment W is N. In another preferred embodiment W is CH. In another preferred embodiment W is CR_{c} with R_{c} being —CH₂—OH, —O—CH₃ or F.

In one preferred embodiment Y is NH. In another preferred embodiment Y is O. In another embodiment Y is N—CH₃. In another preferred embodiment Y is a bond.

In a preferred embodiment, R_{c} is selected from the group consisting of —O—C₁-C₄alkyl, C₁-C₄alkyl-OH, and halo. In a further preferred embodiment, R_{c} is selected from the group consisting of —O—C₁-C₂alkyl, C₁-C₄alkyl-OH, and F, such as C₁-C₄alkyl-OH, OCH₃, and F.

In a preferred embodiment, R_{d} is selected from the group consisting of C₁-C₄alkyl.

In another preferred embodiment, R₃ is

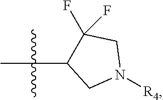

wherein R₄ is independently selected from —H or C₁-C₃alkyl. In a preferred embodiment R₄ is C₁-C₃alkyl, for instance methyl, ethyl or propyl, preferably methyl.

In another preferred embodiment, R₃ is

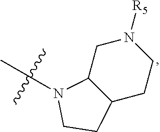

wherein R₅ is H or C₁-C₃alkyl.

In another preferred embodiment R₃ is C₁-C₃alkyl.

In another preferred embodiment there is provided a compound of formula (II), having the formula (IIb)

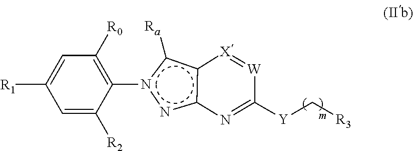

or a stereoisomer, a racemic mixture, a tautomer, a polymorph, a pharmaceutically acceptable salt, a prodrug, a hydrate, or a solvate thereof;
wherein X′, W, R₀, R₁, R₂, R₃, Rₐ, m and Y are as defined in any of the above embodiments.

In another preferred embodiment there is provided a compound of formula (II), having the formula (IIb)

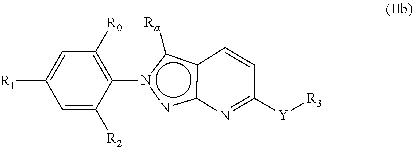

or a stereoisomer, a racemic mixture, a tautomer, a polymorph, a pharmaceutically acceptable salt, a prodrug, a hydrate, or a solvate thereof;
wherein R₀, R₁, R₂, R₃, Rₐ and Y are as defined in any of the above embodiments.

The following paragraphs apply to formulas (II′b) and/or (IIb).

In a preferred embodiment R₀ is selected from —H and C₁-C₃alkyl, more preferably —H or methyl. In one preferred embodiment R₀ is —H. In another preferred embodiment R₀ is C₁-C₃alkyl, preferably methyl or ethyl, more preferably methyl.

In a preferred embodiment, R₁ is selected from —CF₃, —OCF₃ and —OCHF₂. In one preferred embodiment R₁ is —CF₃. In another preferred embodiment R₁ is —OCF₃. In another preferred embodiment R₁ is —OCHF₂.

In another embodiment R₁ is halo Where R₁ is halo, the halogen (halo) is preferably chloro.

In one preferred embodiment R₂ is —OH or H, more preferably —OH. In another embodiment R₂ is —H. In another embodiment R₂ is —CF₃.

In one preferred embodiment R₀ is —H, R₁ is selected from —CF₃, —OCF₃, —OCHF₂ and —Cl, and R₂ is —OH or —H. In one preferred embodiment R₀ is —H, R₁ is —CF₃, and R₂ is —OH.

In one embodiment R₃ is selected from the group consisting of
4-, 5- or 6-membered heterocycloalkyl containing one or two heteroatoms, wherein said heteroatom(s) is/are independently selected from N and/or O, optionally substituted with one or two substituents independently selected from the group consisting of —NR₅R₆, C₁-C₄alkyl or OH;
8-, 9- or 10-membered bicyclic fused heterocycloalkyl containing one to three heteroatoms, wherein said heteroatom(s) is/are independently selected from N and O, optionally substituted at any available position with one or two substituents independently selected from the group consisting of C₁-C₄alkyl, haloC₁-C₄alkyl, hydroxyC₁-C₄alkyl, —OH, —NR₅R₆ and halo; or
C₃-C₆cycloalkyl optionally substituted with one or two substituents independently selected from the group consisting of C₁-C₄alkyl, —OH and halo; or
hydroxyC₁-C₆alkyl; or
haloC₁-C₄alkyl, preferably —CF₃, or
C₁-C₆alkyl optionally substituted with —NR₅R₆,
R₅ and R₆ are independently selected from H and C₁-C₃alkyl; and
m is 0 or 1.

In one preferred embodiment R₃ is selected from the group consisting of
4-, 5- or 6-membered heterocycloalkyl containing one heteroatom, wherein said heteroatom is N or O, optionally substituted with one or two substituents independently selected from the group consisting of C₁-C₄alkyl, haloC₁-C₄alkyl, hydroxyC₁-C₄alkyl, —OH and halo, preferably optionally substituted with one or two substituents independently selected from the group consisting of C₁-C₄alkyl;
C₃-C₆cycloalkyl optionally substituted with one or two substituents independently selected from the group consisting of C₁-C₄alkyl, —OH and halo;
hydroxyC₁-C₆alkyl;
and C₁-C₆alkyl optionally substituted with —NR₅R₆, wherein R₅ and R₆ are independently selected from H and C₁-C₃alkyl.

In one preferred embodiment R₃ is selected from a 4-, 5- or 6-membered heterocycloalkyl containing one or two heteroatoms, preferably one heteroatom, wherein said heteroatom(s) is/are N or O. The 4-, 5- or 6-membered heterocycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the heterocyclic group.

In one preferred embodiment R₃ is selected from the group consisting of

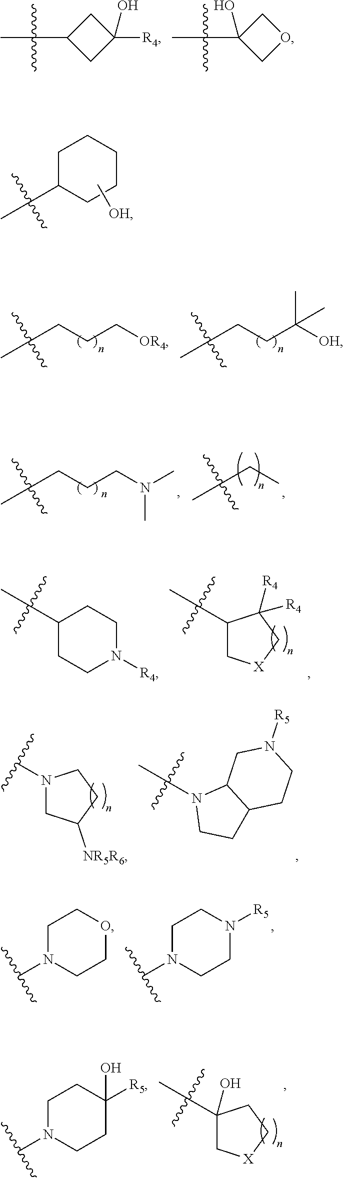

### Me, and CF₃;

wherein
X is selected from O and NR₄;
R₄ is independently selected from H, halo or C₁-C₃alkyl;
R₅ is independently selected from H or C₁-C₃alkyl, preferably H or Me;
R₆ is selected from the group consisting of C₁-C₄alkyl; and
n is selected from 0, 1 or 2.

In one preferred embodiment, R₃ is selected from the group consisting of

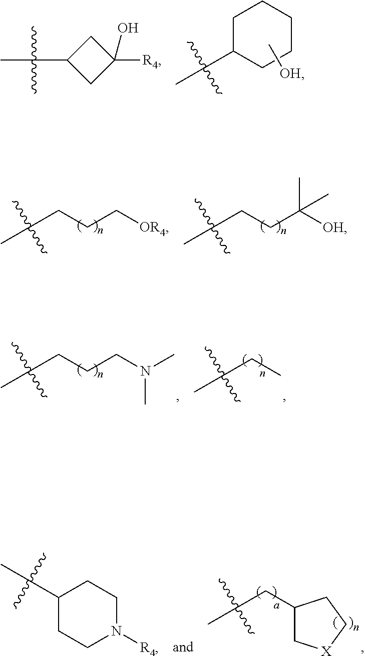

wherein X is selected from O and NR₄;
R₄ is independently selected from —H or —C₁-C₃alkyl;
a is selected from 0 and 1; and
n is selected from 0, 1 or 2.

In one preferred embodiment R₃ is selected from the group consisting of

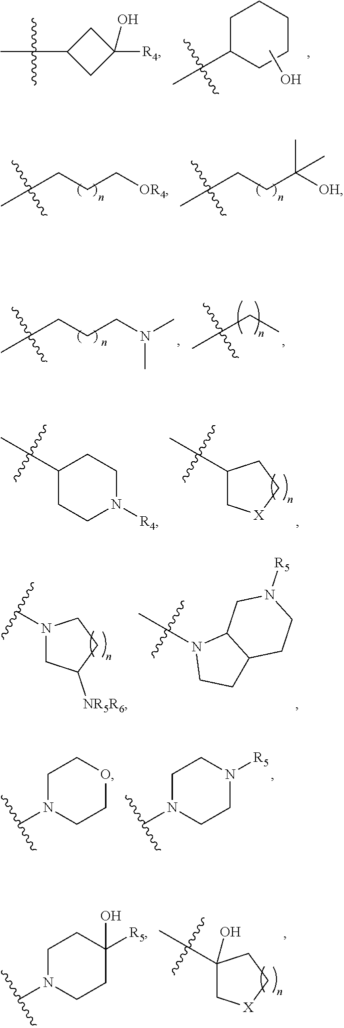

### Me, and CF₃;

wherein
X is selected from O and NR₄;
R₄ is independently selected from H, halo or C₁-C₃alkyl;
R₅ is independently selected from —H or -Me;
R₆ is selected from the group consisting of C₁-C₄alkyl; and
n is selected from 0, 1 or 2.

In one preferred embodiment, R₃ is selected from the group consisting of

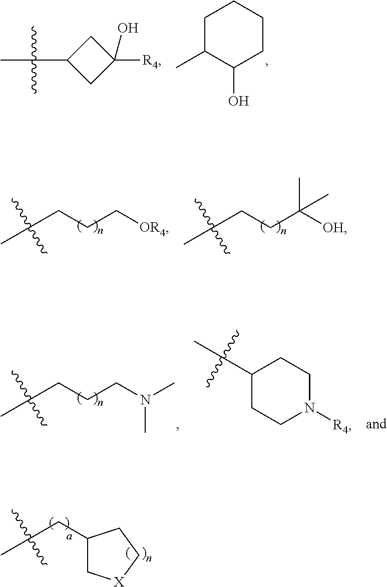

wherein X is selected from O and NR₄;
R₄ is independently selected from —H or —C₁-C₃alkyl;
a is selected from 0 and 1; and
n is selected from 0, 1 or 2.

In a further preferred embodiment, R₃ is selected from the group consisting of

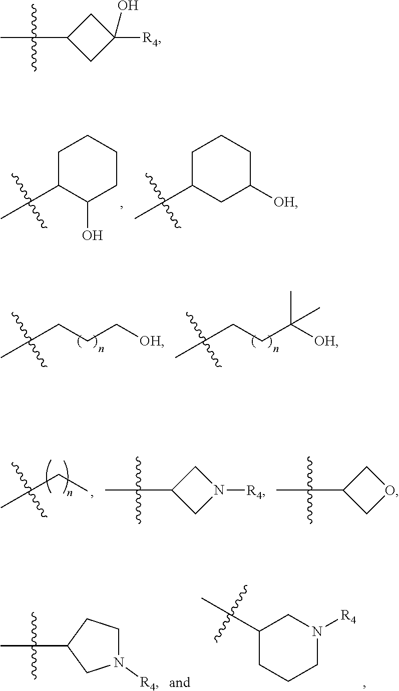

wherein R₄ is independently selected from —H or —C₁-C₃alkyl; and
n is selected from 0, 1 or 2.

In a further preferred embodiment, R₃ is selected from the group consisting of

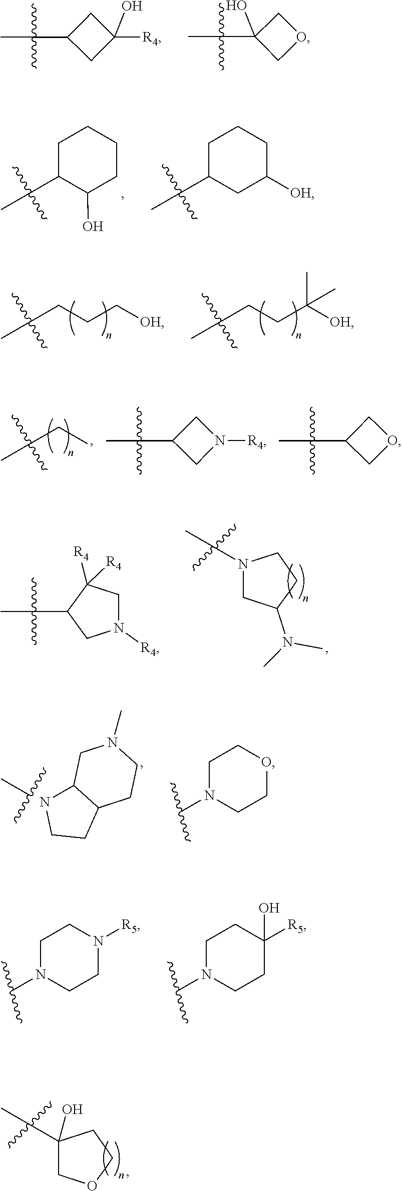

### Me, and CF₃;

wherein
R₄ is independently selected from H, F, or C₁-C₃alkyl; and
R₅ is methyl; and
n is selected from 0, 1 or 2.

In a further preferred embodiment, R₃ is selected from the group consisting of

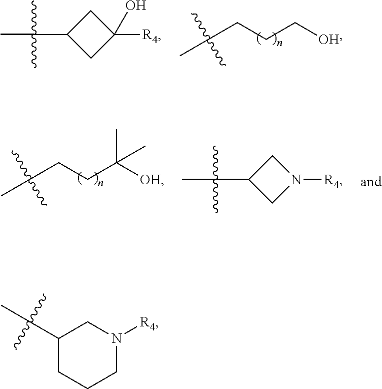

wherein R₄ is independently selected from —H or —C₁-C₃alkyl; and
n is selected from 0, 1 or 2.

In one preferred embodiment R₃ is a 4-, 5- or 6-membered heterocycloalkyl containing one heteroatom, wherein said heteroatom is N. The 4-, 5- or 6-membered heterocycloalkyl can be substituted with one or two substituents independently selected from the group consisting of C₁-C₄alkyl, haloC₁-C₄alkyl, hydroxyC₁-C₄alkyl, —OH and halo (preferably C₁-C₄alkyl). The substituents can be at any available position on the heterocyclic group.

In one preferred embodiment R₃ is

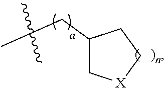

wherein X is selected from O and NR₄, preferably X is NR₄;
R₄ is independently selected from —H or —C₁-C₃alkyl;
a is selected from 0 and 1 (preferably a is 0); and
n is selected from 0, 1 or 2. In one preferred embodiment n is 2. In one preferred embodiment n is 1. In another preferred embodiment n is 0.

In one preferred embodiment R₃ is

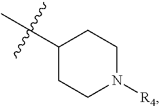

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In another preferred embodiment R₃ is

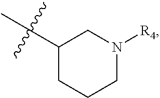

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In another embodiment R₃ is

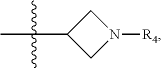

wherein R₄ is independently selected from —H or C₁-C₃alkyl. In a preferred embodiment R₄ is H. In another embodiment R₄ is C₁-C₃alkyl, for instance methyl, ethyl or propyl. In a one embodiment R₄ is methyl, whereby R₃ is

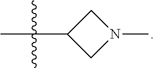 [SMILES: *C1CN(C)C1]

In another embodiment R₃ is

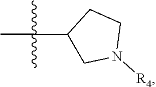

wherein R₄ is independently selected from —H or C₁-C₃alkyl. In a preferred embodiment R₄ is H. In another embodiment R₄ is C₁-C₃alkyl, for instance methyl, ethyl or propyl. In one preferred embodiment R₄ is methyl, whereby R₃ is

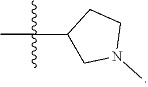 [SMILES: *C1CCN(C)C1]

In another preferred embodiment R₃ is a 4, 5 or 6-membered heterocycloalkyl containing one heteroatom, wherein said heteroatom is O. In one preferred embodiment R₃ is

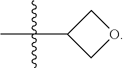 [SMILES: *C1COC1]

In another embodiment R₃ is a C₃-C₆cycloalkyl, preferably cyclohexyl, cyclopropyl or cyclobutyl, more preferably cyclopropyl or cyclobutyl. The C₃-C₆cycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the cycloalkyl group.

In one preferred embodiment R₃ is

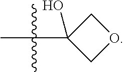 [SMILES: *C1(O)COC1]

In one preferred embodiment R₃ is

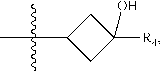

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl. In one preferred embodiment R₃ is

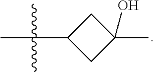 [SMILES: *C1CC(C)(O)C1]

In another preferred embodiment R₃ is

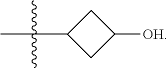 [SMILES: *C1CC(O)C1]

In another preferred embodiment R₃ is

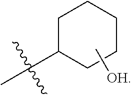

In one preferred embodiment R₃ is

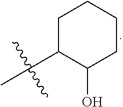 [SMILES: *C1CCCCC1O]

In another preferred embodiment R₃ is

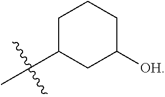 [SMILES: *C1CCCC(O)C1]

In another preferred embodiment R₃ is C₁-C₆alkyl, for example C₁-C₃alkyl, for example methyl, ethyl or propyl. In one preferred embodiment R₃ is methyl. In another preferred embodiment, the alkyl can be substituted with one or two substituents independently selected from the group consisting of —OH, halo, haloC₁-C₄alkyl, C₁-C₄alkoxy and —NR₅R₆, wherein R₅ and R₆ are independently selected from H and C₁-C₃alkyl. In one preferred embodiment R₃ is hydroxyC₁-C₆alkyl (i.e. C₁-C₆alkyl substituted with —OH), more preferably hydroxyC₁-C₃alkyl, preferable hydroxyethyl.

In one preferred embodiment R₃ is

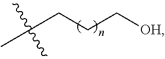

wherein n is 0, 1 or 2. In another preferred embodiment R₃ is

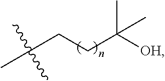

wherein n is 0, 1 or 2.

In another embodiment R₃ is C₁-C₄alkoxyC₁-C₆alkyl (i.e. C₁-C₆alkyl substituted with C₁-C₄alkoxy), preferably C₁-C₃alkoxyC₁-C₄alkyl.

In one embodiment R₃ is

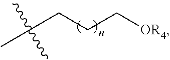

wherein R₄ is —C₁-C₃alkyl; and n is selected from 0, 1 or 2.

In another preferred embodiment, R₃ is C₁-C₆alkyl substituted with —NR₅R₆, wherein R₅ and R₆ are independently selected from H and C₁-C₃alkyl. In a preferred embodiment R₅ and R₆ are both C₁-C₃alkyl, e.g. methyl, ethyl or propyl, more preferably R₅ and R₆ are both methyl. In another embodiment R₅ and R₆ are both H.

In one embodiment R₃ is

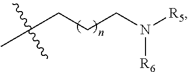

wherein R₅ and R₆ are independently selected from H and C₁-C₃alkyl and wherein n is 0, 1 or 2. In a preferred embodiment R₅ and R₆ are both C₁-C₃alkyl, e.g. methyl, ethyl or propyl, more preferably R₅ and R₆ are both methyl. In another embodiment R₅ and R₆ are both H.

In one preferred embodiment R₃ is

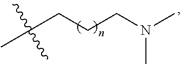

wherein n is 0, 1 or 2.

Rₐ is selected from the group consisting of —H and —C₁-C₃alkyl. In some embodiments Rₐ is H. In other embodiments Rₐ is —C₁-C₃alkyl, for example methyl, ethyl, or propyl, preferably methyl.

In a preferred embodiment X′ is N. In another preferred embodiment X′ is CH.

In a preferred embodiment W is N. In another preferred embodiment W is CH. In another preferred embodiment W is CR_{c} with R_{c} being —CH₂—OH, —O—CH₃ or F.

In one preferred embodiment Y is NH. In another preferred embodiment Y is O. In another embodiment Y is N—CH₃. In another preferred embodiment Y is a bond.

In a preferred embodiment, R_{c} is selected from the group consisting of —O—C₁-C₄alkyl, C₁-C₄alkyl-OH, and halo. In a further preferred embodiment, R_{c} is selected from the group consisting of —O—C₁-C₂alkyl, C₁-C₄alkyl-OH, and F, such as C₁-C₄alkyl-OH, OCH₃, and F.

In a preferred embodiment, R_{d} is selected from the group consisting of C₁-C₄alkyl.

In another preferred embodiment, R₃ is

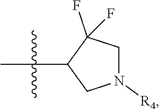

wherein R₄ is independently selected from —H or C₁-C₃alkyl. In a preferred embodiment R₄ is C₁-C₃alkyl, for instance methyl, ethyl or propyl, preferably methyl.

In another preferred embodiment, R₃ is

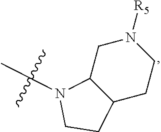

wherein R₅ is H or C₁-C₃alkyl.

In another preferred embodiment R₃ is C₁-C₄alkyl.

In another preferred embodiment there is provided a compound of formula (IV), having the formula (IV)

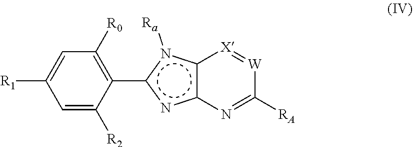

or a stereoisomer, a racemic mixture, a tautomer, a polymorph, a pharmaceutically acceptable salt, a prodrug, a hydrate, or a solvate thereof;
wherein X′, W, R₀, R₁, R₂, R_{d} and Rₐ are as defined in the above embodiments and R_{A} is

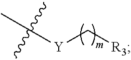

Y is selected from NH, NR_{d}, O or is a bond;
R₃ is selected from the group consisting of
4-, 5- or 6-membered heterocycloalkyl containing one or two heteroatoms, preferably one heteroatom, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH; —NR₅R₆ and halo;
8-, 9- or 10-membered bicyclic fused heterocycloalkyl containing one to three heteroatoms, wherein said heteroatom(s) is/are independently selected from N and O, optionally substituted at any available position with one or two substituents independently selected from the group consisting of C₁-C₄alkyl, haloC₁-C₄alkyl, hydroxyC₁-C₄alkyl, —OH, —NR₅R₆ and halo; or
C₃-C₆cycloalkyl optionally substituted with one or two substituents independently selected from the group consisting of C₁-C₄alkyl, haloC₁-C₄alkyl, hydroxyC₁-C₄alkyl, —OH and halo; or
C₁-C₆alkyl optionally substituted with one or two substituents independently selected from the group consisting of —OH, halo, haloC₁-C₄alkyl, C₁-C₄alkoxy and —NR₅R₆, wherein R₅ and R₆ are independently selected from H and C₁-C₃alkyl; and
m is 0, 1 or 2.

In another preferred embodiment there is provided a compound of formula (V), having the formula (V)

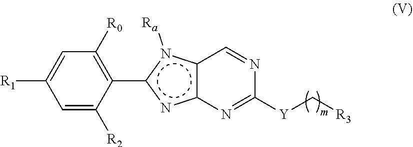

or a stereoisomer, a racemic mixture, a tautomer, a polymorph, a pharmaceutically acceptable salt, a prodrug, a hydrate, or a solvate thereof;
wherein R₀, R₁, R₂, R₃, Rₐ, m and Y are as defined in the above embodiments.

In a preferred embodiment, the present invention relates to the following compounds of formula (I′), (I), (II′), (II), (II′a), (II′b), (IIa), (IIb), (III′), (III), (IV) or (V), above, wherein
R₀ is —H or —CH₃;
R₁ is —CF₃ or halo, preferably Cl; and
R₂ is —OH.

Throughout the present application, if Q is N and R_{B} is

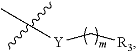

then Y is preferably a bond.

In a further embodiment, the present invention relates to the following compounds of formula (I)

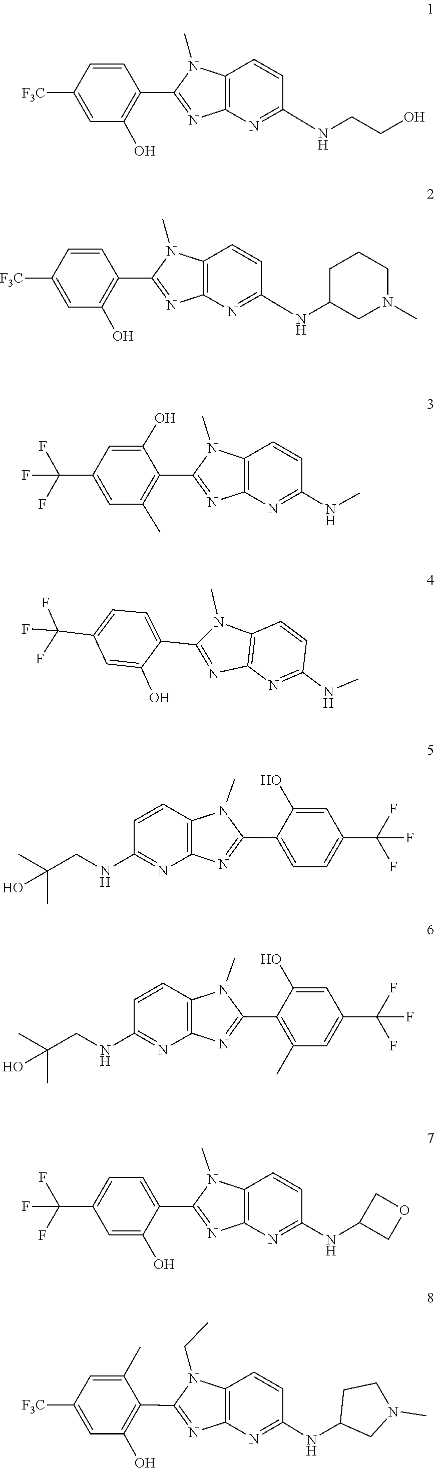

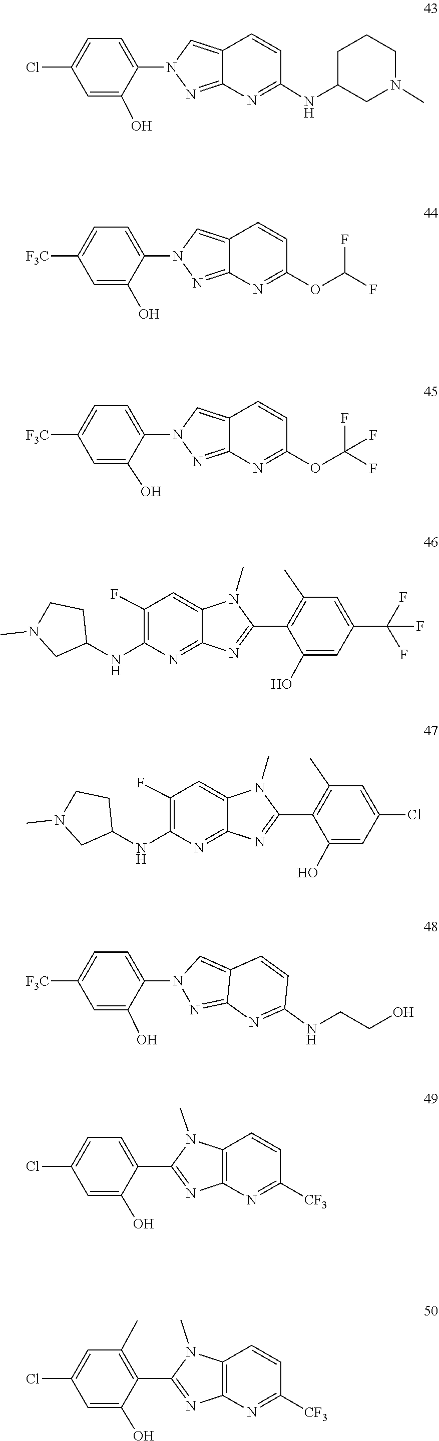

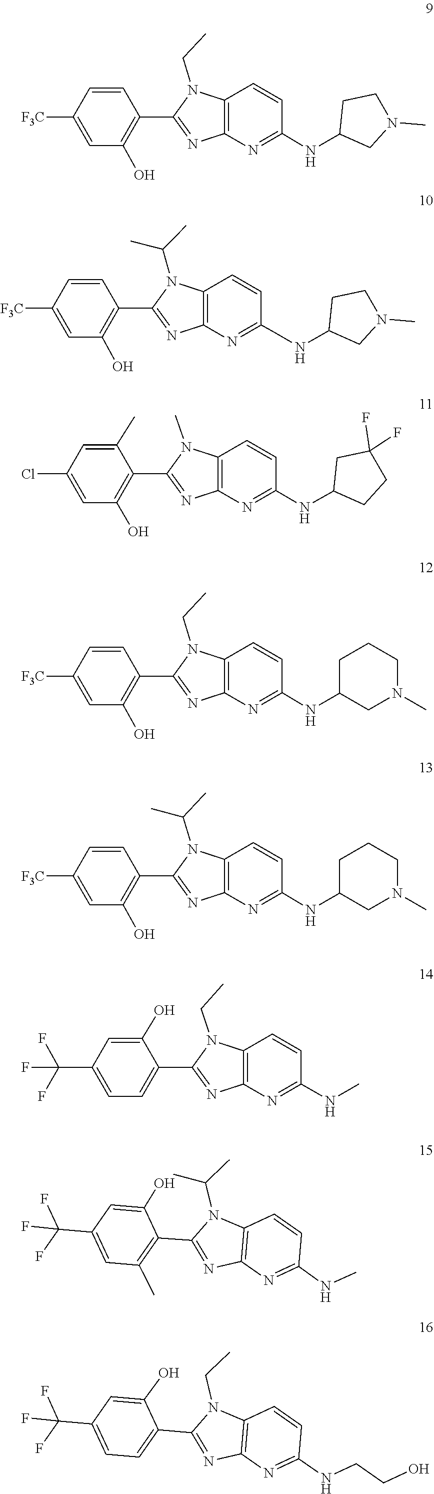

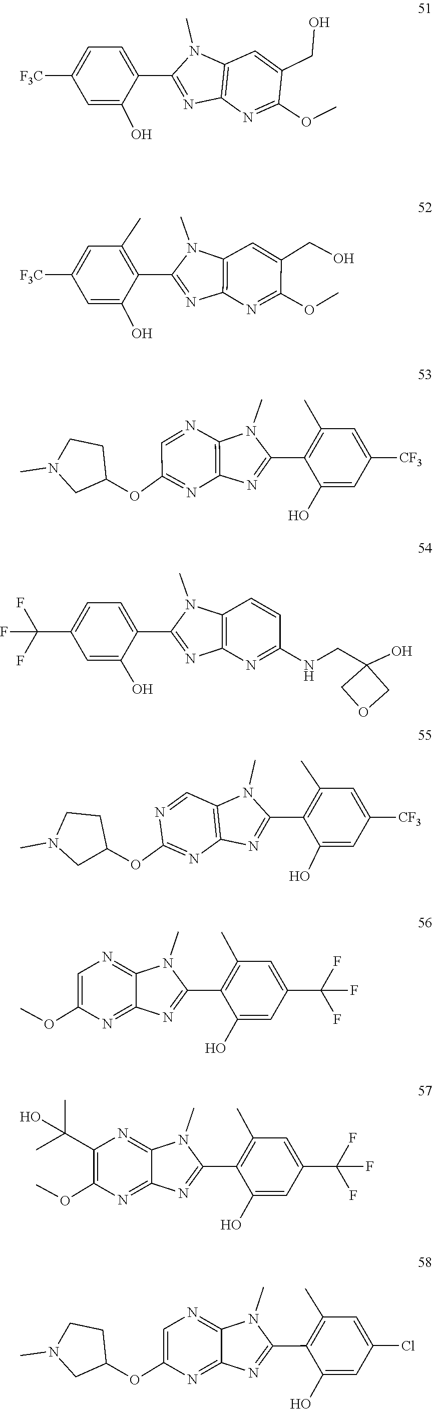

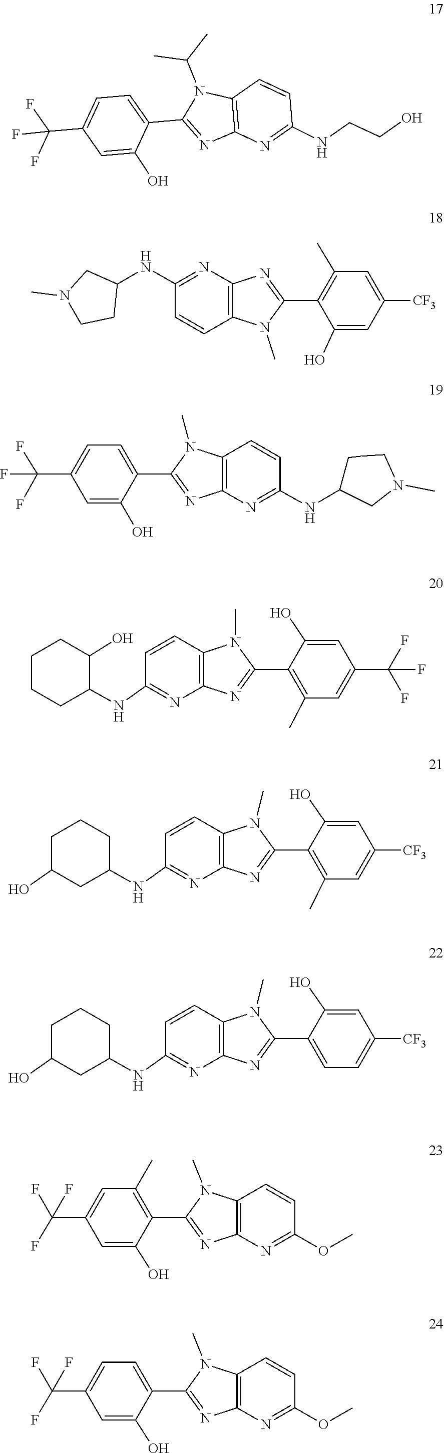

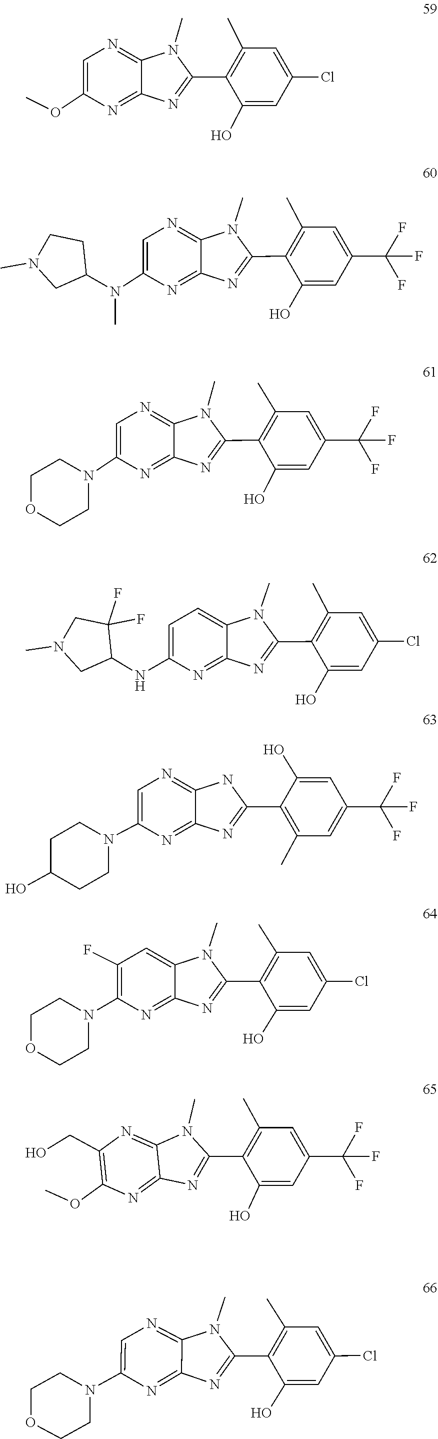

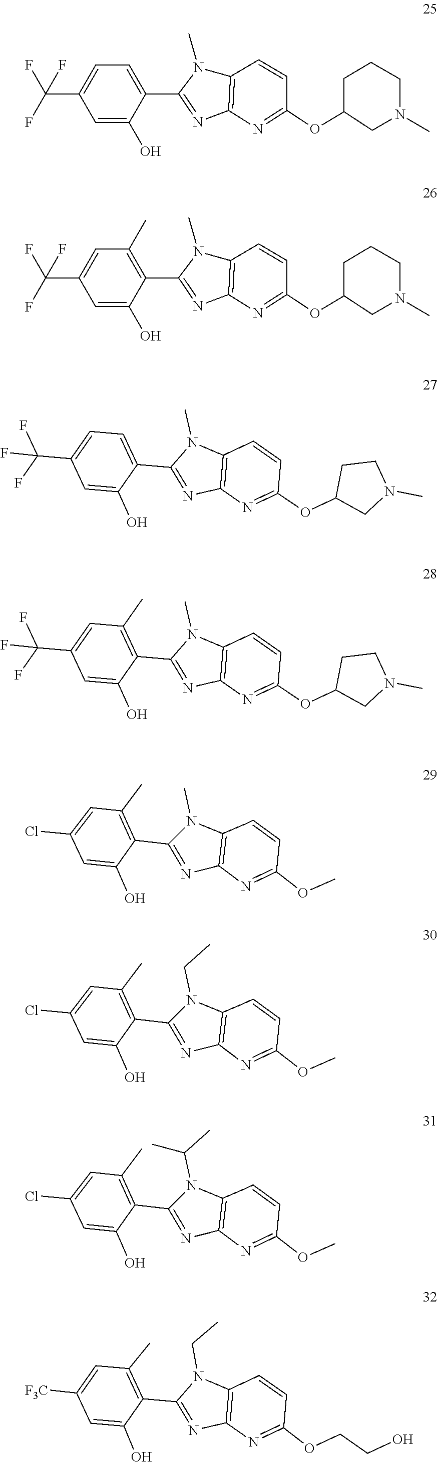

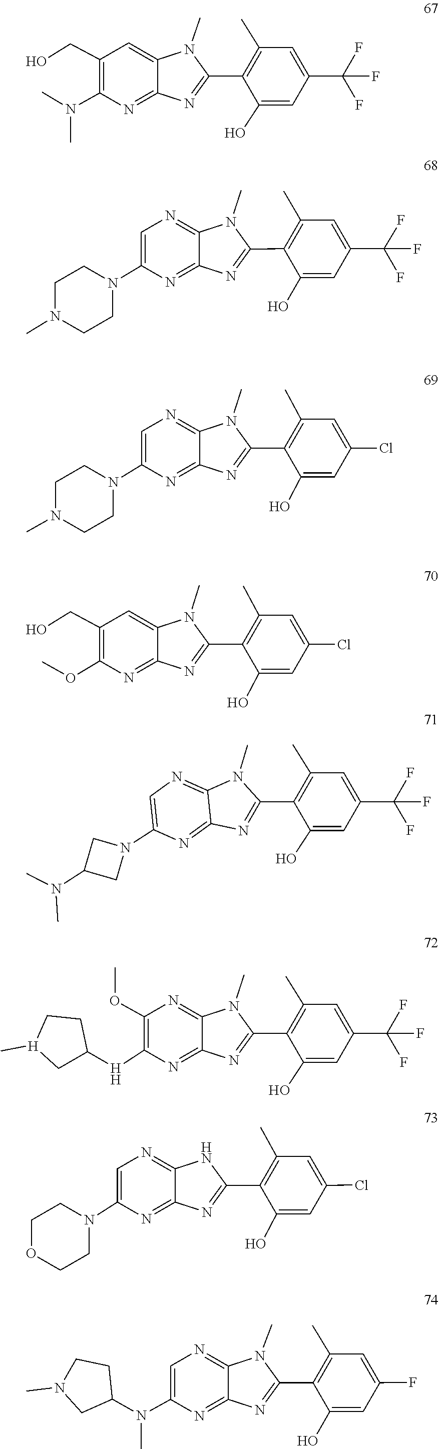

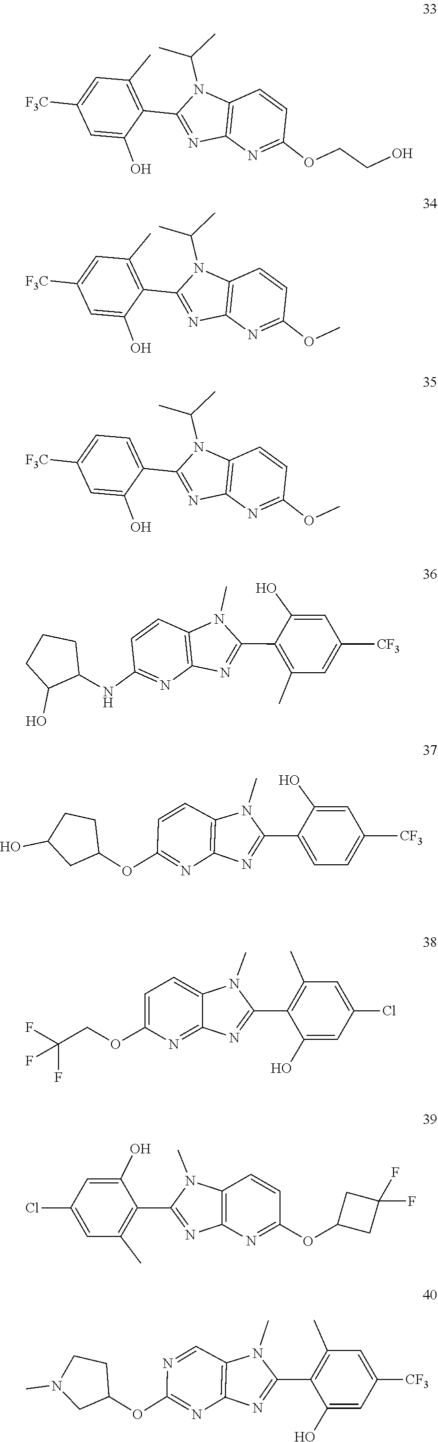

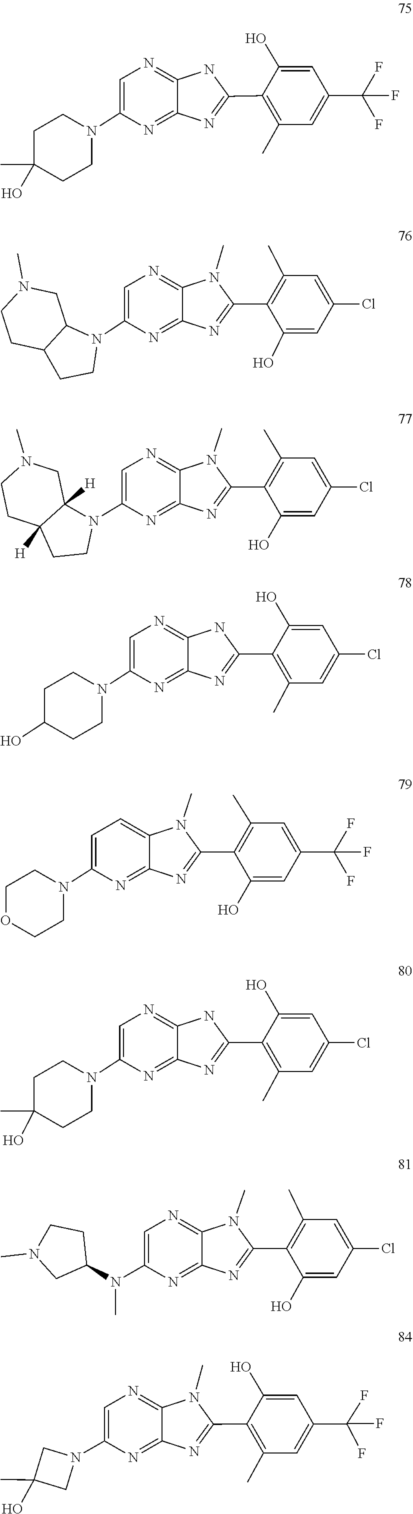

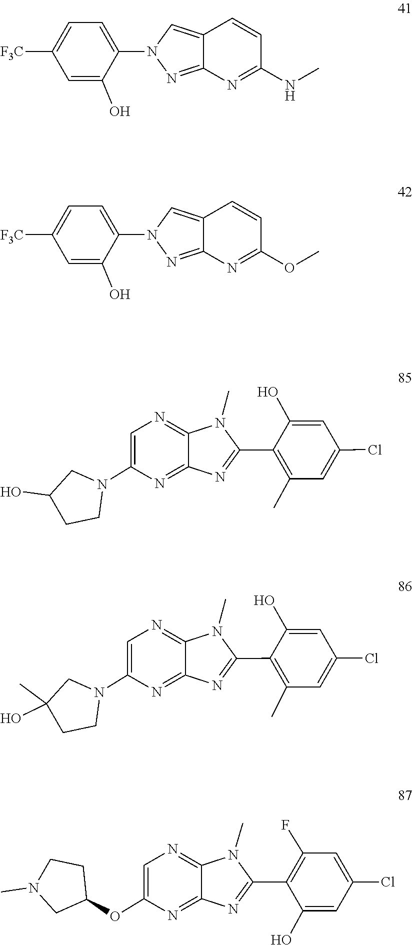

or a stereoisomer, a racemic mixture, a tautomer, a polymorph, a pharmaceutically acceptable salt, a prodrug, a hydrate, or a solvate thereof.

The present invention relates further to a pharmaceutical composition comprising a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, and optionally at least one pharmaceutically acceptable carrier, diluent, adjuvant or excipient.

Various embodiments of the invention are described herein, it will be recognized that features specified in each embodiment may be combined with other specified features to provide further embodiments of the present invention.

The present invention relates to a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for use as a medicament.

The present invention relates to a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a disease, disorder or abnormality which is responsive to the modulation, in particular inhibition of activation, of a component of the NLRP3 inflammasome pathway and/or which is responsive to the modulation, in particular decrease, of IL-1 beta and/or IL-18 levels. In one embodiment, the modulation is the reduction and/or inhibition of IL-1 beta and/or IL-18 beta levels. Particularly, the modulation is the reduction and/or inhibition of IL-1 beta.

The present invention relates to a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a disease, disorder or abnormality which is responsive to the modulation, in particular decrease, of IL-1 beta and/or IL-18 levels.

In another embodiment, the present invention relates to a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for use in a method of reducing and/or inhibiting IL-1 beta and/or IL-18. In one embodiment, the present invention relates to a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for use in a method of reducing and/or inhibiting IL-1 beta. In particular, inhibiting IL-1 beta.

The present invention relates to a compound of formula (I′), (I), (II′), (II), (II′a), (II′b), (IIa), (IIb), (III′), (III), (IV) or (V) defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a disease, disorder or abnormality which is responsive to the modulation, in particular inhibition of activation, of a component of the NLRP3 inflammasome pathway.

The present invention relates to a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a disease, disorder or abnormality which is responsive to the modulation, in particular inhibition of activation, of NLRP3 inflammasome.

In other words, the present invention relates to a method for treating, alleviating or preventing of a disease, disorder or abnormality which is responsive to the modulation, in particular inhibition of activation, of a component of the NLRP3 inflammasome pathway and/or which is responsive to the modulation, in particular decrease, of the IL-1 beta and/or IL-18 levels, wherein the method comprises administering a therapeutically effective amount of a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, to a subject in need thereof (e.g. patient). In one embodiment, the modulation is the reduction and/or inhibition of IL-1 beta and/or IL-18 beta levels. Particularly, the modulation is the reduction and/or inhibition of IL-1 beta.

In one embodiment, the present invention relates to a method for treating, preventing or alleviating a disease, a disorder or abnormality which is responsive to the modulation, in particular inhibition of activation, of a component of the NLRP3 inflammasome pathway, wherein the method comprises administering a therapeutically effective amount of a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, to a subject in need thereof (e.g. a patient).

The present invention further relates to a method for treating, preventing or alleviating a disease, a disorder or abnormality which is responsive to the modulation, in particular inhibition of activation, of NLRP3 inflammasome, wherein the method comprises administering a therapeutically effective amount of a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, to a subject in need thereof (e.g. a patient).

In one embodiment, the present invention relates to a method for treating, preventing or alleviating a disease, disorder or abnormality responsive to a modulation, in particular a decrease, of IL-1 beta and/or IL-18 levels, wherein the method comprises administering a therapeutically effective amount of a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, to a patient in need thereof. In one embodiment, the modulation is the reduction and/or inhibition of IL-1 beta and/or IL-18 beta levels. Particularly, the modulation is the reduction and/or inhibition of IL-1 beta.

The present invention relates to the use of a compound of (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for the manufacture of a medicament. In a further embodiment, the present invention relates to the use of a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for the manufacture of a medicament for treating, alleviating or preventing a disease, disorder or abnormality which is responsive to the modulation, in particular inhibition of activation, of a component of the NLRP3 inflammasome pathway and/or which is responsive to the modulation, in particular decrease, of IL-1 beta and/or IL-18 levels. In one embodiment, the modulation is the reduction and/or inhibition of IL-1 beta and/or IL-18 beta levels. Particularly, the modulation is the reduction and/or inhibition of IL-1 beta.

The present invention relates to the use of a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for the manufacture of a medicament for treating, alleviating or preventing a disease, disorder or abnormality which is responsive to the modulation, in particular inhibition of activation, of a component of the NLRP3 inflammasome pathway.

The present invention relates to the use of a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for the manufacture of a medicament for treating, alleviating or preventing a disease, disorder or abnormality which is responsive to the modulation, in particular inhibition of activation, of NLRP3 inflammasome.

The present invention relates to the use of a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for the manufacture of a medicament for treating, alleviating or preventing a disease, disorder or abnormality which is responsive to the modulation, in particular decrease, of IL-1 beta and/or IL-18 levels.

The present invention relates to the use of a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for the manufacture of a medicament for reducing and/or inhibiting IL-1 beta and/or IL-18 beta levels. In one embodiment, the present invention relates to the use of a compound of the invention, as defined herein, for the manufacture of a medicament for reducing and/or inhibiting IL-1 beta. In another embodiment, the present invention relates to the use of a compound of the invention, as defined herein, for the manufacture of a medicament for reducing or inhibiting IL-1 beta.

In one embodiment, the present invention relates to a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a tauopathy by modulating a component of the inflammasome pathway, in particular, by modulating the NLRP3 inflammasome pathway.

In another embodiment, the disease, the disorder or the abnormality is responsive to modulation of one or more of IL-1 β, IL-17, IL-18, IL-1 a, IL-37, IL-33 and Th17 cells, preferably: IL-1 β and IL-18.

In yet another embodiment, the disease, disorder, or abnormality is a disease, disorder, or abnormality selected from Alzheimer's disease, Parkinson's disease, cryopyrin-associated periodic syndromes (CAPS), non-alcoholic fatty liver disease (NAFLD), non-alcoholic steatohepatitis (NASH), and gout.

In a further embodiment, the disease, disorder, or abnormality is a disease, a disorder or an abnormality of the immune system. In an embodiment, the disease, disorder, or abnormality is an inflammatory disease, disorder, or abnormality. In yet another embodiment, the disease, disorder, or abnormality is an autoimmune disease, disorder, or abnormality. In yet another embodiment, the disease, the disorder, or the abnormality is a disease, a disorder, or an abnormality of the central nervous system (CNS). In yet another embodiment, the disease, the disorder, or the abnormality can be a disease, disorder or abnormality or condition of the skin. The disease, the disorder or the abnormality can be a disease, disorder or abnormality or condition of the cardiovascular system. The disease, the disorder or the abnormality or condition can be a cancer, tumor or other malignancy. The disease, the disorder or the abnormality or condition can be a disease, disorder, or abnormality of the renal system. The disease, the disorder or the abnormality or condition can be a disease, disorder, or abnormality of the gastrointestinal tract. The disease, the disorder or the abnormality or condition can be a disease, disorder, or abnormality of the respiratory system. The disease, the disorder or the abnormality or condition can be a disease, disorder, or abnormality of the endocrine system. The disease, the disorder or the abnormality or condition can be liver related disease, disorder, or abnormality.

In one embodiment, the diseases, the disorders or the abnormalities which are responsive to the modulation, in particular inhibition of activation, of a component of the NLRP3 inflammasome pathway can be selected from Alzheimer's disease, Parkinson's disease, amyotrophic lateral sclerosis, demyelination, viral encephalitis, epilepsy, stroke, atherosclerosis, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease (IBD) (including Crohn's disease, ulcerative colitis), hepatitis, nonalcoholic fatty liver disease, nonalcoholic steatohepatitis, hypertension, myocardial infarction, heart failure, coronary artery disease, oxalate-induced nephropathy, graft-versus host disease, type 1 diabetes, type 2 diabetes, Edema (DME), Geographic Atrophy (GA), rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, skin contact hypersensitivity, sunburn, psoriasis, hidradenitis suppurativa (HS), epidermolysis bullosa, acne, eczema, alopecia areata, actinic keratosis, hypercstosis, osteitis syndrome (SAPHO), vitiligo, atopic dermatitis, cutaneous lupus, multiple sclerosis (MS), Behcet's disease, Sjogren's syndrome, Schnitzler syndrome, chronic obstructive pulmonary disorder (COPD), asthma, steroid-resistant asthma, Coronavirus-associated inflammatory pathologies including Coronavirus-associated respiratory distress syndrome (CARDS), asbestosis, silicosis, cystic fibrosis, motor neuron disease, Huntington's disease, cerebral malaria, brain injury from pneumococcal meningitis, obesity, age-related macular degeneration (AMD), comeal infection, uveitis, dry eye, acute kidney injury, chronic kidney disease, lupus nephritis diabetic nephropathy, alcoholic liver disease, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, primary biliary cholangitis, antiphospholipid syndrome, refractory celiac disease, pancreatitis, autoimmune pancreatitis, mucocutaneous lymph node syndrome, lung cancer metastasis, pancreatic cancers, gastric cancers, myelodisplastic syndrome, leukemia; polymyositis, colitis, helminth infection, bacterial infection, abdominal aortic aneurism, wound healing, migraine, depression, psychological stress, pain, neuropathic pain, periodontitis, pericarditis including Dressler's syndrome, ischaemia reperfusion injury, frontotemporal dementia, HIV-associated neurocognitive disorder, traumatic brain injury, traumatic spinal cord injury, ankylosing spondylitis, cytokine release syndrome.

Preferably, the diseases, the disorders or the abnormalities are selected from Alzheimer's disease, Parkinson's disease, amyotrophic lateral sclerosis, demyelination, multiple sclerosis, encephalomyelitis, leukoencephalopathy, viral encephalitis, epilepsy, stroke, traumatic brain and spinal cord injury, atherosclerosis, asthma and allergic inflammation, cryopyrin-associated periodic syndromes (CAPS), gout, inflammatory bowel disease (IBD), non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, rheumatoid arthritis, acute kidney disease, chronic kidney disease, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, Hereditary Recurrent Fevers (HRFs) acne, atopic dermatitis and hidradenitis suppurativa (HS).

More preferably, the diseases, the disorders or the abnormalities are selected from Alzheimer's disease, Parkinson's disease, amyotrophic lateral sclerosis, demyelination, multiple sclerosis, viral encephalitis, epilepsy, stroke, atherosclerosis, asthma and allergic inflammation, cryopyrin-associated periodic syndromes (CAPS), gout, inflammatory bowel disease (IBD), non-alcoholic fatty liver disease (NAFLD), non-alcoholic steatohepatitis (NASH), hypertension, myocardial infarction, oxalate-induced nephropathy, graft-versus host disease, type 1 and type 2 diabetes, hidradenitis suppurativa (HS), gout, rheumatoid arthritis, acute kidney disease, chronic kidney disease and myelodysplastic syndrome.

Even more preferably, the diseases, the disorders or the abnormalities are selected from Alzheimer's disease, Parkinson's disease, multiple sclerosis, cryopyrin-associated periodic syndromes (CAPS), non-alcoholic fatty liver disease (NAFLD), non-alcoholic steatohepatitis (NASH), chronic kidney disease, Inflammatory Bowel Disease (IBD), hidradenitis suppurativa (HS), rheumatoid arthritis and gout. Even more preferably, the diseases, the disorders or the abnormalities are selected from Alzheimer's disease, Parkinson s disease, multiple sclerosis, cryopyrin-associated periodic syndromes (CAPS), non-alcoholic fatty liver disease (NAFLD), non-alcoholic steatohepatitis (NASH), hidradenitis suppurativa (HS), chronic kidney disease, and gout.

In one embodiment, the diseases, the disorders or the abnormalities which are responsive to the modulation, in particular inhibition of activation, of a component of the NLRP3 inflammasome pathway can be selected from Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease, nonalcoholic fatty liver disease, nonalcoholic steatohepatitis, hypertension, myocardial infarction, oxalate-induced nephropathy, graft-versus host disease, type 1 diabetes, type 2 diabetes, rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, acne, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, acne, eczema, alopecia areata, actinic keratosis, hyperostosis, osteitis syndrome (SAPHO), multiple sclerosis (MS), psoriasis, Behcet's disease, Sjogren's syndrome, Schnitzler syndrome, chronic obstructive pulmonary disorder (COPD), steroid-resistant asthma, asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, chronic kidney disease, diabetic nephropathy, alcoholic liver disease, skin contact hypersensitivity, sunburn, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, hidradenitis suppurativa (HS), lung cancer metastasis, pancreatic cancers, gastric cancers, myelodisplastic syndrome, leukemia; polymyositis, colitis, helminth infection, bacterial infection, abdominal aortic aneurism, wound healing, depression, psychological stress, pericarditis including Dressier's syndrome, ischaemia reperfusion injury, frontotemporal dementia, HIV-associated neurocognitive disorder, Coronavirus-associated inflammatory pathologies, and traumatic brain injury.

In one embodiment, the diseases, the disorders or the abnormalities are selected from Alzheimer's disease, Parkinson's disease, amyotrophic lateral sclerosis, demyelination, viral encephalitis, epilepsy, stroke, atherosclerosis, asthma and allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, rheumatoid arthritis, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, and Hereditary Recurrent Fevers (HRFs).

In one embodiment, the diseases, the disorders or the abnormalities are selected from Alzheimer's disease, Parkinson's disease, amyotrophic lateral sclerosis, demyelination, viral encephalitis, epilepsy, stroke, atherosclerosis, asthma and allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 and type 2 diabetes, rheumatoid arthritis, and myelodysplastic syndrome.

In one embodiment, the diseases, the disorders or the abnormalities are selected from Alzheimer's disease, Parkinson's disease, cryopyrin-associated periodic syndromes (CAPS), non-alcoholic fatty liver disease (NAFLD), non-alcoholic steatohepatitis (NASH), rheumatoid arthritis and gout. Even more preferably, the diseases, the disorders or the abnormalities are selected from Alzheimer's disease, Parkinson's disease, cryopyrin-associated periodic syndromes (CAPS), rheumatoid arthritis and gout.

In one embodiment, the disease, the disorder or the abnormality is selected from Alzheimer's disease and Parkinson's disease. In another embodiment, the disease, the disorder or the abnormality is selected from cryopyrin-associated periodic syndromes (CAPS), non-alcoholic fatty liver disease (NAFLD), non-alcoholic steatohepatitis (NASH) and gout.

In one embodiment, the present invention relates to a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a IL-18 and/or IL-1 beta related disease by modulating a component of the NLRP3 inflammasome pathway, in particular, by modulating NLRP3 inflammasome pathway. The IL-18 and/or IL-1 beta levels in a subject are decreased as a result of the administration of a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof.

IL-18 and/or IL-1 beta related diseases, disorders or abnormalities are selected from chronic obstructive pulmonary disease (COPD), transfusion-related lung injury, bronchopulmonary dysplasia (BPD), acute respiratory distress syndrome (ARDS), Coronavirus-associated respiratory distress syndrome (CARDS), pediatric autoinflammatory disease or condition, Still's disease, particularly Adult Still's disease or juvenile Still's disease, juvenile rheumatoid arthritis (JRA), juvenile idiopathic arthritis (JIA), systemic juvenile onset idiopathic arthritis (SoJIA), systemic juvenile idiopathic arthritis (sJIA), interstitial lung disease (ILD), macrophage activation syndrome (MAS) including primary, secondary and recurrent MAS, hemophagocytic lymphohistiocytosis (HLH), Familial (hereditary) hemophagocytic lymphohistiocytosis (FHLH) associated with gene defects in perforin, munc 13-4 and 18-2, synthaxin 11, immune deficiencies such as Chediak-Higashi syndrome (CHS), Griscelli syndrome (GS), X-linked lymphoproliferative syndrome (XLP2), X-linked inhibitor of apoptosis protein deficiency (XIAP), acquired hemophagocytic lymphohistiocytosis associated with infectious conditions especially Herpes virus such as EBV and other pathogens, autoinflammatory syndrome associated with NLRC4 mutations, Giant Cell Arteritis (GCA), acne, Hidradenitis Suppurativa (HS), pyogenic arthritis pyoderma gangrenosum and acne (PAPA), pulmonary sarcoidosis, Edema (DME), Geographic Atrophy (GA), heart failure, ischemic heart disease, dry eye disease (DED), keratitis, corneal ulcer and abrasion, iritis, glaucoma, Sjogren's syndrome, autoimmune uveitis, Behcet's disease, conjunctivitis, allergic conjunctivitis, diabetes type 2, solid organ and hematologic stem cell transplantation, ischemia reperfusion injury, familial Mediterranean fever (FMF), tumor necrosis factor receptor 1-associated periodic syndromes (TRAPS), hyper-IgD syndromes (mevalonate kinase gene mutation), gout, Schnitzler syndrome, Wegener's granulomatosis also called granulomatosis with polyangitis (GPA), Hashimoto's thyroiditis, Crohn's disease, early onset inflammatory bowel disease (EOIBD), very EOIBD (VEOIBD), infantile IBD, neonatal IBD, ulcerative colitis and Blau syndrome (NOD-2 mutation).

The modulation of NLRP3 inflammasome pathway appears to be beneficial in diseases or disorders or abnormalities with altered IL-18 levels and/or IL-1 beta, which lead to pathological inflammation.

The present invention relates to compound of formula (I′), (I), (II′), (II), (II′a), (II′b), (IIa), (IIb), (III′), (III), (IV) or (V) defined in the present invention that are modulators of NLRP3 inflammasome activity and/or modulators of IL-18 and/or IL-1b levels in a subject.

In one embodiment, the invention provides a pharmaceutical composition comprising a compound of (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, and at least one further biologically active compound. Optionally, the pharmaceutical combination may comprise a pharmaceutically acceptable carrier, diluent, adjuvant or excipient as described herein.

In another embodiment, the present invention relates to a pharmaceutical composition comprising a combination of a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, and at least one further biologically active compound differing from the compound of formula (I′), (I), (II′), (II), (II′a), (II′b), (IIa), (IIb), (III′), (III), (IV) or (V) and optionally comprising at least one pharmaceutically acceptable carrier, diluent, adjuvant or excipient.

In another embodiment, the present invention relates to a pharmaceutical composition comprising a combination of a compound of formula (I(I′), (I), (II′), (II), (II′a), (II′b), (IIa), (IIb), (III′), (III), (IV) or (V) defined in the present invention, or a stereoisomer, a racemic mixture, a tautomer, a polymorph, a pharmaceutically acceptable salt, a prodrug, a hydrate, or a solvate thereof, and at least one further biologically active compound differing from the compound of formula (I′), (I), (II′), (II), (II′a), (II′b), (IIa), (IIb), (III′), (III), (IV) or (V) and optionally comprising at least one pharmaceutically acceptable carrier, diluent, adjuvant or excipient.

In particular the further biologically active compound can be one used for the treatment of a disease, disorder or abnormality which targets a different pathomechanism, e.g. an anti-amyloid beta antibody, anti-Tau antibody, amyloid beta small molecule inhibitor, Tau aggregation small molecule inhibitor, anti-alpha synuclein antibody or alpha-synuclein aggregation small molecule inhibitor, anti-TDP-43 antibody or anti-TDP-43 aggregation small molecule inhibitor, among others. When a compound of the invention is used in combination with a further biologically active compound, the dose of each compound may differ from the dose if the compound is used as a monotherapy. Such biologically active compounds are well known from the literature. Such biological active compound is, for example, a chemical compound, peptide, antibody, antibody fragment, or nucleic acid, which is therapeutically active or enhances the therapeutic activity when administered to a subject (e.g., patient) in combination with a compound of the invention.

In another embodiment, the present invention relates to a pharmaceutical composition comprising a combination comprising a compound of formula (I′), (I), (II′), (II), (II′a), (II′b), (IIa), (IIb), (III′), (III), (IV) or (V) defined in the present invention, or a stereoisomer, a racemic mixture, a tautomer, a polymorph, a pharmaceutically acceptable salt, a prodrug, a hydrate, or a solvate thereof, and at least one further biologically active compound differing from the compound of formula (I′), (I), (II′), (II), (II′a), (II′b), (IIa), (IIb), (III′), (III), (IV) or (V) and optionally comprising at least one pharmaceutically acceptable carrier, diluent, adjuvant or excipient, for use as a medicament.

The term “combination” refers to either a fixed combination in one dosage unit form, or a combined administration where a compound of the present invention and a combination partner (e.g. another drug as explained above, also referred to as “therapeutic agent” or “further biologically active compound”) may be administered independently at the same time or separately within time intervals.

In another embodiment, the present invention relates to combination, in particular a pharmaceutical combination, comprising a therapeutically effective amount of a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, and at least one further biologically active compound, and optionally at least one pharmaceutically acceptable carrier, diluent, adjuvant or excipient. In particular, the at least one further biologically active compound is a compound differing from a compound of formula (I′), (I), (II′), (II), (II′a), (II′b), (IIa), (IIb), (III′), (III), (IV) or (V).

In another embodiment, the present invention relates to a combination comprising a therapeutically effective amount of a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, and at least one further biologically active compound differing from the compound of formula (I′), (I), (II′), (II), (II′a), (II′b), (IIa), (IIb), (III′), (III), (IV) or (V) and optionally comprising at least one pharmaceutically acceptable carrier, diluent, adjuvant or excipient, for use as a medicament.

The present invention relates to the use of a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, as an analytical reference or an in vitro screening tool. The compounds of the present invention, or a stereoisomer, a racemic mixture, a tautomer, a polymorph, a pharmaceutically acceptable salt, a prodrug, a hydrate, or a solvate thereof, can be used as an analytical reference or an in vitro screening tool for characterization of cells with activated NLRP3 inflammasome pathway and for testing of compounds targeting the NLRP3 inflammasome pathway.

Accordingly, the invention provides the use of a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for treating, alleviating or preventing a disorder or an abnormality which is responsive to the modulation of a component of the NLRP3 inflammasome pathway or which is responsive to the modulation, in particular decrease, of IL-1 beta and/or IL-18 levels, wherein the medicament is prepared for administration with further biologically active agent. The invention also provides the use of further biologically active agent for treating alleviating or preventing a disorder or an abnormality which is responsive to the modulation of a component of the NLRP3 inflammasome pathway or which is responsive to the modulation, in particular decrease, of IL-1 beta and/or IL-18 levels, wherein the further biologically active agent is administered with a compound of the present invention, or a stereoisomer, a racemic mixture, a tautomer, a polymorph, a pharmaceutically acceptable salt, a prodrug, a hydrate, or a solvate thereof.

In another embodiment, the invention provides the use of a compound of (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for treating, alleviating or preventing a disorder or an abnormality which is responsive to the modulation of a component of the NLRP3 inflammasome pathway or which is responsive to the modulation, in particular decrease, of IL-1 beta and/or IL-18 levels, wherein the modulation is the reduction and/or the inhibition of IL-1 beta and/or IL-1 beta levels. Preferably, the modulation is the reduction and/or the inhibition of IL-1 beta. Preferably, the modulation is the inhibition of IL-1 beta. In another embodiment, the invention provides a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for use as a medicament, in particular for inhibiting IL-1 beta.

In another embodiment, the invention also provides a compound of formula (I′), (I), (II′), (II), (II′a), (II′b), (IIa), (IIb), (III′), (III), (IV) or (V) as defined in the present invention, or a stereoisomer, a racemic mixture, a tautomer, a polymorph, a pharmaceutically acceptable salt, a prodrug, a hydrate, or a solvate thereof, for use in a method of treating, alleviating or preventing a disease, disorder or abnormality which is responsive to the modulation of a component of the NLRP3 inflammasome pathway or which is responsive to the modulation, in particular decrease, of IL-1 beta and/or IL-18 levels, wherein said compound of formula (I′), (I), (II′), (II), (II′a), (II′b), (IIa), (IIb), (III′), (III), (IV) or (V) is prepared for administration with further biologically active compound (as defined herein).

In another embodiment, the present invention also provides a method of treating alleviating or preventing a disease, disorder or abnormality which is responsive to the modulation of a component of the NLRP3 inflammasome pathway or which is responsive to the modulation, in particular decrease, of IL-1 beta and/or IL-18 levels, selected from Alzheimer's disease, Parkinson's disease, amyotrophic lateral sclerosis, demyelination, viral encephalitis, epilepsy, stroke, atherosclerosis, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease (including Crohn's disease, ulcerative colitis), nonalcoholic fatty liver disease, nonalcoholic steatohepatitis, hypertension, myocardial infarction, heart failure, coronary artery disease, oxalate-induced nephropathy, graft-versus host disease, type 1 diabetes, type 2 diabetes, Edema (DME), Geographic Atrophy (GA), rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, skin contact hypersensitivity, sunburn, psoriasis, hidradenitis suppurativa (HS), epidermolysis bullosa, acne, eczema, alopecia areata, actinic keratosis, hyperostosis, osteitis syndrome (SAPHO), vitiligo, atopic dermatitis, cutaneous lupus, multiple sclerosis (MS), Behcet's disease, Sjogren's syndrome, Schnitzler syndrome, chronic obstructive pulmonary disorder (COPD), Coronavirus-associated inflammatory pathologies, Coronavirus-associated respiratory distress syndrome (CARDS), steroid-resistant asthma, asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, acute kidney disease, chronic kidney disease, diabetic nephropathy, alcoholic liver disease, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, hidradenitis suppurativa (HS), lung cancer metastasis, pancreatic cancers, gastric cancers, myelodisplastic syndrome, leukemia; polymyositis, colitis, helminth infection, bacterial infection, abdominal aortic aneurism, wound healing, migraine, depression, psychological stress, pain, neuropathic pain, periodontitis, pericarditis including Dressier's syndrome, ischaemia reperfusion injury, frontotemporal dementia, HIV-associated neurocognitive disorder, traumatic brain injury, traumatic spinal cord injury, ankylosing spondylitis and cytokine release syndrome; preferably the disorder is selected from Alzheimer's disease, Parkinson's disease, amyotrophic lateral sclerosis, demyelination, multiple sclerosis, viral encephalitis, epilepsy, stroke, traumatic brain injury, spinal cord injury, atherosclerosis, asthma, allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, rheumatoid arthritis, acute kidney disease, chronic kidney disease, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), hidradenitis suppurativa (HS), Psoriatic Arthritis, and Hereditary Recurrent Fevers (HRFs), comprising administering to the subject a therapeutically effective amount of a compound of formula (I), (II′), (II), (II′a), (IIa), (II′b), (IIb), (III, (IV) or (V) as defined herein, or stereoisomers, or racemic mixtures, or tautomers, or polymorph, or pharmaceutically acceptable salts, or hydrates, or solvates thereof.

In one embodiment, the disease, disorder or abnormality which is responsive to the modulation of a component of the NLRP3 inflammasome pathway or which is responsive to the modulation, in particular decrease, of IL-1 beta and/or IL-18 levels, is selected from Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease, nonalcoholic fatty liver disease, nonalcoholic steatohepatitis, hypertension, myocardial infarction, oxalate-induced nephropathy, graft-versus host disease, type 1 diabetes, type 2 diabetes, rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, acne, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, acne, eczema, alopecia areata, actinic keratosis, hyperostosis, osteitis syndrome (SAPHO), multiple sclerosis (MS), psoriasis, Behcet's disease, Sjogren's syndrome, Schnitzler syndrome, chronic obstructive pulmonary disorder (COPD), steroid-resistant asthma, asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, chronic kidney disease, diabetic nephropathy, alcoholic liver disease, skin contact hypersensitivity, sunburn, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, hidradenitis suppurativa (HS), lung cancer metastasis, pancreatic cancers, gastric cancers, myelodisplastic syndrome, leukemia; polymyositis, colitis, helminth infection, bacterial infection, abdominal aortic aneurism, wound healing, depression, psychological stress, pericarditis including Dressler's syndrome, ischaemia reperfusion injury, frontotemporal dementia, HIV-associated neurocognitive disorder, Coronavirus-associated inflammatory pathologies, and traumatic brain injury; preferably the disorder is selected from Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, rheumatoid arthritis, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, and Hereditary Recurrent Fevers (HRFs), comprising administering to the subject a therapeutically effective amount of a compound of (I′), (I), (II′), (II), (II′a), (II′b), (IIa), (IIb), (III′), (III), (IV) or (V) as defined herein, or stereoisomers, or racemic mixtures, or tautomers, or polymorph, or pharmaceutically acceptable salts, or hydrates, or solvates thereof.

In another embodiment, the present invention also provides a method of inhibiting IL-1 beta in a subject in need, the method comprising administering to a subject in need thereof a therapeutically effective amount of a compound of formula (I′), (I), (II′), (II), (II′a), (II′b), (IIa), (IIb), (III′), (III), (IV) or (V) as defined herein, or a stereoisomer, a racemic mixture, a tautomer, a polymorph, a pharmaceutically acceptable salt, a prodrug, a hydrate, or a solvate thereof.

In particular, the disease, disorder or abnormality is one which is responsive to the inhibition of activation of the NLRP3 inflammasome pathway. More particularly, the disease, disorder or abnormality is responsive to the modulation of one or more of, for example, but not limited to, IL-1 β or IL-18. For example, the disease, disorder, or abnormality is responsive to the modulation of one or more of IL-1 β, IL-17, IL-18, IL-1a, IL-37, IL-33 and Th17 cells, preferably the disease, disorder, or abnormality is responsive to the modulation of IL-1 β and/or IL-18.

Any combination of the embodiments, preferred embodiments and more preferred embodiments disclosed herein is also envisaged in the present invention.

### Pharmaceutical Compositions

While it is possible for the compounds of the present invention, or a stereoisomer, a racemic mixture, a tautomer, a polymorph, a pharmaceutically acceptable salt, a prodrug, a hydrate, or a solvate thereof, to be administered alone, it is preferable to formulate them into a pharmaceutical composition in accordance with standard pharmaceutical practice. Thus, the invention also provides a pharmaceutical composition which comprises a therapeutically effective amount of a compound of (I′), (I), (II′), (II), (II′a), (II′b), (IIa), (IIb), (III′), (III), (IV) or (V) or a stereoisomer, a racemic mixture, a tautomer, a polymorph, a pharmaceutically acceptable salt, a prodrug, a hydrate, or a solvate thereof, optionally in admixture with a pharmaceutically acceptable carrier, diluent, adjuvant or excipient.

The term “a therapeutically effective amount” of a compound of the present invention refers to an amount of the compound of the present invention (i.e. a compound of (I′), (I), (II′), (II), (II′a), (II′b), (IIa), (IIb), (III′), (III), (IV) or (V) or a stereoisomer, a racemic mixture, a tautomer, a polymorph, a pharmaceutically acceptable salt a prodrug, a hydrate, or a solvate thereof) that will elicit the biological or medical response of a subject, for example, reduction or inhibition of an enzyme or a protein activity, or ameliorate symptoms, alleviate conditions, slow or delay disease progression, or prevent a disease, a disorder or an abnormality, etc. In one embodiment, the term “a therapeutically effective amount” refers to the amount of the compound of the present invention that, when administered to a subject in need thereof (e.g. a patient), is effective to at least partially alleviate, prevent and/or ameliorate a disease, a disorder, or an abnormality which is responsive to the modulation of a component of the NLRP3 inflammasome pathway or which is responsive to the modulation, in particular decrease, of IL-1 beta and/or IL-18.

Pharmaceutically acceptable carriers, diluents, adjuvants and excipients are well known in the pharmaceutical art and are described, for example, in Remington's Pharmaceutical Sciences, 18^{th} Ed. (Alfonso R. Gennaro, ed.; Mack Publishing Company, Easton, P A, 1990); Remington: the Science and Practice of Pharmacy 19^{th} Ed. (Lippincott, Williams & Wilkins, 1995); Handbook of Pharmaceutical Excipients, 3^{rd} Ed. (Arthur H. Kibbe, ed.; Amer. Pharmaceutical Assoc, 1999); Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); Fiedler's “Lexikon der Hilfsstoffe” 5^{th} Ed., Edition Cantor Verlag Aulendorf 2002; “The Handbook of Pharmaceutical Excipients”, 4^{th} Ed., American Pharmaceuticals Association, 2003; and Goodman and Gilman's: the Pharmacological Basis of Therapeutics (Louis S. Goodman and Lee E. Limbird, eds.; McGraw Hill, 1992), the disclosures of which are hereby incorporated by reference.

The carriers, diluents, adjuvants and pharmaceutical excipients can be selected with regard to the intended route of administration and standard pharmaceutical practice. These compounds must be acceptable in the sense of being not deleterious to the recipient thereof.

Pharmaceutically useful excipients that may be used in the formulation of the pharmaceutical composition of the present invention may comprise, for example, vehicles, solvents (such as monohydric alcohols such as ethanol, isopropanol and polyhydric alcohols such as glycols), edible oils (such as soybean oil, coconut oil, olive oil, safflower oil, and cottonseed oil), oily esters (such as ethyl oleate and isopropyl myristate), binders (such as hydroxypropylmethyl cellulose (HPMC), hydroxypropyl cellulose (HPC), pregelatinized starch and combinations thereof), solubilizers, thickening agents, stabilizers, disintegrants (such as carboxymethylcellulose calcium (CMC-Ca), carboxymethylcellulose sodium (CMC-Na), crosslinked PVP (e.g., crospovidone, Polyplasdone® or Kollidon® XL), alginic acid, sodium alginate, guar gum, cross-linked CMC (croscarmellose sodium, e.g. Ac-Di-Sol®), carboxymethyl starch-Na (sodium starch glycolate) (e.g., Primojel® or Explotab®), preferably crosslinked PVP and/or croscarmellose sodium), glidants (such as colloidal SiO₂ (e.g., Aerosil® 200), magnesium trisilicate, powdered cellulose, talc and combinations thereof), lubricating agents (such as magnesium stearate, aluminium or calcium silicate, stearic acid, hydrogenated castor oil, talc, glyceryl behenate, sodium stearate fumarate and combinations thereof), buffering agents, emulsifiers, wetting agents, suspending agents, sweetening agents, colorants, flavors, coating agents, preservatives, antioxidants, processing agents, drug delivery modifiers and enhancers (such as calcium phosphate), magnesium stearate, talc, monosaccharides, disaccharides, starch, gelatine, cellulose, methylcellulose, sodium carboxymethyl cellulose, dextrose, hydroxypropyl-R-cyclodextrin, polyvinylpyrrolidone, low melting waxes, and ion exchange resins.

The carrier is not particularly limited and will depend on the route of administration as well as the form of the pharmaceutical composition (i.e., solid, liquid, etc.). Suitable carriers include, without limitation, polyols such as mannitol, sorbitol, xylitol; disaccharides such as lactose, sucrose, dextrose and maltose; polysaccharides such as maltodextrin and dextran; starches such as corn starch; celluloses such as microcrystalline cellulose, sodium carboxy methylcellulose, low-substituted hydroxypropyl cellulose, hydroxyl ethyl cellulose, hydroxypropyl cellulose or mixtures thereof; cyclodextrins and inorganic agents such as dicalcium phosphate, calcium hydrogen phosphate; hydroxyapatite, tricalcium phosphate, talcum and silica. Microcrystalline cellulose, sucrose and/or lactose are preferred as carriers. Combinations thereof can also be employed. Carriers can include also protein and cell penetrating peptides which should be selected depending on the route of administration and target.

The diluent is not particularly limited and will depend on the route of administration as well as the form of the pharmaceutical composition (i.e., solid, liquid, etc.). Diluents include, for instance, water, ethanol, propylene glycol and glycerin, and combinations thereof.

An adjuvant is an additive which has few or no pharmacological effects by themselves, but that increases the efficacy or potency of the compounds of the invention if they are administered together.

The routes for administration (delivery) of the compounds of the invention include, but are not limited to, one or more of the following routes of administration: oral (e.g., as a tablet, capsule, or as an ingestible solution), topical, mucosal (e. g. as a nasal spray or aerosol for inhalation), nasal, parenteral (e.g., by an injectable form), gastrointestinal, intraspinal, intraperitoneal, intramuscular, intravenous, intraarterial, intrathecal, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual.

For example, the compounds can be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatine and acacia. Additionally, lubricating agents such as magnesium stearate, stearic acid, glyceryl behenate and talc may be included. Solid compositions of a similar type may also be employed as fillers in gelatine capsules. Preferred excipients in this regard include starch, cellulose, milk sugar e.g. lactose or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

If the compounds of the present invention, as disclosed herein, are administered parenterally, then examples of such administration include one or more of: intravenously, intraarterially, intraperitoneally, intrathecally, intraventricularly, intraurethrally, intrasternally, intracranially, intramuscularly or subcutaneously administering the compounds; and/or by using infusion techniques. For parenteral administration, the compounds can be used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

As indicated, the compounds of the present invention can be administered intranasally or by inhalation and are conveniently delivered in the form of a dry powder inhaler or an aerosol spray presentation from a pressurized container, pump, spray or nebulizer with the use of a suitable propellant, e.g. dichlorodifluoromethane, trichlorofluoromethane, dichlorotetrafluoroethane, a hydrofluoroalkane such as 1,1,1,2-tetrafluoroethane (HFA134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e. g. using a mixture of ethanol and the propellant as the solvent, which may additionally contain a lubricant, e. g. sorbitan trioleate. Capsules and cartridges (made, for example, from gelatine) for use in an inhaler or insufflator may be formulated to contain a powder mix of the compound and a suitable powder base such as lactose or starch.

Alternatively, the compounds of the present invention, as defined herein, can be administered in the form of a suppository or pessary, or it may be applied topically in the form of a gel, hydrogel, lotion, solution, cream, ointment or dusting powder. The compounds of the present invention, as defined herein, may also be dermally or transdermally administered, for example, by the use of a skin patch.

They may also be administered by the pulmonary or rectal routes. They may also be administered by the ocular route. For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the present invention can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, polyethylene glycol, liquid paraffin, polysorbate 60, cetyl ester wax, cetearyl alcohol, 2-octyldodecanol, benzyl alcohol and water.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

The claimed compounds, as defined herein, can be used for the treatment, alleviation or prevention of the recited conditions alone or in combination with one or more other biologically active compounds, as defined herein. In particular, the other biologically active compound can be one used for the treatment, alleviation, or prevention of the recited diseases.

The combinations referred to above may conveniently be presented for use in the form of a pharmaceutical formulation. The individual components of such combinations may be administered either sequentially or simultaneously in separate or combined pharmaceutical formulations by any convenient route. When administration is sequential, either the compound of the invention or the other biologically active compound may be administered first. When administration is simultaneous, the combination may be administered either in the same or different pharmaceutical composition. When combined in the same formulation it will be appreciated that the two compounds must be stable and compatible with each other and the other components of the formulation. When formulated separately they may be provided in any convenient formulation, conveniently in such manners as are known for such compounds in the art.

The pharmaceutical compositions of the invention can be produced in a manner known per se to the skilled person as described, for example, in Remington's Pharmaceutical Sciences, 15^{th} Ed., Mack Publishing Co., New Jersey (1975).

The compounds according to the present invention, as disclosed herein, can also be provided in the form of a mixture with at least one further biologically active compound and/or a pharmaceutically acceptable carrier, diluent, adjuvant, or excipient. The compound and/or the further biologically active compound are preferably present in a therapeutically effective amount.

The nature of the further biologically active compound will depend on the intended use of the mixture. The further biologically active substance or compound may exert its biological effect by the same or a similar mechanism as the compound according to the invention or by an unrelated mechanism of action or by a multiplicity of related and/or unrelated mechanisms of action.

The invention also includes all suitable isotopic variations of the compounds of the invention. An isotopic variation of the compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, sulfur, fluorine and chlorine such as ²H, ³H, ¹³C, ¹⁴C, ¹⁵N, ¹⁷O, ¹⁸O, ³⁵, ¹⁸F and ³⁶Cl respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and delectability. ¹⁸F-labeled compounds are particularly suitable for imaging applications such as PET. Further, substitution with isotopes such as deuterium, i.e., ²F, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Examples and Preparations hereafter using appropriate isotopic variations of suitable reagents.

### Methods of Use of the Invention

There is evidence for a role of NLRP3-induced IL-1 and IL-18 in the inflammatory responses occurring in connection with, or as a result of a multitude of different diseases, disorders or abnormalities which is responsive to the modulation of a component of the NLRP3 inflammasome pathway and/or which is responsive to the modulation of IL-1 beta and/or IL-18 levels. (Menu et al., Clinical and Experimental Immunology, 2011, 166, 1-15; Strowig et al., Nature, 2012, 481, 278-286). The invention provides a compound of (I′), (I), (II′), (II), (II′a), (II′b), (IIa), (IIb), (III′), (III), (IV) or (V) as defined herein, or a stereoisomer, a racemic mixture, a tautomer, a polymorph, a pharmaceutically acceptable salt, a prodrug, a hydrate, or a solvate thereof, which exhibits valuable pharmacological properties, e.g. NRLP3 inhibiting properties on the NLRP3 inflammasome pathway. Said compounds of the invention may be useful in the treatment, alleviation or prevention of a disease, or a disorder or an abnormality which is responsive to the modulation of a component of the NLRP3 inflammasome pathway and/or which is responsive to the modulation of IL-1 beta and/or IL-18 levels. A number of diseases, disorders or abnormalities have been shown to be involve in NLRP3 including, for example, one of the following:
A. Central nervous system disease (CNS), disorder, or abnormality, such as Alzheimer's disease, Parkinson's disease, dementia, frontotemporal dementia, Huntington's disease, cerebral malaria, brain injury from pneumococcal meningitis, motor neuron disease, traumatic brain injury, spinal cord injury, neuropathic pain, migraine, amyotrophic lateral sclerosis, or multiple sclerosis (MS);
B. Immune disease, disorder, or abnormality (e.g. autoimmune disease, disorder or abnormality, and disease, disorder, or abnormality, involving the immune system), such as type 1 diabetes, hidradenitis suppurativa (HS), Schnitzler syndrome, multiple sclerosis (MS) including primary progressive multiple sclerosis (PPMS), Sjogren's syndrome, secondary progressive multiple sclerosis (SPMS), TNF receptor associated periodic syndrome (TRAPS), graft-versus host disease antiphospholipid syndrome, refractory celiac disease, autoimmune pancreatitis, or relapsing remitting multiple sclerosis (RRMS);
C. Inflammatory disease, including auto-inflammation and inflammation occurring as a result of an inflammatory disease, disorder, or abnormality, such as mevalonate kinase deficiency (MKD), hyperimmunoglobulinemia D, cryopyrin-associated periodic syndromes (CAPS), Muckle-Wells syndrome (MWS), familial cold autoinflammatory syndrome (FCAS), neonatal-onset multisystem inflammatory disease (NOMID), familial Mediterranean fever (FMF), acne, pyogenic arthritis, pyoderma gangrenosum and acne (PAPA), adult-onset Still's disease (AOSD), Majeed syndrome, PLCG2-associated antibody deficiency and immune dysregulation (PLAID), PLCG2-associated autoinflammation, antibody deficiency and immune dysregulation (APLAID), pyogenic arthritis, haploinsufficiency of A20 (HA20), pediatric granulomatous arthritis (PGA), or sideroblastic anemia with B-cell immunodeficiency, periodic fevers, developmental delay (SIFD);
D. Skin disease, disorder, or abnormality, including skin inflammatory conditions, such as hidradenitis suppurativa (HS), dermatitis, psoriasis, skin contact hypersensitivity, acne, periodic fever syndrome (HIDS), Sweet's syndrome, eczema, skin lesions, burn, wound, wound healing, trauma, sunburn, actinic keratosis, deficiency of interleukin 1 receptor (DIRA) antagonist, epidermolysis bullosa, vitiligo, atopic dermatitis, cutaneous lupus, or alopecia areata;
E. Ocular disease, disorder, or abnormality, such as age-related macular degeneration (AMD), corneal infection, uveitis, glaucoma, dry eye, Geographic Atrophy (GA), or demyelination;
F. Cardiovascular disease, disorder, or abnormality (e.g. disease, disorder, or abnormality of the cardiovascular system) such as myocardial infarction, hypertension, ischaemia reperfusion injury, pericarditis including Dressler's syndrome, aneurysms including abdominal aortic aneurism, heart failure, coronary artery disease, or stroke;
G. Metabolic disease, disorder, or abnormality, such as type 2 diabetes, obesity, Edema (DME), atherosclerosis, gout, or pseudo-gout;
H. Respiratory disease, disorder, or abnormality (e.g. disease, disorder or abnormality of the respiratory system), such as asbestosis, silicosis, cystic fibrosis, allergic inflammation, chronic obstructive pulmonary disorder (COPD), Coronavirus-associated respiratory distress syndrome (CARDS), steroid-resistant asthma, or asthma;
I. Liver disease, disorder, or abnormality, (e.g. hepatic disease, disorder or abnormality) such as hepatitis, primary biliary cholangitis, cytokine release syndrome, alcoholic liver disease, alcoholic fatty liver disease (AFLD), alcoholic steatohepatitis (ASH), non-alcoholic fatty liver disease (NAFLD), or non-alcoholic steatohepatitis (NASH) including advanced fibrosis stages F3 and F4;
J. Renal disease, disorder, or abnormality (e.g. disease, disorder or abnormality of the renal system) such as oxalate-induced nephropathy, diabetic nephropathy, lupus nephritis, chronic kidney disease, or acute kidney disease;
K. Cancer disease, disorder, or abnormality (e.g. cancer, tumor, or malignancy), such as lung cancer (e.g. lung cancer metastasis), pancreatic cancers, gastric cancers, leukemia, myelodysplastic syndrome (MOS), skin cancer, tumors of the endocrine system, or thyroid cancer;
L. Infections including viral infections, such as helminth infections (e.g. from schistosoma, roundworms, tapeworms or flukes), viral encephalitis, bacterial infection, periodontitis, human immunodeficiency virus (HIV), HIV-associated neurocognitive disorder, chronic nonbacterial osteomyelitis (CNO), chronic bacterial osteomyelitis, deficiency of interleukin 1 receptor (DIRA) antagonist, or epilepsy; alphavirus (e.g. Chikungunya virus and Ross River virus), flaviviruses (e.g. Dengue and Zika virus), Coronavirus-associated inflammatory pathologies, Coronaviruses, or influenza virus;
M. Psychological disease, disorder, or abnormality, such as depression, and psychological stress;
N. Inflammation, including inflammation occurring as a result of an inflammatory disease, disorder, or abnormality, such as an autoinflammatory disease, inflammation occurring as a symptom of a non-inflammatory disorder, inflammation occurring as a result of infection, or inflammation secondary to trauma, injury or autoimmunity. Examples of inflammation include inflammatory responses occurring in connection with, or as a result of:
   i. A joint disease, disorder, or abnormality, such as periodic fever syndrome (HIDS), rheumatoid arthritis, pustulosis, synovitis, osteoarthritis, chronic recurrent multifocal osteomyelitis (CRMO), systemic juvenile idiopathic arthritis, osteitis syndrome (SAPHO), hyperostosis, relapsing polychondritis, ankylosing spondylitis, or adult-onset Still's disease;
   ii. A gastrointestinal disease, disorder, or abnormality (e.g. disease, disorder or abnormality of the gastrointestinal tract) such as colitis, ulcerative colitis, or inflammatory bowel disease;
   iii. A muscular disease, disorder, or abnormality, such as polymyositis, or myasthenia gravis;
   iv. A disease, disorder or abnormality of the endocrine system, such as, diabetes, parathyroid disease (e.g. hypothyroidism), tumors of the endocrine system, thyroid cancer, or hypoglycemia; and/or
   v. A vascular disease, disorder or abnormality, such as Behcet's disease or mucocutaneous lymph node syndrome.

In one embodiment, the disease, disorder, or abnormality is selected from Alzheimer's disease, Parkinson's disease, cryopyrin-associated periodic syndromes (CAPS), non-alcoholic fatty liver disease (NAFLD), non-alcoholic steatohepatitis (NASH), and hidradenitis suppurativa (HS).

In particular, the disease, disorder or abnormality is selected from: Alzheimer's disease, Parkinson's disease, amyotrophic lateral sclerosis, multiple sclerosis, demyelination, viral encephalitis, epilepsy, stroke, brain haemorrhage, atherosclerosis, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease (IBD) (including Crohn's disease, ulcerative colitis), hepatitis, nonalcoholic fatty liver disease, nonalcoholic steatohepatitis, hypertension, myocardial infarction, heart failure, coronary artery disease, oxalate-induced nephropathy, graft-versus host disease, type 1 diabetes, type 2 diabetes, Edema (DME), Geographic Atrophy (GA), rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, skin contact hypersensitivity, sunburn, psoriasis, hidradenitis suppurativa (HS), epidermolysis bullosa, acne, eczema, alopecia areata, actinic keratosis, hyperostosis, osteitis syndrome (SAPHO), vitiligo, atopic dermatitis, cutaneous lupus, multiple sclerosis (MS), Behcet's disease, Sjogren's syndrome, Schnitzler syndrome, chronic obstructive pulmonary disorder (COPD), asthma, steroid-resistant asthma, Coronavirus-associated inflammatory pathologies including Coronavirus-associated respiratory distress syndrome (CARDS), asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, acute kidney injury, chronic kidney disease, lupus nephritis diabetic nephropathy, alcoholic liver disease, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, primary biliary cholangitis, antiphospholipid syndrome, refractory celiac disease, pancreatitis, autoimmune pancreatitis, mucocutaneous lymph node syndrome, lung cancer metastasis, pancreatic cancers, gastric cancers, myelodisplastic syndrome, leukemia; polymyositis, colitis, helminth infection, bacterial infection, abdominal aortic aneurism, wound healing, migraine, depression, psychological stress, pain, neuropathic pain, periodontitis, pericarditis including Dressler's syndrome, ischaemia reperfusion injury, frontotemporal dementia, HIV-associated neurocognitive disorder, traumatic brain injury, traumatic spinal cord injury, inflammatory pain, chronic pain, neuropathic pain, metastatic cancer-induced bone pain, chemotherapy induced peripheral neuropathy and migraine; ankylosing spondylitis, cytokine release syndrome. Preferably, the diseases, the disorders or the abnormalities are selected from Alzheimer's disease, Parkinson's disease, amyotrophic lateral sclerosis, demyelination, multiple sclerosis, encephalomyelitis, leukoencephalopathy, viral encephalitis, epilepsy, stroke, traumatic brain and spinal cord injury, atherosclerosis, asthma and allergic inflammation, cryopyrin-associated periodic syndromes (CAPS), gout, inflammatory bowel disease (IBD), non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, hidradenitis suppurativa (HS), rheumatoid arthritis, acute kidney disease, chronic kidney disease, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, and Hereditary Recurrent Fevers (HRFs), and amyloidosis (including AL amyloidosis, AA amyloidosis, ATTR amyloidosis, hereditary amyloidosis (including apolipoprotein A-I (AApoAI), apolipoprotein A-II (AApoAII), gelsolin (AGel), fibrinogen (AFib), and lysozyme (ALys)), Beta-2 Microglobulin amyloidosis, iAPP amyloidosis).

In one embodiment, the diseases, the disorders or the abnormalities which are responsive to the modulation, in particular inhibition of activation, of a component of the NLRP3 inflammasome pathway can be selected from Alzheimer's disease, Parkinson's disease, amyotrophic lateral sclerosis, demyelination, viral encephalitis, epilepsy, stroke, atherosclerosis, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease (IBD) (including Crohn's disease, ulcerative colitis), hepatitis, nonalcoholic fatty liver disease, nonalcoholic steatohepatitis, hypertension, myocardial infarction, heart failure, coronary artery disease, oxalate-induced nephropathy, graft-versus host disease, type 1 diabetes, type 2 diabetes, Edema (DME), Geographic Atrophy (GA), rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, skin contact hypersensitivity, sunburn, psoriasis, hidradenitis suppurativa (HS), epidermolysis bullosa, acne, eczema, alopecia areata, actinic keratosis, hyperostosis, osteitis syndrome (SAPHO), vitiligo, atopic dermatitis, cutaneous lupus, multiple sclerosis (MS), Behcet's disease, Sjogren's syndrome, Schnitzler syndrome, chronic obstructive pulmonary disorder (COPD), asthma, steroid-resistant asthma, Coronavirus-associated inflammatory pathologies including Coronavirus-associated respiratory distress syndrome (CARDS), asbestosis, silicosis, cystic fibrosis, motor neuron disease, Huntington's disease, cerebral malaria, brain injury from pneumococcal meningitis, obesity, age-related macular degeneration (AMD), comeal infection, uveitis, dry eye, acute kidney injury, chronic kidney disease, lupus nephritis diabetic nephropathy, alcoholic liver disease, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, primary biliary cholangitis, antiphospholipid syndrome, refractory celiac disease, pancreatitis, autoimmune pancreatitis, mucocutaneous lymph node syndrome, lung cancer metastasis, pancreatic cancers, gastric cancers, myelodisplastic syndrome, leukemia; polymyositis, colitis, helminth infection, bacterial infection, abdominal aortic aneurism, wound healing, migraine, depression, psychological stress, pain, neuropathic pain, periodontitis, pericarditis including Dressler's syndrome, ischaemia reperfusion injury, frontotemporal dementia, HIV-associated neurocognitive disorder, traumatic brain injury, traumatic spinal cord injury, ankylosing spondylitis, cytokine release syndrome. Preferably, the diseases, the disorders or the abnormalities are selected from Alzheimer's disease, Parkinson's disease, amyotrophic lateral sclerosis, demyelination, multiple sclerosis, encephalomyelitis, leukoencephalopathy, viral encephalitis, epilepsy, stroke, traumatic brain and spinal cord injury, atherosclerosis, asthma and allergic inflammation, cryopyrin-associated periodic syndromes (CAPS), gout, inflammatory bowel disease (IBD), non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, hidradenitis suppurativa (HS), rheumatoid arthritis, acute kidney disease, chronic kidney disease, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, and Hereditary Recurrent Fevers (HRFs).

### Definitions

Within the meaning of the present application the following definitions apply unless specified otherwise, and when appropriate, terms used in the singular will also include the plural and vice versa:

“Alkyl” refers to a saturated straight or branched organic moiety consisting of carbon and hydrogen atoms. Examples of suitable alkyl groups have 1 to 4 carbon atoms, preferably 1 to 3 carbon atoms, and (as appropriate) include methyl, ethyl, propyl, isopropyl, n-butyl, t-butyl, and isobutyl. The term “C₁-C₆alkyl” refers to an alkyl group having 1 to 6 carbon atoms. The terms “C₁-C₄alkyl”, “C₁-C₃alkyl”, or “C₁alkyl” are to be construed accordingly.

“Hal”, “halo” or “halogen” refers to F, Cl, Br, and I. Preferably halogen is F or Cl. More preferably, halogen is Cl. Even more preferably, halogen is F.

“—O—C₁-C₆alkyl” where “C₁-C₆alkyl” is as generally defined above. Examples of “—O—C₁-C₆alkyl” include, but are not limited to methoxy, ethoxy, propoxy, isopropoxy, pentoxy, and hexoxy.

The term “C₃-C₆cycloalkyl” refers to saturated monocyclic hydrocarbyl groups having 3 to 6 carbon atoms. The terms “C₅-C₆cycloalkyl” is to be construed accordingly. Examples include, but are not limited to cyclopropyl, cyclobutyl, cyclopentyl, and cyclohexyl.

“4-, 5- or 6-membered heterocycloalkyl” refers to a stable 4-, 5- or 6-membered non-aromatic monocyclic ring radical which comprises 1 or 2 heteroatoms. The heteroatom is independently selected from nitrogen and oxygen. Examples include azetidine, oxetane, pyrrolidine, tetrahydrofurane, oxazolidine, isoxazolidine, piperidine, and morpholine, preferably pyrrolidine, and piperidine.

“8-, 9- or 10-membered bicyclic heterocycloalkyl” refers to a stable 8-, 9- or 10-membered non-aromatic fused bicyclic ring radical which comprises 1, 2 or 3 heteroatoms. The heteroatom(s) is/are preferably independently selected from nitrogen and oxygen. Examples of the 8-, 9- or 10-membered bicyclic heterocycloalkyl include 6-methyloctahydro-pyrrolo[2,3-c]pyridine such as

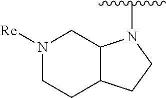

with Re being selected from C₁-C₆alkyl, haloC₁-C₆alkyl, hydroxyC₁-C₆alkyl or —OH, preferably.

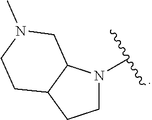 [SMILES: *N1CCC2CCN(C)CC21]

The dashed circle in the five-membered ring means that double bonds can be optionally present at any available position. According to the rules of chemistry C which is an option of Z, E, and Q has four bonds to adjacent atoms and N which is an option of Z, E and Q has three bonds to adjacent atoms. The bonds can be either single or double bonds. Examples of the five-membered ring include; pyrazolo

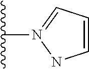

or imidazo,

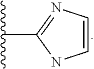 [SMILES: *c1ncc[nH]1]

[symbol]

identifies the point of bonding. For instance, with respect to the compound having the formula (I′) the wavy line [symbol] indicates the atom of R_{A} and R_{B} at which R_{A} and R_{B} are bound to the core structure of the compound having the formula (I′).

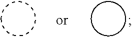

the circles (dashed or closed) mean that double bonds can be present at any available position in the ring.

“Optionally substituted” in reference to a certain group refers to said group as to optionally be substituted with one or more substituents (i.e. the substituent may be present or not).

Unless specified otherwise, the term “compound of the present invention” refers to compounds of formula (I′), (I), (II′), (II), (II′a), (II′b), (IIa), (IIb), (III′), (III), (IV) or (V) as disclosed herein, or subformulae thereof, as disclosed herein, or stereoisomers thereof, or racemic mixtures thereof, or tautomers thereof, or polymorphs thereof, or pharmaceutically acceptable salts thereof, or prodrugs thereof, or hydrates thereof, or solvates thereof. Compounds of the present invention having one or more optically active carbons can exist as racemates and racemic mixtures (including mixtures in all ratios), stereoisomers (including diastereomeric mixtures and individual diastereomers, enantiomeric mixtures and single enantiomers, mixtures of conformers and single conformers), tautomers, atropisomers, and rotamers. All isomeric forms are included in the present invention. Compounds described in this invention containing olefinic double bonds include E and Z geometric isomers. Also included in this invention are all pharmaceutically acceptable salts, prodrugs, hydrates and solvates of compounds of formula (I′), (I), (II′), (II), (II′a), (II′b), (IIa), (IIb), (III), (III), (IV) or (V).

Tautomers are isomers of a compound which differ only in the position of the protons and electrons. The skeleton of the compound is unchanged. Common tautomeric pairs include: ketone-enol (H—O—C═CH⇄O═C—CH₂), enamine-imine (H₂N—C═N⇄HN═C—NH).

Solvates, hydrates as well as anhydrous forms of the salt are also encompassed by the invention. The solvent included in the solvates is not particularly limited and can be any pharmaceutically acceptable solvent. Examples include water and C₁₋₄ alcohols (such as methanol or ethanol).

“Pharmaceutically acceptable salts” are defined as derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric acid and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic acid, and the like. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts can be found in Remington's Pharmaceutical Sciences, 18^{th} ed., Mack Publishing Company, Easton, P A, 1990, p. 1445, the disclosure of which is hereby incorporated by reference.

The compounds of the present invention, as defined herein, can also be provided in the form of a prodrug, namely a compound which is metabolized in vivo to the active metabolite. As used hereinafter in the description of the invention and in the claims, the term “prodrug” means any covalently bonded compound which releases the active parent pharmaceutical due to in vivo biotransformation. The reference by Goodman and Gilman (The Pharmacological Basis of Therapeutics, 8 ed, McGraw-Hill, Int. Ed. 1992, “Biotransformation of Drugs”, p 13-15) describing prodrugs generally is hereby incorporated herein by reference.

“Pharmaceutically acceptable” is defined as those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

A used herein, the terms “patient” or “subject” mentioned in the present invention typically refer to an animal, particularly a mammal (e.g. rabbits, rats, dogs, mice, guinea pigs, pigs), more particularly primates (e.g. humans, male or female). In certain embodiments, the subject is a human.

“NLRP3” as used herein refers to NOD-like receptor (NLR) family, pyrin-domain containing protein 3 component of inflammasome. Inflammasomes are intracellular supramolecular complexes comprising a sensor molecule, the adaptor apoptosis-associated speck-like protein containing a CARD (ASC) and the effector protease caspase 1. Upon activation of the inflammasome sensor molecule, ASC self-associates into a helical fibrillary assembly resulting in formation of the so-called ASC speck or pyroptosome, which acts as a molecular platform for the activation of pro-caspase 1 via proximity-induced autocatalytic activation. Active caspase 1 triggers the activation and release of interleukin-1 (IL-1) family proteins and enables the non-conventional secretion of numerous cytosolic proteins. Among the pro-inflammatory mediators released upon NLRP3 activation are IL-1 beta (3), IL-18, high-mobility group protein B1 (HMGB1), leukotrienes and prostaglandins.

NLRP3 inflammasome pathway activation is an important driver of inflammation interacting with the different cytokine pathways shaping the immune response to infection and injury. Formation of some pro-inflammatory cytokines is triggered by NLRP3 inflammasome pathway activation.

The terms “inhibit”, “inhibition” or “inhibiting” refer to the reduction or suppression of a given condition, symptom, or disorder, or disease, or abnormality which is responsive to the modulation of a component of the NLRP3 inflammasome pathway, or a significant decrease in the baseline activity of a biological activity or process.

The terms “treat”, “treating” or “treatment” of any disease, disorder or abnormality refer to alleviating or ameliorating or modulating the disease or disorder or abnormality (i.e., slowing or arresting the development of the disease, disorder or abnormality or at least one of the clinical symptoms thereof); or alleviating or ameliorating or modulating at least one physical parameter or biomarker associated with the disease or disorder or abnormality, including those which may not be discernible to the subject (e.g., patient).

The terms “prevent”, “preventing” or “prevention” of any disease or disorder or abnormality which is responsive to the modulation of a component of the NLRP3 inflammasome pathway refer to the prophylactic treatment of the disease or disorder or abnormality; or delaying the onset or progression of the disease or disorder.

The term “in need of” a treatment if such subject would benefit biologically, medically or in quality of life from such treatment.

As used herein, “modulation” refers to alteration, e.g., up-regulation, down-regulation, increase or decrease, preferably decrease.

| Abbreviation | Meaning |
|---|---|
| NALP1-14 | NACHT Leucine-rich-repeat Protein 1-14 (a synonym of NLRP) |
| IPAF | Ice Protease-Activating Factor |
| NAIP | Neuronal Apoptosis Inhibitory Protein |
| ASC | Apoptosis-associated Speck-like protein containing a CARD |
| nucleotide- | NACHT: NAIP (neuronal apoptosis inhibitory protein), CIITA (MHC class |
| binding NACHT | Il transcription activator), HET-E (incompatibility locus protein from |
| domain | Podospora anserina) and TP1 (telomerase-associated protein) |
| IL | Interleukin |
| TNF-alpha | Tumor Necrosis Factor-alpha |

The definitions and preferred definitions given in the “Definition”-section apply to all of the embodiments described herein unless stated otherwise.

General Synthetic Scheme for the Preparation of Compounds of this Invention:

The compounds of the present invention can be synthesized by those skilled in the art by using commonly known preparation steps, for instance those of the general methods shown in the following schemes. These methods are only given for illustrative purposes and should not be construed as limiting.

In all of the methods it is understood that protecting groups for sensitive or reactive groups may be employed where necessary in accordance with general principles of chemistry. Protecting groups are manipulated according to standard methods of organic synthesis (T. W. Green and P. G. M. Wuts (2014) Protective Groups in Organic Synthesis, 5th edition, John Wiley & Sons). These groups are removed at a convenient stage of the compound synthesis using methods that are readily apparent to those skilled in the art.

In the following general methods, R₀, R₁, R₂, R₃, Rₐ, X′ and Y are as previously defined in the above embodiments.

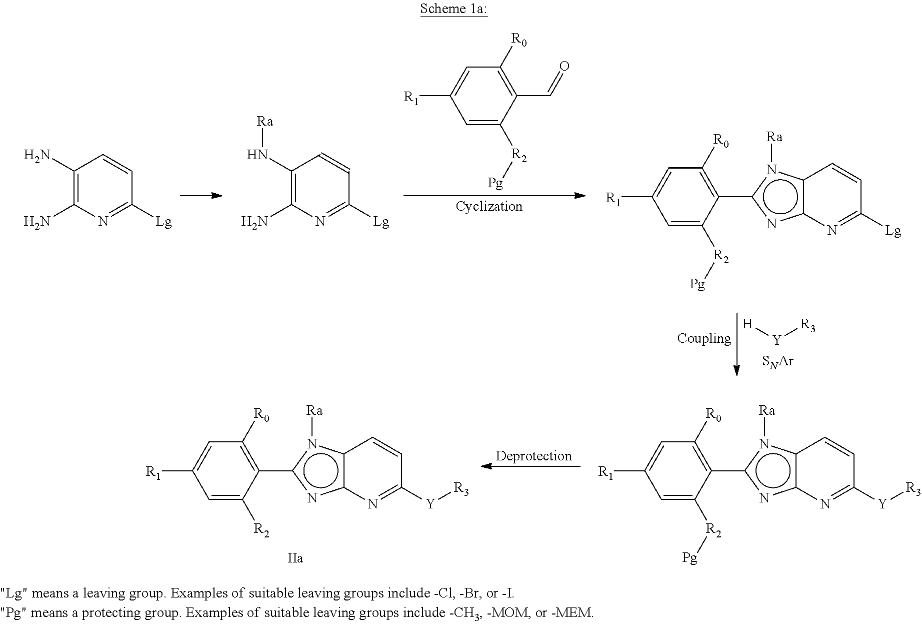

Commercially available diamino pyridines can be functionalized. In the case of Rₐ being methyl, a two-step strategy can be applied. First, selective carbamate formation using CbzCl followed by reduction using for example LAH can deliver the desired intermediate. Alternatively, commercially available diamino pyridine can be selectively functionalized with ethyl iodide or 2-bromopropane to obtain the desired intermediate with Rₐ being ethyl or isopropyl. Then, cyclization using commercially aldehydes using an appropriate solvent in the presence of sodium bisulfite can provide the bicyclic intermediate after purification. In case the aromatic aldehydes are not commercially available, they can be prepared via multistep reactions from commercially available starting materials involving Suzuki coupling reactions, nucleophilic substitutions, halogen-lithium exchange reactions employing BuLi and DMF, or palladium mediated introduction of —OCH₃ with dipotassium; sulfonatooxy sulfate and 3-(trifluoromethyl)aniline. Finally, the bicyclic intermediates can be further functionalized using palladium catalysed Buchwald reactions employing suitable amines and alcohols or by nucleophilic substitution to afford compounds of formula (IIa) after purification. In case a protecting group is present at R2, the protecting group can be cleaved under acidic conditions (boron tribromide, TFA) to deliver compounds of formula (IIb) after purification. In case compounds of formula (IIa) contain a secondary amine, employing reductive amination conditions compounds of formula (IIa) containing a tertiary amine can be obtained. In case racemic mixtures are obtained, they can be separated by SFC to afford compounds of formula (IIa).

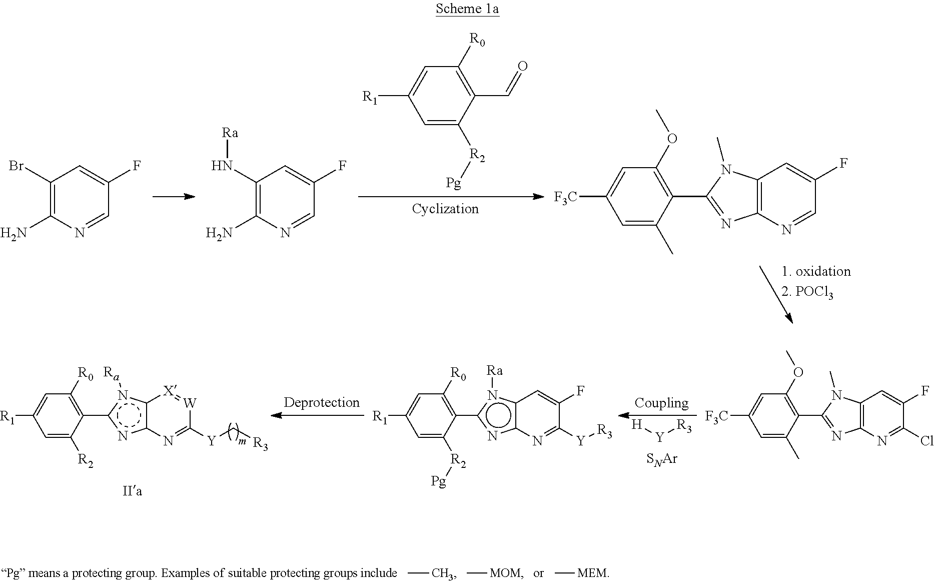

In case a diamino pyridine without the desired substitution pattern is not commercially available, an amino pyridine derivative containing a bromo atom can be treated with suitable amines to afford the diamino pyridine derivative. Then, cyclization using commercially aldehydes using an appropriate solvent in the presence of sodium bisulfite can provide the bicyclic intermediate after purification. The bicyclic intermediates can be oxidized (3-chloroperoxybenzoic acid), followed by phosphoryl chloride treatment to afford bicyclic intermediates containing a chloro leaving group. Finally, the bicyclic intermediates can be further functionalized using palladium catalysed Buchwald reactions or by nucleophilic substitution to afford compounds of formula (IIa) after cleavage of the protecting group (BBr3). In case racemic mixtures are obtained, they can be separated by SFC to afford compounds of formula (II′a).

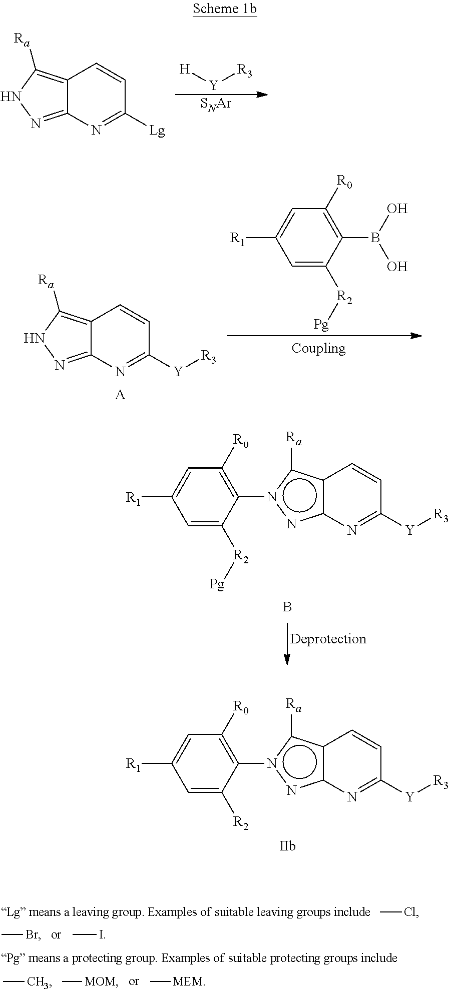

Commercially available bicyclic scaffolds can be functionalized by S_{N}Ar using appropriate amines or alcohols or sodium; 2-chloro-2,2-difluoro-acetate. Then, reacting the pyrazole ring with appropriate boronic acids or esters employing a suitable copper catalyst can yield intermediates B. In case a protecting group is present at R2, the protecting group can be cleaved under acidic conditions (boron tribromide) can deliver compounds of formula (IIb) after purification.

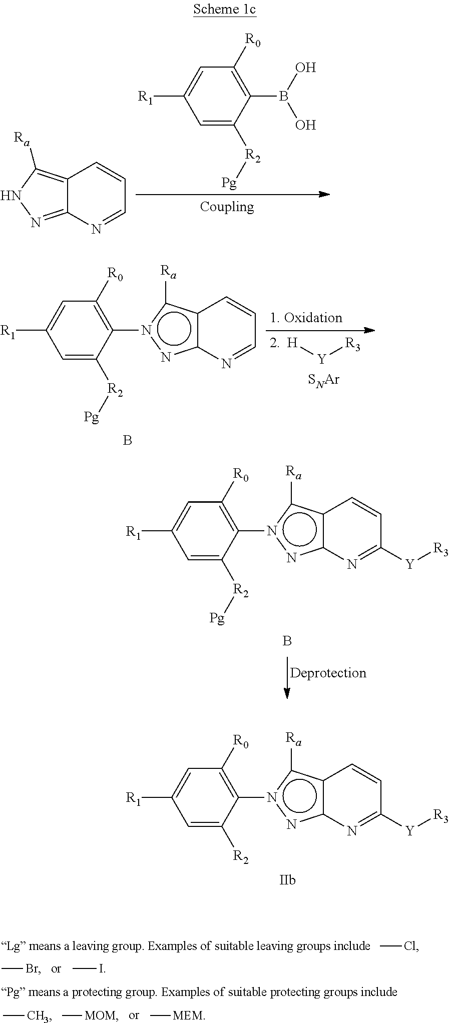

Commercially available bicyclic scaffolds can be functionalized by S_{N}Ar using appropriate thiols. Then, reacting the pyrazole ring with appropriate boronic acids or esters employing a suitable copper catalyst can yield intermediates B. In case intermediate B contains a thioether moiety at the —Y—R₃ position, the thioether can be converted to a leaving group by oxidation (3-chloroperoxybenzoic acid), which can then be replaced with a suitable amine via nucleophilic substitution to afford intermediates B. In case a protecting group is present at R₂, the protecting group can be cleaved under acidic conditions (boron tribromide) can deliver compounds of formula (IIb) after purification.

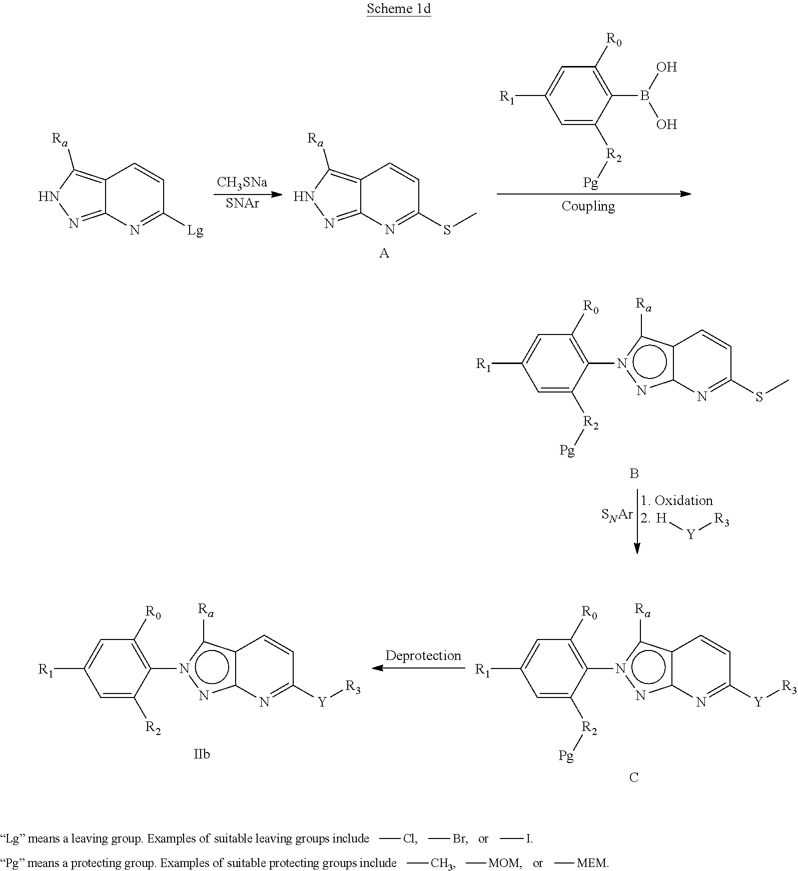

Commercially available bicyclic scaffolds can be functionalized by S_{N}Ar using appropriate thiols. Then, reacting the pyrazole ring with appropriate boronic acids or esters employing a suitable copper catalyst can yield intermediates B. In case intermediate B contains a thioether moiety, the thioether can be converted to a leaving group by oxidation (3-chloroperoxybenzoic acid), which can then be replaced with a suitable amine via nucleophilic substitution to afford intermediates C. In case a protecting group is present at R2, the protecting group can be cleaved under acidic conditions (boron tribromide) can deliver compounds of formula (IIb) after purification.

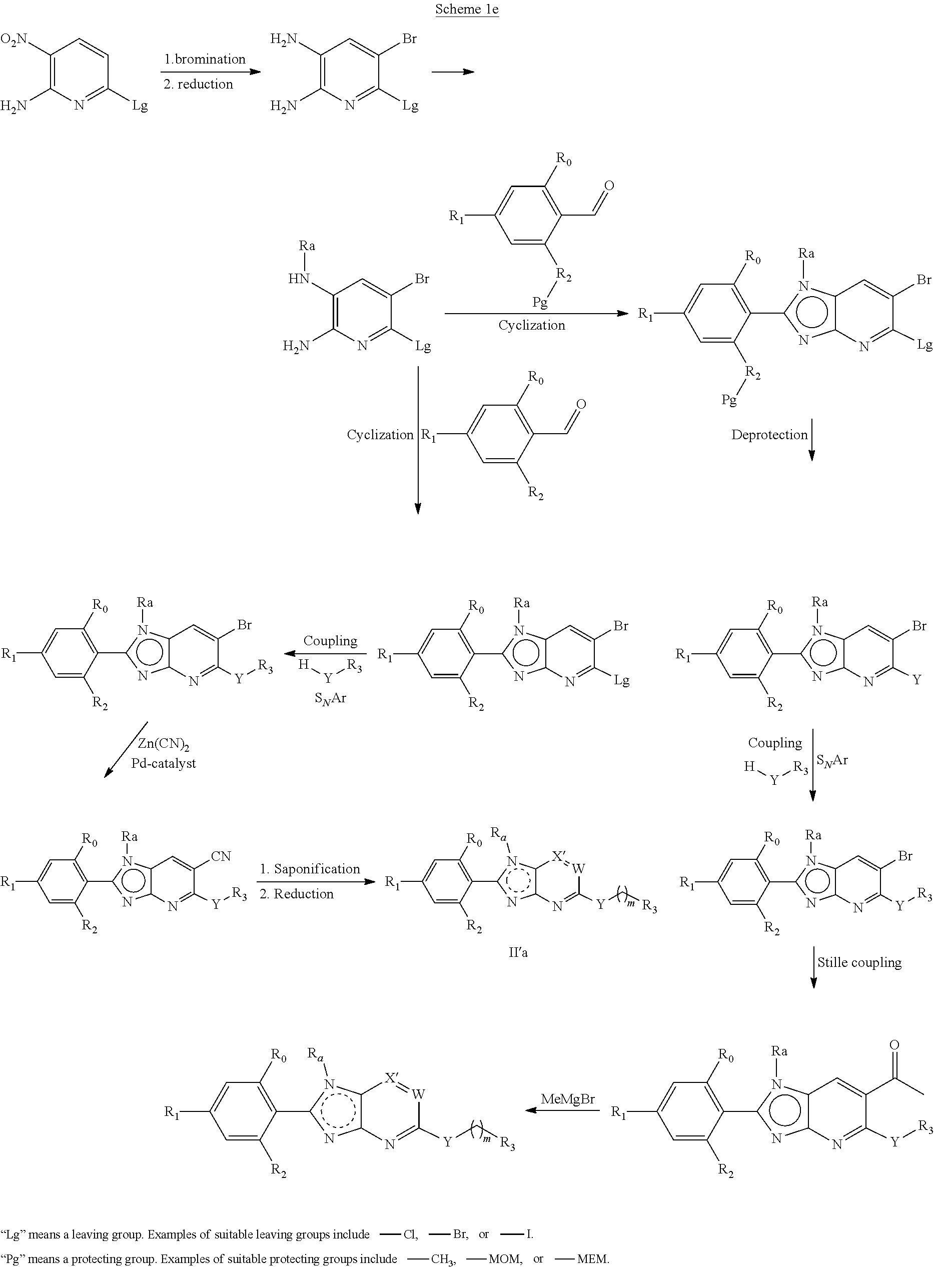

Commercially available amino-nitro pyridines can be functionalized to diamino pyridines by bromination (NBS) and subsequent reduction of the nitro group (Fe, acid). In the case of Rₐ being methyl, a two-step strategy can be applied. First, selective carbamate formation using CbzCl followed by reduction using for example LAH can deliver the desired intermediate. Then, cyclization using commercially aldehydes using an appropriate solvent in the presence of sodium bisulfite can provide the bicyclic intermediates after purification. Finally, the bicyclic intermediates can be further functionalized by nucleophilic substitution. Palladium mediated exchange of the bromo-moiety by a nitrile afforded the nitrile containing bicyclic intermediates. Saponification of the nitrile moiety to an acid followed by borane reduction afforded compounds of formula (II′a) containing a primary alcohol moiety. Alternatively, cyclization using aldehydes containing protecting groups using an appropriate solvent in the presence of sodium bisulfite can provide the bicyclic intermediates after purification. Acid mediated (boron tribromide) cleavage of the protection afforded bicyclic intermediates after purification. Nucleophilic substitution followed by Stille coupling using a suitable tin-reagent afforded compounds containing a methyl-ketone moiety. Treating the methyl-ketone derivatives with a Grignard reagent (CH₃MgBr) afforded compounds of formula (II′a) containing a tertiary alcohol moiety.

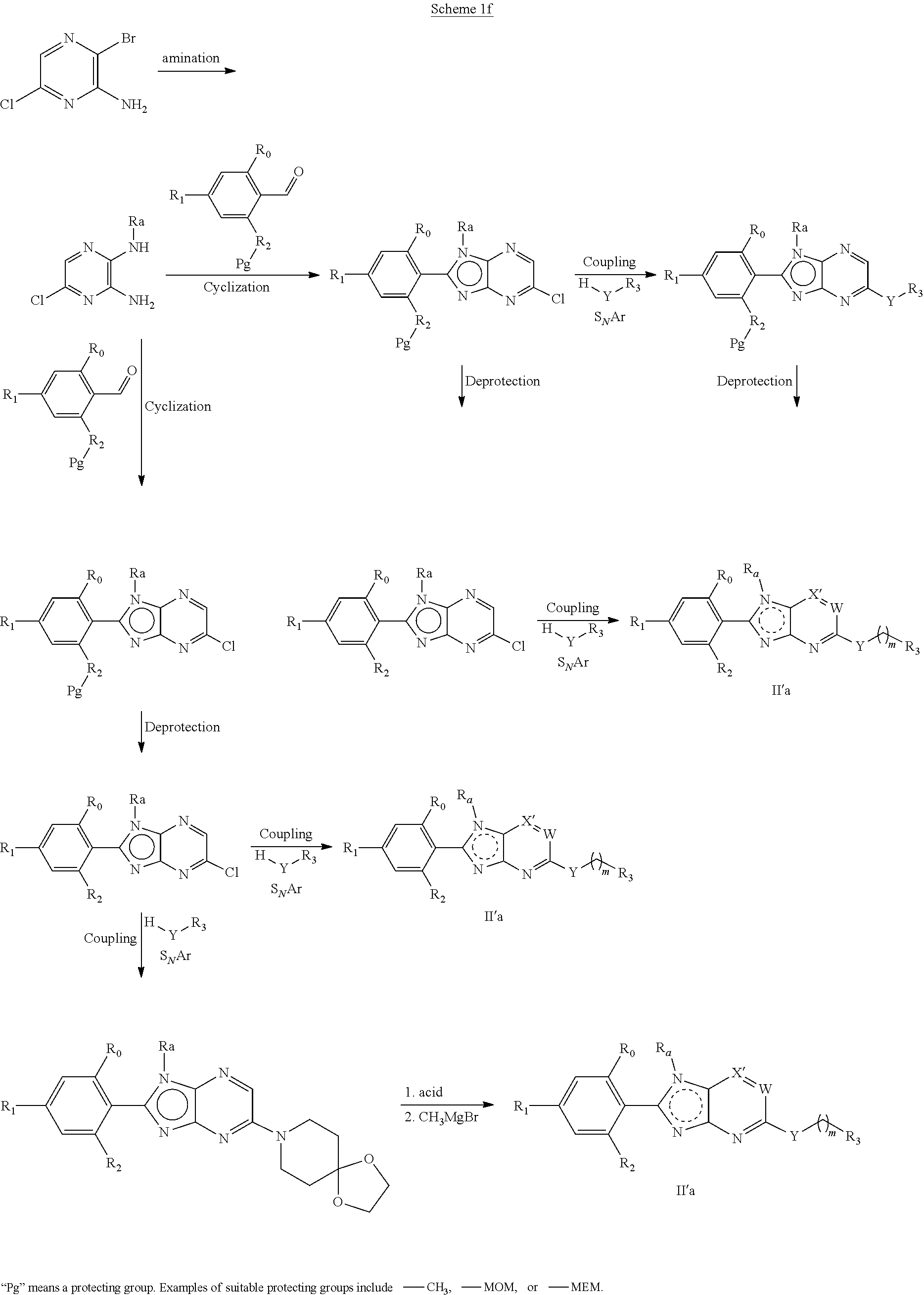

Commercially available 3-bromo-6-chloropyrazin-2-amine can be functionalized to diamino pyrazines by amination with suitable amines. Then, cyclization using aldehydes, containing alcohol protecting groups as MEM of MOM, employing an appropriate solvent in the presence of sodium bisulfite can provide the bicyclic intermediates after purification. Acid mediated cleavage of the protecting group (TFA) afforded the bicyclic intermediates after purification. Finally, the bicyclic intermediates can be further functionalized by nucleophilic substitution with suitable alcohols and amines to afford compounds of formula (II′a). In case the nucleophilic substitution product contains an acetal moiety, compounds of formula (II′a) containing a tertiary alcohol can be obtained after acetal cleavage and subsequent Grignard reaction. In case racemic mixtures are obtained, they can be separated by SFC to afford compounds of formula (II′a). Alternatively, cyclization using aldehydes, containing alcohol protecting groups as methyl, employing an appropriate solvent in the presence of sodium bisulfite can provide the bicyclic intermediates after purification. The bicyclic intermediates can be further functionalized by nucleophilic substitution with suitable alcohols and amine or palladium catalysed Buchwald conditions employing suitable amines. Finally, the methyl protecting group can be cleaved under Lewis-acid conditions (boron tribromide) to afford compounds of formula of formula (II′a). Alternatively, cyclization using aldehydes, containing alcohol protecting groups as methyl, employing an appropriate solvent in the presence of sodium bisulfite can provide the bicyclic intermediates after purification. The methyl protecting group can be cleaved under Lewis-acid conditions (borontribromide) to afford bicyclic intermediates containing a chloro leaving group. Finally, the bicyclic intermediates can be further functionalized by nucleophilic substitution with suitable alcohols and amine or palladium catalysed Buchwald conditions employing suitable amines to afford compounds of formula (II′a). In case compounds of formula (II′a) contain a secondary amine, employing reductive amination conditions compounds of formula (II′a) containing a tertiary amine can be obtained.

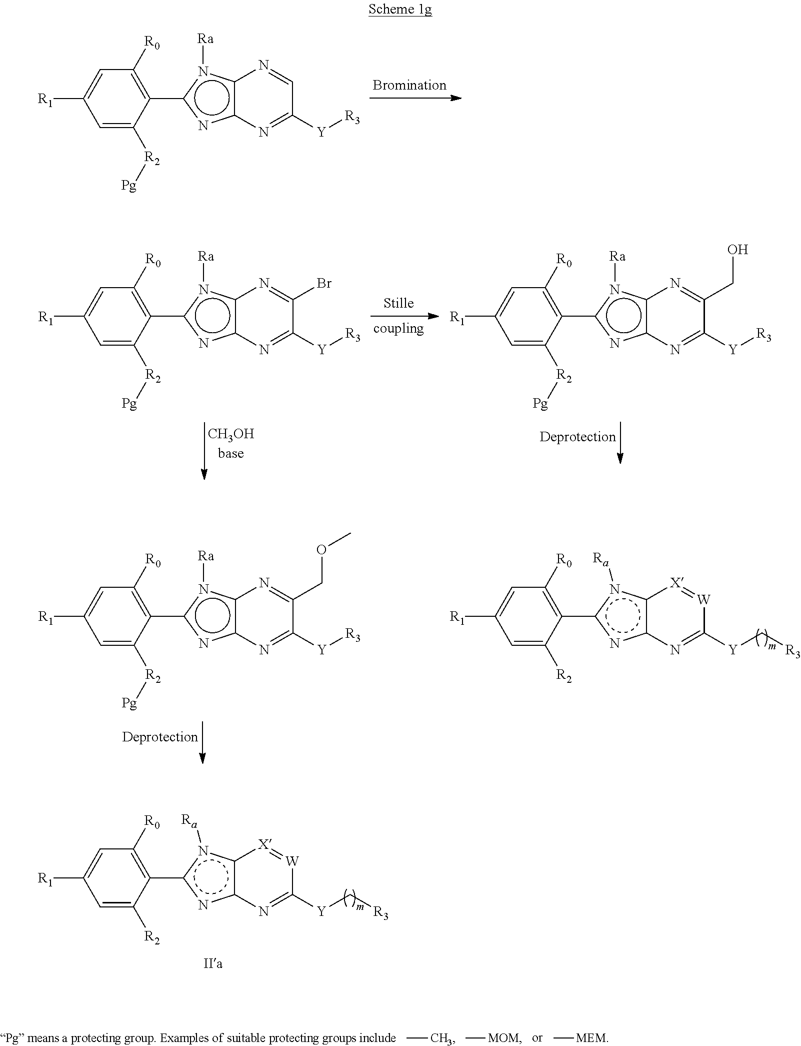

Bicyclic intermediates containing a methyl protecting group can be brominated (NBS) to afford the bromo-containing bicyclic intermediates. Bromo versus —CH₂OH exchange employing Stille coupling conditions afforded compounds of formula (II′a) after acid mediated (boron tribromide) cleavage of the methyl protecting group. Alternatively, bromo-containing bicyclic intermediates can be treated with methanol under basic conditions to afford —CH₂OCH₃ containing compounds of formula (II′a) after acid mediated (boron tribromide) cleavage of the methyl protecting group. In case compounds of formula (II′a) contain a secondary amine, employing reductive amination conditions compounds of formula (II′a) containing a tertiary amine can be obtained.

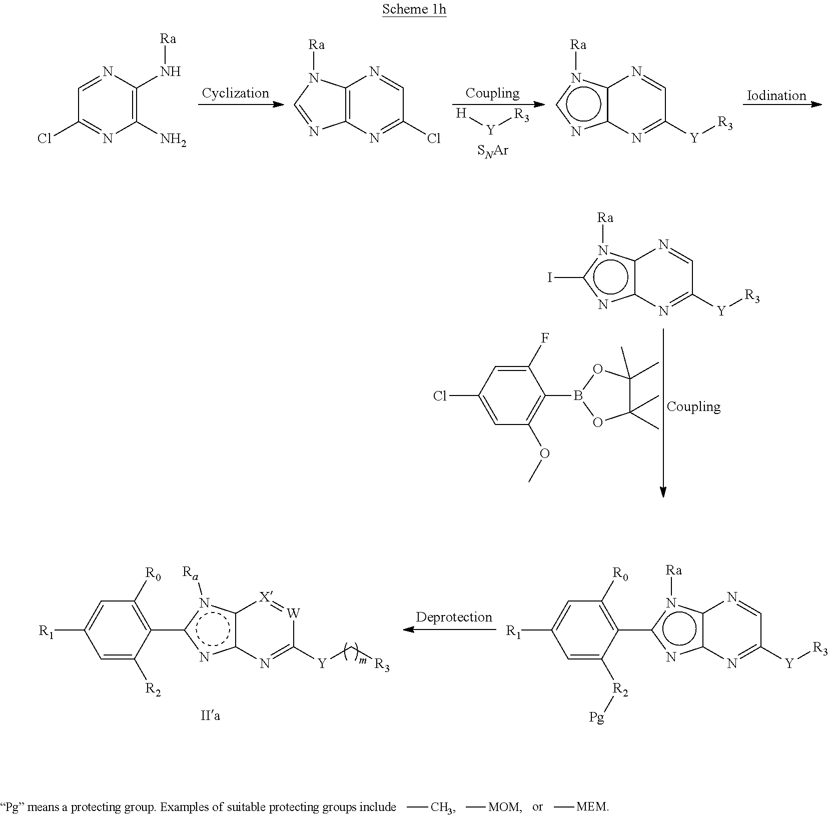

Suitable diamino pyrazines can be cyclized with ortho-esters to afford bicyclic intermediates. The bicyclic intermediates can be further functionalized by nucleophilic substitution with suitable aminoalcohols containing an amine protecting group. Iodination under basic conditions with 1,2,3,4,5-pentafluoro-6-iodo-benzene afforded the bicyclic intermediates containing an iodo moiety. Suzuki coupling employing a suitable boronic ester afforded the corresponding coupling products, which can be converted to compounds of formula of formula (II′a) after acid (boron tribromide) mediated deprotection. In case compounds of formula (II′a) contain a secondary amine, employing reductive amination conditions compounds of formula (II′a) containing a tertiary amine can be obtained.

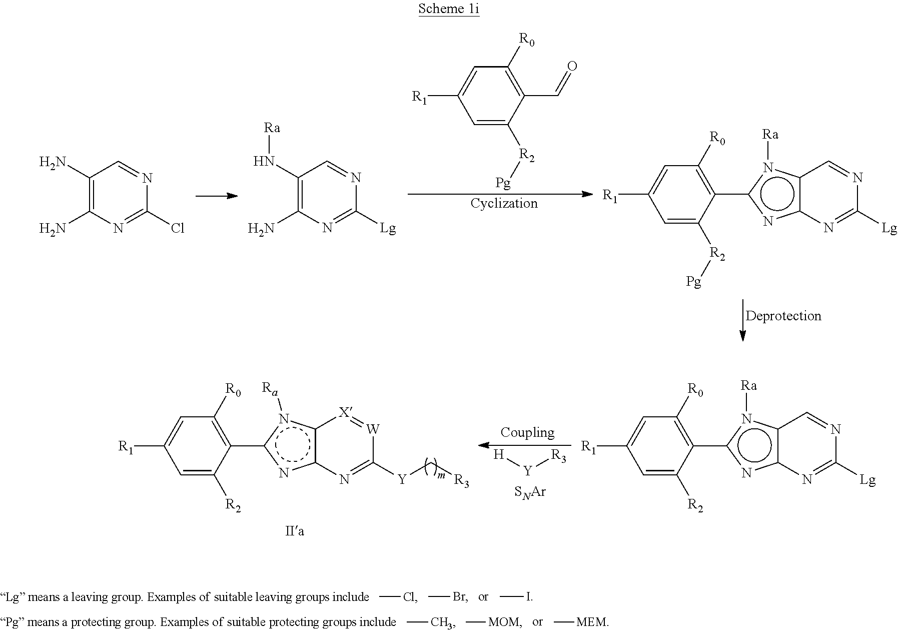

Commercially available 2-chloropyrimidine-4,5-diamine can be selectively functionalized with methyl iodide to obtain the desired intermediate with Rₐ being methyl. Then, cyclization using commercially aldehydes using an appropriate solvent in the presence of sodium bisulfite can provide the bicyclic intermediate after purification. In case the aromatic aldehydes are not commercially available, they can be prepared via multistep reactions from commercially available starting materials involving Suzuki coupling reactions, nucleophilic substitutions, halogen-lithium exchange reactions employing BuLi and DMF, or palladium mediated introduction of —OCH₃ with dipotassium; sulfonatooxy sulfate and 3-(trifluoromethyl)aniline. The protecting group present at R2 can be cleaved under acidic conditions (TFA) to obtain the deprotected bicyclic intermediates after purification. Finally, the bicyclic intermediates can be further functionalized by nucleophilic substitution to afford compounds of formula (II′a) after purification.

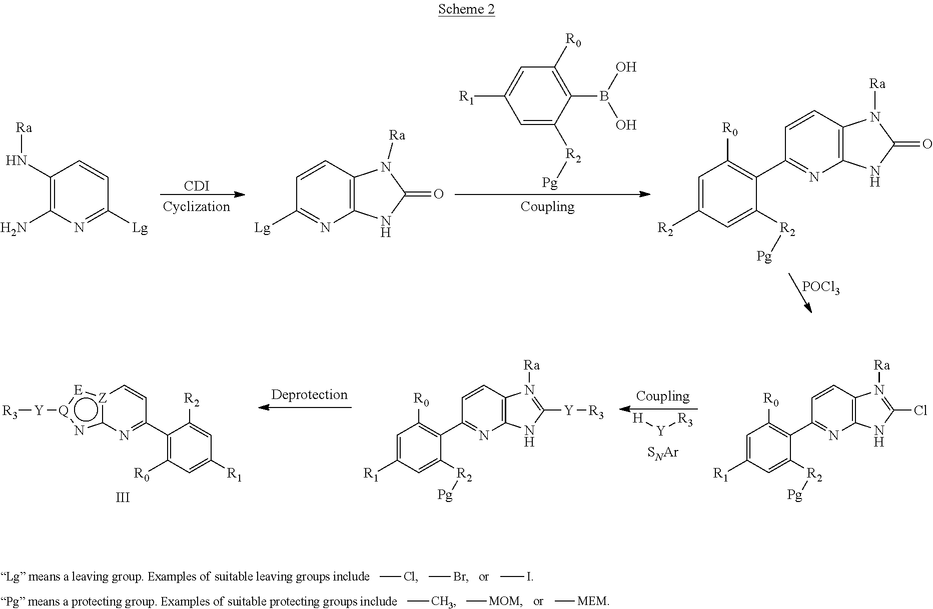

Diamino pyridines containing a leaving group can be cyclized with suitable reagents such as 1,1′-carbonyldiimidazol to afford the cyclization products after purification. Then, palladium mediated Suzuki-coupling of suitable boronic acids using appropriate bases and solvents can provide the coupling products after purification. Treatment of the coupling products with phosphoryl chloride, followed by coupling of chloro-derivatives with suitable amines, alcohols, or amino-alcohols via nucleophilic substitution can afford the coupling products with —Y—R₃ substituents after purification. Finally, acid mediated cleavage (p-toluene sulfonic acid, lithium chloride) of the protecting group under microwave conditions can afford compounds of formula (III) after purification.

### EXAMPLES

The disclosure is further illustrated by the following examples and synthesis schemes, which are not to be construed as limiting the scope of the specific procedures herein described. It is understood that the examples are provided to illustrate certain embodiments and that no limitation to the scope of this disclosure is intended thereby.

| Abbreviation | Meaning |
|---|---|
| ACN | Acetonitrile |
| BBr₃ | Boron tribromide |
| BH₃ × THF | Borane-tetrahydrofuran complex |
| BINAP | 2,2′-Bis(diphenylphosphino)-1,1′-binaphthyl |
| Brettphos Pd G3 | [(2-Di-cyclohexylphosphino-3,6-dimethoxy-2′,4′,6′-triisopropyl-1,1′- |
| | biphenyl)-2-(2′-amino-1,1′-biphenyl)]palladium(II)methanesulfonate |
| | methanesulfonate |
| CbzCl | Benzyl chloroformate |
| CDl | 1,1′-Carbonyldiimidazole |
| CH₃l/Mel | Methyl iodide |
| Cu(OAc)₂ | Copper (II)-acetate |
| CuBr₂ | Copper (II)-bromide |
| CuCN | Copper(I) cyanide |
| DCE | 1,2-Dichloroethane |
| DCM | Dichloromethane |
| DIEA/DIPEA | N,N-Diisopropylethylamine |
| DMA | N,N-Dimethylacetamide |
| DMSO | Dimethylsulfoxide |
| EtOAc/EA | Ethyl acetate |
| Et₃N | Triethylamine |
| FA | Formic Acid |
| HCHO | Formaldehyde |
| HPLC | High performance liquid chromatography |
| Int | Intermediate |
| LAH | Lithium aluminium hydride |
| LIHMDS | Lithium hexamethyldisilazide |
| m-CPBA | 3-Chloroperoxybenzoic acid |
| MeMgBr | Methylmagnesium bromide |
| MS (ESI) | Electrospray ionization mass spectrometry |
| 4 A MS | Four-ångström (4Å) molecular sieves |
| NaH | Sodium hydride |
| n-BuLi | n-Butyllithium |
| NBS | N-Bromosuccinimide |
| NMR | Nuclear magnetic resonance |
| PE | Petroleum ether |
| Pd₂(dba)₃ | Tris-(dibenzylidenaceton)-dipalladium(0) |
| Pd(dppf)Cl₂ | [1,1′-bis(diphenylphosphino)ferrocene]dichloropalladium (II) |
| Pd(PPh₃)₄ | Tetrakis(triphenylphosphine)palladium(0) |
| Pd(PPh₃)₂Cl₂ | Bis(triphenylphosphine)palladium(II) chloride |
| Pd(OAc)₂ | Palladium (II)-acetate |
| POCl₃ | Phosphorus oxychloride |
| rt or RT | Room temperature |
| SFC | Supercritical fluid chromatography |
| SM | Starting material |
| t-BuOK | Potassium tert-butoxide |
| t-BuONa | Sodium tert-butoxide |
| THF | Tetrahydrofuran |
| TLC | Thin-layer chromatography |
| UPLC | Ultra performance liquid chromatography |
| XPhos | 2-Dicyclohexylphosphino-2′,4′,6′-triisopropylbiphenyl |
| XPhos Pd G4 | (SP-4-3)-[Dicyclohexyl[2′,4′,6′-tris(1-methylethyl)[1,1′-biphenyl]-2- |
| | yl]phosphine](methanesulfonato-κO)[2′-(methylamino-κN)[1,1′- |
| | biphenyl]-2-yl-κC]palladium |
| Zn(CN)₂ | Zinc cyanide |

Unless otherwise noted, all reagents and solvents were obtained from commercial sources and used without further purification. The chemical names were generated using ChemDraw from CambridgeSoft. Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. ¹H-NMR spectra were recorded on Bruker 400 MHz-Avance Neo Nanobay NMR spectrometers in deuterated solvents. Chemical shifts (δ) are reported in parts per million and coupling constants (J values) in hertz. Spin multiplicities are indicated by the following symbols: s (singlet), d (doublet), t (triplet), q (quartet), m (multiplet), bs (broad singlet). Mass spectra (MS) were obtained on a Waters Alliance HPLC (Waters e2695 separation module), Waters Arc HPLC or Waters Acquity UPLC. Chromatography was performed using silica gel (SRL: Silica gel 100-200 mesh) and suitable solvents as indicated in specific examples. Flash purification was conducted with a CombiFlash system and the solvent gradient indicated in specific examples. Thin layer chromatography (TLC) was carried out on silica gel plates with UV detection.

### Synthesis of Intermediates

### Intermediate 1: 6-chloro-N3-methylpyridine-2,3-diamine

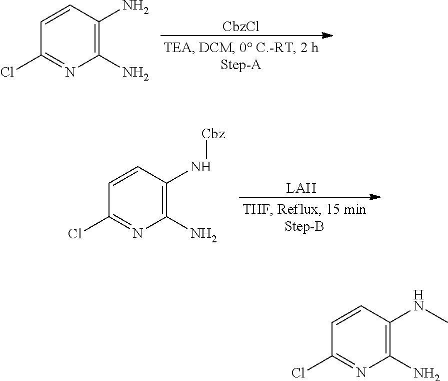

### Step A:

To a stirred solution of 6-chloropyridine-2,3-diamine (5 g, 34.83 mmol) in DCM (50 mL) was added Et₃N (14.56 mL, 104.48 mmol) followed by benzyl chloroformate (4.91 mL, 34.83 mmol) at 0° C. and the mixture was allowed to stir at room temperature for 2 h. The reaction was monitored by TLC. After the consumption of the starting material, the reaction mixture was diluted with DCM (100 mL) and washed with brine water (2×100 mL). The organic phase was dried with Na₂SO₄ and concentrated under reduced pressure to obtain the crude compound. The crude compound was purified by flash chromatography (silica 100-200 mesh; eluted with ethyl acetate in hexane (40 to 50%)). The collected pure fractions were concentrated under reduced pressure to afford benzyl (2-amino-6-chloropyridin-3-yl)carbamate (5 g, 52%) as an off-white solid.

MS (ESI): 278.24 [M+H]⁺.

¹H NMR (400 MHz, CDCl₃): δ=7.51-7.35 (m, 6H), 6.72-6.70 (d, 1H), 6.17 (brs, 1H), 5.22 (s, 2H), 4.68 (brs, 2H).

### Step B:

To a stirred solution of benzyl (2-amino-6-chloropyridin-3-yl) carbamate (obtained from Step-A) (5 g, 18.01 mmol) in dry THF (100 mL) was added 1.0 M LAH in THF (72.02 mL, 72.02 mmol) dropwise at 0° C. over a period of 20 min. The mixture was stirred at 70° C. for 15 min under N₂ atmosphere. After consumption of the starting material, the reaction mixture was cooled to RT, and quenched with saturated NH₄Cl solution (100 mL) and extracted with EtOAc (2×100 mL). The organic phase was dried with Na₂SO₄ and concentrated under reduced pressure to obtain the crude compound. The crude compound was purified by flash chromatography (silica 100-200 mesh; eluted with ethyl acetate in hexane (45 to 50%)). Collected pure fractions were concentrated under reduced pressure to afford 6-chloro-N3-methylpyridine-2,3-diamine (1.5 g, 53%) as a brown solid.

MS (ESI): 158.16 [M+H]⁺.

¹H NMR (400 MHz, DMSO-d6): δ=6.52 (d, 1H), 6.45 (d, 1H), 5.86 (s, 2H), 4.95-4.96 (m, 1H), 2.68 (d, 3H).

### Intermediate 2: 6-chloro-N3-ethylpyridine2,3-diamine

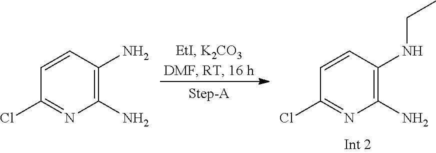

### Step A:

To a stirred solution of 6-chloropyridine-2,3-diamine (5.0 g, 34.3 mmol) in N,N-Dimethylformamide (100 mL) were added potassium carbonate (14.44 g, 104.48 mmol) and ethyl iodide (8.400 mL, 104.48 mmol) at RT and stirred for 16 h. The progress of the reaction was monitored by TLC. TLC showed completion of SM. The reaction mixture was diluted with water (300 mL) and extracted with EtOAc (2×400 mL). The combined organic layers were washed with brine solution (100 mL), dried over anhydrous sodium sulphate, filtered and concentrated under reduced pressure to get crude product as a black oil. The crude was purified by column chromatography using silica gel and eluted with EtOAc/Hexane as a gradient. The product was eluted with 30% EtOAc in Hexane. The pure fractions were collected and concentrated under reduced pressure to afford 6-chloro-N3-ethylpyridine2,3-diamine (2.0 g, 33%) as a light pink solid.

MS (ESI): 172.02 [M+H]⁺.

### Intermediate 3: 6-chloro-N3-isopropylpyridine-2,3-diamine

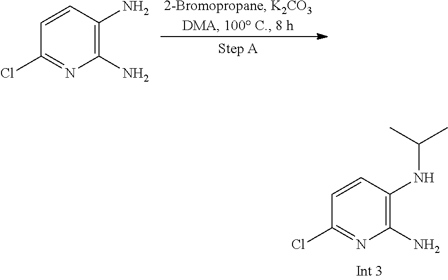

### Step A:

To a stirred solution of 6-chloropyridine-2,3-diamine (5.0 g, 34.83 mmol) in N,N-Dimethylacetamide (100 mL) were added potassium carbonate (14.44 g, 104.479 mmol) and 2-Bromopropane (9.8 mL, 104.48 mmol) at RT and stirred at 100° C. for 8 h. The progress of the reaction was monitored by TLC. TLC showed completion of SM. The reaction mixture was diluted with water (300 mL) and extracted with EtOAc (2×400 mL). The combined organic layers were washed with brine solution (100 mL), dried over anhydrous sodium sulphate, filtered and concentrated under reduced pressure to get crude product as a black oil. The crude was purified by column chromatography using silica gel (100-200) and eluted with 0-50% EtOAC/Hexane as a gradient. The product was eluted at 20% EtOAc/Hexane. The pure fractions were collected and concentrated under reduced pressure to afford 6-chloro-N3-isopropylpyridine-2,3-diamine (2.5 g, 39%) as an off white solid.

MS (ESI): 185.81 [M+H]⁺.

### Intermediate 4: 2-(methoxymethoxy)-6-methyl-4-(trifluoromethyl)benzaldehyde

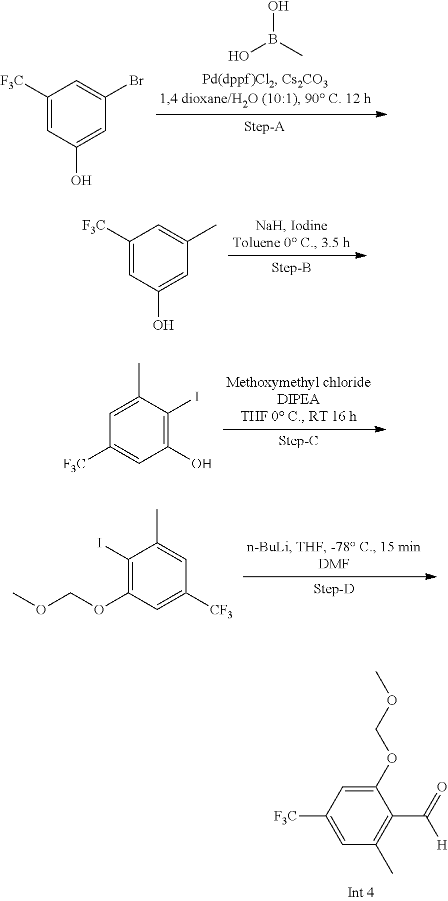

### Step-A:

To a stirred solution of 3-bromo-5-(trifluoromethyl)phenol (30 g, 124.48 mmol) in 1,4 dioxane (300 mL) and water (30 mL), was added methylboronic acid (14.9 g, 248.95 mmol) and cesium carbonate (121.670 g, 373.43 mmol). The mixture was degassed with N₂ for 20 min and [1,1′-bis(diphenylphosphino)ferrocene]dichloropalladium (II) (4.55 g, 6.22 mmol) was added at RT and the mixture was heated to 90° C. for 12 h. The progress of the reaction was monitored by TLC. After the consumption of the starting material, the reaction mixture was filtered through a Celite pad, the filtrate was concentrated under reduced pressure to get a crude compound. The crude compound was purified by column chromatography using silica gel (100-200 mesh) and eluted with 0-50% EtOAc in hexane as a gradient. The product was eluted at 15% EtOAc in hexane. The pure fractions were collected and concentrated under reduced pressure to afford 3-methyl-5-(trifluoromethyl)phenol (21 g, 96%) as a pale yellow liquid.

MS (ESI): 175.05 [M−H]⁻.

¹H NMR (400 MHz, CDCl₃): δ=7.00 (s, 1H), 6.88 (s, 1H), 6.82 (s, 1H), 5.00 (s, 1H), 2.36 (s, 3H).

### Step-B:

To a stirred solution of 3-methyl-5-(trifluoromethyl)phenol (obtained from Step-A) (12 g, 68.13 mmol) in toluene (420 mL) was added sodium hydride 57-63% oil dispersion (6.86 g, 204.39 mmol) at 0° C. The reaction mixture was stirred for 30 min at 0° C. Thenilodine was added portionwise (19 g, 74.94 mmol) at 0° C. and stirring was continued for 3 h. After consumption of the starting material, the reaction mixture was quenched with ice cold water (80 mL), acidified with 1N HCl solution and extracted with EtOAc (2×200 mL). The combined organic layers were washed with brine solution (80 mL), dried over anhydrous sodium sulfate, filtered and concentrated under reduced pressure to get the crude product. The crude product was purified by column chromatography using silica gel (100-200 mesh) and eluted with 0-50% EtOAC in hexane as a gradient. The product was eluted at 15% EtOAc in hexane. The pure fractions were collected and concentrated under reduced pressure to afford 2-iodo-3-methyl-5-(trifluoromethyl)phenol (13.5 g, 65%) as a yellow oil.

MS (ESI): 301.17 [M−H]⁻.

### Step-C:

To a stirred solution of 2-iodo-3-methyl-5-(trifluoromethyl)phenol (obtained from Step-B) (13.5 g, 44.70 mmol) in THF (135 mL) were added N,N-diisopropylethylamine (31 mL, 178.79 mmol) and methoxymethyl chloride (8.49 mL, 111.74 mmol) dropwise at 0° C. under argon atmosphere. The reaction mixture was stirred at RT for 16 h. After consumption of the starting material, the reaction mixture was concentrated under reduced pressure to get the crude product. The crude product was purified by column chromatography using silica gel (100-200 mesh) and eluted with hexane as a gradient. The pure fractions were collected and concentrated under reduced pressure to afford 2-iodo-1-(methoxymethoxy)-3-methyl-5-(trifluoromethyl)benzene (12 g, 78%) as a colorless oil.

MS (ESI): 346.0 [M+H]⁺.

¹H NMR (400 MHz, CDCl₃): δ=7.17 (s, 1H), 7.08 (s, 1H), 5.27 (s, 2H), 3.52 (s, 3H), 2.53 (s, 3H).

### Step-D:

To a stirred solution of 2-iodo-1-(methoxymethoxy)-3-methyl-5-(trifluoromethyl)benzene (obtained from Step-C) (12.0 g, 31.08 mmol) in THF (120 mL) was added n-butyllithium (1.6M) solution in hexane (25 mL, 40.34 mmol) at −78° C. and the mixture was stirred for 5 min. N,N-Dimethylformamide (2.9 mL, 37.29 mmol) was added and the mixture was allowed to stir for 10 min at the same temperature. After consumption of the starting material, the reaction mixture was quenched with saturated NH₄Cl solution and extracted with EtOAc (2×40 mL). The combined organic layers were washed with brine solution (60 mL), dried over anhydrous sodium sulfate, filtered and concentrated under reduced pressure to get a crude 2-(methoxymethoxy)-6-methyl-4-(trifluoromethyl)benzaldehyde (6.5 g, 84%) as an oil.

¹H NMR (400 MHz, DMSO-d6): δ=10.55 (s, 1H), 7.43 (s, 1H), 7.32 (s, 1H), 5.43 (s, 2H), 3.38 (s, 3H), 2.36 (s, 3H).

### Intermediate 5: 4-chloro-2-methoxy-6-methylbenzaldehyde

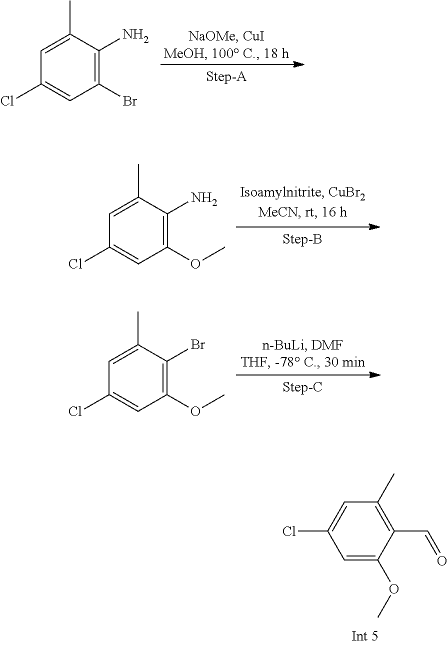

### Step-A:

To a stirred solution of 2-bromo-4-chloro-6-methylaniline (20 g, 90.71 mmol) in methanol (100 mL) were added Sodium methoxide, ca 30% w/w in methanol (60 mL) and Copper (I)iodide (19 g, 99.78 mmol) at rt and heated to 100° C. for 18 h. The progress of the reaction was monitored by TLC. TLC showed SM completed. The reaction mixture was diluted with water (500 mL) and extracted with DCM (2×500 mL). The combined organic layer was washed with brine solution (100 mL), dried over anhydrous sodium sulphate, filtered and concentrated under reduced pressure to get crude product as a liquid. The crude was purified by column chromatography using silica gel and eluted with EtOAc in Hexane as a gradient. The product was eluted with 5% EtOAc in Hexane. The pure fractions were collected and concentrated under reduced pressure to get the pure compound (5.9 g, 38%).

MS (ESI): 172.27 [M+H]⁺.

### Step-B:

To a stirred solution of 4-chloro-2-methoxy-6-methylaniline (6.0 g, 34.96 mmol) in acetonitrile (150 mL) was added Isoamyl nitrite, 97%, stabilized (7.06 mL, 52.44 mmol) at 0° C. The RM was stirred for 30 min. Then CuBr₂ (8.43 g, 37.78 mmol) was added at 0° C. The RM was allowed to stirred RT for 16 h. The progress of the reaction was monitored by TLC. TLC showed completion of SM. The reaction mixture was diluted with water (250 mL) and extracted with EtOAc (2×250 mL). The combined organic layer was washed with brine solution (100 mL), dried over anhydrous sodium sulphate, filtered and concentrated under reduced pressure to get crude product as a liquid. The crude was purified by column chromatography using silica gel (100-200) and eluted with EtOAc in Hexane as a gradient. The product was eluted at 2% EtOAc in Hexane. The pure fractions were collected and concentrated under reduced pressure to get the pure compound (4.47 g, 55%).

MS (ESI): 233.94 [M+H]⁺.

### Step-C:

To a stirred solution of 2-bromo-5-chloro-1-methoxy-3-methylbenzene (4.2 g, 17.83 mmol) in THF (50 mL) was added n-BuLi (1.6 M in Hexane) (16.71 mL, 26.75 mmol) at −78° C. and stirred the reaction mixture for 10 min. Then N,N-dimethylformamide (4.11 mL, 53.502 mmol) was added at −78° C. allowed to stirred the RM for 30 min. The progress of the reaction was monitored by TLC. TLC showed completion. The reaction mixture was quenched with saturated NH4Cl solution, extracted with EtOAc (2×250 mL). The combined organic layer was washed with brine solution (100 mL), dried over anhydrous sodium sulphate, filtered and concentrated under reduced pressure to get crude product as a solid. The crude was purified by column chromatography using silica gel (100-200) and eluted with EtOAc in Hexane as a gradient. The product was eluted at 5% EtOAc in Hexane. The pure fractions were collected and concentrated under reduced pressure to get the pure compound (2.87 g, 88%).

MS (ESI): 183.9 [M+H]⁺.

### Intermediate 6: 2-(5-chloro-1-methyl-1H-imidazo[4,5-b]pyridin-2-yl)-3-methyl-5-(trifluoromethyl)phenol

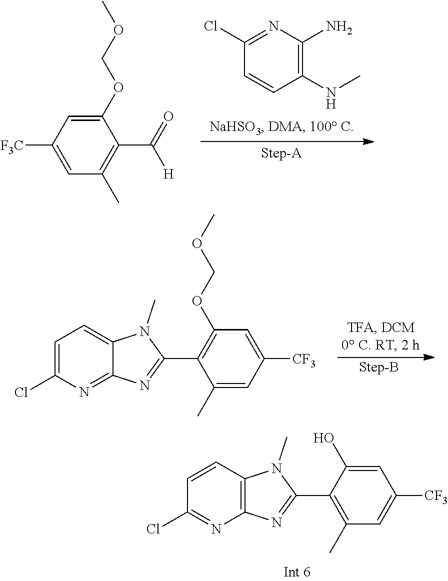

### Step A:

To a stirred solution of 6-chloro-N3-methylpyridine-2,3-diamine (Int 1) (1.8 g, 11.42 mmol) and 2-(methoxymethoxy)-6-methyl-4-(trifluoromethyl)benzaldehyde (Int 2) (6.52 g, 26.27 mmol) in DMA (10 mL) was added sodium bisulfite (1.43 g, 13.71 mmol) at RT, and the reaction mixture was stirred at 100° C. for 16 h. After consumption of the starting material, the reaction mixture was quenched with ice cold water (30 mL) and extracted with EtOAc (2×100 mL). The combined organic layers were washed with brine solution (100 mL), dried over anhydrous sodium sulfate, filtered and concentrated under reduced pressure to get the crude product. The crude product was purified by column chromatography using silica gel (100-200 mesh) and eluted with EtOAC in hexane as a gradient. The product was eluted at 30% EtOAc in hexane. The pure fractions were collected and concentrated under reduced pressure to afford 5-chloro-2-(2-(methoxymethoxy)-6-methyl-4-(trifluoromethyl)phenyl)-1-methyl-1H-imidazo[4,5-b]pyridine (4.2 g, 95%) as an oil.

MS (ESI): 386.29 [M+H]+.

¹H NMR (400 MHz, DMSO-d6): δ=8.23-8.21 (d, 1H), 7.49-7.42 (d, 3H), 5.32-5.31 (d, 1H), 5.23-5.21 (d, 1H), 3.63 (s, 3H), 3.24 (s, 3H), 2.15 (s, 3H).

### Step B:

To a stirred solution of 5-chloro-2-(2-(methoxymethoxy)-6-methyl-4-(trifluoromethyl)phenyl)-1-methyl-1H-imidazo[4,5-b]pyridine (obtained from Step-A) (4.2 g, 10.89 mmol) in DCM (84 mL) was added trifluoroacetic acid (63 mL) at 0° C. and the reaction mixture was stirred at RT for 2 h. After consumption of the starting material, the reaction mixture was quenched with ice cold water (50 mL) and stirred for 10 min. The precipitated solid was filtered, washed with water (100 mL) and dried under vacuum to afford 2-(5-chloro-1-methyl-1H-imidazo[4,5-b]pyridin-2-yl)-3-methyl-5-(trifluoromethyl)phenol (3.5 g, 94%) as an off-white solid.

MS (ESI): 342.25 [M+H]⁺.

¹H NMR (400 MHz, DMSO-d6): δ=10.79 (s, 1H) 8.22-8.20 (d, 1H), 7.44-7.424 (d, 1H), 7.25 (s, 1H), 7.15 (s, 1H), 3.64 (s, 3H), 2.16 (s, 3H).

### Intermediate 7: 2-(5-chloro-1-methyl-1H-imidazo[4,5-b]pyridin-2-yl)-5-(trifluoromethyl)phenol

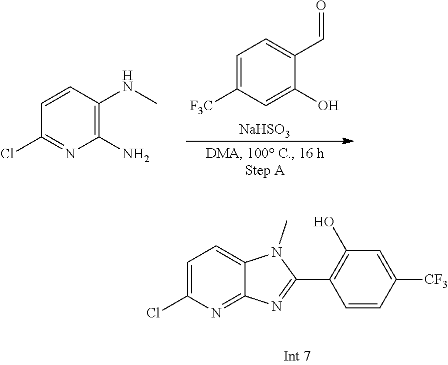

### Step A:

To a stirred solution of 6-chloro-N3-methylpyridine-2,3-diamine (Int 1) (0.2 g, 1.27 mmol) and 2-hydroxy-4-(trifluoromethyl)benzaldehyde (0.241 g, 1.27 mmol) in DMA (5 mL) was added sodium bisulfite (0.158 g, 1.52 mmol) at RT, and the reaction mixture was allowed to stir at 100° C. for 16 h. After consumption of the starting material, the reaction mixture was diluted with ice water (50 mL) and stirred for 10 min. The precipitate was collected by filtration and dried to obtain 2-(5-chloro-1-methyl-1H-imidazo[4,5-b]pyridin-2-yl)-5-(trifluoromethyl)phenol (0.2 g, 48%) as a light yellow solid.

MS (ESI): 328.22 [M+H]⁺.

¹H NMR (400 MHz, DMSO-d6): δ=11.24 (brs, 1H), 8.21-8.19 (d, 1H), 7.75-7.73 (d, 1H), 7.43-7.41 (d, 1H), 7.36-7.34 (m, 2H), 3.77 (s, 3H).

The following intermediates were prepared analogous to the above using the corresponding amines and the appropriate aldehydes optionally followed by a deprotection step as shown in Table 1:

TABLE 1

| | | | 1. Yield |
|---|---|---|---|
| | | | 2. MH⁺ (ESI) |
| Amine | Aldehyde | Intermediate | 3. 1H-NMR |
| 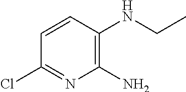 Int 2 | 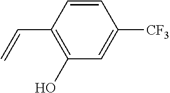 | 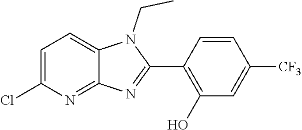 [SMILES: CCn1c(-c2ccc(C(F)(F)F)cc2O)nc2nc(Cl)ccc21] Int 8 | 1. 61% 2. 342.17 3. ¹H NMR (400 MHz, DMSO-d₆) δ 11.13 (s, 1H), 8.25 (d, 1H), 7.68 (d, 1H), 7.41 (d, 1H), 7.38 − 7.30 (m, 2H), 4.20 (q, 2H), 1.24 (t, 3H). |
| | | 2-(5-chloro-1-ethyl-1H-imidazo[4,5- | |
| | | b]pyridin-2-yl)-5-(trifluoromethyl)phenol | |
| 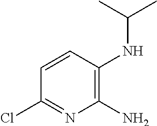 Int 3 | 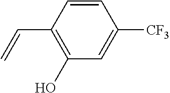 | 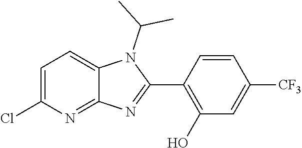 [SMILES: CC(C)n1c(-c2ccc(C(F)(F)F)cc2O)nc2nc(Cl)ccc21] | 1. 63% 2. 356.2 3. ¹H NMR (400 MHz, DMSO-d₆) δ 11.03 (s, 1H), 8.39 (d, 1H), 7.65 (dd, 1H), 7.37 - 7.31 (m, 2H), 7.30 (d, 1H), 4.40 (hept, 1H), 1.53 (d, 6H). |
| | | Int 9 | |
| | | 2-(5-chloro-1-isopropyl-1H-imidazo[4,5- | |
| | | b]pyridin-2-yl)-5-(trifluoromethyl)phenol | |
| 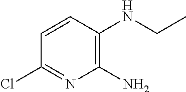 Int 2 | 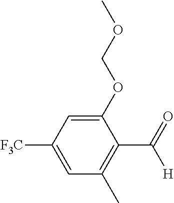 [SMILES: COCOc1cc(C(F)(F)F)cc(C)c1C=O] Int 4 | 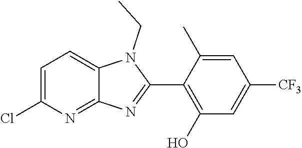 [SMILES: CCn1c(-c2c(C)cc(C(F)(F)F)cc2O)nc2nc(Cl)ccc21] Int 10 2-(5-chloro-1-ethyl-1H-imidazo[4,5- b]pyridin-2-yl)-3-methyl-5- (trifluoromethyl)phenol | 1. 86% (over 2 steps) 2. 356.49 |
| 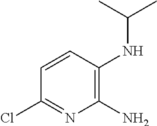 Int 3 | 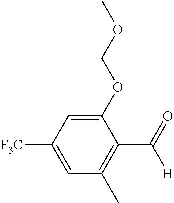 [SMILES: COCOc1cc(C(F)(F)F)cc(C)c1C=O] Int 4 | 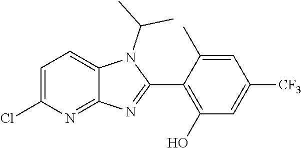 [SMILES: Cc1cc(C(F)(F)F)cc(O)c1-c1nc2nc(Cl)ccc2n1C(C)C] Int 11 2-(5-chloro-1-isopropyl-1H-imidazo[4,5- b]pyridin-2-yl)-3-methyl-5- (trifluoromethyl)phenol | 1. 90% (over 2 steps) 2. 370.25 |
| 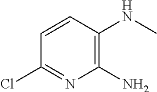 Int 1 | 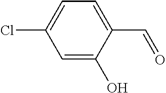 | 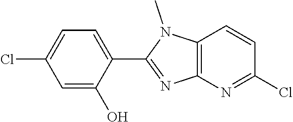 [SMILES: Cn1c(-c2ccc(Cl)cc2O)nc2nc(Cl)ccc21] | 1. 85% 2. 293.98 3. ¹H NMR (400 MHz, DMSO-d₆) δ 11.13 (s, 1H), 8.17 (d, 1H), 7.55 (d, 1H), 7.40 (d, 1H), 7.12 − 7.04 (m, 2H), 3.76 (s, 3H). |
| | | Int 12 | |
| | | 5-chloro-2-(5-chloro-1-methyl-1H- | |
| | | imidazo[4,5-b]pyridin-2-yl)phenol | |
| 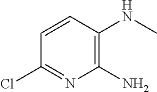 Int 1 | 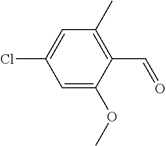 Int 5 | 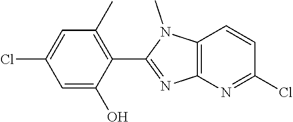 [SMILES: Cc1cc(Cl)cc(O)c1-c1nc2nc(Cl)ccc2n1C] Int 13 5-chloro-2-(5-chloro-1-methyl-1H- imidazo[4,5-b]pyridin-2-yl)-3-methylphenol | 1. 29% (over 2 steps) 2. 308.11 |
| 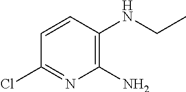 Int 2 | 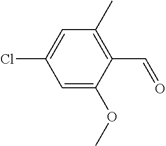 Int 5 | 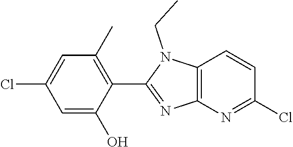 [SMILES: CCn1c(-c2c(C)cc(Cl)cc2O)nc2nc(Cl)ccc21] Int 14 | 1. 29% (over 2 steps) 2. 322.10 |
| | | 5-chloro-2-(5-chloro-1-ethyl-1H- | |
| | | imidazo[4,5-b]pyridin-2-yl)-3- | |
| | | methylphenol | |
| 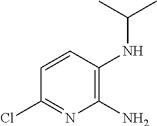 Int 3 | 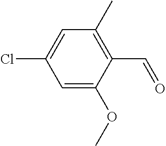 Int 5 | 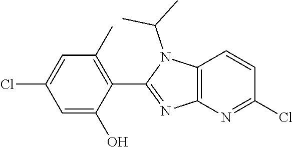 [SMILES: Cc1cc(Cl)cc(O)c1-c1nc2nc(Cl)ccc2n1C(C)C] Int 15 | 1. 48% (over 2 steps) 2. 351.22 |
| | | 5-chloro-2-(5-chloro-1-isopropyl-1H- | |
| | | imidazo[4,5-b]pyridin-2-yl)-3- | |
| | | methylphenol | |

### Intermediate 16: 2-methoxy-6-methyl-4-(trifluoromethyl)benzaldehyde

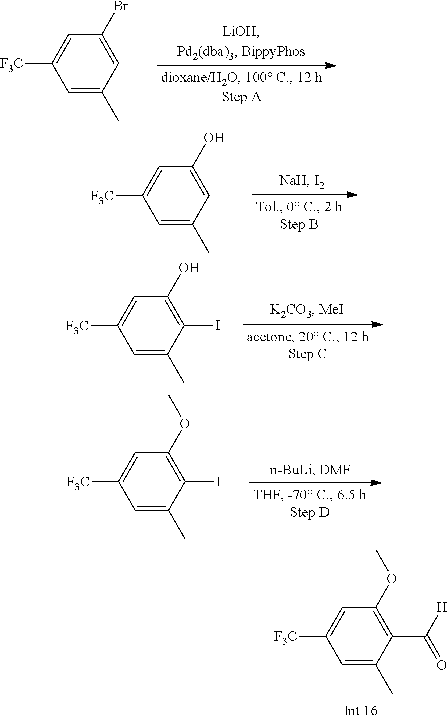

### Step A:

To the solution of 1-bromo-3-methyl-5-(trifluoromethyl)benzene (45 g, 188.26 mmol, 1.0 eq) in the mixed solvent of dioxane (450 mL) and H₂O (45 mL) was successively added LiOH·H₂O (13.53 g, 564.78 mmol, 3.0 eq), Pd₂(dba)₃ (3.45 g, 3.77 mmol, 0.02 eq) and ditert-butyl-[2-(1,3,5-triphenylpyrazol-4-yl)pyrazol-3-yl] phosphane (3.82 g, 7.53 mmol, 0.04 eq), then degassed with N₂ for 3 times. The resulting reaction mixture was heated to 100° C. and stirred at 100° C. for 12 hr under N₂. TLC (PE/EA=10/1, product Rf=0.50, Color Developing Reagent: UV254 nm) indicated that the reaction was completed. The reaction mixture was poured onto 500 mL H₂O and followed by 50 mL EA, and then adjusting the pH to 4 with 2 M HCl. After that, the aqueous phase was separated and extracted with EA (80 mL*3). The combined organic layer was washed successively with water (20 mL*2) and brine (20 ml*1), dried over anhydrous Na₂SO₄, filtered, and concentrated to give a residue. The residue was purified by column chromatography (SiO₂, Petroleum ether/Ethyl acetate=100/1 to 10/1). -methyl-5-(trifluoromethyl)phenol (44 g, 249.81 mmol, 66.35% yield, N/A purity) was obtained as a yellow oil.

¹H NMR (400 MHz, CHLOROFORM-d) δ=7.01 (s, 1H), 6.89 (s, 1H), 6.82 (s, 1H), 5.04 (s, 1H), 2.37 (s, 3H)

### Step B:

To the solution of 3-methyl-5-(trifluoromethyl)phenol (39 g, 221.42 mmol, 1.0 eq) in Tol. (800 mL) was added NaH (17.71 g, 442.84 mmol, 60% purity, 2.0 eq) at 0° C. and the reaction mixture was stirred at 0° C. for 30 min, then I₂ (39.34 g, 154.99 mmol, 31.22 mL, 0.7 eq) was added. The resulting reaction mixture was stirred at 0° C. for 1.5 hr. TLC (PE/EA=10/1, product Rf=0.45, Color Developing Reagent: UV254 nm) indicated that the reaction was completed. The reaction mixture was poured onto 200 mL sat. NH₄Cl. and then adjusting the pH to 4 with 2 M HCl. Then the resulting solution was extracted with EA (100 mL*3). The combined organic layer was washed successively with water (200 mL*2) and brine (200 ml*1), dried over anhydrous Na₂SO₄, filtered, and concentrated to give a residue. The residue was purified by column chromatography (SiO₂, Petroleum ether/Ethyl acetate=100/1 to 10/1). 2-iodo-3-methyl-5-(trifluoromethyl)phenol (38.6 g, 127.80 mmol, 57.72% yield, N/A purity) was obtained as a yellow solid.

¹H NMR (400 MHz, CHLOROFORM-d) δ=7.07 (s, 2H), 5.62 (s, 1H), 2.52 (s, 3H)

### Step C:

To the solution of 2-iodo-3-methyl-5-(trifluoromethyl)phenol (5.6 g, 18.54 mmol, 1.0 eq) in acetone (60 mL) was added K₂CO₃ (5.12 g, 37.08 mmol, 2.0 eq), CH₃I (5.26 g, 37.08 mmol, 2.31 mL, 2.0 eq) was added. The resulting reaction mixture was stirred at 20° C. for 12 hr. TLC (PE/EA=10/1, product Rf=0.70, Color Developing Reagent: UV254 nm) indicated that the reaction was completed. The reaction solution was filtered to remove K₂CO₃ then concentrated to give the residue. The residue was purified by column chromatography (SiO₂, Petroleum ether/Ethyl acetate=100/0 to 98/2). 2-iodo-1-methoxy-3-methyl-5-(trifluoromethyl)benzene (5.3 g, 16.77 mmol, 90.44% yield, N/A purity) was obtained as a white solid.

¹H NMR (400 MHz, CHLOROFORM-d) δ=7.14 (s, 1H), 6.82 (s, 1H), 3.94 (s, 3H), 2.54 (s, 3H)

### Step D:

To the solution of 2-iodo-1-methoxy-3-methyl-5-(trifluoromethyl)benzene (8 g, 25.31 mmol, 1.0 eq) in THF (80 mL) was added n-BuLi (2.5 M, 25.31 mL, 2.5 eq) and the reaction mixture was stirred at −70° C. for 30 min, then DMF (18.50 g, 253.12 mmol, 19.48 mL, 10 eq) was added. The resulting reaction mixture was stirred at −70° C. for 6 hr. TLC (PE/EA=4/1, product Rf=0.60, Color Developing Reagent: UV254 nm) indicated that the reaction was completed. The reaction mixture was quenched with 100 mL H₂O at 0° C. and followed by 30 mL DCM. After that, the aqueous phase was separated and extracted with DCM (30 mL*3). The combined organic layer was washed successively with water (20 mL*2) and brine (20 ml*1), dried over anhydrous Na₂SO₄, filtered, and concentrated to give a residue. The residue was purified by column chromatography (SiO₂, Petroleum ether/Ethyl acetate=100/1 to 10/1). 2-methoxy-6-methyl-4-(trifluoromethyl)benzaldehyde (4 g, 18.33 mmol, 72.43% yield) was obtained as a yellow oil.

¹H NMR (400 MHz, CHLOROFORM-d) δ=10.65 (s, 1H), 7.07 (d, J=8.8 Hz, 2H), 3.97 (s, 3H), 2.62 (s, 3H)

### Intermediate 18: 4-chloro-2-methoxy-6-methylbenzaldehyde

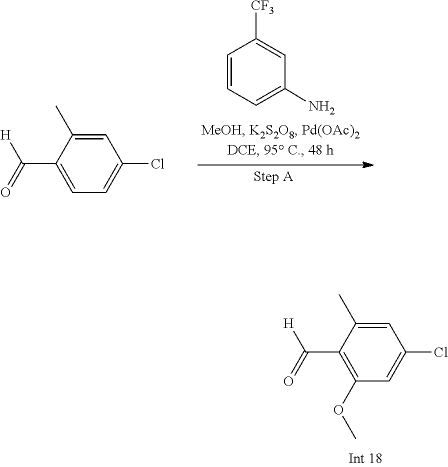

### Step A:

To the solution of 4-chloro-2-methyl-benzaldehyde (2 g, 12.94 mmol, 1.0 eq) in DCE (70 mL) was added MeOH (8.29 g, 258.74 mmol, 10.47 mL, 20.0 eq), Pd(OAc)₂ (435.67 mg, 1.94 mmol, 0.15 eq), dipotassium; sulfonatooxy sulfate (6.99 g, 25.87 mmol, 5.18 mL, 2.0 eq), 3-(trifluoromethyl)aniline (2.08 g, 12.94 mmol, 1.62 mL, 1.0 eq) and the resulting reaction mixture was stirred at 95° C. for 48 hr in sealed tube. LCMS indicated that 29% starting material remained and 46% desired product. The reaction mixture was poured onto 300 mL H₂O and followed by 100 mL DCM. After that, the aqueous phase was separated and extracted with DCM (100 mL*2). The combined organic layer was washed successively with water (20 mL*2) and brine (20 ml*1), dried over anhydrous Na₂SO₄, filtered, and concentrated to give a residue. The residue was purified by column chromatography (SiO₂, Petroleum ether/Ethyl acetate=100/1 to 20/1) and then trituration with PE 20 mL to get pure product. The residue was purified by prep-HPLC (column: Waters Xbridge C18 150*50 mm*10 um; mobile phase: [H₂O(10 mM NH₄HCO₃)-ACN]; gradient: 35%-65% B over 8.0 min) to give the title product. The residue was purified by prep-HPLC (column: Waters Xbridge C18 150*50 mm*10 um; mobile phase: [H₂O(10 mM NH₄HCO₃)-ACN]; gradient: 35%-65% B over 8.0 min) to give the title product. 4-chloro-2-methoxy-6-methyl-benzaldehyde (1.5 g, 8.12 mmol, 20.93% yield, N/A purity) was obtained as a white solid.

LC-MS (ES+, m/z): 185.1 [(M+H)⁺].

¹H NMR (400 MHz, DMSO-d₆) δ=10.43 (s, 1H), 7.18 (s, 1H), 6.99 (s, 1H), 3.91 (s, 3H), 2.46 (s, 3H)

### Intermediate 19

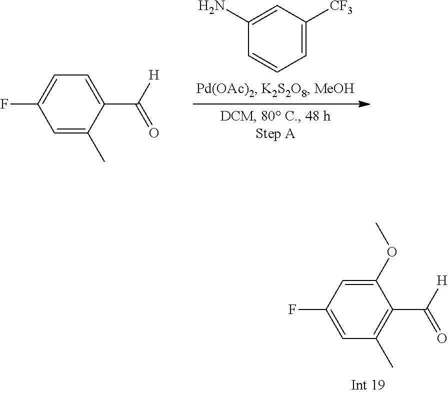

### Step A:

To the solution of 4-fluoro-2-methyl-benzaldehyde (500 mg, 3.62 mmol, 1.0 eq) in DCE (15 mL) was added Pd(OAc)₂ (81.26 mg, 361.96 μmol, 0.1 eq), dipotassium; sulfonatooxy sulfate (1.96 g, 7.24 mmol, 1.45 mL, 2.0 eq), MeOH (2.32 g, 72.39 mmol, 2.93 mL, 20.0 eq), 3-(trifluoromethyl)aniline (233.28 mg, 1.45 mmol, 180.84 μL, 0.4 eq) and the reaction mixture was stirred at 80° C. for 48 h in sealed tube. The reaction mixture was poured onto 20 mL H₂O and followed by 10 mL EtOAc. After that, the aqueous phase was separated and extracted with EtOAc (20 mL×2). The combined organic layer was washed successively with water (20 mL×2) and brine (20 mL×1), dried over anhydrous Na₂SO₄, filtered, and concentrated to give a residue. The residue was purified by prep-TLC (SiO₂, PE:EtOAc=10:1). 4-fluoro-2-methoxy-6-methyl-benzaldehyde (0.5 g, 2.97 mmol, 82.14% yield, N/A purity) was obtained as a white solid.

¹H NMR (400 MHz, CDCl₃) δ=10.54 (s, 1H), 6.58-6.50 (m, 2H), 3.90 (s, 3H), 2.59 (s, 3H).

MS: 168.7 [(M+H)⁺].

### SYNTHESIS OF INVENTIVE EXAMPLES

### Example 1: 2-(5-((2-hydroxyethyl)amino)-1-methyl-1H-imidazo[4,5-b]pyridin-2-yl)-5-(trifluoromethyl)phenol

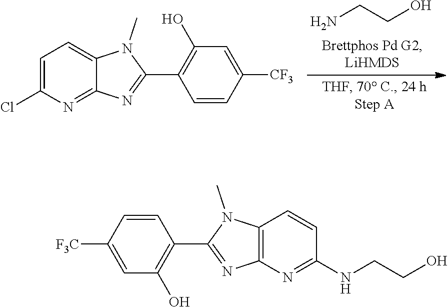

### Step A:

A stirred solution of 2-(5-chloro-1-methyl-1H-imidazo[4,5-b]pyridin-2-yl)-5-(trifluoromethyl)phenol (Int 7) (0.2 g, 0.61 mmol) and 2-aminoethan-1-ol (0.075 g, 1.22 mmol) in THF (10 mL) was degassed with nitrogen gas for 10 min. Then BrettPhos Pd G3 (0.028 g, 0.031 mmol) was added followed by LiHMDS (1.0 M in THF) (1.83 mL, 1.83 mmol) at RT. The reaction mixture was stirred at 70° C. for 24 h. After consumption of the starting material, the reaction mixture was diluted with ice cold water (50 mL) and extracted with ethyl acetate (2×50 mL). The combined organic layers were dried over Na₂SO₄ and concentrated under reduced pressure to obtain the crude compound. The crude compound was purified by Preparative HPLC (Column XBridge C18 (150 mm×19 mm, 5 μm), buffer: 10 mM ammonium bicarbonate, mobile phase: acetonitrile, flow: 14 mL/min), and the fractions containing the product were collected and lyophilized to obtain 2-(5-((2-hydroxyethyl)amino)-1-methyl-1H-imidazo[4,5-b]pyridin-2-yl)-5-(trifluoromethyl)phenol (22 mg, 6%) as an off-white solid.

MS (ESI): 353.27 [M+H]⁺.

¹H NMR (400 MHz, DMSO-d6): δ=12.40 (brs, 1H), 7.91-7.89 (d, 1H), 7.77-7.75 (d, 1H), 7.36-7.29 (m, 2H), 6.59-6.48 (m, 2H), 4.76 (brs, 1H), 3.83 (s, 3H), 3.57-3.56 (m, 2H), 3.40-3.36 (q, 2H).

Following the procedure of Example 1, the following examples were prepared, using the haloaromatic intermediates and the corresponding commercially available amines as shown in Table 2:

TABLE 2

| | | | 1. Yield |
|---|---|---|---|
| Halo- | | | 2. ¹H-NMR |
| aromatic | Amine | Example | 3. MH⁺ (ESI) |
| Int 7 | 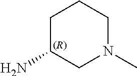 | 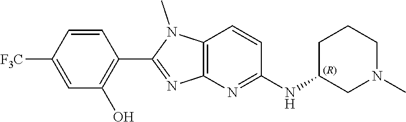 | 1. 24% 2. ¹H NMR (400 MHz, DMSO-d6): δ = 12.27 (brs, 1H), 7.89-7.87 (d, 1H), 7.81-7.79 (d, 1H), 7.33-7.30 (m, 2H), 6.64- 6.57 (m, 2H), 4.16 (brs, 1H), 3.82 (s, 3H), 3.22- |
| | | 2 | 2.67 (m, 4H), 2.49-2.32 |
| | | ((R)-2-(1-methyl-5-((1-methylpiperidin-3- | (m, 3H), 1.90 (brs, 2H), |
| | | yl)amino)-1H-imidazo[4,5-b]pyridin-2-yl)- | 1.71 (brs, 1H), 1.38-1.35 |
| | | 5-(trifluoromethyl)phenol | (brs, 1H). |
| | | | 3. 406.37 |
| Int 6 | —NH₂ | 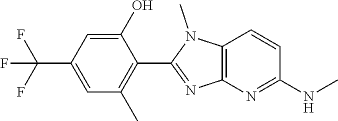 [SMILES: CNc1ccc2c(n1)nc(-c1c(C)cc(C(F)(F)F)cc1O)n2C] | 1. 33% 2. ¹H NMR (400 MHz, DMSO-d6): δ = 10.72 (brs, 1H), 7.67-7.65 (d, 1H), 7.15 (s, 1H), 7.09 (s, 1H), 6.45-6.43 (d, 1H), 3.47 (s, 3H), 2.82-2.80 (d, 3H), 2.13 (s, 3H). |
| | | 3 | 3. 337.13 |
| | | 3-methyl-2-(1-methyl-5-(methylamino)- | |
| | | 1H-imidazo[4,5-b]pyridin-2-yl)-5- | |
| | | (trifluoromethyl) phenol | |
| Int 7 | —NH₂ | 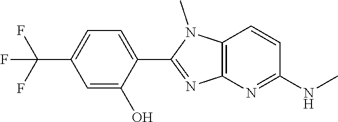 [SMILES: CNc1ccc2c(n1)nc(-c1ccc(C(F)(F)F)cc1O)n2C] | 1. 63% 2. ¹H NMR (400 MHz, DMSO-d6): δ = 12.48 (brs, 1H), 7.92-7.90 (d, 1H), 7.78-7.76 (d, 1H), 7.32-7.30 (m, 2H), 6.54- 6.51 (m, 2H), 3.85 (s, 3H), 2.83-2.82 (d, 3H). |
| | | 4 | 3. 323.12 |
| | | 2-(1-methyl-5-(methylamino)-1H- | |
| | | imidazo[4,5-b]pyridin-2-yl)-5- | |
| | | (trifluoromethyl)phenol | |
| Int 7 | 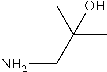 | 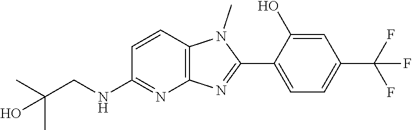 [SMILES: Cn1c(-c2ccc(C(F)(F)F)cc2O)nc2nc(NCC(C)(C)O)ccc21] | 1. 26% 2. ¹H NMR (400 MHz, DMSO-d6): δ = 12.48 (brs, 1H), 7.87-7.85 (d, 1H), 7.76-7.74 (d, 1H), 7.29-7.25 (m, 2H), 6.68- 6.66 (d, 1H), 6.44-6.41 (t, 1H), 4.82 (s, 1H), 3.82 (s, 3H), 3.10 (s, 2H), 1.14 (s, |
| | | 5 | 6H). |
| | | 2-(5-((2-hydroxy-2-methylpropyl)amino)- | 3. 381.4 |
| | | 1-methyl-1H-imidazo[4,5-b]pyridin-2-yl)- | |
| | | 5-(trifluoromethyl)phenol | |
| Int 6 | 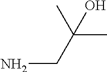 | 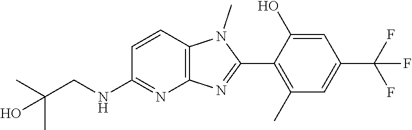 [SMILES: Cc1cc(C(F)(F)F)cc(O)c1-c1nc2nc(NCC(C)(C)O)ccc2n1C] | 1. 46% 2. 1H NMR (400 MHz DMSO-d6): δ = 10.54 (brs, 1H), 7.69-7.66 (d, 1H), 7.18 (s, 1H), 7.10 (s, 1H), 6.60-6.58 (d, 1H), 6.29-6.26 (t, 1H), 4.95 (s, 1H), 4.95 (s, 1H), 3.47 (s, 3H), 3.26-3.23 (m, 1H), |
| | | 6 | 2.14 (s, 3H), 1.14 (s, 6H). |
| | | 2-(5-((2-hydroxy-2-methylpropyl)amino)- | 3. 395.43 |
| | | 1-methyl-1H-imidazo[4,5-b]pyridin-2-yl)- | |
| | | 3-methyl-5-(trifluoromethyl)phenol | |
| Int 7 | 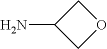 | 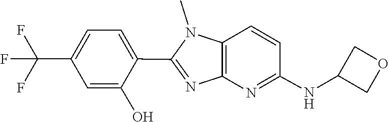 | 1. 18% 2. ¹H NMR (400 MHz, DMSO-d₆): δ = 12.41 (brs, 1H), 7.78-7.72 (m, 2H), 7.20-7.19 (m, 2H), 7.14- 7.12 (d, 1H), 6.54-6.52 (d, 1H), 4.94-4.91 (m, 1H), 4.87-4.83 (m, 2H), 4.46- |
| | | 7 | 4.43 (t, 2H), 3.75 (s, 3H). |
| | | 2-(1-methyl-5-(oxetan-3-ylamino)-1H- | 3. 365.13 |
| | | imidazo[4,5-b]pyridin-2-yl)-5- | |
| | | (trifluoromethyl)phenol | |
| Int 10 | 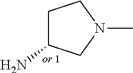 | 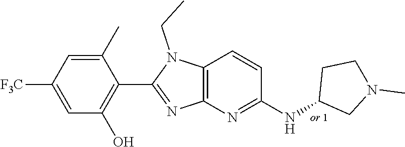 [SMILES: CCn1c(-c2c(C)cc(C(F)(F)F)cc2O)nc2nc(N[C@@H]3CCN(C)C3)ccc21] | 1. 58% 2. ¹H NMR (400 MHz, DMSO-d₆) δ 10.54 (s, 1H), 7.70 (d, 1H), 7.18 (s, 1H), 7.09 (d, 1H), 6.58- 6.36 (m, 2H), 4.35 (ddd, 1H), 3.88 (dt, 2H), 2.75 (dt, 1H), 2.59 (td, 1H), 2.43-2.34 (m, 2H), 2.25 |
| | | 8 | (d, 3H), 2.24-2.17 (m, |
| | | enantiopure 2-(1-ethyl-5-((1- | 1H), 2.11 (s, 3H), 1.69- |
| | | methylpyrrolidin-3-yl)amino)-1H- | 1.54 (m, 1H), 1.15 (t, 3H). |
| | | imidazo[4,5-b]pyridin-2-yl)-3-methyl-5- | 3. 420.34 |
| | | (trifluoromethyl)phenol | |
| Int 8 | 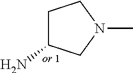 | 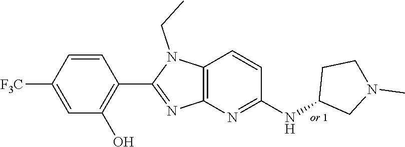 [SMILES: CCn1c(-c2ccc(C(F)(F)F)cc2O)nc2nc(N[C@@H]3CCN(C)C3)ccc21] | 1. 25% 2. ¹H NMR (400 MHz, DMSO-d₆) δ 12.17 (brs, 1H), 7.74 (d, 1H), 7.68 (d, 1H), 7.28-7.16 (m, 2H), 6.59 (d, 1H), 6.52 (d, 1H), 4.36 (dtt, 1H), 4.20 (q, 2H), 2.81-2.70 (m, 1H), 2.60 (td, 1H), 2.44-2.31 (m, 2H), 2.25 (s, 4H), 1.61 |
| | | 9 | (dddd, 1H), 1.32-1.19 |
| | | enantiopure 2-(1-ethyl-5-((1- | (m, 3H). |
| | | methylpyrrolidin-3-yl)amino)-1H- | 3. 406.14 |
| | | imidazo[4,5-b]pyridin-2-yl)-5- | |
| | | (trifluoromethyl)phenol | |
| Int 9 | 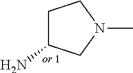 | 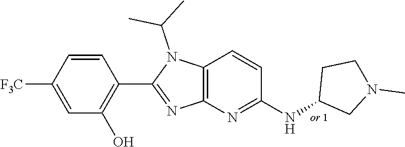 [SMILES: CC(C)n1c(-c2ccc(C(F)(F)F)cc2O)nc2nc(N[C@@H]3CCN(C)C3)ccc21] | 1. 35% 2. ¹H NMR (400 MHz, DMSO-d₆) δ 11.13 (s, 1H), 7.87 (d, 1H), 7.57 (d, 1H), 7.26 (d, 2H), 6.50 (d, 1H), 6.47 (d, 1H), 4.46- 4.18 (m, 2H), 2.75 (dd, 1H), 2.63-2.55 (m, 1H), 2.42-2.33 (m, 2H), 2.29- 2.16 (m, 4H), 1.68- |
| | | 10A | 1.55 (m, 1H), 1.53-1.42 |
| | | enantiopure 2-(1-isopropyl-5-((1- | (m, 6H). |
| | | methylpyrrolidin-3-yl)amino)-1H- | 3. 420.34 |
| | | imidazo[4,5-b]pyridin-2-yl)-5- | |
| | | (trifluoromethyl)phenol | |
| Int 11 | 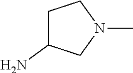 | 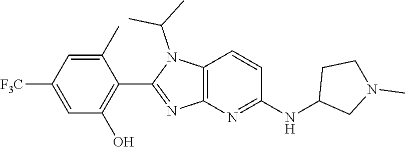 [SMILES: Cc1cc(C(F)(F)F)cc(O)c1-c1nc2nc(NC3CCN(C)C3)ccc2n1C(C)C] | 1. 34% 2. ¹H NMR (400 MHz, DMSO-d₆) δ 10.53 (s, 1H), 7.85 (d, 1H), 7.18 (s, 1H), 7.08 (d, 1H), 6.47 (d, 1H), 6.46-6.42 (m, 1H), 4.34 (dtt, 1H), 4.08 (p, 1H), 2.76 (dt, 1H), 2.59 (ddd, 1H), 2.45-2.34 (m, 2H), 2.26 (d, 3H), 2.24- |
| | | 10 | 2.17 (m, 1H), 2.10 (s, 3H), |
| | | 2-(1-isopropyl-5-((1-methylpyrrolidin-3- | 1.69-1.56 (m, 1H), 1.51 |
| | | yl)amino)-1H-imidazo[4,5-b]pyridin-2-yl)- | (d, 3H), 1.39 (d, 3H). |
| | | 3-methyl-5-(trifluoromethyl)phenol | 3. 434.37 |
| Int 11 | 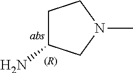 | 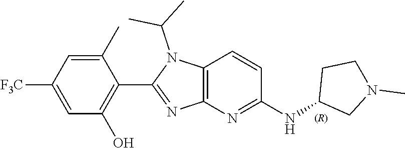 | 1. 66% 2. ¹H NMR (400 MHz, DMSO-d₆) δ 10.49 (s, 1H), 7.86 (d, 1H), 7.19 (d, 1H), 7.08 (d, 1H), 6.53- 6.39 (m, 2H), 4.35 (dddd, 1H), 4.08 (p, 1H), 2.78 (q, 1H), 2.69-2.56 (m, 1H), 2.46-2.31 (m, 2H), 2.31- 2.17 (m, 4H), 2.11 (d, |
| | | 10B | 3H), 1.71-1.56 (m, 1H), |
| | | (R)-2-(1-isopropyl-5-((1-methylpyrrolidin- | 1.51 (d, 3H), 1.39 (d, 3H). |
| | | 3-yl)amino)-1H-imidazo[4,5-b]pyridin-2- | 3. 434.31 |
| | | yl)-3-methyl-5-(trifluoromethyl)phenol | |
| Int 13 | 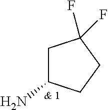 | 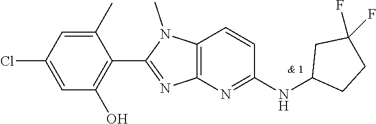 [SMILES: Cc1cc(Cl)cc(O)c1-c1nc2nc(NC3CCC(F)(F)C3)ccc2n1C] | 1. 20% 2. ¹H NMR (400 MHz, CDCl3) δ 7.46 (d, 1H), 6.95 (d, 1H), 6.83 (dd, 1H), 6.36 (d, 1H), 4.60 (dt, 1H), 3.59 (s, 3H), 2.70 (qd, 1H), 2.46-2.24 (m, 2H), 2.20 (s, 3H), 2.18- |
| | | 11 | 1.96 (m, 2H), 1.78 (dt, |
| | | 5-chloro-2-(5-((3,3- | 1H). |
| | | difluorocyclopentyl)amino)-1-methyl-1H- | 3. 391.29 [M − H]− |
| | | imidazo[4,5-b]pyridin-2-yl)-3- | |
| | | methylphenol | |
| Int 8 | 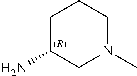 | 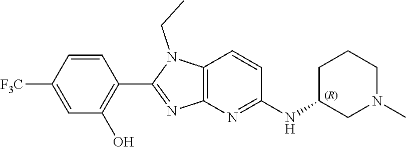 | 1. 32% 2. ¹H NMR (400 MHz, DMSO-d₆) δ 11.98 (brs, 1H), 7.75 (d, 1H), 7.72 (d, 1H), 7.29 (d, 2H), 6.54 (d, 1H), 6.33 (d, 1H), 4.20 (q, 2H), 3.98 (tq, 1H), 2.88 (d, 1H), 2.60-2.52 (m, 1H), 2.17 (s, 3H), 1.97 (dt, 1H), 1.83 (d, 2H), 1.70 |
| | | 12 | (dt, 1H), 1.54 (dtt, 1H), |
| | | (R)-2-(1-ethyl-5-((1-methylpiperidin-3- | 1.33-1.17 (m, 4H). |
| | | yl)amino)-1H-imidazo[4,5-b]pyridin-2-yl)- | 3. 420.41 |
| | | 5-(trifluoromethyl)phenol | |
| Int 9 | 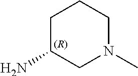 | 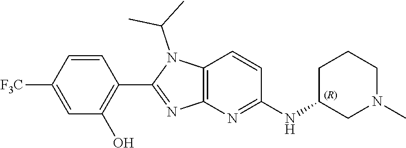 | 1. 47% 2. ¹H NMR (400 MHz, DMSO-d₆) δ 10.94 (s, 1H), 7.86 (d, 1H), 7.57 (d, 1H), 7.30 (d, 1H), 7.27 (s, 1H), 6.48 (d, 1H), 6.22 (d, 1H), 4.32 (h, 1H), 3.95 (dq, 1H), 2.89 (d, 1H), 2.63-2.55 (m, 1H), 2.17 (s, 3H), 2.03-1.92 (m, |
| | | 13 | 1H), 1.87-1.76 (m, 2H), |
| | | (R)-2-(1-isopropyl-5-((1-methylpiperidin- | 1.75-1.64 (m, 1H), 1.61- |
| | | 3-yl)amino)-1H-imidazo[4,5-b]pyridin-2- | 1.51 (m, 1H), 1.48 (dd, |
| | | yl)-5-(trifluoromethyl)phenol | 6H), 1.30-1.16 (m, 1H). |
| | | | 3. 434.44 |
| Int 8 | —NH₂ | 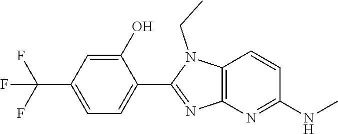 [SMILES: CCn1c(-c2ccc(C(F)(F)F)cc2O)nc2nc(NC)ccc21] | 1. 43% 2. ¹H NMR (400 MHz, DMSO-d₆) δ 12.48 (s, 1H), 7.75 (d, 1H), 7.68 (d, 1H), 7.24 (s, 1H), 7.20 (d, 1H), 6.48 (d, 1H), 6.43 (q, 1H), 4.22 (q, 2H), 2.82 (d, 3H), 1.27 (t, 3H). |
| | | 14 | 3. 337.09 |
| | | 2-(1-ethyl-5-(methylamino)-1H- | |
| | | imidazo[4,5-b]pyridin-2-yl)-5- | |
| | | (trifluoromethyl)phenol | |
| Int 11 | —NH₂ | 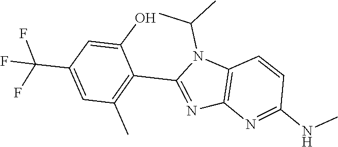 [SMILES: CNc1ccc2c(n1)nc(-c1c(C)cc(C(F)(F)F)cc1O)n2C(C)C] | 1. 60% 2. ¹H NMR (400 MHz, DMSO-d₆) δ 10.55 (s, 1H), 7.86 (d, 1H), 7.17 (s, 1H), 7.07 (s, 1H), 6.41 (d, 1H), 6.29 (q, 1H), 4.09 (p, 1H), 2.80 (d, 3H), 2.10 (s, 3H), 1.52 (d, 3H), 1.39 (d, 3H). |
| | | 15 | 3. 365.22 |
| | | 2-(1-isopropyl-5-(methylamino)-1H- | |
| | | imidazo[4,5-b]pyridin-2-yl)-3-methyl-5- | |
| | | (trifluoromethyl)phenol | |
| Int 8 | 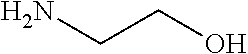 | 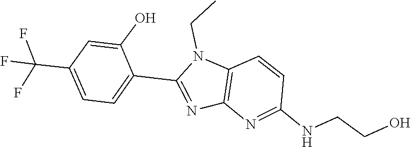 [SMILES: CCn1c(-c2ccc(C(F)(F)F)cc2O)nc2nc(NCCO)ccc21] | 1. 42% 2. ¹H NMR (400 MHz, DMSO-d₆) δ 11.81 (s, 1H), 7.78 (d, 1H), 7.74 (d, 1H), 7.36-7.25 (m, 2H), 6.56 (d, 1H), 6.52 (t, 1H), 4.78 (t, 1H), 4.22 (q, 2H), 3.63-3.53 (m, 2H), 1.29 (q, 3H) 2H under DMSO |
| | | 16 | peak. |
| | | 2-(1-ethyl-5-((2-hydroxyethyl)amino)-1H- | 3. 367.22 |
| | | imidazo[4,5-b]pyridin-2-yl)-5- | |
| | | (trifluoromethyl)phenol | |
| Int 9 | 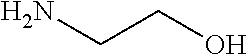 | 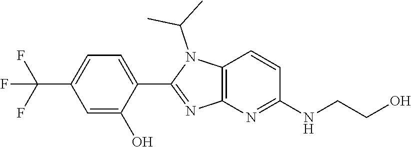 [SMILES: CC(C)n1c(-c2ccc(C(F)(F)F)cc2O)nc2nc(NCCO)ccc21] | 1. 58% 2. ¹H NMR (400 MHz, DMSO-d₆) δ 7.88 (d, 1H), 7.57 (d, 1H), 7.30-7.22 (m, 2H), 6.48 (d, 1H), 6.40 (t, 1H), 4.84-4.76 (m, 1H), 4.35 (p, 1H), 3.60-3.53 (m, 2H), 3.42- 3.37 (m, 2H), 1.48 (d, 6H). |
| | | 17 | 3. 381.36 |
| | | 2-(5-((2-hydroxyethyl)amino)-1-isopropyl- | |
| | | 1H-imidazo[4,5-b]pyridin-2-yl)-5- | |
| | | (trifluoromethyl)phenol | |

### Examples 18-18A and 18B: enantiopure 3-methyl-2-(1-methyl-5-((1-methylpyrrolidin-3-yl)amino)-1H-imidazo[4,5-b]pyridin-2-yl)-5-(trifluoromethyl)phenol

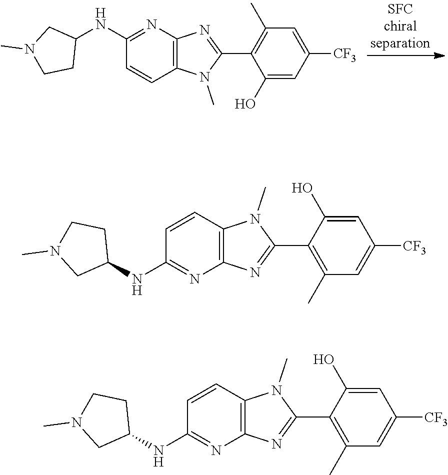 [SMILES: Cc1cc(C(F)(F)F)cc(O)c1-c1nc2nc(NC3CCN(C)C3)ccc2n1C ; Cc1cc(C(F)(F)F)cc(O)c1-c1nc2nc(N[C@@H]3CCN(C)C3)ccc2n1C ; Cc1cc(C(F)(F)F)cc(O)c1-c1nc2nc(N[C@H]3CCN(C)C3)ccc2n1C]

The racemate product (prepared as described for Ex 1 starting from Int 6 and commercially available racemate amine 1-methylpyrrolidin-3-amine) was obtained in 78% yield and separated by chiral SFC (column: Chiralpak IK, 250 mm×30 mm, 5 μm, 30° C., eluent B: MeOH 15%+0.5% diethylamine, flow: 90 g/min, pressure: 100 bar, cycle time: 18 min) to provide the two enantiomers Ex 18A (first eluting) and Ex 18B (second eluting).

Ex 18A ¹H NMR (400 MHz, DMSO-d6): δ=10.53 (brs, 1H), 7.70-7.68 (d, 1H), 7.19 (s, 1H), 7.11 (s, 1H), 6.57-6.55 (d, 1H), 6.51-6.49 (d, 1H), 4.41-4.38 (m, 1H), 3.47 (s, 3H), 3.00-2.98 (m, 1H), 2.92-2.88 (m, 1H), 2.67-2.66 (m, 2H), 2.43 (s, 3H), 2.33-2.26 (m, 1H), 2.14 (s, 3H), 1.76-1.73 (m, 1H).

MS (ESI): 406.34 [M+H]⁺.

Ex 18B ¹H NMR (400 MHz, DMSO-d6): δ=10.62 (brs, 1H), 7.66-7.64 (d, 1H), 7.15 (s, 1H), 7.08 (s, 1H), 6.49-6.44 (m, 2H), 4.36-4.35 (m, 1H), 3.46 (s, 3H), 2.75-2.74 (m, 1H), 2.66-2.58 (m, 1H), 2.38-2.35 (m, 2H), 2.30-2.29 (m, 4H), 2.13 (s, 3H), 1.60-1.63 (m, 1H).

MS (ESI): 406.34 [M+H]⁺.

### Example 19: enantiopure 2-(1-methyl-5-((1-methylpyrrolidin-3-yl)amino)-1H-imidazo[4,5-b]pyridin-2-yl)-5-(trifluoromethyl)phenol

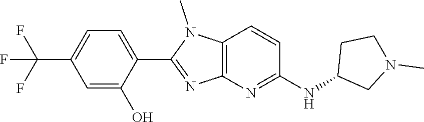 [SMILES: CN1CC[C@@H](Nc2ccc3c(n2)nc(-c2ccc(C(F)(F)F)cc2O)n3C)C1]

The racemate product (prepared as described for Ex 1 starting from Int 7 and commercially available racemate 1-methylpyrrolidin-3-amine) was separated by chiral SFC (column: Chiralpak IE, 250 mm×10 mm, 5 μm, 30° C., eluent B: MeOH 15%+0.2% diethylamine, flow: 2 mL/min, cycle time: 18 min) to provide the pure enantiomer Ex 19 (second eluting).

¹H NMR (400 MHz, DMSO-d6): δ=12.40 (brs, 1H), 7.89-7.87 (d, 1H), 7.75-7.73 (d, 1H), 7.30-7.27 (m, 2H), 6.67-6.65 (d, 1H), 6.56-6.54 (d, 1H), 4.39-4.34 (m, 1H), 3.82 (s, 3H), 2.76-2.72 (m, 1H), 2.67-2.57 (m, 1H), 2.40-2.32 (m, 2H), 2.27-2.19 (m, 4H), 1.65-1.57 (m, 1H).

MS (ESI): 392.41 [M+H]⁺.

### Example 20: (trans) enantiopure 2-(5-((2-hydroxycyclohexyl)amino)-1-methyl-1H-imidazo[4,5-b]pyridin-2-yl)-3-methyl-5-(trifluoromethyl)phenol

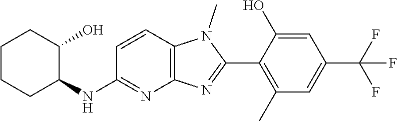 [SMILES: Cc1cc(C(F)(F)F)cc(O)c1-c1nc2nc(N[C@H]3CCCC[C@@H]3O)ccc2n1C]

The racemate product (prepared as described for Ex 1 starting from Int 6 and commercially available trans racemate 2-aminocyclohexan-1-ol) was separated by chiral SFC (column: Chiralpak IC, 150 mm×4.6 mm, 5 μm, 30° C., eluent B: MeOH 20%, flow: 100 g/min, cycle time: 18 min, pressure: 100 bar) to provide the pure enantiomer Ex 20 (first eluting).

¹H NMR (400 MHz, DMSO-d6): δ=10.50 (s, 1H), 7.68-7.66 (d, 1H), 7.10 (s, 1H), 7.10 (s, 1H), 6.54-6.52 (d, 1H), 6.19-6.14 (m, 1H), 5.09-4.94 (d, 1H), 3.64-3.60 (m, 1H), 3.47 (s, 3H), 3.36-3.34 (m, 1H), 2.14 (s, 3H), 2.07-2.05 (m, 1H), 1.91-1.89 (m, 1H), 1.67-1.63 (m, 2H), 1.32-1.12 (m, 4H).

MS (ESI): 421.14 [M+H]⁺.

### Example 21: (trans) enantiopure 2-(5-((3-hydroxycyclohexyl)amino)-1-methyl-1H-imidazo[4,5-b]pyridin-2-yl)-3-methyl-5-(trifluoromethyl)phenol

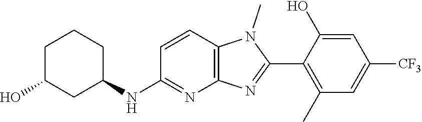 [SMILES: Cc1cc(C(F)(F)F)cc(O)c1-c1nc2nc(N[C@@H]3CCC[C@@H](O)C3)ccc2n1C]

The racemate product (prepared as described for Ex 1 starting from Int 6 and commercially available trans racemate 3-aminocyclohexan-1-ol) was obtained in 93% yield separated by chiral SFC (column: Chiralpak IK, 250 mm×30 mm, 5 μm, 30° C., eluent B: MeOH 20%+0.5% IPA, flow: 90 g/min, cycle time: 18 min, pressure: 100 bar) to provide the pure enantiomer Ex 21 (first eluting).

¹H NMR (400 MHz, DMSO-d6): δ=10.37 (brs, 1H), 7.64-7.62 (d, 1H), 7.17 (s, 1H), 7.09 (s, 1H), 6.48-6.46 (d, 1H), 6.09-6.07 (d, 1H), 4.40 (s, 1H), 4.21-4.18 (m, 1H), 3.93-3.89 (m, 1H), 3.46 (s, 3H), 2.14 (s, 3H), 1.80-1.47 (m, 7H), 1.35-1.28 (m, 1H).

MS (ESI): 421.67 [M+H]⁺.

### Examples 22A and 22B: (trans) enantiopure 2-(5-((3-hydroxycyclohexyl)amino)-1-methyl-1H-imidazo[4,5-b]pyridin-2-yl)-5-(trifluoromethyl)phenol

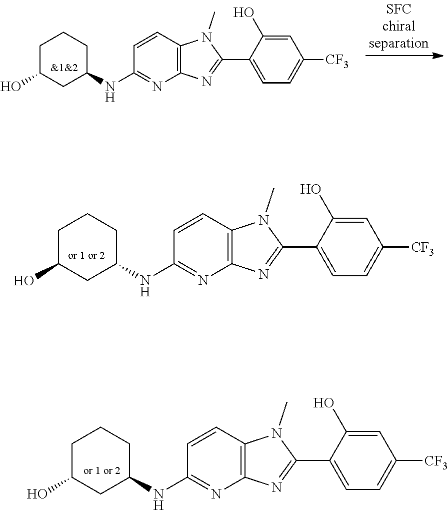 [SMILES: Cn1c(-c2ccc(C(F)(F)F)cc2O)nc2nc(N[C@@H]3CCC[C@@H](O)C3)ccc21 ; Cn1c(-c2ccc(C(F)(F)F)cc2O)nc2nc(N[C@H]3CCC[C@H](O)C3)ccc21 ; Cn1c(-c2ccc(C(F)(F)F)cc2O)nc2nc(N[C@@H]3CCC[C@@H](O)C3)ccc21]

The racemate product (prepared as described for Ex 1 starting from Int 7 and commercially available trans racemate 3-aminocyclohexan-1-ol) was obtained in 38% yield and separated by chiral SFC (column: Chiralpak IK, 250 mm×4.6 mm, 5 μm, 30° C., eluent B: MeOH 25%+0.5% IPA, flow: 3 mL/min, pressure: 1500 psi, cycle time: 26 min) to provide the two enantiomers Ex 22A (first eluting) and Ex 22B (second eluting).

Ex 22A ¹H NMR (400 MHz, DMSO-d₆) δ 12.48 (s, 1H), 7.91 (d, 1H), 7.74 (d, 1H), 7.35-7.28 (m, 2H), 6.55 (d, 1H), 6.32 (d, 1H), 4.43 (d, 1H), 4.21 (td, 1H), 4.01-3.90 (m, 1H), 3.84 (s, 3H), 1.89-1.64 (m, 3H), 1.63-1.40 (m, 4H), 1.37-1.25 (m, 1H).

MS (ESI): 407.38 [M+H]⁺.

Ex 22B ¹H NMR (400 MHz, DMSO-d₆) δ 12.42 (s, 1H), 7.90 (d, 1H), 7.73 (d, 1H), 7.32 (s, 1H), 7.29 (dd, 1H), 6.55 (d, 1H), 6.31 (d, 1H), 4.52-4.34 (m, 1H), 4.21 (t, 1H), 3.98-3.90 (m, 1H), 3.84 (s, 3H), 1.88-1.63 (m, 3H), 1.62-1.40 (m, 4H), 1.35-1.25 (m, 1H).

MS (ESI): 407.38 [M+H]⁺.

### Example 23: (2-(5-methoxy-1-methyl-1H-imidazo[4,5-b]pyridin-2-yl)-3-methyl-5-(trifluoromethyl)phenol

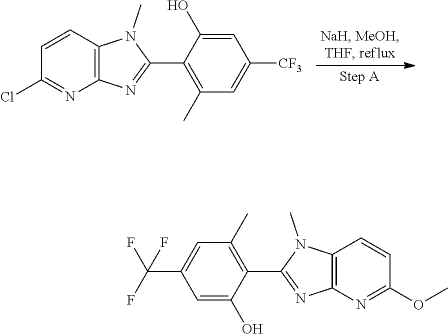 [SMILES: Cc1cc(C(F)(F)F)cc(O)c1-c1nc2nc(Cl)ccc2n1C ; COc1ccc2c(n1)nc(-c1c(C)cc(C(F)(F)F)cc1O)n2C]

### Step A:

To a stirred solution of 2-(5-chloro-1-methyl-1H-imidazo[4,5-b]pyridin-2-yl)-3-methyl-5-(trifluoromethyl)phenol (Int 6) (0.200 g, 0.59 mmol) and methanol (0.038 g, 1.17 mmol) in THF (10 mL) was added sodium hydride, 60% dispersion in mineral oil (0.4 g, 3.51 mmol), at RT and the reaction mixture was heated to 70° C. for 96 h. After consumption of the starting material, the reaction mixture was diluted with water (10 mL) and extracted with EtOAc (2×20 mL). The combined organic layers were washed with brine solution (20 mL), dried over anhydrous sodium sulfate, filtered and concentrated under reduced pressure to get the crude product as a yellow solid. The crude compound was purified by Prep HPLC (mobile phase A: 10 mM ammonium bicarbonate (aq), mobile phase B: acetonitrile: MeOH (60:40), Column: INERTSIL ODS, Flow: 19 ml/min, Method: (t/% B): 0/40, 1/40, 11/60, 15/20, 15.1/100, 17/100, 17.1/40, 19/40, Solubility: ACN+THF+water temperature: ambient). The pure fraction was lyophilized to get (2-(5-methoxy-1-methyl-1H-imidazo[4,5-b]pyridin-2-yl)-3-methyl-5-(trifluoromethyl)phenol (78 mg, 39%) as an off-white solid.

MS (ESI): 338.13 [M+H]⁺.

¹H NMR (400 MHz, DMSO-d6): δ=10.65 (brs, 1H), 8.02-7.99 (d, 1H), 7.21 (s, 1H), 7.12 (s, 1H), 6.77-6.75 (d, 1H), 3.90 (s, 3H), 3.57 (s, 3H), 2.14 (s, 3H).

Following the procedure of Example 23, the following examples were prepared, using the haloaromatic intermediates and alcohols as shown in Table 3:

TABLE 3

| | | | 1. Yield |
|---|---|---|---|
| Halo- | | | 2. ¹H-NMR |
| aromatic | Alcohol | Example | 3. MH⁺ (ESI) |
| 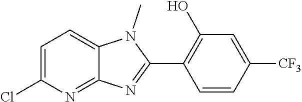 [SMILES: Cn1c(-c2ccc(C(F)(F)F)cc2O)nc2nc(Cl)ccc21] | CH₃OH | 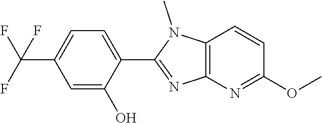 [SMILES: COc1ccc2c(n1)nc(-c1ccc(C(F)(F)F)cc1O)n2C] | 1. 21% 2. ¹H NMR (400 MHz DMSO-d6): δ = 8.03- 8.01 (d, 1H), 7.49-7.47 (d, 1H), 6.92 |
| Int 7 | | 24 | (s, 1H), |
| | | 2-(5-methoxy-1-methyl-1H- | 6.79-6.67 (m, |
| | | imidazo[4,5-b]pyridin-2-yl)-5- | 2H), 3.89 |
| | | (trifluoromethyl)phenol | (s, 3H), 3.74 |
| | | | (s, 3H). |
| | | | 3. 324.21 |
| 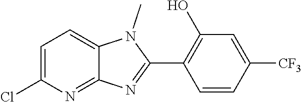 [SMILES: Cn1c(-c2ccc(C(F)(F)F)cc2O)nc2nc(Cl)ccc21] | 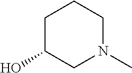 | 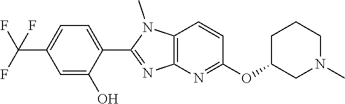 [SMILES: CN1CCC[C@@H](Oc2ccc3c(n2)nc(-c2ccc(C(F)(F)F)cc2O)n3C)C1] | 1. 16% 2. ¹H NMR (400 MHz DMSO-d6): δ = 11.80 (brs, 1H), 8.03-8.01(d, |
| Int 7 | | 25 | 1H), 7.77- |
| | | (R)-2-(1-methyl-5-((1- | 7.75 (d, 1H), |
| | | methylpiperidin-3-yl)oxy)-1H- | 7.34-7.30 |
| | | imidazo[4,5-b]pyridin-2-yl)-5- | (m, 2H), |
| | | (trifluoromethyl)phenol | 6.74-6.72 |
| | | | (d, 1H), |
| | | | 5.15-5.08 (m, |
| | | | 1H), 3.77 |
| | | | (s, 3H), 2.95- |
| | | | 2.93 (d, |
| | | | 1H), 2.57- |
| | | | 2.55 (d, 1H), |
| | | | 3.18 (s, 3H), |
| | | | 2.11-1.98 |
| | | | (m, 3H), |
| | | | 1.77-1.72 (m, |
| | | | 1H), 1.61- |
| | | | 1.53 (m, 1H), |
| | | | 1.46-1.56 |
| | | | (m, 1H). |
| | | | 3. 407.30 |
| 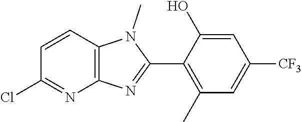 [SMILES: Cc1cc(C(F)(F)F)cc(O)c1-c1nc2nc(Cl)ccc2n1C] | 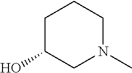 | 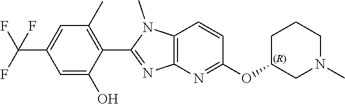 | 1. 12% 2. ¹H NMR (400 MHz, DMSO-d₆) δ 7.85 (d, 1H), 6.81 (d, 2H), 6.63 (d, 1H), 5.14 |
| Int 6 | | 26 | (tt, 1H), |
| | | (R)-3-methyl-2-(1-methyl-5-((1- | 3.56 (s, |
| | | methylpiperidin-3-yl)oxy)-1H- | 3H), 2.98- |
| | | imidazo[4,5-b]pyridin-2-yl)-5- | 2.90 (m, |
| | | (trifluoromethyl)phenol | 1H), 2.61- |
| | | | 2.53 (m, |
| | | | 1H), 2.21 (s, |
| | | | 3H), 2.17- |
| | | | 1.98 (m, |
| | | | 6H), 1.81- |
| | | | 1.72 (m, |
| | | | 1H), 1.66- |
| | | | 1.54 (m, |
| | | | 1H), 1.50- |
| | | | 1.39 (m, |
| | | | 1H). |
| | | | 3. 421.2 |
| 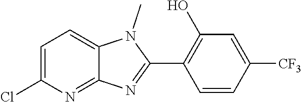 [SMILES: Cn1c(-c2ccc(C(F)(F)F)cc2O)nc2nc(Cl)ccc21] | 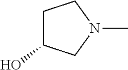 | 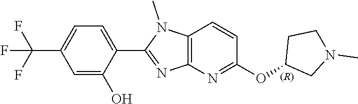 | 1. 36% 2. ¹H NMR (400 MHz, DMSO-d₆) δ 11.48 (s, 1H), 8.04 (d, 1H), 7.85- |
| Int 7 | | 27 | 7.72 (m, |
| | | (R)-2-(1-methyl-5-((1- | 1H), 7.44- |
| | | methylpyrrolidin-3-yl)oxy)-1H- | 7.26 (m, 2H), |
| | | imidazo[4,5-b]pyridin-2-yl)-5- | 6.76 (d, |
| | | (trifluoromethyl)phenol | 1H), 5.42 |
| | | | (ddd, 1H), |
| | | | 3.78 (s, 3H), |
| | | | 2.83 (dd, |
| | | | 1H), 2.74- |
| | | | 2.60 (m, |
| | | | 2H), 2.43- |
| | | | 2.30 (m, |
| | | | 2H), 2.27 (s, |
| | | | 3H), 1.89- |
| | | | 1.76 (m, 1H). |
| | | | 3. 393.32 |
| 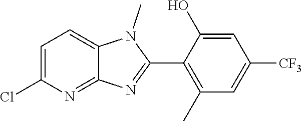 [SMILES: Cc1cc(C(F)(F)F)cc(O)c1-c1nc2nc(Cl)ccc2n1C] | 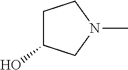 | 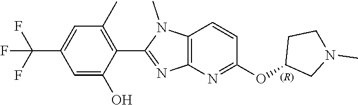 | 1. 26% 2. ¹H NMR (400 MHz, DMSO-d₆) δ 7.98 (d, 1H), 7.17 (s, 1H), 7.09 (s, 1H), 6.72 |
| Int 6 | | 28 | (d, 1H), |
| | | (R)-3-methyl-2-(1-methyl-5-((1- | 5.48-5.35 |
| | | methylpyrrolidin-3-yl)oxy)-1H- | (m, 1H), |
| | | imidazo[4,5-b]pyridin-2-yl)-5- | 3.56 (s, 3H), |
| | | (trifluoromethyl)phenol | 2.82 (dd, |
| | | | 1H), 2.72- |
| | | | 2.53 (m, |
| | | | 3H), 2.41- |
| | | | 2.30 (m, |
| | | | 2H), 2.26 (d, |
| | | | 3H), 2.14 |
| | | | (s, 3H). |
| | | | 3. 407.28 |
| 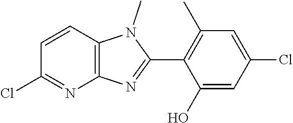 [SMILES: Cc1cc(Cl)cc(O)c1-c1nc2nc(Cl)ccc2n1C] | CH₃OH | 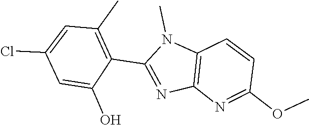 [SMILES: COc1ccc2c(n1)nc(C1=C(C)C=C(Cl)C=C(O)C1)n2C] | 1. 20% 2. ¹H NMR (400 MHz, DMSO-d₆) δ 10.29 (s, 1H), 7.98 (d, 1H), 6.94 (dd, 1H), |
| Int 13 | | 29 | 6.88 |
| | | 5-chloro-2-(5-methoxy-1-methyl- | (d, 1H), |
| | | 1H-imidazo[4,5-b]pyridin-2-yl)-3- | 6.74 (d, 1H), |
| | | methylphenol | 3.89 (s, |
| | | | 3H), 3.55 (s, |
| | | | 3H), 2.06 |
| | | | (s, 3H). |
| | | | 3. 304.07 |
| 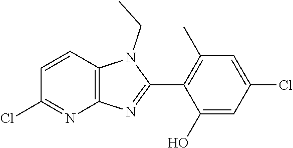 [SMILES: CCn1c(-c2c(C)cc(Cl)cc2O)nc2nc(Cl)ccc21] | CH₃OH | 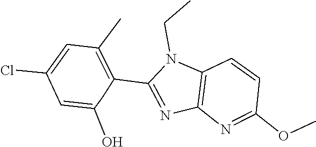 [SMILES: CCn1c(C2=C(C)C=C(Cl)C=C(O)C2)nc2nc(OC)ccc21] | 1. 51% 2. ¹H NMR (400 MHz, DMSO-d₆) δ 10.49 (s, 1H), 8.02 (d, 1H), 6.89 (dd, 2H), 6.73 (d, 1H), 4.04-3.93 |
| Int 14 | | 30 | (m, 2H), |
| | | 5-chloro-2-(1-ethyl-5-methoxy- | 3.89 (s, 3H), |
| | | 1H-imidazo[4,5-b]pyridin-2-yl)-3- | 2.02 (s, |
| | | methylphenol | 3H), 1.17 |
| | | | (t, 3H). |
| | | | 3. 318.18 |
| 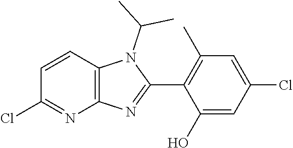 [SMILES: Cc1cc(Cl)cc(O)c1-c1nc2nc(Cl)ccc2n1C(C)C] | CH₃OH | 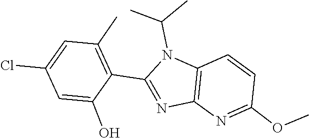 [SMILES: COc1ccc2c(n1)nc(-c1c(C)cc(Cl)cc1O)n2C(C)C] | 1. 77% 2. ¹H NMR (400 MHz, DMSO-d₆) δ 10.37 (s, 1H), 8.18 (d, 1H), 6.94 (d, 1H), 6.86 (d, 1H), |
| Int 15 | | 31 | 6.69 (d, 1H), |
| | | 5-chloro-2-(1-isopropyl-5- | 4.19 (p, |
| | | methoxy-1H-imidazo[4,5- | 1H), 3.88 (s, |
| | | b]pyridin-2-yl)-3-methylphenol | 3H), 2.01 |
| | | | (s, 3H), 1.54 |
| | | | (d, 3H), |
| | | | 1.42 (d, 3H). |
| | | | 3. 332.24 |
| 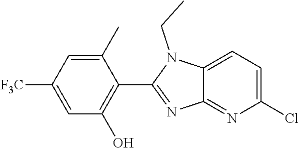 [SMILES: CCn1c(-c2c(C)cc(C(F)(F)F)cc2O)nc2nc(Cl)ccc21] | 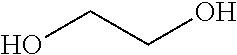 | 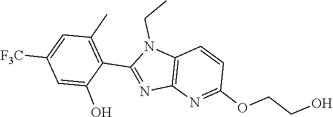 [SMILES: CCn1c(-c2c(C)cc(C(F)(F)F)cc2O)nc2nc(OCCO)ccc21] | 1. 23% 2. ¹H NMR (400 MHz, DMSO-d₆) δ 10.64 (s, 1H), 8.05 (d, 1H), 7.29- 7.03 (m, 2H), 6.75 (d, 1H), 4.83 (t, |
| Int 10 | | 32 | 1H), 4.32 |
| | | 2-(1-ethyl-5-(2-hydroxyethoxy)- | (dd, 2H), |
| | | 1H-imidazo[4,5-b]pyridin-2-yl)-3- | 3.98 |
| | | methyl-5-(trifluoromethyl)phenol | (dh, 2H), |
| | | | 3.75 (q, 2H), |
| | | | 2.11 (s, |
| | | | 3H), 1.19 |
| | | | (t, 3H). |
| | | | 3. 382.22 |
| 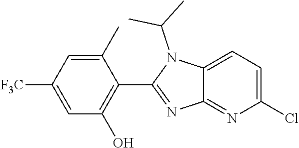 [SMILES: Cc1cc(C(F)(F)F)cc(O)c1-c1nc2nc(Cl)ccc2n1C(C)C] | 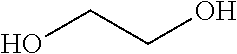 | 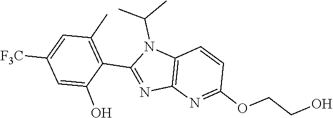 [SMILES: Cc1cc(C(F)(F)F)cc(O)c1-c1nc2nc(OCCO)ccc2n1C(C)C] | 1. 26% 2. ¹H NMR (400 MHz, DMSO-d₆) δ 10.74 (s, 1H), 8.20 (d, 1H), 7.14 (d, 2H), 6.71 (d, 1H), 4.96-4.78 |
| Int 11 | | 33 | (m, 1H), |
| | | 2-(5-(2-hydroxyethoxy)-1- | 4.32 (t, |
| | | isopropyl-1H-imidazo[4,5- | 2H), 4.24- |
| | | b]pyridin-2-yl)-3-methyl-5- | 4.04 |
| | | (trifluoromethyl)phenol | (m, 1H), |
| | | | 3.75 (q, |
| | | | 2H), 2.10 (s, |
| | | | 3H), 1.55 |
| | | | (d, 3H), |
| | | | 1.43 (d, 3H). |
| | | | 3. 396.3 |
| 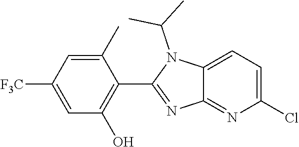 [SMILES: Cc1cc(C(F)(F)F)cc(O)c1-c1nc2nc(Cl)ccc2n1C(C)C] | CH₃OH | 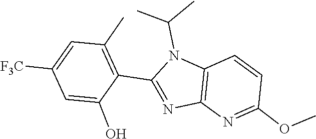 [SMILES: COc1ccc2c(n1)nc(-c1c(C)cc(C(F)(F)F)cc1O)n2C(C)C] | 1. 78% 2. ¹H NMR (400 MHz, DMSO-d₆) δ 10.62 (s, 1H), 8.20 (d, 1H), 7.21 (s, 1H), 7.10 (d, 1H), 6.71 (d, |
| Int 11 | | 34 | 1H), 4.17 |
| | | 2-(1-isopropyl-5-methoxy-1H- | (hept, 1H), |
| | | imidazo[4,5-b]pyridin-2-yl)-3- | 3.89 (s, |
| | | methyl-5-(trifluoromethyl)phenol | 3H), 2.11 (s, |
| | | | 3H), 1.55 |
| | | | (d, 3H), 1.43 |
| | | | (d, 3H). |
| | | | 3. 366.29 |
| 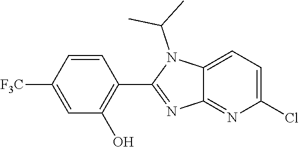 [SMILES: CC(C)n1c(-c2ccc(C(F)(F)F)cc2O)nc2nc(Cl)ccc21] | CH₃OH | 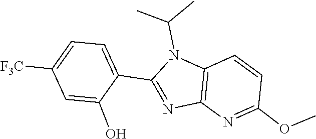 [SMILES: COc1ccc2c(n1)nc(-c1ccc(C(F)(F)F)cc1O)n2C(C)C] | 1. 73% 2. ¹H NMR (400 MHz, DMSO-d₆) δ 8.10 (d, 1H), 7.36 (d, 1H), 6.85 (s, 1H), 6.68 (s, 1H), |
| Int 9 | | 35 | 6.63 (d, 1H), |
| | | 2-(1-isopropyl-5-methoxy-1H- | 4.54 (h, |
| | | imidazo[4,5-b]pyridin-2-yl)-5- | 1H), 3.88 (s, |
| | | (trifluoromethyl)phenol | 3H), 1.49 |
| | | | (d, 6H). |
| | | | 3. 352.27 |

### Example 36: cis enantiopure 2-(5-(hydroxycyclopentyl)amino)-1-methyl-1H-imidazo[4,5-b]pyridin-2-yl)-3-methyl-5-(trifluoromethyl)phenol

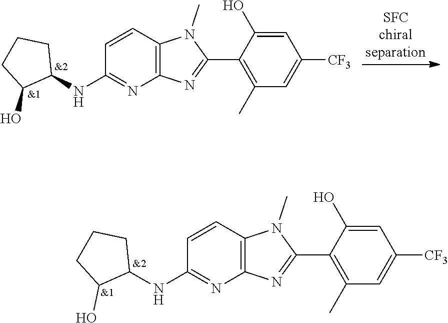 [SMILES: Cc1cc(C(F)(F)F)cc(O)c1-c1nc2nc(N[C@@H]3CCC[C@@H]3O)ccc2n1C ; Cc1cc(C(F)(F)F)cc(O)c1-c1nc2nc(NC3CCCC3O)ccc2n1C]

The racemate product (prepared as described for Ex 1 starting from Int 6 and commercially available cis racemate 2-aminocyclopentan-1-ol) was obtained in 50% yield and was separated by chiral SFC (column: Lux-cellulose-5C, 150 mm×4.6 mm, 3 μm, 30° C., eluent B: MeOH 25%+0.5% IPA, flow: 3 mL/min, pressure: 1500 psi, run time: 14 min) to provide the pure cis enantiomer Ex 25 (second eluting).

¹H NMR (400 MHz, DMSO-d₆) δ 10.63 (s, 1H), 7.70 (d, 1H), 7.15 (s, 1H), 7.08 (d, 1H), 6.53 (d, 1H), 6.43 (dd, 1H), 5.57 (d, 1H), 3.93-3.77 (m, 2H), 3.48 (s, 3H), 2.19-2.05 (m, 4H), 1.89 (ddt, 1H), 1.77-1.60 (m, 2H), 1.59-1.44 (m, 2H).

MS (ESI): 407.3 [M+H]⁺.

### Example 37A and 37B: trans-2-(5-((3-hydroxycyclopentyl)oxy)-1-methyl-1H-imidazo[4,5-b]pyridin-2-yl)-5-(trifluoromethyl)phenol

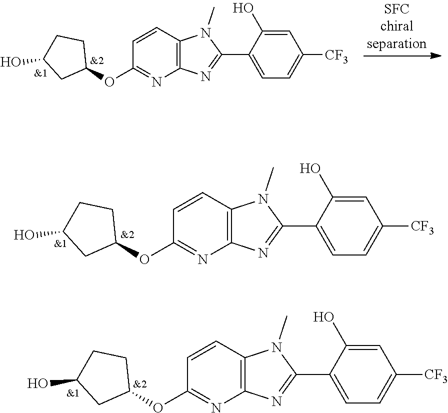 [SMILES: Cn1c(-c2ccc(C(F)(F)F)cc2O)nc2nc(O[C@@H]3CC[C@@H](O)C3)ccc21 ; Cn1c(-c2ccc(C(F)(F)F)cc2O)nc2nc(O[C@@H]3CC[C@@H](O)C3)ccc21 ; Cn1c(-c2ccc(C(F)(F)F)cc2O)nc2nc(O[C@H]3CC[C@H](O)C3)ccc21]

The racemate product (prepared as described for Ex 1 starting from Int 7 and commercially available trans racemate cyclopentane-1,3-diol) was obtained in 13% yield and was separated by chiral SFC (column: Chiralpak IG, 250 mm×4.6 mm, 5 μm, 30° C., eluent B: MeOH 40%+0.5% IPA, flow: 3 mL/min, pressure: 1500 psi, run time: 18 min) to provide the pure trans enantiomers.

### Ex 37A (First Eluting)

¹H NMR (400 MHz, DMSO-d₆) δ 11.58 (s, 1H), 8.01 (d, 1H), 7.79 (d, 1H), 7.31 (d, 2H), 6.72 (d, 1H), 5.51 (tt, 1H), 4.60 (d, 1H), 4.35-4.21 (m, 1H), 3.78 (s, 3H), 2.23 (dtd, 1H), 2.04 (dddd, 1H), 1.99-1.82 (m, 2H), 1.74-1.63 (m, 1H), 1.61-1.47 (m, 1H).

MS (ESI): 394.22 [M+H]⁺.

### Ex 37B (Second Eluting)

¹H NMR (400 MHz, DMSO-d₆) δ 11.58 (s, 1H), 8.01 (d, 1H), 7.79 (d, 1H), 7.34-7.28 (m, 2H), 6.72 (d, 1H), 5.51 (tt, 1H), 4.60 (d, 1H), 4.34-4.25 (m, 1H), 3.78 (s, 3H), 2.23 (dtd, 1H), 2.04 (dddd, 1H), 1.99-1.83 (m, 2H), 1.74-1.62 (m, 1H), 1.60-1.48 (m, 1H).

MS (ESI): 394.23 [M+H]⁺.

### Example 38: 5-chloro-3-methyl-2-(1-methyl-5-(2,2,2-trifluoroethoxy)-1H-imidazo[45-b]pyridin-2-yl)phenol

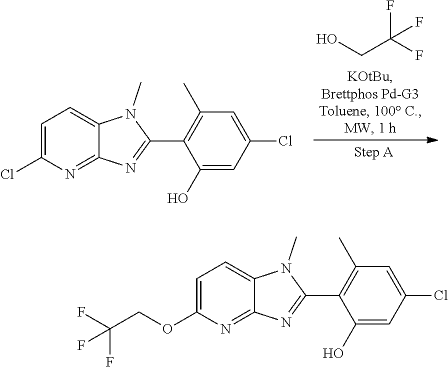 [SMILES: OCC(F)(F)F ; Cc1cc(Cl)cc(O)c1-c1nc2nc(Cl)ccc2n1C ; Cc1cc(Cl)cc(O)c1-c1nc2nc(OCC(F)(F)F)ccc2n1C]

### Step A:

To a stirred solution of 5-chloro-2-(5-chloro-1-methyl-1H-imidazo[4,5-b]pyridin-2-yl)-3-methylphenol (200 mg, 0.649 mmol) (Int 13) and 2,2,2-trifluoroethan-1-ol (325 mg, 3.245 mmol) in toluene (10 mL) was added potassium tert-butoxide (218 mg, 1.947 mmol) at RT and the mixture was purged with nitrogen gas for 5 min. BrettPhos-Pd G3 (59 mg, 0.065 mmol) was added to the above mixture and the mixture was irradiated under microwave irradiation at 100° C. for 1 h. The progress of the reaction was monitored by TLC. The reaction mixture was cooled to rt, filtered on a celite bed and washed with EtOAc (50 mL). The resulting filtrate was concentrated under reduced pressure to get a crude product. The crude compound was purified by Prep HPLC. Conditions: mobile phase A: 10 mM ammonium bicarbonate aq., mobile phase B: acetonitrile, column: UNISIL, flow: 19 mL/min. Method: (T/% of B): 0/45, 3/50, 13/60, 15/60, 15.1/99, 20/99, 20.1/45, 24/45. Solubility: ACN+THF+water, temperature: ambient.

The pure fractions were collected and lyophilized to get the pure 5-chloro-3-methyl-2-(1-methyl-5-(2,2,2-trifluoroethoxy)-1H-imidazo[4,5-b]pyridin-2-yl)phenol (38 mg, 156%) as an off-white solid.

MS (ESI): 372.17 [M+H]⁺.

¹H NMR (400 MHz, DMSO-d₆) δ 10.64 (s, 1H), 8.09 (d, 1H), 6.91-6.83 (m, 3H), 5.04 (q, 2H), 3.58 (s, 3H), 2.05 (s, 3H).

### Example 39: 5-chloro-2-(5-(3,3-difluorocyclobutoxy)-1-methyl-1H-imidazo[4,5-b]pyridin-2-yl)-3-methylphenol

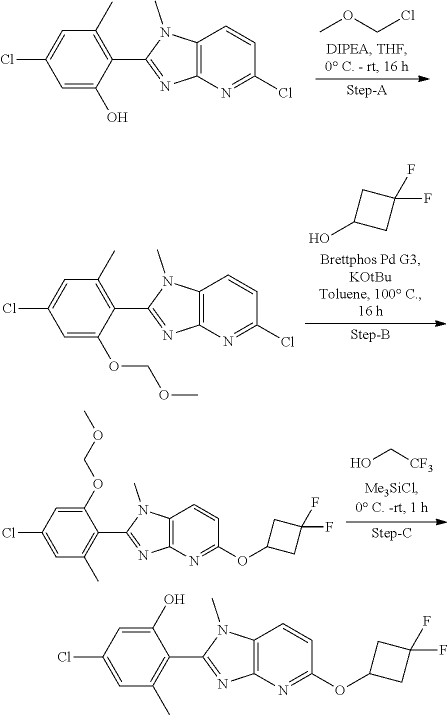

### Step A:

To a stirred solution of 5-chloro-2-(5-chloro-1-methyl-1H-imidazo[4,5-b]pyridin-2-yl)-3-methylphenol (800 mg, 2.596 mmol) (Int 13) in THF (8 mL) were added N, N-diisopropylethylamine (1.8 mL, 10.384 mmol), and methoxymethyl chloride (0.59 mL, 7.79 mmol) dropwise at 0° C. under argon atmosphere. The reaction mixture was stirred at RT for 16 h. The progress of the reaction was monitored by TLC. TLC showed consumption of the starting material. The reaction mixture was diluted with ice water (100 mL) and extracted with EtOAc (2×100 mL). The combined organic layers were washed with brine solution (100 mL), dried over anhydrous sodium sulfate, filtered and concentrated under reduced pressure to get a crude product as a liquid. The crude product was purified by column chromatography using silica gel. The product was eluted at 40% EtOAc in hexane. The pure fractions were collected and concentrated under reduced pressure to get the pure 5-chloro-2-(4-chloro-2-(methoxymethoxy)-6-methylphenyl)-1-methyl-1H-imidazo[4,5-b]pyridine (501 mg, 55%).

MS (ESI): 352.14 [M+H]⁺.

### Step B:

To a stirred solution of 5-chloro-2-(4-chloro-2-(methoxymethoxy)-6-methylphenyl)-1-methyl-1H-imidazo[4,5-b]pyridine (400 mg, 1.136 mmol) and 3,3-difluorocyclobutan-1-ol (613.76 mg, 5.678 mmol) in toluene (10 mL) was added potassium tert-butoxide (382.30 mg, 3.407 mmol) at RT. The mixture was purged with nitrogen gas for 5 min and then BrettPhos Pd G3 (103 mg, 0.114 mmol) was added and the mixture was heated to 100° C. for 16 h in a sealed condition. The progress of the reaction was monitored by TLC. TLC showed completion of reaction. The reaction mixture was cooled to rt, filtered on a celite bed, washed with EtOAc (250 mL) and concentrated under reduced pressure to get a crude product 2-(4-chloro-2-(methoxymethoxy)-6-methylphenyl)-5-(3,3-difluorocyclobutoxy)-1-methyl-1H-imidazo[4,5-b]pyridine (400 mg, 15%).

MS (ESI): 424.24 [M+H]⁺.

### Step C:

To a stirred solution of 2-(4-chloro-2-(methoxymethoxy)-6-methylphenyl)-5-(3,3-difluorocyclobutoxy)-1-methyl-1H-imidazo[4,5-b]pyridine (400 mg, 0.944 mmol) in trifluoroethanol (12.5 mL) was added chlorotrimethylsilane (5.0 mL) at 0° C. and the reaction mixture was stirred at RT for 1 h. The progress of the reaction was monitored by TLC. TLC showed completion of reaction. The reaction mixture was evaporated under reduced pressure then diluted with ice/water (100 mL) and extracted with DCM (2×100 mL). The combined organic layer was washed with brine solution (100 mL), dried over anhydrous sodium sulfate, filtered and concentrated under reduced pressure to get a crude product as a solid. The crude compound was purified by Prep HPLC. Prep HPLC Conditions: mobile phase A: ammonium bicarbonate, mobile phase B: acetonitrile, column: HYPERSIL BDS (20 mm×250 mm) 5 μM, flow: 19 mL/min. Method: (T/% of B): −0/45, 2/45, 6/65, 10.50/65, 10.51/100, 16/100, 16.01/45, 20/45 Solubility: ACN+THF+water. The pure fractions were collected and lyophilized to get the pure 5-chloro-2-(5-(3,3-difluorocyclobutoxy)-1-methyl-1H-imidazo[4,5-b]pyridin-2-yl)-3-methylphenol (6.5 mg, 11%) as a yellow solid.

MS (ESI): 378.19 [M+H]⁺.

¹H NMR (400 MHz, DMSO-d₆) δ 7.99 (d, 1H), 6.80-6.69 (m, 3H), 5.18 (qd, 1H), 3.55 (s, 3H), 3.23-3.12 (m, 2H), 2.82-2.64 (m, 2H), 2.03 (s, 3H).

### Example 40: (R)-2-(6-((1-methylpiperidin-3-yl)amino)-2H-pyrazolo[3,4-b]pyridin-2-yl)-5-(trifluoromethyl)phenol formate

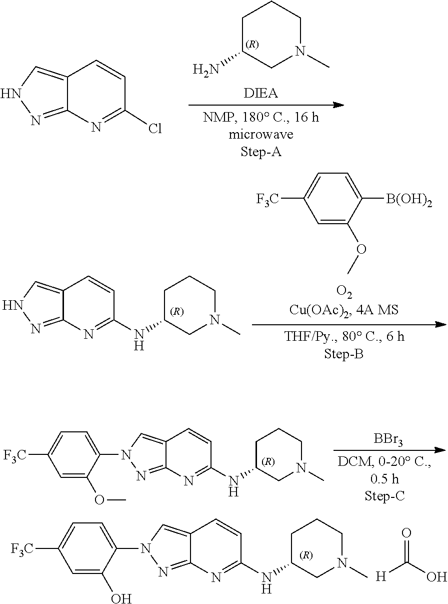

### Step A:

To the solution of 6-chloro-2H-pyrazolo[3,4-b]pyridine (600 mg, 3.91 mmol, 1.0 eq) in NMP (6 mL) was added (3R)-1-methylpiperidin-3-amine (1.34 g, 11.72 mmol, 3.0 eq) and DIEA (1.51 g, 11.72 mmol, 2.04 mL, 3.0 eq) and the resulting reaction mixture was stirred at 180° C. for 16 h under microwave irradiation. LC-MS indicated that the reaction was completed. The residue was purified by prep-HPLC (column: Waters Xbridge BEH C18, 100×30 mm, 10 μm; mobile phase: [water (NH4HCO₃)-can]; B %: 10%-40%, 8 min) to give the title product. (R)—N-(1-methylpiperidin-3-yl)-2H-pyrazolo[3,4-b]pyridin-6-amine (400 mg, 1.73 mmol, 44% yield) was obtained as a yellow solid.

MS (ESI): 232.4 [M+H]⁺.

¹H NMR (400 MHz, DMSO-d6) δ=12.71 (br s, 1H), 7.-0-7.62 (m, 2H), 6.80 (br d, 1H), 6.37 (d, 1H), 4.-4-3.93 (m, 1H), 2.85 (br d, 1H), 2.-8-2.52 (m, 1H), 2.15 (s, 3H), 1.95 (br t, 1H), 1.-4-1.65 (m, 3H), 1.-2-1.42 (m, 1H), 1.33-1.15 (m, 1H).

### Step B:

To the solution of [2-methoxy-4-(trifluoromethyl)phenyl]boronic acid (172 mg, 778.22 μmol, 1.2 eq) in the mixed solvent of pyridine (0.1 mL) and THF (0.4 mL) was successively added N-[(3R)-1-methyl-3-piperidyl]-2H-pyrazolo[3,4-b]pyridin-6-amine (150 mg, 648.52 μmol, 1 eq), Cu(OAc)2 (235.58 mg, 1.30 mmol, 2 eq) and molecular sieve 4 Å (140 mg) then the mixture was degassed 3 times with N₂. The resulting reaction mixture was heated to 80° C. and stirred at 80° C. for 6 h under N₂. LCMS indicated that the reaction was completed. The reaction solution was filtered to remove molecular sieve 4 Å, then concentrated to give the crude product. The residue was purified by prep-HPLC (column: Waters Xbridge BEH C18 100 mm×30 mm, 10 μm; mobile phase: [water(NH₄HCO₃)-ACN]; B %: 35%-55%, 8 min) to give the title product. 2-[2-Methoxy-4-(trifluoromethyl)phenyl]-N-[(3R)-1-methyl-3-piperidyl]pyrazolo[3,4-b]pyridin-6-amine (42 mg, 104 μmol, 16% yield) was obtained as a light yellow solid.

MS (ESI): 406.3 [M+H]⁺.

### Step C:

The solution of 2-[2-methoxy-4-(trifluoromethyl)phenyl]-N-[(3R)-1-methyl-3-piperidyl]pyrazolo[3,4-b]pyridin-6-amine (40 mg, 98.66 μmol, 1.0 eq) in DCM (4 mL) was cooled to 0° C., then BBr₃ (74.15 mg, 295.99 μmol, 28.52 μL, 3 eq) was added dropwise at 0° C. After the completion of the addition, the reaction mixture was stirred at 20° C. for 0.5 h. LCMS indicated that the reaction was completed. The reaction solution was concentrated to give the crude product. The residue was purified by prep-HPLC (column: Phenomenex Luna C18 100 mm×30 mm, 5 μm; mobile phase: [water (FA)-ACN]; B %: 1%-35%, 8 min) to give the title product. 2-[6-[[(3R)-1-Methyl-3-piperidyl]amino]pyrazolo[3,4-b]pyridin-2-yl]-5-(trifluoromethyl)phenol (10 mg, 25 μmol, 25% yield, 98% purity) was obtained as a white solid. LC-MS (ESI): 392.3 [M+H]⁺.

¹H NMR (400 MHz, DMSO-d6) δ=11.92 (br s, 1H), 8.84 (s, 1H), 8.18 (s, 1H), 8.09 (d, 1H), 7.77 (d, 1H), 7.36 (s, 1H), 7.32 (br d, 1H), 7.14 (d, 1H), 6.54 (d, 1H), 4.21-4.00 (m, 1H), 2.90 (br d, 1H), 2.65-2.56 (m, 1H), 2.21 (s, 3H), 2.04 (br s, 1H), 1.92 (br s, 1H), 1.84 (br dd, 12.1 Hz, 1H), 1.73 (br dd, 1H), 1.56 (br dd, 1H), 1.30 (br d, 1H).

Following the procedure of Example 40, the following examples were prepared, using the corresponding commercially available amines as shown in Table 4:

TABLE 4

| | | | 1. Yield |
|---|---|---|---|
| | | | 2. ¹H-NMR |
| Amine | Example | | 3. MH+ (ESI) |
| 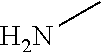 | 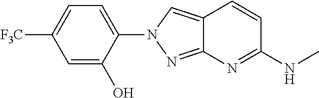 [SMILES: CNc1ccc2cn(-c3ccc(C(F)(F)F)cc3O)nc2n1] | 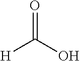 | 1. 62 % 2. ¹H NMR (400 MHz, DMSO-d₆) δ = 12.15 − 11.82 (m, 1H), 8.86 (s, 1H), 8.09 (d, J = 8.3 Hz, 1H), 7.77 (d, J = 9.1 Hz, 1H), 7.39 − 7.30 (m, 2H), 7.26 (br d, J = 4.6 Hz, 1H), 6.49 (d, J = 9.1 Hz, 1H), 2.87 (d, J = 4.8 Hz, 3H). 3. 309.3 |
| | 41 | | |

| | | |
|---|---|---|
| | 2-[6-(methylamino)pyrazolo[3,4-b]pyridin- | |
| | 2-yl]-5-(trifluoromethyl)phenol | |
| 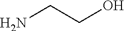 | 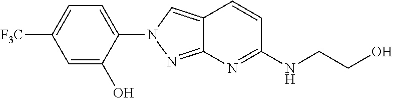 [SMILES: OCCNc1ccc2cn(-c3ccc(C(F)(F)F)cc3O)nc2n1] | 1. 39 % 2. ¹H NMR (400 MHz, DMSO-d6) δ = 8.87 (s, 1H), 8.08 (d, 1H), 7.77 (d, 1H), 7.36 − 7.34 (m, 1H), 7.33 − 7.27 (m, 2H), 6.54 (d, 1H), 4.90 − 4.77 (m, 1H), 3.58 (br d, 2H), 3.49 − 3.47 (m, 2H). 3. 339.1 |
| | 48 | |
| | 2-(6-((2-hydroxyethyl)amino)-2H-pyrazolo[3,4-b]pyridin-2-yl)-5- | |
| | (trifluoromethyl)phenol | |

### Example 42: 2-(6-methoxypyrazolo[3,4-b]pyridin-2-yl)-5-(trifluoromethyl)cyclohexa-2,4-dien-1-ol

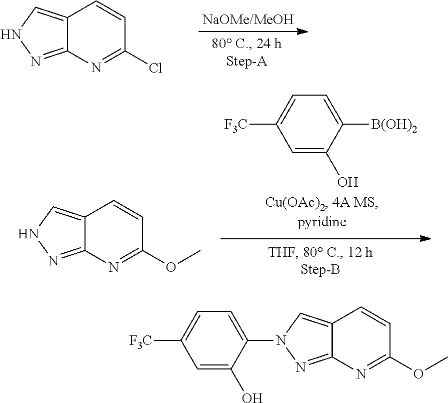 [SMILES: Clc1ccc2c[nH]nc2n1 ; OB(O)c1ccc(C(F)(F)F)cc1O ; COc1ccc2c[nH]nc2n1 ; COc1ccc2cn(-c3ccc(C(F)(F)F)cc3O)nc2n1]

### Step A:

To a solution NaOMe 30% in MeOH (19.54 mL, 6.51 mmol, 1 eq) was added 6-chloro-2H-pyrazolo[3,4-b]pyridine (1 g, 6.51 mmol, 1 eq) and the resulting reaction mixture was stirred at 80° C. for 24 h. LC-MS indicated that the reaction was completed. The reaction mixture was poured onto 30 mL H₂O, followed by 30 mL EtOAc. After that, the aqueous phase was separated and extracted with EtOAc (2×30 mL). The combined organic layer was washed successively with water (2×20 mL) and brine (20 mL), dried over anhydrous Na₂SO₄, filtered, and concentrated to give a residue. The residue was purified by column chromatography (SiO₂, petroleum ether/ethyl acetate=100/1 to 10/1) to give the desired product 6-methoxy-2H-pyrazolo[3,4-b]pyridine (0.7 g, 4.69 mmol, 72%) as a yellow solid.

MS (ESI): 150.0 [M+H]⁺.

### Step B:

Following Example 43 Step B, 2-(6-methoxypyrazolo[3,4-b]pyridin-2-yl)-5-(trifluoromethyl)-cyclohexa-2,4-dien-1-ol (5.5 mg, 17.67 μmol, 14%) was obtained as a white solid.

MS (ESI): 309.8 [M+H]⁺.

¹H NMR (400 MHz, DMSO-d₆) δ=11.61 (br s, 1H), 9.04 (s, 1H), 8.15 (d, J=8.9 Hz, 1H), 8.10 (d, J=8.4 Hz, 1H), 7.38 (s, 1H), 7.33 (br d, J=8.5 Hz, 1H), 6.69 (d, J=8.9 Hz, 1H), 3.95 (s, 3H)

### Example 43: (R)-5-chloro-2-(6-((1-methylpiperidin-3-yl)amino)-2H-pyrazolo[3,4-b]pyridin-2-yl)phenol

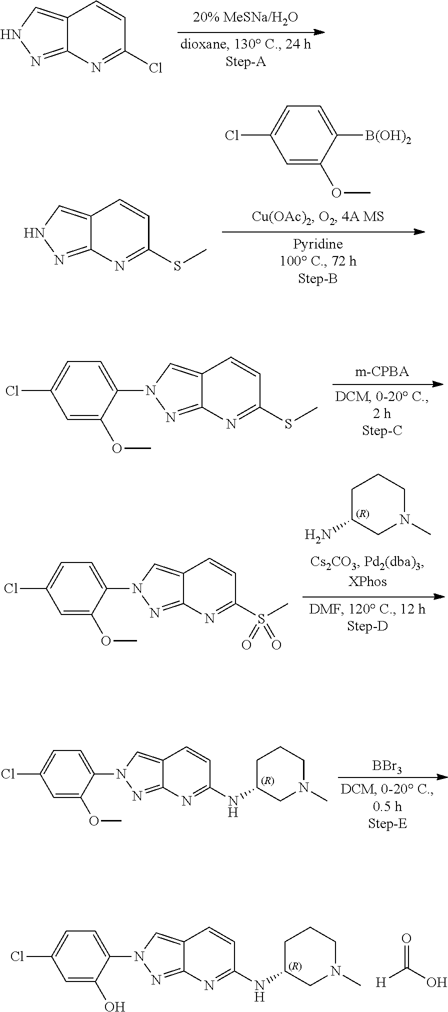

### Step A:

To the solution of 6-chloro-2H-pyrazolo[3,4-b]pyridine (8 g, 52.09 mmol, 1.0 eq) in dioxane (20 mL) was added NaSMe (365 g, 1.04 mol, 332 mL, 20 eq) and the resulting reaction mixture was stirred at 130° C. for 24 h. LC-MS indicated that 34% starting material remained and 64% desired product was obtained. The reaction mixture was poured onto 500 mL H₂O, followed by 120 mL EtOAc. After that, the aqueous phase was separated and extracted with EtOAc (2×120 mL). The combined organic layer was washed successively with water (2×200 mL) and brine (120 mL), dried over anhydrous Na₂SO₄, filtered, and concentrated to give a residue. The residue was purified by prep-HPLC (column: Agela DuraShell C18, 250 mm×70 mm, 10 μm; mobile phase: [H₂O (10 mM NH₄HCO₃)-ACN]; gradient: 12%-42% B over 17 min) to give the title product. 6-Methylsulfanyl-2H-pyrazolo[3,4-b]pyridine (3 g, 18.16 mmol, 35%) was obtained as a white solid.

MS (ESI): 166.1 [M+H]⁺.

¹H NMR (400 MHz, DMSO-d₆) δ=13.53 (br s, 1H), 8.02 (t, 2H), 7.05 (d, 1H), 2.58 (s, 3H).

### Step B:

To the solution of 6-methylsulfanyl-2H-pyrazolo[3,4-b]pyridine (700 mg, 4.24 mmol, 1.0 eq) in pyridine (8 mL) was added (4-chloro-2-methoxy-phenyl)boronic acid (1.03 g, 5.51 mmol, 1.3 eq), Cu(OAc)₂ (769.55 mg, 4.24 mmol, 1.0 eq), 4A MS (700 mg, 4.24 mmol, 1.0 eq) and the resulting reaction mixture was stirred at 100° C. for 72 h under O₂ atmosphere. LC-MS indicated that the reaction was completed. The reaction mixture was poured onto 20 mL H₂O, followed by 10 mL EtOAc. After that, the aqueous phase was separated and extracted with EtOAc (2×10 mL). The combined organic layer was washed successively with water (2×20 mL) and brine (10 mL), dried over anhydrous Na₂SO₄, filtered, and concentrated to give a residue. The residue was purified by column chromatography (SiO₂, petroleum ether/ethyl acetate=100/1 to 0/1). 2-(4-chloro-2-methoxy-phenyl)-6-methylsulfanyl-pyrazolo[3,4-b]pyridine (150 mg, 491 μmol, 12%) was obtained as a yellow oil.

MS (ESI): 306.0 [M+H]⁺.

¹H NMR (400 MHz, DMSO-d₆) δ=8.75 (s, 1H), 8.25 (s, 1H), 8.10 (d, 1H), 8.04 (d, 1H), 7.84 (d, 1H), 7.48 (d, 1H), 7.44 (d, 1H), 7.39 (d, 1H), 7.23-7.23 (m, 1H), 7.23 (dd, 1H), 7.21-7.14 (m, 2H), 7.01 (d, 1H), 3.94 (s, 2H), 3.77 (s, 3H), 2.59 (s, 2H), 2.45 (s, 3H).

### Step C:

To the solution of 2-(4-chloro-2-methoxy-phenyl)-6-methylsulfanyl-pyrazolo[3,4-b]pyridine (350 mg, 1.14 mmol, 1 eq) in DCM (8 mL) was added m-CPBA (465 mg, 2.29 mmol, 85% purity, 2 eq) at 0° C. and the resulting reaction mixture was stirred at 20° C. for 2 h. LC-MS indicated that the reaction was completed. The reaction mixture was poured onto 20 mL H₂O, followed by 10 mL DCM. After that, the aqueous phase was separated and extracted with DCM (2×10 mL). The combined organic layer was washed successively with water (2×20 mL) and brine (10 mL), dried over anhydrous Na₂SO₄, filtered, and concentrated to give a residue. The residue was purified by prep-TLC (SiO₂, petroleum ether/ethyl acetate=1:1). 2-(4-Chloro-2-methoxy-phenyl)-6-methylsulfonyl-pyrazolo[3,4-b]pyridine (90 mg, 266 μmol, 23%) was obtained as a yellow solid. MS (ESI): 338.0 [M+H]⁺.

### Step D:

To the solution of 2-(4-chloro-2-methoxy-phenyl)-6-methylsulfonyl-pyrazolo[3,4-b]pyridine (10 mg, 29.60 μmol, 1 eq) in DMF (1 mL) was added (3R)-1-methylpiperidin-3-amine (17 mg, 148 μmol, 5 eq), Pd₂(dba)₃ (27 mg, 30 μmol, 1 eq), Cs₂CO₃ (19 mg, 59 μmol, 2 eq), and XPhos (28 mg, 59 μmol, 2 eq) and the resulting reaction mixture was stirred at 120° C. for 12 h. LC-MS indicated that the reaction was completed. The reaction mixture was poured onto 15 mL H₂O, followed by 10 mL EtOAc. After that, the aqueous phase was separated and extracted with EtOAc (2×10 mL). The combined organic layer was washed successively with water (2×15 mL) and brine (10 mL), dried over anhydrous Na₂SO₄, filtered, and concentrated to give a residue. The residue was purified by prep-TLC (SiO₂, DCM:MeCOH=5:1). 2-(4-Chloro-2-methoxy-phenyl)-N-[(3R)-1-methyl-3-piperidyl]pyrazolo[3,4-b]pyridin-6-amine (15 mg, 40.34 μmol, 23%) was obtained as a yellow oil.

MS (ESI): 372.2 [M+H]⁺.

### Step E:

Following example 46 Step F, (R)-5-chloro-2-(6-((1-methylpiperidin-3-yl)amino)-2H-pyrazolo[3,4-b]pyridin-2-yl)phenol (3 mg, 9 μmol, 23%) was obtained as a white solid.

MS (ESI): 358.2 [M+H]⁺.

¹H NMR (400 MHz, DMSO-d₆) δ=12.22-11.49 (m, 1H), 8.70 (s, 1H), 7.85 (d, 1H), 7.75 (d, 1H), 7.11 (d, 1H), 7.07 (d, 1H), 7.03 (dd, 1H), 6.52 (d, 1H), 4.17-4.03 (m, 1H), 2.90 (br d, 1H), 2.60 (br d, J=10.3 Hz, 1H), 2.21 (s, 3H), 2.04 (br s, 1H), 1.97-1.79 (m, 2H), 1.78-1.66 (m, 1H), 1.56 (br dd, 1H), 1.29 (br d, 1H).

### Example 44: 2-[6-(difluoromethoxy)pyrazolo[3,4-b]pyridin-2-yl]-5-(trifluoromethyl)phenol

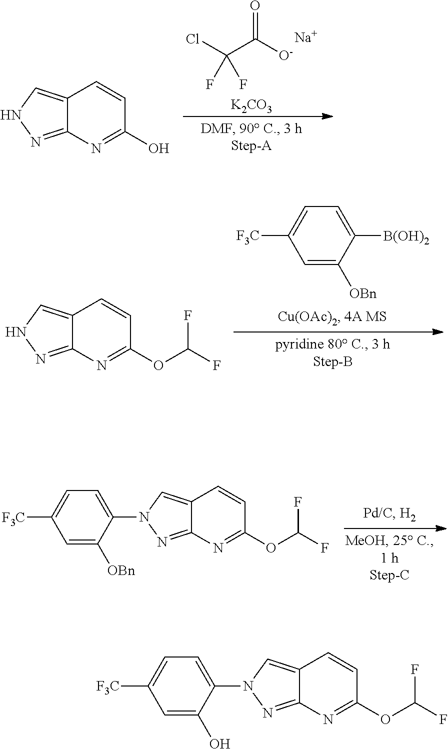

### Step A:

To a solution of 2,7-dihydropyrazolo[3,4-b]pyridin-6-one (330 mg, 2.44 mmol, 1 eq) and sodium 2-chloro-2,2-difluoro-acetate (559 mg, 3.66 mmol, 1.5 eq) in DMF (5 mL) was added K₂CO₃ (169 mg, 1.22 mmol, 0.5 eq). The mixture was stirred at 90° C. for 3 h. LC-MS indicated that the reaction was completed. After completion, the precipitate was collected by filtration to give the crude product. The residue was purified by prep-HPLC (column: Waters Xbridge C18, 150 mm×50 mm, 10 μm; mobile phase: [H₂O (10 mM NH₄HCO₃)-ACN]; gradient: 1-55% B over 8 min) to give the compound 6-(difluoromethoxy)-2H-pyrazolo[3,4-b]pyridine (140 mg, 756 μmol, 31%) as a white solid.

MS (ESI): 184.7 [M−H]⁻.

¹H NMR (400 MHz, DMSO-d₆) δ=13.77-13.65 (m, 1H), 8.33 (d, 1H), 8.16-8.10 (m, 1H), 7.94-7.58 (m, 1H), 6.86 (d, 1H)

### Step B:

Following example 43 Step B, using [2-benzyloxy-4-(trifluoromethyl)phenyl]boronic acid, 2-[2-benzyloxy-4-(trifluoromethyl)phenyl]-6-(difluoromethoxy)pyrazolo[3,4-b]pyridine (45 mg, 104 μmol, 14%) was obtained as a white solid. MS (ESI): 436.1 [M+H]⁺.

### Step C:

To a mixture of 2-[2-benzyloxy-4-(trifluoromethyl)phenyl]-6-(difluoromethoxy)pyrazolo[3,4-b]pyridine (30 mg, 68.9 μmol, 1 eq) in MeOH (5 mL), was added Pd/C (73 mg, 69 μmol, 10% purity, 1 eq), and the mixture was stirred at 25° C. for 1 h under H₂ atmosphere. LCMS indicated that the reaction was completed. After completion, the reaction mixture was filtered and the filtrate was collected to give the crude product. The crude product was purified by prep-HPLC (Waters Xbridge BEH C18 100×30 mm, 10 μm; mobile phase: [H₂O (10 mM NH₄HCO₃)-ACN]; gradient: 40-70% B over 8 min) to give the title compound 2-[6-(difluoromethoxy)pyrazolo[3,4-b]pyridin-2-yl]-5-(trifluoromethyl)phenol (4.5 mg, 13 μmol, 19%) as a white solid.

MS (ESI): 346.0 [M+H]⁺.

¹H NMR (400 MHz, DMSO-d₆) δ=11.66 (br s, 1H), 9.18 (s, 1H), 8.41 (d, 1H), 8.09 (d, 1H), 8.10-7.70 (m, 1H), 7.39 (s, 1H), 7.33 (br d, 1H), 6.88 (d, 1H)

### Example 45: 2-[6-(trifluoromethoxy)pyrazolo[3,4-b]pyridin-2-yl]-5-(trifluoromethyl)phenol

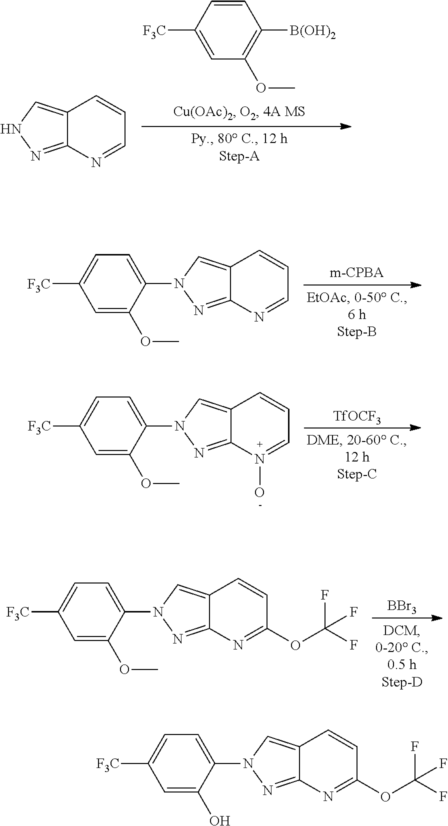

### Step A:

Following Ex 47 Step B, 2-[2-methoxy-4-(trifluoromethyl)phenyl]pyrazolo[3,4-b]pyridine (450 mg, 1.53 mmol, 4%) was obtained as a white solid.

MS (ESI): 294.2 [M+H]⁺.

¹H NMR (400 MHz, DMSO-d₆) δ=8.97 (s, 1H), 8.70 (dd, 1H), 8.30 (dd, J=1.6, 8.4 Hz, 1H), 8.10 (d, 1H), 7.66 (s, 1H), 7.56 (d, 1H), 7.17 (dd, 1H), 4.02 (s, 3H).

### Step B:

To a solution of 2-[2-methoxy-4-(trifluoromethyl)phenyl]pyrazolo[3,4-b]pyridine (450 mg, 1.53 mmol, 1 eq) in EtOAc (20 mL) was added m-CPBA (779 mg, 3.84 mmol, 85% purity, 2.5 eq) at 0° C. The mixture was stirred at 50° C. for 6 h. LC-MS showed the reaction was completed. The reaction mixture was diluted with 30 mL H₂O, and extracted with EtOAc (3×20 mL). The combined organic layers were washed with brine (2×20 mL), dried over Na₂SO₄, filtered and concentrated under reduced pressure to give a residue. The residue was purified by prep-TLC (SiO₂, DCM:MeOH=10:1) to give 2-[2-methoxy-4-(trifluoromethyl)phenyl]-7-oxido-pyrazolo[3,4-b]pyridin-7-ium (200 mg, 647 μmol, 42%) as a light yellow solid.

MS (ESI): 310.2 [M+H]⁺.

### Step C:

To a solution of 2-[2-methoxy-4-(trifluoromethyl)phenyl]-7-oxido-pyrazolo[3,4-b]pyridin-7-ium (150 mg, 485 μmol, 1 eq) in DME (15 mL) was added trifluoromethyl trifluoromethanesulfonate (212 mg, 970 μmol, 2 eq) and the mixture was stirred for 2 h at 20° C. The mixture was stirred at 60° C. for 10 h. LC-MS showed the reaction was completed. The reaction mixture was concentrated by vacuo. The residue was purified by prep-TLC (SiO₂, DCM:MeOH=20:1) to give the product. 2-[2-Methoxy-4-(trifluoromethyl)phenyl]-6-(trifluoromethoxy)pyrazolo[3,4-b]pyridine (20 mg, 53.02 μmol, 11%) was obtained as light yellow solid. MS (ESI): 378.1 [M+H]⁺.

### Step D:

Following example 46 Step F, 2-[6-(trifluoromethoxy)pyrazolo[3,4-b]pyridin-2-yl]-5-(trifluoromethyl) phenol (6 mg, 16 μmol, 30%) was obtained as a white solid.

MS (ESI): 363.9 [M+H]⁺.

¹H NMR (400 MHz, DMSO-d₆) δ=11.57 (br s, 1H), 9.17 (s, 1H), 8.53 (d, 1H), 8.11 (d, 1H), 7.43 (s, 1H), 7.39 (br d, 1H), 7.02 (d, 1H).

### Example 46 (R)-2-(6-fluoro-1-methyl-5-((1-methylpyrrolidin-3-yl)amino)-1H-imidazo[4,5-b]pyridin-2-yl)-3-methyl-5-(trifluoromethyl)phenol

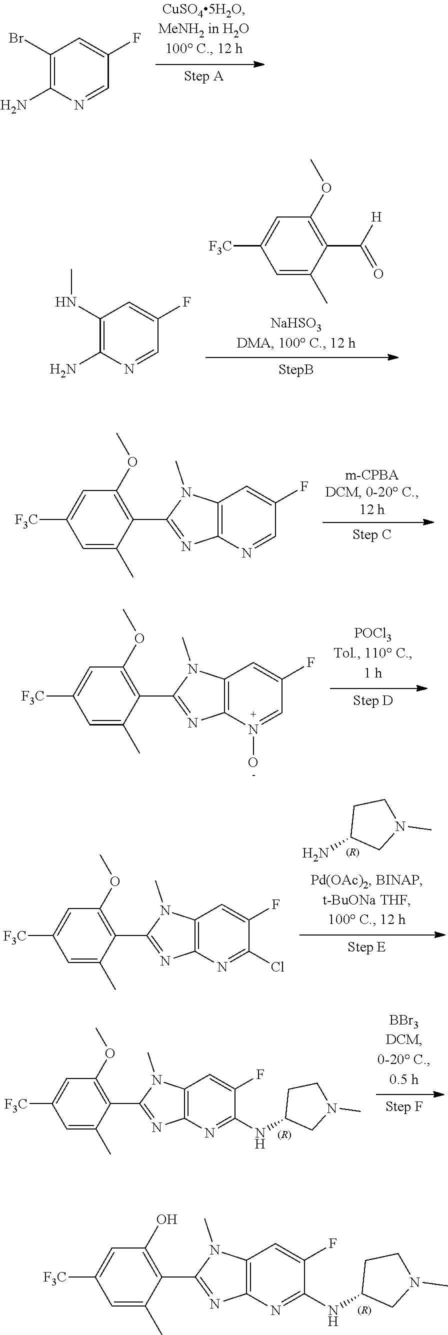

### Step A:

To an aqueous solution of 3-bromo-5-fluoro-pyridin-2-amine (3 g, 15.71 mmol, 1.0 eq) in 40% MeNH₂ (9.32 g, 120.00 mmol, 20 mL, 40% purity, 7.64 eq) was added copper sulfate pentahydrate (250.69 mg, 1.57 mmol, 241.05 μL, 0.1 eq) and the reaction mixture was stirred at 100° C. for 12 hr. The reaction mixture was filtered through celite. The celite pad was rinsed with DCE:MeOH=9:1 (3×50 mL) and concentrated in vacuo to get the crude product. The crude product was purified by column chromatography (SiO₂, petroleum ether/ethyl acetate=100/1 to 0/1. The crude product was purified by column chromatography (SiO₂, petroleum ether/ethyl acetate=100/1 to 0/1) to afford 5-fluoro-N3-methyl-pyridine-2,3-diamine (2 g, 14.17 mmol, 90.21% yield) as a black brown solid.

### Step B:

To the solution of 5-fluoro-N3-methyl-pyridine-2,3-diamine (1.5 g, 10.63 mmol, 1.0 eq) in DMA (15 mL) was added NaHSO₃ (1.33 g, 12.75 mmol, 896.66 μL, 1.2 eq) and 2-methoxy-6-methyl-4-(trifluoromethyl)benzaldehyde (2.32 g, 10.63 mmol, 1 eq). The resulting reaction mixture was stirred at 100° C. for 12 hr. LCMS indicated that the reaction was completed. The reaction mixture was poured onto 50 mL H₂O, followed by 20 mL DCM. After that, the aqueous phase was separated and extracted with DCM (20 mL×3). The combined organic layer was washed successively with water (20 mL×2) and brine (20 mL×1), dried over anhydrous Na₂SO₄, filtered, and concentrated to give a residue. The residue was purified by column chromatography (SiO₂, petroleum ether/ethyl acetate=100/1 to 1/1). 6-Fluoro-2-[2-methoxy-6-methyl-4-(trifluoromethyl)phenyl]-1-methyl-imidazo[4,5-b]pyridine (2.6 g, 7.66 mmol, 72.11% yield, N/A purity) was obtained as a yellow solid.

LC-MS (ES⁺, m/z): 340.4 [(M+H)⁺].

¹H NMR (400 MHz, DMSO-d6) δ=8.45 (s, 1H), 8.16 (dd, J=2.8, 8.9 Hz, 1H), 7.43 (s, 1H), 7.36 (s, 1H), 3.84 (s, 3H), 3.56 (s, 3H), 2.17 (s, 3H)

### Step C:

To the solution of 6-fluoro-2-[2-methoxy-6-methyl-4-(trifluoromethyl)phenyl]-1-methyl-imidazo[4,5-b]pyridine (2.6 g, 7.66 mmol, 1.0 eq) in DCM (30 mL) was added m-CPBA (3.11 g, 15.33 mmol, 85% purity, 2.0 eq) at 0° C. The resulting reaction mixture was stirred at 20° C. for 12 hr. LCMS indicated that the reaction was completed. The reaction mixture was poured onto 50 mL H₂O, followed by 20 mL DCM. After that, the aqueous phase was separated and extracted with DCM (20 mL×3). The combined organic layer was washed successively with water (20 mL×2) and brine (20 mL×1), dried over anhydrous Na₂SO₄, filtered, and concentrated to give a residue. The residue was purified by column chromatography (SiO₂, DCM/MeOH=100/1 to 15/1). 6-Fluoro-2-[2-methoxy-6-methyl-4-(trifluoromethyl)phenyl]-1-methyl-4-oxido-imidazo[4,5-b]pyridin-4-ium (2.4 g, 6.08 mmol, 79.34% yield, 90% purity) was obtained as yellow solid.

LC-MS (ES⁺, m/z): 356.2 [(M+H)⁺].

¹H NMR (400 MHz, DMSO-d6) δ=8.54 (dd, J=1.8, 5.8 Hz, 1H), 7.97-7.82 (m, 1H), 7.44 (s, 1H), 7.37 (s, 1H), 3.85 (s, 3H), 3.56 (s, 3H), 2.18 (s, 3H)

### Step D:

To the solution of 6-fluoro-2-[2-methoxy-6-methyl-4-(trifluoromethyl)phenyl]-1-methyl-4-oxido-imidazo[4,5-b]pyridin-4-ium (2.4 g, 6.08 mmol, 1.0 eq) in toluene (25 mL) was added POCl₃ (2.80 g, 18.24 mmol, 1.70 mL, 3.0 eq). The resulting reaction mixture was stirred at 110° C. for 1 hr. LCMS indicated that the reaction was completed. The reaction mixture was poured onto 50 mL H₂O, followed by 20 mL DCM. After that, the aqueous phase was separated and extracted with DCM (20 mL×3). The combined organic layer was washed successively with water (20 mL×2) and brine (20 mL×1), dried over anhydrous Na₂SO₄, filtered, and concentrated to give a residue. The residue was purified by column chromatography (SiO₂, petroleum ether/ethyl acetate=100/1 to 4/1). 5-chloro-6-fluoro-2-[2-methoxy-6-methyl-4-(trifluoromethyl)phenyl]-1-methyl-imidazo[4,5-b]pyridine (1.8 g, 1.20 mmol, 19.81% yield, 25% purity) was obtained as a white solid.

LC-MS (ES⁺, m/z): 374.4 [(M+H)⁺].

¹H NMR (400 MHz, DMSO-d6) δ=8.59 (d, J=2.0 Hz, 1H), 7.44 (s, 1H), 7.37 (s, 1H), 3.85 (s, 3H), 3.77 (s, 3H), 2.18 (s, 3H)

### Step E:

To the solution of 5-chloro-6-fluoro-2-[2-methoxy-6-methyl-4-(trifluoromethyl)phenyl]-1-methyl-imidazo[4,5-b]pyridine (100.00 mg, 66.89 μmol, 25% purity, 1.0 eq) in THF (2 mL) was added (3R)-1-methylpyrrolidin-3-amine (40.20 mg, 401.36 μmol, 2.0 eq), BINAP (24.99 mg, 40.14 μmol, 0.2 eq), t-BuONa (57.86 mg, 602.04 μmol, 3.0 eq) and Pd(OAc)₂ (9.01 mg, 40.14 μmol, 0.2 eq). The resulting reaction mixture was stirred at 100° C. for 12 hr. LCMS indicated that the reaction was completed. The reaction mixture was poured onto 20 mL sat. NH₄Cl at 0° C., followed by 10 mL DCM. After that, the aqueous phase was separated and extracted with DCM (20 mL×2). The combined organic layer was washed successively with water (20 mL×2) and brine (20 mL×1), dried over anhydrous Na₂SO₄, filtered, and concentrated to give a residue. The residue was purified by prep-HPLC (column: Phenomenex Luna C18 100 mm×30 mm×5 μm; mobile phase: [H₂O (0.2% FA)-ACN]; gradient: 10%-40% B over 8.0 min) to give the title product. 6-fluoro-2-[2-methoxy-6-methyl-4-(trifluoromethyl) phenyl]-1-methyl-N-[(3R)-1-methylpyrrolidin-3-yl]imidazo[4,5-b]pyridin-5-amine (batchx 2 total, 40 mg, 91.44 μmol, 68.4% yield, N/A purity) as a white solid.

LC-MS (ES⁺, m/z): 438.2 [(M+H)⁺].

¹H NMR (400 MHz, DMSO-d6) δ=7.86 (d, J=11.0 Hz, 1H), 7.39 (s, 1H), 7.31 (s, 1H), 6.30 (br s, 1H), 4.45 (br s, 1H), 3.81 (s, 3H), 3.44 (s, 3H), 2.90 (br t, J=6.6 Hz, 1H), 2.68 (br s, 1H), 2.56 (br s, 2H), 2.35-2.29 (m, 3H), 2.24 (br d, J=5.3 Hz, 1H), 2.15 (s, 3H), 1.88-1.75 (m, 1H)

### Step F:

To the solution of 6-fluoro-2-[2-methoxy-6-methyl-4-(trifluoromethyl)phenyl]-1-methyl-N-[(3R)-1-methylpyrrolidin-3-yl]imidazo[4,5-b]pyridin-5-amine (40 mg, 91.44 μmol, 1.0 eq) in DCM (1 mL) was added BBr₃ (114.54 mg, 457.21 μmol, 44.05 μL, 5.0 eq) at 0° C. The resulting reaction mixture was stirred at 20° C. for 0.5 hr. LCMS indicated that the reaction was completed. After completion, the solvent was removed by concentration to give the residue. The residue was purified by prep-HPLC (column: Waters Xbridge BEH C18 100 mm×30 mm×10 μm; mobile phase: [H₂O (10 mM NH₄HCO₃)-ACN]; gradient: 20%-50% B over 8.0 min) to give the title product. 2-[6-Fluoro-1-methyl-5-[[(3R)-1-methylpyrrolidin-3-yl]amino]imidazo[4,5-b]pyridin-2-yl]-3-methyl-5-(trifluoromethyl)phenol (17.0 mg, 39.70 μmol, 43.42% yield, 98.88% purity) was obtained as a white solid.

LC-MS (ES⁺, m/z): 424.2 [(M+H)⁺].

¹H NMR (400 MHz, DMSO-d6) δ=10.76-10.43 (m, 1H), 7.84 (d, J=11.0 Hz, 1H), 7.18 (s, 1H), 7.10 (s, 1H), 6.22 (br d, J=6.5 Hz, 1H), 4.50-4.38 (m, 1H), 3.48 (s, 3H), 2.84-2.79 (m, 1H), 2.62-2.54 (m, 1H), 2.45-2.38 (m, 2H), 2.26 (d, J=3.9 Hz, 3H), 2.24-2.18 (m, 1H), 2.14 (s, 3H), 1.83-1.71 (m, 1H)

### Example 47 (R)-5-chloro-2-(6-fluoro-1-methyl-5-((1-methylpyrrolidin-3-yl)amino)-1H-imidazo[4,5-b]pyridin-2-yl)-3-methylphenol formate Salt

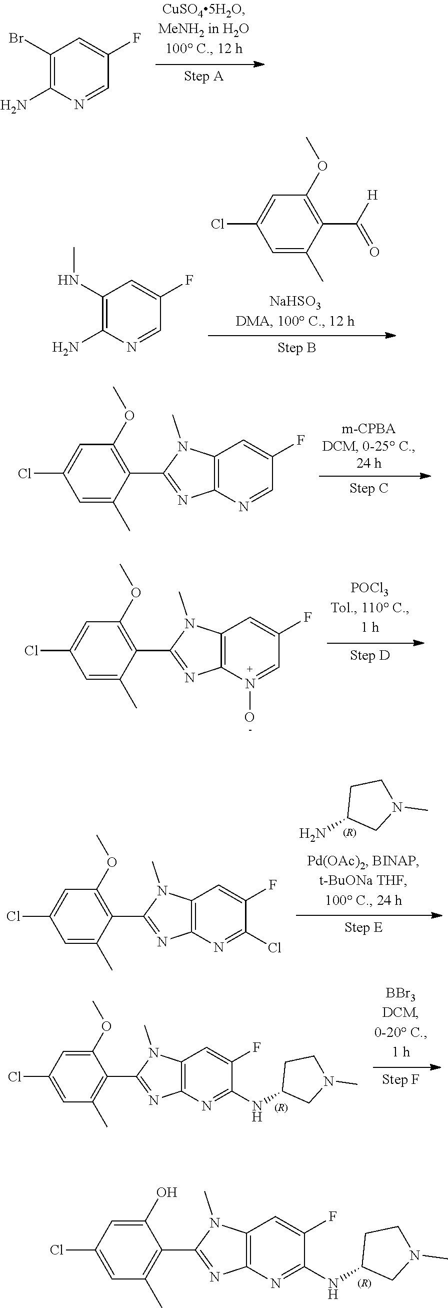

### Step A:

To 3-bromo-5-fluoro-pyridin-2-amine (3 g, 15.71 mmol, 1.0 eq) in MeNH₂ in H₂O (3.00 g, 28.98 mmol, 30% purity, 1.85 eq) was added CuSO₄·5H₂O (392.17 mg, 1.57 mmol, 0.1 eq) and the resulting reaction mixture was stirred at 100° C. for 12 hr. LCMS indicated that the reaction was completed. The reaction mixture was poured onto 100 mL H₂O, followed by 60 mL EA. After that, the aqueous phase was separated and extracted with EA (60 mL×2). The combined organic layer was washed successively with water (50 mL×2) and brine (50 mL×1), dried over anhydrous Na₂SO₄, filtered, and concentrated to give a residue. The residue was purified by column chromatography (SiO₂, petroleum ether/ethyl acetate=100/1 to 0/1). 5-Fluoro-N3-methyl-pyridine-2,3-diamine (2.9 g, 20.55 mmol, 65.41% yield, N/A purity) was obtained as a gray solid.

LC-MS (ES⁺, m/z): 142.1 [(M+H)⁺].

¹H NMR (400 MHz, DMSO-d₆) δ=7.15 (d, J=2.6 Hz, 1H), 6.42 (dd, J=2.5, 11.0 Hz, 1H), 5.33 (s, 2H), 5.26 (br d, J=3.1 Hz, 1H), 2.69 (d, J=4.9 Hz, 3H)

### Step B:

To the solution of 5-fluoro-N3-methyl-pyridine-2,3-diamine (600 mg, 4.25 mmol, 1.0 eq) in DMA (6 mL) was added 4-chloro-2-methoxy-6-methyl-benzaldehyde (784.80 mg, 4.25 mmol, 1.0 eq), and NaHSO₃ (884.71 mg, 8.50 mmol, 597.78 μL, 2.0 eq) and the resulting reaction mixture was stirred at 100° C. for 12 hr. LCMS indicated that the reaction was completed. The reaction mixture was poured onto 20 mL H₂O, followed by 10 mL EA. After that, the aqueous phase was separated and extracted with EA (10 mL×2). The combined organic layer was washed successively with water (20 mL×2) and brine (20 mL×1), dried over anhydrous Na₂SO₄, filtered, and concentrated to give a residue. The residue was purified by column chromatography (SiO₂, petroleum ether/ethyl acetate=100/1 to 0/1). 2-(4-Chloro-2-methoxy-6-methyl-phenyl)-6-fluoro-1-methyl-imidazo[4,5-b]pyridine (1 g, 3.27 mmol, 76.94% yield, N/A purity) was obtained as a white solid.

LC-MS (ES⁺, m/z): 306.1 [(M+H)⁺].

¹H NMR (400 MHz, DMSO-d₆) δ=8.43-8.43 (m, 1H), 8.43 (dd, J=2.0, 2.6 Hz, 1H), 7.16 (dd, J=1.4, 12.2 Hz, 2H), 3.77 (s, 3H), 3.54 (s, 3H), 2.08 (s, 3H)

### Step C:

To the solution of DCM (9 mL) was added 2-(4-chloro-2-methoxy-6-methyl-phenyl)-6-fluoro-1-methyl-imidazo[4,5-b]pyridine (1 g, 3.27 mmol, 1.0 eq), then m-CPBA (1.33 g, 6.54 mmol, 85% purity, 2.0 eq) was added at 0° C. and the resulting reaction mixture was stirred at 25° C. for 24 hr. LCMS indicated that the reaction was completed. The reaction mixture was poured onto 20 mL H₂O, followed by 10 mL DCM. After that, the aqueous phase was separated and extracted with DCM (10 mL×2). The combined organic layer was washed successively with water (20 mL×2) and brine (20 mL×1), dried over anhydrous Na₂SO₄, filtered, and concentrated to give a residue. The residue was purified by column chromatography (SiO₂, DCM/MeOH=100/1 to 1/1). 2-(4-Chloro-2-methoxy-6-methyl-phenyl)-6-fluoro-1-methyl-4-oxido-imidazo[4,5-b]pyridin-4-ium (950 mg, 2.95 mmol, 90.28% yield, N/A purity) was obtained as a white solid.

LC-MS (ES⁺, m/z): 322.0 [(M+H)⁺].

¹H NMR (400 MHz, DMSO-d₆) δ=8.52 (dd, J=2.1, 5.9 Hz, 1H), 7.87 (dd, J=2.1, 7.8 Hz, 1H), 7.21-7.12 (m, 2H), 3.78 (s, 3H), 3.54 (s, 3H), 2.09 (s, 3H)

### Step D:

To the solution of 2-(4-chloro-2-methoxy-6-methyl-phenyl)-6-fluoro-1-methyl-4-oxido-imidazo[4,5-b]pyridin-4-ium (950 mg, 2.95 mmol, 1.0 eq) in toluene. (10 mL) was added POCl₃ (905.50 mg, 5.91 mmol, 550.46 μL, 2.0 eq) and the resulting reaction mixture was stirred at 110° C. for 1 hr. LCMS indicated that the reaction was completed. The reaction mixture was poured onto 20 mL H₂O, followed by 10 mL EA. After that, the aqueous phase was separated and extracted with EA (10 mL×2). The combined organic layer was washed successively with water (20 mL×2) and brine (20 mL×1), dried over anhydrous Na₂SO₄, filtered, and concentrated to give a residue. The residue was purified by column chromatography (SiO₂, petroleum ether/ethyl acetate=100/1 to 0/1). 5-Chloro-2-(4-chloro-2-methoxy-6-methyl-phenyl)-6-fluoro-1-methyl-imidazo[4,5-b]pyridine (760 mg, 558.53 μmol, 18.92% yield, 25% purity) was obtained as a white solid.

LC-MS (ES⁺, m/z): 340.1 [(M+H)⁺].

¹H NMR (400 MHz, chloroform-d) δ=7.53 (d, J=7.6 Hz, 1H), 7.00 (s, 1H), 6.87 (d, J=1.4 Hz, 1H), 3.84 (s, 3H), 3.79-3.74 (m, 3H), 2.18-2.17 (m, 3H)

### Step E:

To the solution of 5-chloro-2-(4-chloro-2-methoxy-6-methyl-phenyl)-6-fluoro-1-methyl-imidazo[4,5-b]pyridine (200 mg, 146.98 μmol, 1.0 eq) in THF (2 mL) was added (3R)-1-methylpyrrolidin-3-amine (29.44 mg, 293.96 μmol, 2.0 eq), BINAP (18.30 mg, 29.40 μmol, 0.2 eq), t-BuONa (28.25 mg, 293.96 μmol, 2 eq), and Pd(OAc)₂ (3.30 mg, 14.70 μmol, 0.1 eq) and the resulting reaction mixture was stirred at 100° C. for 24 hr. LCMS indicated that the reaction was completed. The reaction mixture was poured onto 20 mL H₂O, followed by 10 mL EA. After that, the aqueous phase was separated and extracted with EA (10 mL×2). The combined organic layer was washed successively with water (20 mL×2) and brine (20 mL×1), dried over anhydrous Na₂SO₄, filtered, and concentrated to give a residue. The residue was purified by prep-TLC (SiO₂, DCM:MeOH=10:1). The residue was purified by prep-HPLC (column: Phenomenex Luna C18 100 mm×30 mm×5 μm; mobile phase: [H₂O (0.2% FA)-ACN]; gradient: 1%-35% B over 8.0 min) to give the title product. 2-(4-Chloro-2-methoxy-6-methyl-phenyl)-6-fluoro-1-methyl-N-[(3R)-1-methylpyrrolidin-3-yl]imidazo[4,5-b]pyridin-5-amine (10 mg, 24.76 μmol, 16.85% yield, N/A purity) was obtained as a yellow gum.

LC-MS (ES⁺, m/z): 404.2 [(M+H)⁺].

¹H NMR (400 MHz, DMSO-d₆) δ=7.82 (d, J=11.0 Hz, 1H), 7.11 (d, J=6.8 Hz, 2H), 6.24 (br d, J=4.4 Hz, 1H), 4.51-4.37 (m, 1H), 3.75 (s, 3H), 3.42 (s, 3H), 2.86 (ddd, J=2.6, 6.8, 9.3 Hz, 1H), 2.69-2.58 (m, 2H), 2.48-2.42 (m, 1H), 2.29 (d, J=3.9 Hz, 3H), 2.26-2.18 (m, 1H), 2.06 (s, 3H), 1.86-1.71 (m, 1H)

### Step F:

To the solution of 2-(4-chloro-2-methoxy-6-methyl-phenyl)-6-fluoro-1-methyl-N-[(3R)-1-methylpyrrolidin-3-yl]imidazo[4,5-b]pyridin-5-amine (8 mg, 19.81 μmol, 1 eq) in DCM (0.5 mL) was added BBr₃ (24.81 mg, 99.04 μmol, 9.54 μL, 5.0 eq) at 0° C. and the resulting reaction mixture was stirred at 20° C. for 1 hr. LCMS indicated that the reaction was completed. After completion, the solvent was removed by concentration to give the residue. The residue was purified by prep-HPLC (column: Phenomenex Luna C18 100 mm×30 mm×5 μm; mobile phase: [H₂O (0.2% FA)-ACN]; gradient: 1%-20% B over 8.0 min) to give the title product. 5-Chloro-2-[6-fluoro-1-methyl-5-[[(3R)-1-methylpyrrolidin-3-yl]amino]imidazo[4,5-b]pyridin-2-yl]-3-methyl-phenol (4.65 mg, 11.91 μmol, 60.14% yield, 99.87% purity) was obtained as a white solid.

LC-MS (ES⁺, m/z): 390.3 [(M+H)⁺].

¹H NMR (400 MHz, DMSO-d₆) δ=10.39 (br s, 1H), 7.82 (d, J=11.0 Hz, 1H), 6.89 (br d, J=19.1 Hz, 2H), 6.24 (br d, J=6.4 Hz, 1H), 4.53-4.39 (m, 1H), 3.46 (s, 3H), 2.91 (br t, J=8.3 Hz, 2H), 2.67 (br s, 2H), 2.33 (br s, 3H), 2.24 (br dd, J=6.2, 13.2 Hz, 1H), 2.05 (s, 3H), 1.81 (br d, J=6.9 Hz, 1H).

### Example 49: 5-chloro-2-(1-methyl-5-(trifluoromethyl)-1H-imidazo[4,5-b]pyridin-2-yl)phenol

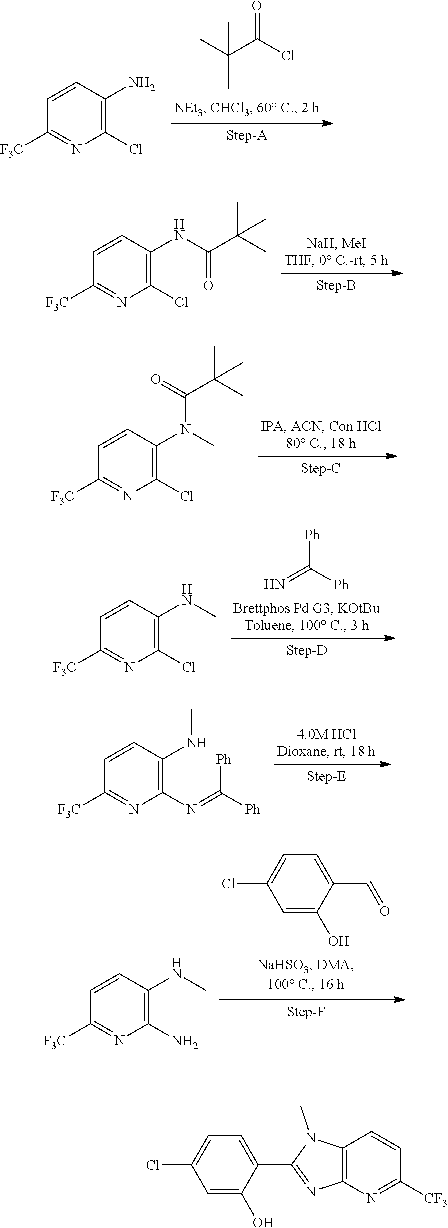

### Step A:

To a stirred solution of 2-chloro-6-(trifluoromethyl)pyridin-3-amine (4.0 g, 20.35 mmol) and NEt₃ (5.67 ml, 40.70 mmol) in chloroform (30 mL) was added pivaloyl chloride (2.70 g, 22.39 mmol) at 0° C. and the mixture was stirred for 30 min at 0° C. The reaction mixture was heated to 60° C. for 2 h. The progress of the reaction was monitored by TLC. TLC showed completion of the reaction. The reaction mixture was cooled to rt., diluted with DCM (100 mL), washed with water (100 mL) and concentrated to get a crude product. The crude product was purified by Combiflash chromatography. The product was eluted at 10% EtOAc in hexane. The pure fractions were collected and concentrated under reduced pressure to afford N-(2-chloro-6-(trifluoromethyl)pyridin-3-yl)pivalamide (5.6 g, 98%) as an oil.

MS (ESI): 281.31 [M+H]⁺.

### Step B:

To a stirred solution of N-(2-chloro-6-trifluoromethylpyridin-3-yl)-2,2-dimethylpropionamide (4.5 g, 16.03 mmol) in THF (45 mL) was added sodium hydride, 57-63% oil dispersion (1.54 g, 32.07 mmol) at 0° C., and the mixture was stirred at room temperature for 30 minutes. The reaction mixture was cooled, and then methyl iodide (6.83 g, 48.10 mmol) was added, and the mixture was stirred at room temperature for 5 h. The progress of the reaction was monitored by TLC. TLC showed completion of reaction. The reaction mixture was poured into ice/water (200 ml) and extracted with ethyl acetate (2×100 mL). The combined organic layers were dried over sodium sulfate and concentrated under reduced pressure to get N-(2-chloro-6-(trifluoromethyl)pyridin-3-yl)-N-methylpivalamide (4.2 g, 88%) as a pale yellow solid.

MS (ESI): 295.08 [M+H]⁺.

### Step C:

A solution of N-(2-chloro-6-(trifluoromethyl)pyridin-3-yl)-N-methylpivalamide (4.2 g, 14.252 mmol) in IPA (21 mL):acetonitrile (42 mL):conc. HCl (21 mL) was stirred at RT for 2 h and heated to 80° C. for 18 h. The progress of the reaction was monitored by TLC. TLC showed completion of reaction. The reaction mixture was cooled to room temperature. The reaction mixture was quenched with a saturated aqueous sodium hydrogen carbonate solution (200 mL) and extracted with ethyl acetate (2×200 mL). The organic layers were dried over sodium sulfate and concentrated under reduced pressure to get a crude product. The crude product was purified by combiflash chromatography. The product was eluted at 10% EtOAc in hexane. The pure fractions were collected and concentrated under reduced pressure to afford 2-chloro-N-methyl-6-(trifluoromethyl)pyridin-3-amine (2.1 g, 70%) as an oil.

MS (ESI): 211.03 [M+H]⁺.

### Step D:

A stirred solution of 2-chloro-N-methyl-6-(trifluoromethyl)pyridin-3-amine (500 mg, 2.374 mmol) and diphenylmethanimine (860 mg, 4.75 mmol) in toluene (15 mL) was added potassium tert-butoxide (800 mg, 7.123 mmol) and the mixture was degassed for 5 min with N₂. BrettPhos Pd G3 (215 mg, 0.24 mmol) was added and the mixture was heated to 100° C. for 3 h. The progress of the reaction was monitored by TLC. TLC showed completion of reaction. The reaction mixture was diluted with DCM (30 mL) and washed with water (30 mL). The organic layer was concentrated to get the crude product. The crude product was purified by column chromatography using silica gel. The product was eluted at 10% EtOAc in hexane. The pure fractions were collected and concentrated under reduced pressure to get the desired compound as an oil (500 mg).

MS (ESI): 356.34 [M+H]⁺.

### Step E:

To a stirred solution of 2-((diphenylmethylene)amino)-N-methyl-6-(trifluoromethyl) pyridin-3-amine (500 mg, 1.407 mmol) in 1,4-dioxane (10 mL) was added 4.0 M HCl in dioxane (3.0 ml) and the mixture was stirred for 18 h at rt. The progress of the reaction was monitored by TLC. The reaction mixture was diluted with EtOAc (10 ml), washed with water and concentrated to get a crude compound (200 mg).

MS (ESI): 192.22 [M+H]⁺.

### Step F:

To a stirred solution of N3-methyl-6-(trifluoromethyl)pyridine-2,3-diamine (200 mg, 1.046 mmol) and 4-chloro-2-hydroxybenzaldehyde (164 mg, 1.046 mmol) in DMA (5 mL) was added sodium bisulfite (130 mg, 1.256 mmol) at RT, and the reaction mixture was stirred at 100° C. for 16 h. The progress of the reaction was monitored by TLC. TLC showed consumption of the starting material. The reaction mixture was diluted with water (10 mL) and extracted with EtOAc (2×10 mL). The combined organic layers were washed with brine solution (10 mL), dried over anhydrous sodium sulfate, filtered and concentrated under reduced pressure to get a crude product as an oil. The crude product was purified by prep HPLC. Conditions: mobile phase A: 10 mM aq. ammonium bicarbonate, mobile phase B: cetonitrile. Column: INERTIAL, flow: 20 mL/min Method: (T/% of B): 0/10, 2/10, 11/60, 15/60, 15.1/100, 17/100, 17.1/10. Solubility: ACN+THF+H₂O. Temperature: ambient. The pure compound fractions were evaporated and lyophilized to afford 5-chloro-2-(1-methyl-5-(trifluoromethyl)-1H-imidazo[4,5-b]pyridin-2-yl)phenol (22 mg, 6% over 3 steps) as an off-white solid.

MS (ESI): 328.27 [M+H]⁺.

¹H NMR (400 MHz, DMSO-d₆) δ 8.32 (d, 1H), 7.81 (d, 1H), 7.53 (d, 1H), 7.05 (d, 1H), 7.01 (dd, 1H), 3.80 (s, 3H).

Following the preparation of Example 49 Step F, the following example was prepared, using the appropriate aldehyde followed by a deprotection step, as shown in Table 5:

TABLE 5

| | | 1. ¹H-NMR |
|---|---|---|
| Aldehyde | Example | 2. MH⁺ (ESI) |
| Int 5 | 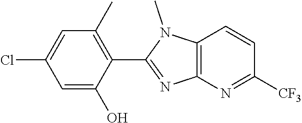 [SMILES: Cc1cc(Cl)cc(O)c1-c1nc2nc(C(F)(F)F)ccc2n1C] | 1. ¹H NMR (400 MHz, DMSO-d₆) δ 10.47 (s, 1H), 8.33 (d, 1H), 7.82 (d, 1H), 6.98 (d, 1H), 6.90 (d, 1H), 3.67 (s,3H), 2.08 (d, 3H). 2. 342.17 |
| | 50 | |
| | 5-chloro-3-methyl-2-(1-methyl-5-(trifluoromethyl)- | |
| | 1H-imidazo[4,5-b]pyridin-2-yl)phenol | |

### Example 51: 2-(6-(hydroxymethyl)-5-methoxy-1-methyl-1H-imidazo[4,5-b]pyridin-2-yl)-5-(trifluoromethyl)phenol

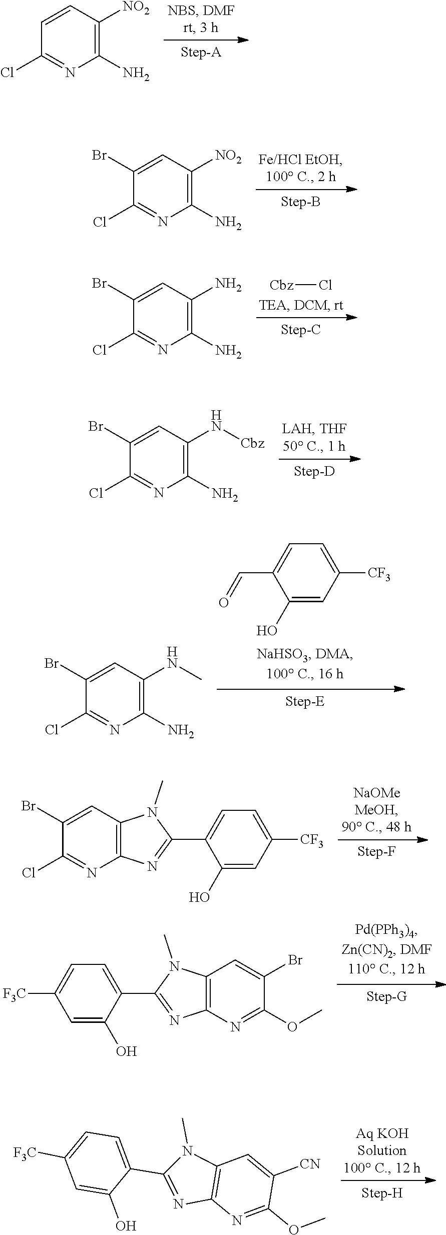

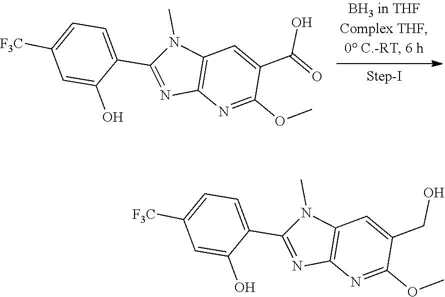 [SMILES: COc1nc2nc(-c3ccc(C(F)(F)F)cc3O)n(C)c2cc1C(=O)O ; COc1nc2nc(-c3ccc(C(F)(F)F)cc3O)n(C)c2cc1CO]

### Step A:

To a stirred solution of 6-chloro-3-nitropyridin-2-amine (15 g, 86.427 mmol) in DMF (300 mL) was added N-bromosuccinimide (16.921 g, 95.070 mmol) at RT and the mixture was stirred at RT for 3 h. Progress of the reaction was monitored by TLC. TLC showed completion of reaction. The reaction mixture was quenched with ice cold water (300 mL) and stirred for 30 min. The solid was filtered off, washed with water (300 mL) and dried under vacuum to afford the desired product (20.0 g, 92%) as a yellow solid.

MS (ESI): 252.1 [M+H]⁺.

### Step B:

To a stirred solution of 5-bromo-6-chloro-3-nitropyridin-2-amine (20 g, 79.224 mmol) in ethanol (400 mL) and water (100 mL), were added iron powder (13.27 g, 237.67 mmol) followed by aq. HCl (10 mL). The reaction mixture was stirred at 100° C. for 2 h. The progress of the reaction was monitored by LC-MS. LC-MS showed completion of reaction. The reaction mixture was filtered through celite pad, and the filtrate was concentrated under reduced pressure to get the crude product. It was purified by column chromatography using silica gel. The product was eluted at 15% EtOAc in petroleum ether. The pure fractions were collected and concentrated under reduced pressure to afford 5-bromo-6-chloropyridine-2,3-diamine (11.0 g, 62%) as a brown solid.

MS (ESI): 222.20 [M+H]⁺.

### Step C:

To a stirred solution of 5-bromo-6-chloropyridine-2,3-diamine (11 g, 49.445 mmol) in THF (220 mL) were added pyridine (12 mL, 148.34 mmol), and benzyl chloroformate (7.02 mL, 49.45 mmol) at 0° C. and the mixture was allowed to stir at rt for 2 h. The progress of the reaction was monitored by TLC. TLC showed completion of reaction. The reaction mixture was diluted with ice/water (200 mL). The pH was adjusted with glacial acetic acid and extracted with EtOAc (2×100 mL). The combined organic layer was washed with brine solution (200 mL), dried over anhydrous sodium sulfate, filtered and concentrated under reduced pressure to get the crude product as a brown solid. The crude product was purified by column chromatography using silica gel. The product was eluted at 10% EtOAc in hexane. The pure fractions were collected and concentrated under reduced pressure to afford benzyl (2-amino-6-chloropyridin-3-yl) carbamate (16 g, 94%) as a brown solid. ¹H NMR (400 MHz, CDCl3) δ 7.39 (s, 6H), 6.18 (s, 1H), 5.21 (s, 2H), 4.64 (s, 2H).

### Step D:

To a stirred solution of benzyl (2-amino-5-bromo-6-chloropyridin-3-yl) carbamate (5.0 g, 14.021 mmol) in THF (100 mL) was added 2M LAH in THF (28.04 mL, 56.085 mmol) at 0° C. and the mixture was allowed to stir at rt for 60 min. The progress of the reaction was monitored by TLC. TLC showed consumption of the starting material. The reaction mixture was quenched with saturated ammonium chloride (200 mL), adjusted to pH 4 with glacial acetic acid and extracted with EtOAc (2×250 mL). The combined organic layers were washed with brine solution (100 mL), dried over anhydrous sodium sulfate, filtered and concentrated under reduced pressure to get the crude product as a liquid. It was purified by column chromatography using silica gel. The product was eluted at 50% EtOAc in hexane. The pure fractions were collected and concentrated under reduced pressure to get 5-bromo-6-chloro-N3-methylpyridine-2,3-diamine (2 g, 60%) as an off-white solid.

MS (ESI): 236.01 [M+H]⁺.

### Step E:

To a stirred solution of 5-bromo-6-chloro-N3-methylpyridine-2,3-diamine (2 g, 8.512 mmol) and 2-hydroxy-4-(trifluoromethyl) benzaldehyde (1.78 g, 9.36 mmol) in DMA (30 mL) was added sodium bisulfite (1.33 g, 12.77 mmol) at RT, and the reaction mixture was stirred at 100° C. for 16 h. The progress of the reaction was monitored by TLC. TLC showed completion of reaction. The reaction mixture was cooled to RT, diluted with water (100 mL) and stirred for 10 min. The solid was filtered off, washed with water (100 mL) and dried under vacuum to get 2-(6-bromo-5-chloro-1-methyl-1H-imidazo[4,5-b]pyridin-2-yl)-5-(trifluoromethyl)phenol (1.9 g, 54.9%) as an off-white solid.

MS (ESI): 406.08 [M+H]⁺.

### Step F:

To a stirred solution of 2-(6-bromo-5-chloro-1-methyl-1H-imidazo[4,5-b] pyridin-2-yl)-5-(trifluoromethyl) phenol (500 mg, 1.23 mmol) in methanol (10 mL) at RT, was added sodium methoxide, ca 30% w/w in methanol (10 mL) and the mixture was stirred at 90° C. for 48 h. The progress of the reaction was monitored by LCMS. The reaction mixture was cooled to RT, diluted with water (100 mL) and extracted with EtOAc (2×300 mL). The combined organic layers were separated, washed with NaCl solution (100 mL), dried over anhydrous sodium sulfate, filtered and concentrated under reduced pressure to get the crude product. The crude product was washed with pentane (10 mL) followed by diethyl ether (10 mL) to get the 2-(6-bromo-5-methoxy-1-methyl-1H-imidazo[4,5-b]pyridin-2-yl)-5-(trifluoromethyl)phenol (270 mg, 55%) as an off-white solid.

MS (ESI): 402.20 [M+H]⁺.

### Step G:

A stirred solution of 2-(6-bromo-5-methoxy-1-methyl-1H-imidazo[4,5-b] pyridin-2-yl)-5-(trifluoromethyl) phenol (500 mg, 1.243 mmol) in N,N-dimethylformamide (10 mL) was purged with nitrogen for 5 min. Tetrakis(triphenylphosphine)palladium(0) (287 mg, 0.249 mmol) and zinc cyanide (145 mg, 1.243 mmol) were added at RT in a sealed tube and the mixture was stirred at 110° C. for 12 h. The progress of the reaction was monitored by LC-MS. The reaction mixture was cooled to RT, diluted with water (100 mL) and extracted with EtOAc (2×300 mL). The combined organic layers were separated, washed with NaCl solution (100 mL), dried over anhydrous sodium sulfate, filtered and concentrated under reduced pressure to get the crude product. The crude product was washed with pentane (20 mL) followed by diethylether (15 mL) to get the 2-(2-hydroxy-4-(trifluoromethyl)phenyl)-5-methoxy-1-methyl-1H-imidazo[4,5-b]pyridine-6-carbonitrile (270 mg, 62%) as an off-white solid.

MS (ESI): 349.25 [M+H]⁺.

### Step H:

A stirred solution of 2-(2-hydroxy-4-(trifluoromethyl)phenyl)-5-methoxy-1-methyl-1H-imidazo[4,5-b]pyridine-6-carbonitrile (270 mg, 0.775 mmol) in 4N KOH aq solution (10 mL) was heated to 100° C. for 12 h. The progress of the reaction was monitored by LC-MS. The reaction mixture was cooled to RT, diluted with water (50 mL) and extracted with EtOAc (2×30 mL). The aqueous layer was acidified with 2N HCl and extracted with EtOAc (2×200 mL). The organic layer was washed with brine (100 mL), dried over anhydrous sodium sulfate, filtered and concentrated under reduced pressure to get the crude product. The crude product was washed with pentane (25 mL) followed by diethylether (15 mL) to get 2-(2-hydroxy-4-(trifluoromethyl)phenyl)-5-methoxy-1-methyl-1H-imidazo[4,5-b]pyridine-6-carboxylic acid (250 mg, 88%) as an off-white solid.

MS (ESI): 368.20 [M+H]⁺.

### Step I:

To a cooled solution of 2-(2-hydroxy-4-(trifluoromethyl) phenyl)-5-methoxy-1-methyl-1H-imidazo[4,5-b] pyridine-6-carboxylic acid (250 mg, 0.136 mmol) in THF (10 mL) was added borane-tetrahydrofuran complex (10 mL) at 0° C. The reaction mixture was allowed to gently reach RT and was stirred at RT for 12 h. The reaction mixture was diluted with water (50 mL) and extracted with EtOAc (2×300 mL). The combined organic layers were separated, washed with NaCl solution (100 mL), dried over anhydrous sodium sulfate, filtered and concentrated under reduced pressure to get a crude compound which was purified by Prep HPLC. Conditions: mobile phase A: 10 mM ammonium bicarbonate aq., mobile phase B: acetonitrile. Column: YMC C18 (25 mm×150 mm, 10 μm), flow: 20 mL/min. Method: (T/% of B): 0/20, 2/30, 8/60. Solvent: acetonitrile+water+THF. Temperature: ambient. Pure fraction was collected and concentrated under reduced pressure and lyophilized to get 2-(6-(hydroxymethyl)-5-methoxy-1-methyl-1H-imidazo[4,5-b]pyridin-2-yl)-5-(trifluoromethyl)phenol (48 mg, 20%).

MS (ESI): 354.21 [M+H]⁺.

¹H NMR (400 MHz, DMSO-d₆) δ 11.78 (s, 1H), 8.02 (d, 1H), 7.80 (d, 1H), 7.34-7.28 (m, 2H), 5.31 (s, 1H), 4.59 (s, 2H), 3.94 (s, 3H), 3.82 (s, 3H).

Following the preparation of Example 51, the following example was prepared, using the appropriate aldehyde followed by a deprotection step with TFA, as shown in Table 6:

TABLE 6

| | | 1. ¹H-NMR |
|---|---|---|
| Aldehyde | Example | 2. MH⁺ (ESI) |
| Int 4 | 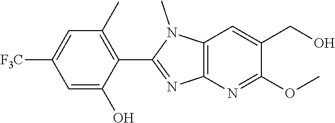 [SMILES: COc1nc2nc(-c3c(C)cc(C(F)(F)F)cc3O)n(C)c2cc1CO] | 1. ¹H NMR (400 MHz, DMSO-d₆) δ 11.78 (s, 1H), 8.02 (d, 1H), 7.80 (d, 1H), 7.34 − 7.28 (m, 2H), 5.31 (s, 1H), 4.59 (s, 2H), 3.94 (s, 3H), 3.82 (s, 3H). 2. 368.28 |
| | 52 | |
| | 2-(6-(hydroxymethyl)-5-methoxy-1- | |
| | methyl-1H-imidazo[4,5-b]pyridin-2-yl)-3- | |
| | methyl-5-(trifluoromethyl)phenol | |

### Example 53: enantiopure 3-methyl-2-(1-methyl-5-((1-methylpyrrolidin-3-yl)oxy)-1H-imidazo[4,5-b]pyrazin-2-yl)-5-(trifluoromethyl)phenol

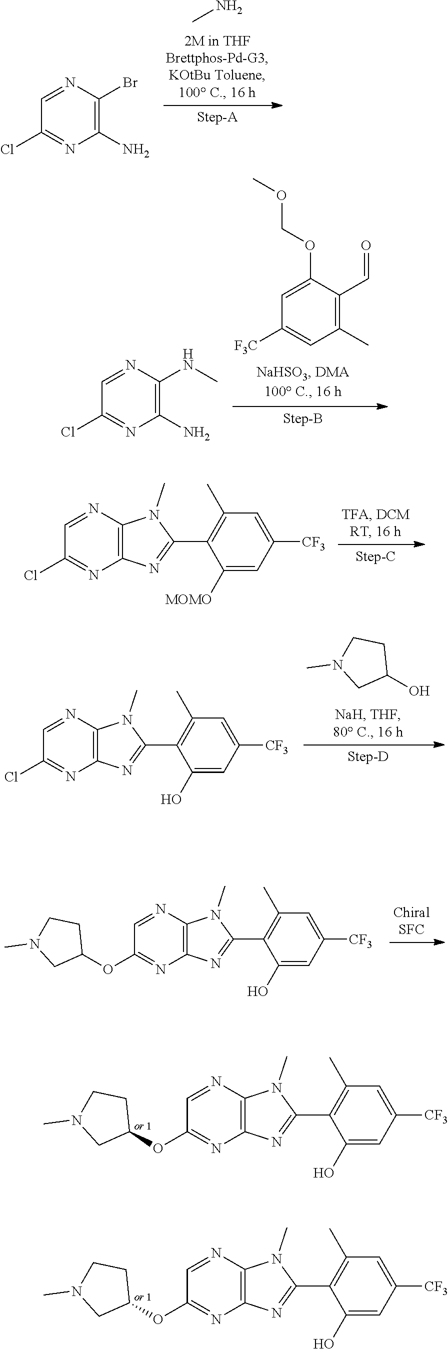

### Step A:

To a degassed solution of 3-bromo-6-chloropyrazin-2-amine (1.0 g, 4.79 mmol) in toluene (50 mL) were added potassium tert-butoxide (1.61 g, 14.39 mmol), BrettPhos Pd G3 (0.435 g, 0.480 mmol) and methylamine solution 2.0 M in THF (7.19 mL, 14.39 mmol) at RT. The reaction mixture was stirred at 100° C. for 16 h in a sealed tube. The progress of the reaction was monitored by TLC. TLC showed consumption of the starting material. The reaction mixture was diluted with water (100 mL) and extracted with EtOAc (2×200 mL). The combined organic layers were washed with brine solution (100 mL), dried over anhydrous sodium sulfate, filtered and concentrated under reduced pressure to get a crude product as a black solid. This was triturated with hexane (20 mL) and diethyl ether (10 mL) to afford 5-chloro-N2-methylpyrazine-2,3-diamine (0.60 g, 79%) as a brown solid.

MS (ESI): 158.93 [M+H]⁺.

### Step B:

To a stirred solution of 5-chloro-N2-methylpyrazine-2,3-diamine (0.60 g, 3.78 mmol) and 2-(methoxymethoxy)-6-methyl-4-(trifluoromethyl)benzaldehyde (Int 4) (0.939 g, 3.78 mmol) in N,N-dimethylacetamide (12 mL) was added sodium bisulfite (0.609 g, 4.54 mmol) at RT and the reaction mixture was stirred at 100° C. for 16 h. The progress of the reaction was monitored by TLC. TLC showed consumption of the starting material. The reaction mixture was diluted with water (100 mL) and extracted with EtOAc (2×100 mL). The combined organic layers were washed with brine solution (50 mL), dried over anhydrous sodium sulfate, filtered and concentrated under reduced pressure to get the crude product as a brown oil.

MS (ESI): 387.27 [M−H]⁻.

### Step C:

To a stirred solution of 5-chloro-2-(2-(methoxymethoxy)-6-methyl-4-(trifluoromethyl)phenyl)-1-methyl-1H-imidazo[4,5-b]pyrazine (0.60 g, 1.55 mmol) in DCM (6 mL) was added trifluoroacetic acid (6 mL) at 0° C. and the reaction mixture was stirred at RT for 16 h. The progress of the reaction was monitored by TLC. TLC showed consumption of the starting material. The reaction mixture was diluted with water (100 mL) and extracted with EtOAc (2×200 mL). The combined organic layers were washed with brine solution (100 mL), dried over anhydrous sodium sulfate, filtered and concentrated under reduced pressure to get a crude product as a brown gummy. Crude product was triturated with n-pentane (10 mL) and diethylether (5 mL) to afford 2-(5-chloro-1-methyl-1H-imidazo[4,5-b]pyrazin-2-yl)-3-methyl-5-(trifluoromethyl)phenol (0.25 g, 47%) as an off-white solid.

MS (ESI): 343.24 [M+H]⁺.

### Step D:

To a stirred solution of 2-(5-chloro-1-methyl-1H-imidazo[4,5-b]pyrazin-2-yl)-3-methyl-5-(trifluoromethyl)phenol (0.22 g, 0.642 mmol) and 1-methylpyrrolidin-3-ol (0.39 g, 3.85 mmol) in THF (11.0 ml) was added Sodium hydride, 60% dispersion in mineral oil (0.092 g, 3.85 mmol) at rt and heated to 80° C. for 16 h. The progress of the reaction was monitored by TLC. TLC showed consumption of the starting material. The reaction mixture was quenched with methanol (5 mL) and concentrated under reduced pressure to get a crude product. Crude product was diluted with ethyl acetate (20 mL) and stirred for 10 min. The solid was filtered off and the filtrate emulsion was concentrated under reduced pressure to get a crude product as a brown gummy. The crude product was purified by Prep HPLC purification (column XS CSH C18 (150 mm×25 mm, 10 μm), buffer: 10 mM ammonium bicarbonate, mobile phase: acetonitrile, flow; 18 mL/min).

The fractions were collected and lyophilized to afford the racemate product 3-methyl-2-(1-methyl-5-((1-methylpyrrolidin-3-yl)oxy)-1H-imidazo[4,5-b]pyrazin-2-yl)-5-(trifluoromethyl)phenol (0.170 g, 65%) as an off-white solid.

The racemate product was separated by chiral SFC (column: Lux-cellulose-5C, 250 mm×30 mm, 5 μm, 30° C., eluent B: MeOH 25%+0.5% diethylamine, flow: 100 g/min, pressure: 100 bar, stack time: 7 min) to provide the pure enantiomer Ex 47 (second eluting) (0.080 g, 47%) as a white solid.

MS (ESI): 408.29 [M+H]⁺.

¹H NMR (400 MHz, DMSO-d₆) δ 10.4 (brs, 1H), 8.06 (s, 1H), 7.19 (s, 1H), 7.11 (d, 1H), 5.47-5.37 (m, 1H), 3.60 (s, 3H), 2.82 (dd, 1H), 2.75-2.64 (m, 2H), 2.41-2.31 (m, 2H), 2.28 (s, 3H), 2.18 (s, 3H), 1.94-1.82 (m, 1H).

### Example 54: 3-(((2-(2-hydroxy-4-(trifluoromethyl)phenyl)-1-methyl-1H-imidazo[4,5-b]pyridin-5-yl)amino)methyl)oxetan-3-ol

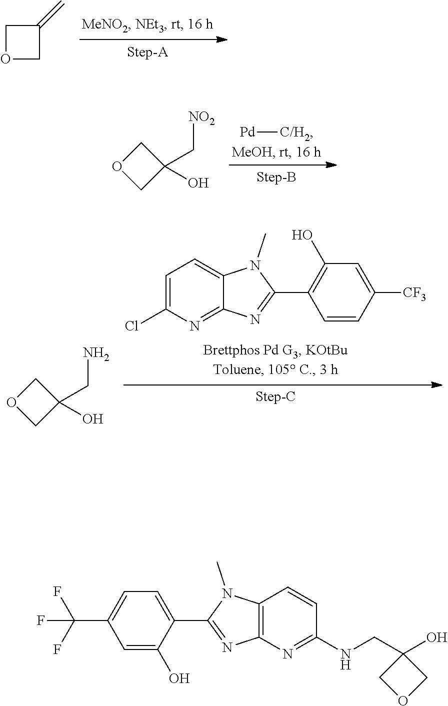

### Step A:

To a stirred solution of oxetan-3-one (1.0 g, 13.88 mmol) in nitromethane (4.0 mL) was added triethylamine, 99%, pure (2.27 mL, 16.65 mmol) at 0° C. and the reaction mixture was stirred at rt for 16 h. The progress of the reaction was monitored by TLC. TLC showed consumption of the starting material. The reaction mixture was diluted with water (250 mL) and extracted with EtOAc (2×250 mL). The combined organic layers were washed with brine solution (100 mL), dried over anhydrous sodium sulfate, filtered and concentrated under reduced pressure to get a crude product as a liquid. The crude product was purified by column chromatography using silica gel and eluted with EtOAc in hexane as a gradient. The product was eluted at 30% EtOAc in hexane. The pure fractions were collected and concentrated under reduced pressure to get the pure compound (1.1 g, 59%).

### Step B:

To a stirred solution of 3-(nitromethyl)oxetan-3-ol (1.1 g, 8.26 mmol) in methanol (25 mL) was added 10% palladium on activated carbon, (0.52 g, 4.96 mmol) at rt and the mixture was stirred under H₂ balloon pressure for 16 h. The progress of the reaction was monitored by TLC. TLC showed consumption of the starting material. The reaction mixture was filtered on a celite bed and washed with methanol (50 mL). The filtrate was concentrated under reduced pressure to get the crude product (91%).

MS (ESI): 104.08 [M+H]⁺.

¹H NMR (400 MHz, CDCl3) δ 4.65 (d, 2H), 4.20 (d, 2H), 3.10 (s, 2H)

### Step C:

To a stirred solution of 2-(5-chloro-1-methyl-1H-imidazo[4,5-b]pyridin-2-yl)-5-(trifluoromethyl)phenol (150 mg, 0.458 mmol) and 3-(aminomethyl)oxetan-3-ol (141.61 mg, 1.373 mmol) in toluene (6 mL) was added potassium tert-butoxide (154 mg, 1.373 mmol) at rt. The mixture was purged with nitrogen gas for 5 min then BrettPhos Pd G3 (46 mg, 0.458 mmol) was added. The mixture was heated to 105° C. for 1 h in a sealed tube. The progress of the reaction was monitored by TLC. TLC showed completion of reaction. The reaction mixture was cooled to RT, filtered on a celite bed, washed with EtOAc (200 mL) and concentrated under reduced pressure to get the crude product as a solid. The crude compound was purified by Prep HPLC purification. Prep HPLC Conditions: mobile phase A: 10 mM ammonium bicarbonate (Aq), mobile phase B: acetonitrile; Column: X-Bridge (25 mm×150 mm), 10 μm, flow: 12 mL/min. Method: (T/% B): 0/40, 2/40, 6/55, 15/100, 15.01/100, 19/100, Solvent: ACN+Water+THF; Temperature: ambient.

The pure fraction were collected and lyophilized to afford 3-(((2-(2-hydroxy-4-(trifluoromethyl)phenyl)-1-methyl-1H-imidazo[4,5-b]pyridin-5-yl)amino)methyl)oxetan-3-ol (20 mg, 11%) as a pale yellow solid.

MS (ESI): 395.47 [M+H]⁺.

¹H NMR (400 MHz, DMSO-d₆) δ 12.32 (s, 1H), 7.84 (d, 1H), 7.79 (d, 1H), 7.29 (s, 1H), 7.26 (d, 1H), 6.76 (t, 1H), 6.65 (d, 1H), 6.38 (s, 1H), 4.46 (d, 2H), 4.41 (d, 2H), 3.80 (s, 3H), 3.68 (d, 2H).

### Example 55: 3-methyl-2-(7-methyl-2-((1-methylpyrrolidin-3-yl)oxy)-7H-purin-8-yl)-5-(trifluoromethyl)phenol

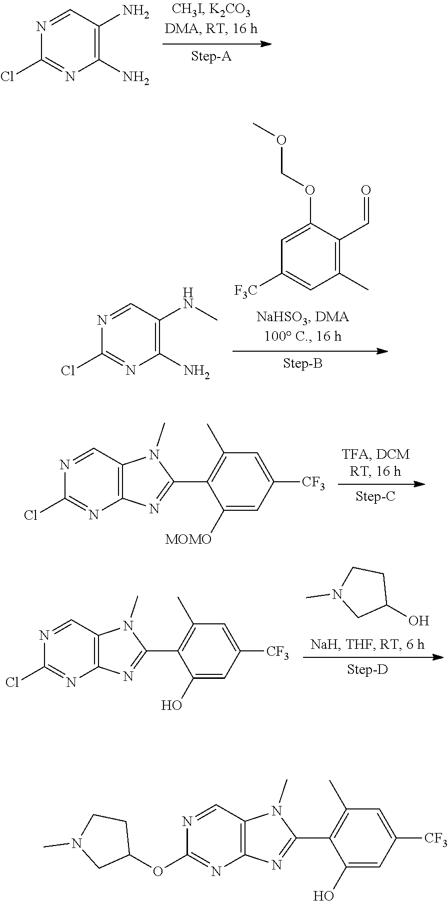

### Step A:

To a stirred solution of 2-chloropyrimidine-4,5-diamine (3.0 g, 20.75 mmol) in N,N-dimethylacetamide (150 mL) were added potassium carbonate (4.302 g, 31.128 mmol) and methyl iodide (1.94 mL, 31.128 mmol) at RT and the mixture was stirred at RT for 16 h. The progress of the reaction was monitored by TLC. TLC showed consumption of the starting material. The reaction mixture was diluted with water (100 mL) and extracted with EtOAc (2×200 mL). The combined organic layers were washed with brine solution (100 mL), dried over anhydrous sodium sulfate, filtered and concentrated under reduced pressure to get a crude product as a black oil. The crude product was purified by column chromatography using silica gel (100-200 mesh) and eluted with 0-100% EtOAC/hexane as a gradient. The product was eluted at 50% EtOAc/hexane. The pure fractions were collected and concentrated under reduced pressure to afford 2-chloro-N5-methylpyrimidine-4,5-diamine (0.60 g, 18%) as an off-white solid.

MS (ESI): 159.32 [M+H]⁺.

### Step B:

Following example 53 Step B, 2-chloro-8-(2-(methoxymethoxy)-6-methyl-4-(trifluoromethyl)phenyl)-7-methyl-7H-purine (0.6 g, 18%) was obtained as a brown gummy residue.

MS (ESI): 387.31 [M+H]⁺.

### Step C:

Following example 53 Step C, 2-(2-chloro-7-methyl-7H-purin-8-yl)-3-methyl-5-(trifluoromethyl)-phenol (0.120 g, 23%) was obtained as an off-white solid.

MS (ESI): 343.15 [M+H]⁺.

### Step D:

Following example 53 Step D, the racemate compound 3-methyl-2-(7-methyl-2-((1-methylpyrrolidin-3-yl)oxy)-7H-purin-8-yl)-5-(trifluoromethyl)phenol (4.7 mg, 4%) was obtained as an off-white solid.

MS (ESI): 408.34 [M+H]⁺.

¹H NMR (400 MHz, DMSO-d₆) δ 8.75 (s, 1H), 6.23 (s, 1H), 6.10-5.99 (m, 1H), 5.37 (ddd, 1H), 3.63 (s, 3H), 2.85 (dd, 1H), 2.70-2.57 (m, 2H), 2.42-2.29 (m, 2H), 2.27 (s, 3H), 2.01 (s, 3H), 1.89-1.77 (m, 1H).

### Example 56 2-(5-methoxy-1-methyl-1H-imidazo[4,5-b]pyrazin-2-yl)-3-methyl-5-(trifluoromethyl)phenol

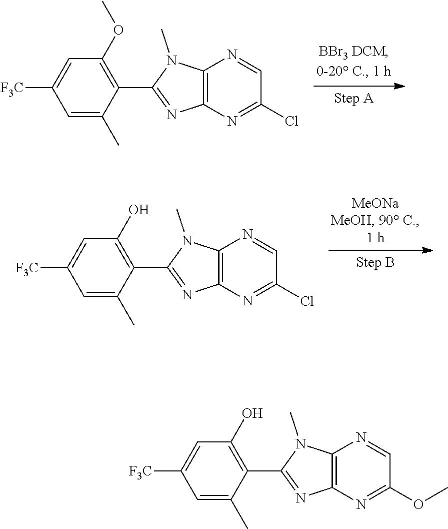 [SMILES: COc1cc(C(F)(F)F)cc(C)c1-c1nc2nc(Cl)cnc2n1C ; Cc1cc(C(F)(F)F)cc(O)c1-c1nc2nc(Cl)cnc2n1C ; COc1cnc2c(n1)nc(-c1c(C)cc(C(F)(F)F)cc1O)n2C]

### Step A:

To the solution of 5-chloro-2-[2-methoxy-6-methyl-4-(trifluoromethyl)phenyl]-1-methyl-imidazo[4,5-b]pyrazine (50 mg, 140.16 μmol, 1 eq) in DCM (2 mL) was added BBr₃ (105.34 mg, 420.49 μmol, 40.52 μL, 3 eq) at 0° C. and the reaction mixture was stirred at 20° C. for 1 hr. LCMS indicated that the reaction was completed. After completion, the solvent was removed by concentration to give the residue. The residue was used for the next step directly without further purification. 2-(5-Chloro-1-methyl-imidazo[4,5-b]pyrazin-2-yl)-3-methyl-5-(trifluoromethyl)phenol (50 mg, crude) was obtained as a yellow solid.

LC-MS (ES⁺, m/z): 343.2 [(M+H)⁺].

### Step B:

To 2-(5-chloro-1-methyl-imidazo[4,5-b]pyrazin-2-yl)-3-methyl-5-(trifluoromethyl)phenol (50 mg, 145.90 μmol, 1 eq) was added a solution of NaOMe in MeOH (21.30 mg, 145.90 μmol, 2 mL, 37%, 1 eq), and the reaction mixture was stirred at 90° C. for 1 h. LCMS indicated that the reaction was completed. The reaction mixture was poured onto 5 mL H₂O. Then the resulting solution was extracted with DCM (5 mL×2). The combined organic layer was washed successively with water (5 mL×2) and brine (5 mL×1), dried over anhydrous Na₂SO₄, filtered, and concentrated to give a residue. The residue was purified by prep-HPLC (column: Waters Xbridge C18 150 mm×50 mm×10 μm; mobile phase: [H₂O (10 mM NH₄HCO₃)-ACN]; gradient: 25%-55% B over 8.0 min) to give the title product. 2-(5-Methoxy-1-methyl-imidazo[4,5-b]pyrazin-2-yl)-3-methyl-5-(trifluoromethyl)phenol (3.03 mg, 8.75 μmol, 6.4% yield for 2 steps, 97.65% purity) as a white solid.

LC-MS (ES⁺, m/z): 339.2 [(M+H)⁺].

¹H NMR (400 MHz, DMSO-d₆) δ=10.80 (br d, J=5.9 Hz, 1H), 8.11 (s, 1H), 7.22 (br s, 1H), 7.13 (s, 1H), 3.97 (s, 3H), 3.61 (s, 3H), 2.18 (s, 3H).

### Example 57 2-[6-(1-hydroxy-1-methyl-ethyl)-5-methoxy-1-methyl-imidazo[4,5-b]pyridin-2-yl]-3-methyl-5-(trifluoromethyl)phenol

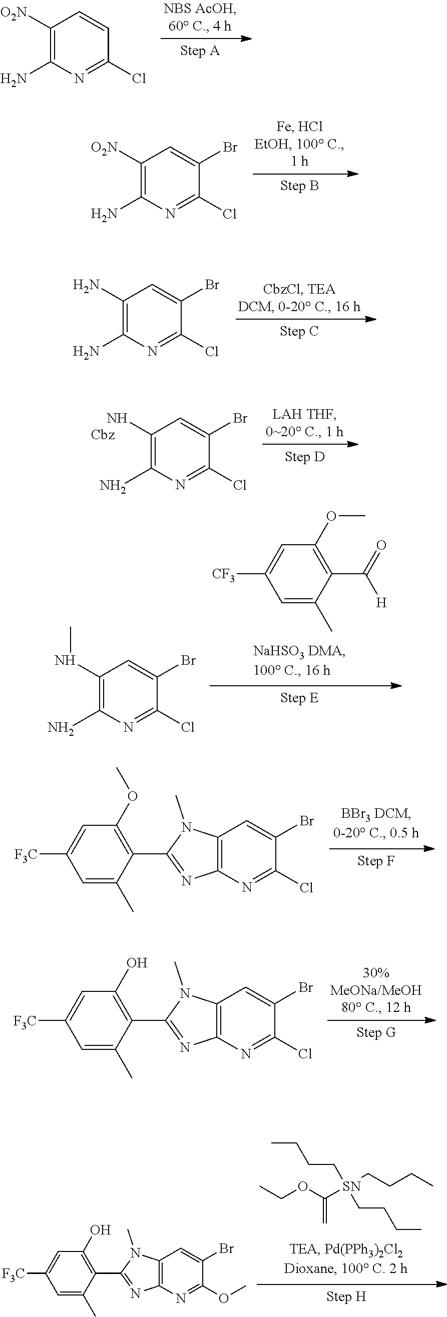

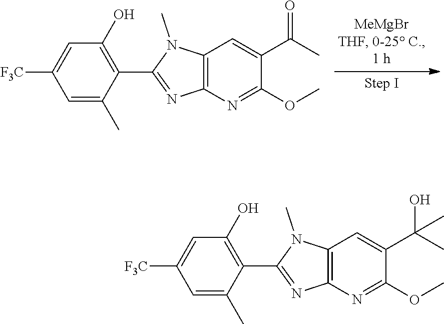 [SMILES: COc1nc2nc(-c3c(C)cc(C(F)(F)F)cc3O)n(C)c2cc1C(C)=O ; COc1nc2nc(-c3c(C)cc(C(F)(F)F)cc3O)n(C)c2cc1C(C)(C)O]

### Step A:

To a solution of 6-chloro-3-nitro-pyridin-2-amine (10 g, 57.62 mmol, 1 eq) in AcOH (200 mL) was added NBS (11.28 g, 63.38 mmol, 1.1 eq). The mixture was stirred at 60° C. for 4 hr. TLC (PE:EA=4:1, product Rf=0.6) indicated that the reaction was completed. After completion, the precipitate was collected by filtration to give the crude product. The crude product was used for the next step directly without further purification. 5-Bromo-6-chloro-3-nitro-pyridin-2-amine (8 g, crude) was obtained as a yellow solid.

LC-MS (ES⁺, m/z): 252.4 [(M−H)⁺]

### Step B:

To the solution of 5-bromo-6-chloro-3-nitro-pyridin-2-amine (7 g, 27.73 mmol, 1 eq) in EtOH (56 mL) and H₂O (14 mL) was added conc. HCl (12 M, 1.16 mL, 0.5 eq), then Fe (15.48 g, 277.28 mmol, 10 eq) was added in 3 portions at 100° C. and the resulting reaction mixture was stirred at 100° C. for 1 hr. LCMS indicated that the reaction was completed. The reaction mixture was filtered through Celite and the filter cake was washed with EA (200 mL×2). The filtrate was concentrated by vacuo to get the residue. The residue was dissolved to 500 mL H₂O and 250 mL EA. After that, the aqueous phase was separated and extracted with EA (200 mL×2). The combined organic layer was washed successively with water (200 mL×2) and brine (200 mL×1), dried over anhydrous Na₂SO₄, filtered, and concentrated to give a residue. The residue was purified by column chromatography (SiO₂, petroleum ether/ethyl acetate=100/1 to 1/1) to give the title product. 5-Bromo-6-chloro-pyridine-2,3-diamine (5.6 g, 25.17 mmol, 90.78% yield, N/A purity) was obtained as a black brown solid.

LC-MS (ES⁺, m/z): 222.0 [(M+H)⁺].

### Step C:

To a solution of 5-bromo-6-chloro-pyridine-2,3-diamine (5 g, 22.47 mmol, 1 eq) in THF (50 mL) was added pyridine (3.56 g, 44.95 mmol, 3.63 mL, 2 eq) and CbzCl (5.75 g, 33.71 mmol, 4.81 mL, 1.5 eq) at 0° C. The mixture was stirred at 20° C. for 12 h. LCMS indicated that the reaction was completed. The reaction mixture was quenched by addition of H₂O 100 mL, and then extracted with EA (50 mL×3). The combined organic layers were washed with brine 100 mL, dried over Na₂SO₄, filtered and concentrated under reduced pressure to give a residue. The residue was purified by column chromatography (SiO₂, petroleum ether/ethyl acetate=1/0 to 85/15). Compound benzyl N-(2-amino-5-bromo-6-chloro-3-pyridyl)carbamate (7 g, 19.63 mmol, 87.34% yield, N/A purity) was obtained as a yellow solid.

LC-MS (ES⁺, m/z): 358.0 [(M+H)⁺].

¹H NMR (400 MHz, DMSO-d₆) δ=9.04 (br s, 1H), 8.00 (br s, 1H), 7.46-7.32 (m, 5H), 6.54 (s, 2H), 5.15 (s, 2H)

### Step D:

To a solution of benzyl N-(2-amino-5-bromo-6-chloro-3-pyridyl)carbamate (3.5 g, 9.81 mmol, 1 eq) in THF (35 mL) was added LAH (2.5 M, 15.70 mL, 4 eq) at 0° C. The mixture was stirred at 20° C. for 1 hr. LCMS indicated that the reaction was completed. The reaction mixture was quenched by addition of H₂O 60 mL at 0° C., and then extracted with EA (30 mL×3). The combined organic layers were washed with brine 100 mL, dried over Na₂SO₄, filtered and concentrated under reduced pressure to give a residue. The residue was purified by column chromatography (SiO₂, petroleum ether/ethyl acetate=1/0 to 80/20). Compound 5-bromo-6-chloro-N3-methyl-pyridine-2,3-diamine (3 g, 8.88 mmol, 90.47% yield, 70% purity) was obtained as an off-white solid.

LC-MS (ES⁺, m/z): 235.9 [(M+H)⁺].

### Step E:

To a solution of 5-bromo-6-chloro-N3-methyl-pyridine-2,3-diamine (300 mg, 1.27 mmol, 1 eq) in DMA (5 mL) was added 2-methoxy-6-methyl-4-(trifluoromethyl) benzaldehyde (304.43 mg, 1.40 mmol, 1.1 eq) and NaHSO₃ (264.01 mg, 2.54 mmol, 178.38 μL, 2 eq). The mixture was stirred at 100° C. for 16 hr. LCMS indicated that the reaction was completed. The reaction mixture was quenched by addition of H₂O 20 mL, and then extracted with EA (20 mL×3). The combined organic layers were washed with brine 100 mL, dried over Na₂SO₄, filtered and concentrated under reduced pressure to give a residue. The residue was purified by column chromatography (SiO₂, petroleum ether/ethyl acetate=1/0 to 85/15). Compound 6-bromo-5-chloro-2-[2-methoxy-6-methyl-4-(trifluoromethyl)phenyl]-1-methyl-imidazo[4,5-b]pyridine (310 mg, 570.59 μmol, 44.98% yield, 80% purity) was obtained as a yellow solid.

LC-MS (ES⁺, m/z): 434.1 [(M+H)⁺].

### Step F:

To a solution of 6-bromo-5-chloro-2-[2-methoxy-6-methyl-4-(trifluoromethyl)phenyl]-1-methyl-imidazo[4,5-b]pyridine (300 mg, 690.23 μmol, 1 eq) in DCM (3 mL) was added BBr₃ (864.59 mg, 3.45 mmol, 332.54 μL, 5 eq) at 0° C. The mixture was stirred at 20° C. for 0.5 hr. LCMS indicated that the reaction was completed. The reaction mixture was concentrated under reduced pressure to remove DCM. The residue was used for the next step directly without further purification. 2-(6-Bromo-5-chloro-1-methyl-imidazo[4,5-b]pyridin-2-yl)-3-methyl-5-(trifluoromethyl)phenol (300 mg, crude) was obtained as a yellow solid.

LC-MS (ES⁺, m/z): 420.0 [(M+H)⁺].

### Step G:

To a solution of 2-(6-bromo-5-chloro-1-methyl-imidazo[4,5-b]pyridin-2-yl)-3-methyl-5-(trifluoro-methyl)phenol (300 mg, 713.25 μmol, 1 eq) in MeOH (1 mL) was added the solution of 30% MeONa (128.44 mg, 713.25 μmol, 1 mL, 30% purity, 1 eq) in MeOH. The mixture was stirred at 80° C. for 12 hr. LCMS indicated that the reaction was completed. The reaction mixture was quenched by addition of H₂O 20 mL, and then extracted with EA (40 mL×3). The combined organic layers were washed with brine 100 mL, dried over Na₂SO₄, filtered and concentrated under reduced pressure to give a residue. The residue was purified by column chromatography by prep-TLC (SiO₂, DCM:MeOH=10:1). 2-(6-Bromo-5-methoxy-1-methyl-imidazo[4,5-b]pyridin-2-yl)-3-methyl-5-(trifluoromethyl)-phenol (190 mg, 456.52 μmol, 64.01% yield, N/A purity) was obtained as a white solid.

LC-MS (ES⁺, m/z): 416.0 [(M+H)⁺].

¹H NMR (400 MHz, DMSO-d₆) δ=10.65 (br s, 1H), 8.48 (s, 1H), 7.22 (s, 1H), 7.12 (s, 1H), 3.97 (s, 3H), 3.57 (s, 3H), 2.14 (s, 3H)

### Step H:

To a solution of 2-(6-bromo-5-methoxy-1-methyl-imidazo[4,5-b]pyridin-2-yl)-3-methyl-5-(trifluoro-methyl)phenol (90 mg, 216.25 μmol, 1 eq) in dioxane (2 mL) was added TEA (43.76 mg, 432.49 μmol, 60.20 μL, 2 eq) and tributyl(1-ethoxyvinyl)stannane (156.19 mg, 432.49 μmol, 146.11 μL, 2 eq), then was added Pd(PPh₃)₂Cl₂ (15.18 mg, 21.62 μmol, 0.1 eq). The mixture was stirred at 100° C. for 2 hr. LCMS indicated that the reaction was completed. The reaction mixture was quenched by addition of saturated KF 10 mL, and then the mixture was extracted with EA (4 mL×3). The combined organic layers were washed with brine 10 mL, dried over Na₂SO₄, filtered and concentrated under reduced pressure to give a residue. The residue was purified by prep-TLC (SiO₂, PE:EA=1:1). 1-[2-[2-Hydroxy-6-methyl-4-(trifluoromethyl)phenyl]-5-methoxy-1-methyl-imidazo[4,5-b]pyridin-6-yl]ethanone (60 mg, 158.17 μmol, 73.14% yield, N/A purity) was obtained as a white solid.

LC-MS (ES⁺, m/z): 380.1 [(M+H)⁺].

### Step I:

To a solution of 1-[2-[2-hydroxy-6-methyl-4-(trifluoromethyl)phenyl]-5-methoxy-1-methyl-imidazo[4,5-b]pyridin-6-yl]ethanone (60 mg, 158.17 μmol, 1 eq) in THF (2 mL) was added MeMgBr (3 M, 158.17 μL, 3 eq) at 0° C. The mixture was stirred at 25° C. for 1 hr. LCMS indicated that the reaction was completed. The reaction mixture was quenched by addition NH₄Cl 10 mL, and then extracted with EA (4 mL×3). The combined organic layers were washed with brine 10 mL, dried over Na₂SO₄, filtered and concentrated under reduced pressure to give a residue. The residue was purified by prep-HPLC column: Waters Xbridge C18 150 mm×50 mm, 10 μm; mobile phase: [H₂O (10 mM NH₄HCO₃)-ACN]; gradient: 30%-65% B over 8.0 min to give the title product. 2-[6-(1-Hydroxy-1-methyl-ethyl)-5-methoxy-1-methyl-imidazo[4,5-b]pyridin-2-yl]-3-methyl-5-(trifluoromethyl)phenol (6.27 mg, 15.86 μmol, 10.03% yield, 100.00% purity) was obtained as a white solid.

LC-MS (ES⁺, m/z): 396.1 [(M+H)⁺].

¹H NMR (400 MHz, DMSO-d₆) δ=10.79-10.44 (m, 1H), 8.13 (s, 1H), 7.19 (br s, 1H), 7.10 (s, 1H), 5.20 (s, 1H), 3.95 (s, 3H), 3.56 (s, 3H), 2.15 (s, 3H), 1.56 (s, 6H).

### Example 58 (R)-5-chloro-3-methyl-2-(1-methyl-5-((1-methylpyrrolidin-3-yl)oxy)-1H-imidazo[4,5-b]pyrazin-2-yl)phenol formate Salt

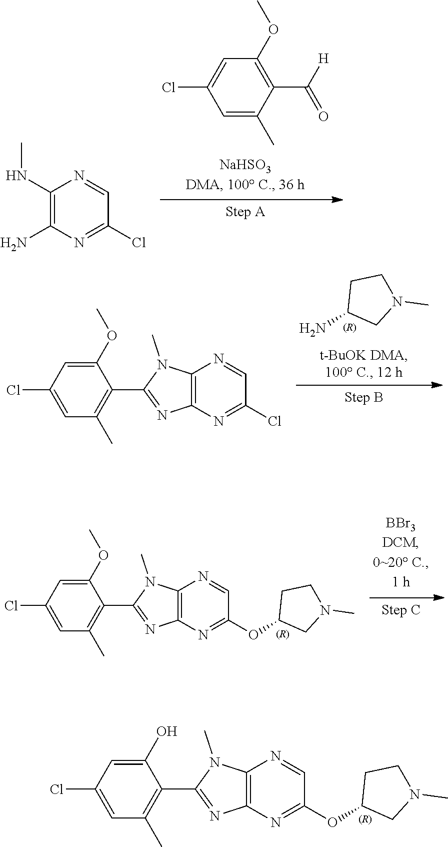

### Step A:

To the solution of 5-chloro-N2-methyl-pyrazine-2,3-diamine (200 mg, 1.26 mmol, 1.0 eq) in DMA (6 mL) was added 4-chloro-2-methoxy-6-methyl-benzaldehyde (232.83 mg, 1.26 mmol, 1.0 eq), and NaHSO₃ (262.47 mg, 2.52 mmol, 177.34 μL, 2.0 eq) and the resulting reaction mixture was stirred at 100° C. for 36 hr. LCMS indicated that the reaction was completed. The reaction mixture was poured onto 20 mL H₂O, followed by 10 mL EA. After that, the aqueous phase was separated and extracted with EA (10 mL×2). The combined organic layers were washed successively with water (20 mL×2) and brine (20 mL×1), dried over anhydrous Na₂SO₄, filtered, and concentrated to give a residue. The residue was purified by prep-TLC (SiO₂, petroleum ether:ethyl acetate=3:1). 5-chloro-2-(4-chloro-2-methoxy-6-methyl-phenyl)-1-methyl-imidazo [4,5-b]pyrazine (80 mg, 247.54 μmol, 19.63% yield, N/A purity) was obtained as a light yellow solid.

LC-MS (ES⁺, m/z): 323.2 [(M+H)⁺].

¹H NMR (400 MHz, DMSO-d₆) δ=8.58 (s, 1H), 7.19 (dd, J=1.1, 15.5 Hz, 2H), 3.79 (s, 3H), 3.59 (s, 3H), 2.13 (s, 3H)

### Step B:

To the solution of 5-chloro-2-(4-chloro-2-methoxy-6-methyl-phenyl)-1-methyl-imidazo[4,5-b]pyrazine (120 mg, 371.31 μmol, 1.0 eq) in DMA (4 mL) was added (3R)-1-methylpyrrolidin-3-ol (112.67 mg, 1.11 mmol, 122.34 μL, 3.0 eq), and t-BuOK (208.33 mg, 1.86 mmol, 5.0 eq) and the reaction mixture was stirred at 100° C. for 12 hr. LCMS indicated that the reaction was completed. The reaction mixture was poured onto 20 mL H₂O, followed by 10 mL EA. After that, the aqueous phase was separated and extracted with EA (20 mL×2). The combined organic layers were washed successively with water (20 mL×2) and brine (20 mL×1), dried over anhydrous Na₂SO₄, filtered, and concentrated to give a residue. The residue was purified by prep-TLC (SiO₂, DCM:MeOH=10:1). 2-(4-Chloro-2-methoxy-6-methyl-phenyl)-1-methyl-5-[(3R)-1-methylpyrrolidin-3-yl]oxy-imidazo[4,5-b]pyrazine (60 mg, 154.69 μmol, 41.66% yield, N/A purity) was obtained as a yellow oil.

LC-MS (ES⁺, m/z): 388.2 [(M+H)⁺].

### Step C:

To the solution of 2-(4-chloro-2-methoxy-6-methyl-phenyl)-1-methyl-5-[(3R)-1-methylpyrrolidin-3-yl]oxy-imidazo[4,5-b]pyrazine (50 mg, 128.91 μmol, 1.0 eq) in DCM (2 mL) was added BBr₃ (161.48 mg, 644.56 μmol, 62.11 μL, 5.0 eq) at 0° C. and the resulting reaction mixture was stirred at 20° C. for 1 hr. LCMS indicated that the reaction was completed. After completion, the solvent was removed by concentration to give the crude product. The residue was purified by prep-HPLC (column: Phenomenex Luna C18 100 mm×30 mm×5 μm; mobile phase: [H₂O (0.2% FA)-ACN]; gradient: 10%-40% B over 8.0 min) to give the title product. 5-Chloro-3-methyl-2-[1-methyl-5-[(3R)-1-methylpyrrolidin-3-yl]oxy-imidazo[4,5-b]pyrazin-2-yl]phenol (18.38 mg, 48.82 μmol, 37.87% yield, 99.29% purity) was obtained as a white solid.

LC-MS (ES⁺, m/z): 374.2 [(M+H)⁺].

¹H NMR (400 MHz, DMSO-d₆) δ=10.57 (br s, 1H), 8.05 (s, 1H), 6.97 (d, J=1.4 Hz, 1H), 6.90 (d, J=1.8 Hz, 1H), 5.42 (br s, 1H), 3.58 (s, 3H), 2.86 (dd, J=6.1, 10.7 Hz, 1H), 2.75 (br d, J=4.9 Hz, 2H), 2.42-2.33 (m, 2H), 2.30 (s, 3H), 2.10 (s, 3H), 1.94-1.82 (m, 1H)

### Example 59 5-chloro-2-(5-methoxy-1-methyl-imidazo[4,5-b]pyrazin-2-yl)-3-methyl-phenol formate Salt

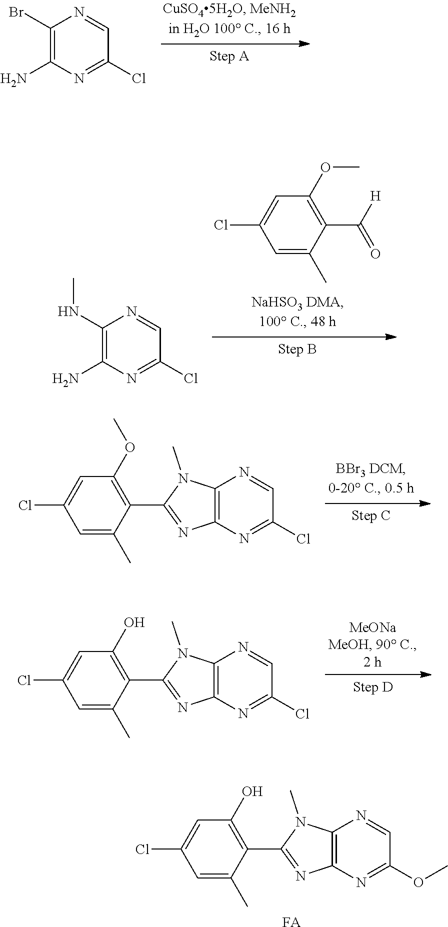

### Step A:

To the solution of 3-bromo-6-chloro-pyrazin-2-amine (3 g, 14.39 mmol, 1.0 eq) in MeNH₂ (6.21 g, 60.00 mmol, 20 mL, 4.17 eq) was added CuSO₄·5 H₂O (359.36 mg, 1.44 mmol, 0.1 eq) and the reaction mixture was stirred at 100° C. for 4 hr under N₂. LCMS indicated that the reaction was completed. The mixture was filtered through Celite. The celite pad was rinsed with DCM (2×200 ml) and concentrated in vacuo to get the crude product. The crude product was purified by column chromatography (SiO₂, petroleum ether/ethyl acetate=10/1 to 1/1). 5-Chloro-N2-methyl-pyrazine-2,3-diamine (1.5 g, 9.46 mmol, 65.72% yield, N/A purity) was obtained as a yellow solid.

LC-MS (ES⁺, m/z): 159.2 [(M+H)⁺].

¹H NMR (400 MHz, DMSO-d6) δ=7.24 (s, 1H), 6.42 (s, 2H), 6.34 (br d, J=4.3 Hz, 1H), 2.82 (d, J=4.6 Hz, 3H).

### Step B:

To the solution of 5-chloro-N2-methyl-pyrazine-2,3-diamine (150 mg, 945.84 μmol, 1.0 eq) in DMA (2 mL) was added NaHSO₃ (118.11 mg, 1.14 mmol, 79.80 μL, 1.2 eq) and 4-chloro-2-methoxy-6-methyl-benzaldehyde (174.62 mg, 945.84 μmol, 1.0 eq). The resulting reaction mixture was stirred at 100° C. for 48 hr. LCMS indicated that the reaction was completed. The reaction mixture was poured onto 20 mL H₂O, followed by 10 mL EA. After that, the aqueous phase was separated and extracted with EA (20 mL×2). The combined organic layers were washed successively with water (20 mL×2) and brine (20 mL×1), dried over anhydrous Na₂SO₄, filtered, and concentrated to give a residue. The residue was purified by column chromatography (SiO₂, petroleum ether/ethyl acetate=100/1 to 1/1). 5-Chloro-2-(4-chloro-2-methoxy-6-methyl-phenyl)-1-methyl-imidazo[4,5-b]pyrazine (0.1 g, 309.43 μmol, 32.71% yield, N/A purity) was obtained as a yellow solid.

LC-MS (ES⁺, m/z): 323.2 [(M+H)⁺].

¹H NMR (400 MHz, DMSO-d6) δ=8.58 (s, 1H), 7.21 (d, J=1.5 Hz, 1H), 7.17 (d, J=1.1 Hz, 1H), 3.79 (s, 3H), 3.59 (s, 3H), 2.13 (s, 3H)

### Step C:

To the solution of 5-chloro-2-(4-chloro-2-methoxy-6-methyl-phenyl)-1-methyl-imidazo[4,5-b]pyrazine (90 mg, 278.49 μmol, 0.01 eq) in DCM (2 mL) was added BBr₃ (348.83 mg, 1.39 mmol, 134.17 μL, 5.0 eq) at 0° C. The resulting reaction mixture was stirred at 20° C. for 0.5 hr. LCMS indicated that the reaction was completed. After completion, the solvent was removed by concentration to give the crude product. The residue was used for the next step directly without further purification. 5-chloro-2-(5-chloro-1-methyl-imidazo[4,5-b]pyrazin-2-yl)-3-methyl-phenol (0.1 g, crude) was obtained as a yellow solid.

LC-MS (ES⁺, m/z): 309.0 [(M+H)⁺].

### Step D:

To the solution of 5-chloro-2-(5-chloro-1-methyl-imidazo[4,5-b]pyrazin-2-yl)-3-methyl-phenol (80 mg, 258.77 μmol, 1.0 eq) in MeOH (0.5 mL) was added NaOMe (233.00 mg, 1.29 mmol, 0.5 mL, 30% purity, 5.0 eq). The resulting reaction mixture was stirred at 90° C. for 2 hr. LCMS indicated that the reaction was completed. The reaction mixture was purified by prep-HPLC (column: Phenomenex Luna C18 100×30 mm×5 μm; mobile phase: [H₂O (0.2% FA)-ACN]; gradient: 20%-50% B over 8.0 min) to give the title product. 5-Chloro-2-(5-methoxy-1-methyl-imidazo[4,5-b]pyrazin-2-yl)-3-methyl-phenol (49.48 mg, 162.37 μmol, 62.75% yield, 100% purity) was obtained as a white solid.

LC-MS (ES⁺, m/z): 305.0 [(M+H)⁺].

¹H NMR (400 MHz, DMSO-d6) δ=8.08 (s, 1H), 6.90 (s, 2H), 3.96 (s, 3H), 3.59 (s, 3H), 2.09 (s, 3H)

### Example 60 (R)-3-methyl-2-(1-methyl-5-(methyl(1-methylpyrrolidin-3-Yl)amino)-1H-imidazo[4,5-b]pyrazin-2-yl)-5-(trifluoromethyl)phenol formate Salt

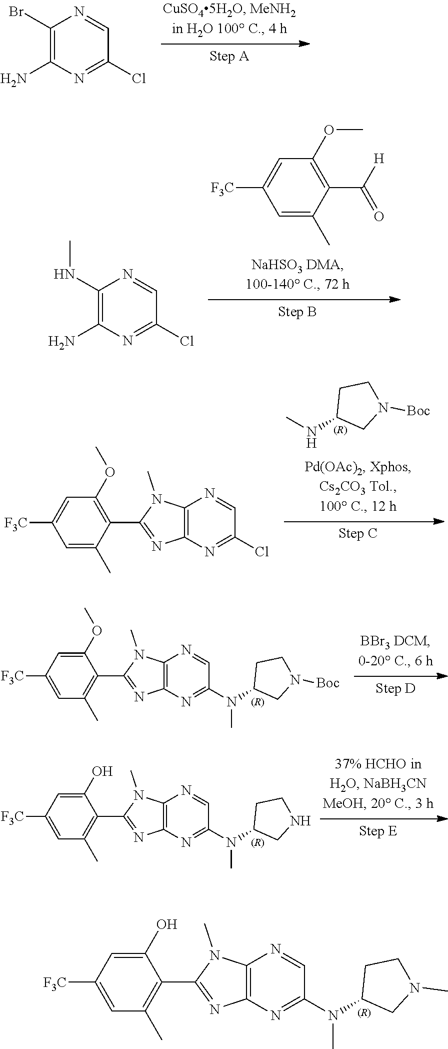

### Step A:

To the solution of 3-bromo-6-chloro-pyrazin-2-amine (3 g, 14.39 mmol, 1 eq) in MeNH₂ in H₂O (6.21 g, 60.00 mmol, 20 mL, 4.17 eq) was added CuSO₄·5 H₂O (359.36 mg, 1.44 mmol, 0.1 eq) and the reaction mixture was stirred at 100° C. for 4 hr under N₂ atmosphere. LCMS indicated that the reaction was completed. The reaction mixture was cooled to room temperature and filtered through Celite. The celite pad was rinsed with DCM (2×200 mL) and concentrated in vacuo to get the residue. The residue was purified by column chromatography (SiO₂, petroleum ether/ethyl acetate=10/1 to 1/1). 5-chloro-N2-methyl-pyrazine-2,3-diamine (Batch×2 total: 4.5 g, 28.38 mmol, 65.72% yield, N/A purity) was obtained as a yellow solid.

LC-MS (ES+, m/z): 159.3 [(M+H)+].

1H NMR (400 MHz, DMSO-d6) δ=7.23 (s, 1H), 6.41 (br s, 2H), 6.33 (br d, J=4.1 Hz, 1H), 2.81 (d, J=4.6 Hz, 3H).

### Step B:

To the solution of 5-chloro-N2-methyl-pyrazine-2,3-diamine (3 g, 18.92 mmol, 1 eq) in DMA (80 mL) was added NaHSO₃ (2.95 g, 28.38 mmol, 2.00 mL, 1.5 eq), and 2-methoxy-6-methyl-4-(trifluoromethyl)benzaldehyde (4.95 g, 22.70 mmol, 1.2 eq). The reaction mixture was stirred at 100° C. for 48 hr and then heated to 140° C., the mixture was stirred at 140° C. for 24 hr. LCMS indicated that the reaction was completed. The reaction mixture was poured onto 100 mL H₂O. Then the resulting solution was extracted with EA (120 mL×2). The combined organic layers were washed successively with water (100 mL×2) and brine (100 mL×1), dried over anhydrous Na₂SO₄, filtered, and concentrated to give a residue. The residue was purified by column (SiO₂, petroleum ether/ethyl acetate=10:1 to 1:1), and then was purified by prep-HPLC (column: Agela DuraShell C18 250 mm×70 mm×10 μm; mobile phase: [H₂O (10 mM NH₄HCO₃)-ACN]; gradient: 45%-65% B over 20.0 min) to give the title product. 5-Chloro-2-[2-methoxy-6-methyl-4-(trifluoromethyl)phenyl]-1-methyl-imidazo[4,5-b] pyrazine (2.7 g, 7.57 mmol, 40.01% yield) was obtained as a yellow solid.

LC-MS (ES+, m/z): 357.0 [(M+H)+].

1H NMR (400 MHz, DMSO-d6) δ=8.60 (s, 1H), 7.46 (s, 1H), 7.40 (s, 1H), 3.86 (s, 3H), 3.61 (s, 3H), 2.22 (s, 3H).

### Step C:

To the solution of 5-chloro-2-[2-methoxy-6-methyl-4-(trifluoromethyl)phenyl]-1-methyl-imidazo[4,5-b]pyrazine (200.00 mg, 560.65 μmol, 1 eq) in toluene (10 mL) was added XPhos (53.45 mg, 112.13 μmol, 0.2 eq), Pd₂(dba)₃ (77.01 mg, 84.10 μmol, 0.15 eq), Cs₂CO₃ (456.68 mg, 1.40 mmol, 2.5 eq), and tert-butyl (3R)-3-(methylamino)pyrrolidine-1-carboxylate (224.57 mg, 1.12 mmol, 2 eq) and the mixture was purged 3 times with N₂, and then the reaction mixture was stirred at 100° C. for 12 hr in a microwave device. LCMS indicated that the reaction was completed. After completion, the solvent was removed by concentration to give the residue. The residue was purified by column chromatography (SiO₂, petroleum ether/ethyl acetate=10/1 to 1/1). tert-Butyl (3R)-3-[[2-[2-methoxy-6-methyl-4-(trifluoromethyl)phenyl]-1-methyl-imidazo[4,5-b]pyrazin-5-yl]-methyl-amino]pyrrolidine-1-carboxylate (550 mg, 950.92 μmol, 33.92% yield, 90% purity) was obtained as a yellow solid.

LC-MS (ES⁺, m/z): 521.2 [(M+H)⁺].

¹H NMR (400 MHz, DMSO-d₆) δ=8.07 (s, 1H), 7.42 (s, 1H), 7.35 (s, 1H), 5.23-5.07 (m, 1H), 3.88-3.80 (m, 4H), 3.61-3.52 (m, 2H), 3.51-3.49 (m, 3H), 3.25-3.20 (m, 1H), 3.00 (s, 3H), 2.19 (s, 3H), 2.12-2.01 (m, 2H), 1.46-1.39 (m, 9H).

### Step D:

To the solution of tert-butyl (3R)-3-[[2-[2-methoxy-6-methyl-4-(trifluoromethyl)phenyl]-1-methyl-imidazo[4,5-b]pyrazin-5-yl]-methyl-amino]pyrolidine-1-carboxylate (500 mg, 960.53 μmol, 1 eq) in DCM (10 mL) was added BBr₃ (721.90 mg, 2.88 mmol, 277.66 μL, 3 eq) at 0° C. and the reaction mixture was stirred at 20° C. for 6 hr. LCMS indicated that the reaction was completed. The reaction mixture was poured onto 25 mL saturated Na₂CO₃ and then the pH was adjusted to 7. Then the resulting solution was extracted with DCM (20 mL×10). The combined organic layers were washed successively with water (5 mL×2) and brine (5 mL×1), dried over anhydrous Na₂SO₄, filtered, and concentrated to give a residue. The residue was used for the next step directly without further purification. 3-Methyl-2-[1-methyl-5-[methyl-[(3R)-pyrrolidin-3-yl]amino]imidazo[4,5-b]pyrazin-2-yl]-5-(trifluoromethyl)phenol (600 mg, crude) was obtained as a yellow solid.

LC-MS (ES⁺, m/z): 407.3 [(M+H)⁺].

### Step E:

To the solution of 3-methyl-2-[1-methyl-5-[methyl-[(3R)-pyrrolidin-3-yl]amino]imidazo[4,5-b]pyrazin-2-yl]-5-(trifluoromethyl)phenol (600 mg, 1.48 mmol, 1 eq) in MeOH (10 mL) was added 37% HCHO in H₂O (599.12 mg, 7.38 mmol, 549.66 μL, 37% purity, 5 eq) at 20° C. for 1 hr and then added NaBH₃CN (463.87 mg, 7.38 mmol, 5 eq) was added. The reaction mixture was stirred at 20° C. for 2 hr. LCMS indicated that the reaction was completed. The reaction mixture was poured onto 5 mL saturated Na₂CO₃. Then the resulting solution was extracted with DCM (5 mL×4). The combined organic layers were washed successively with water (5 mL×2) and brine (5 mL×1), dried over anhydrous Na₂SO₄, filtered, and concentrated to give a residue. The residue was purified by prep-HPLC (column: Phenomenex Luna C18 100 mm×30 mm×5 μm; mobile phase: [H₂O (0.2% FA)-ACN]; gradient: 5%-35% B over 8.0 min) to give the title product. 3-Methyl-2-[1-methyl-5-[methyl-[(3R)-1-methylpyrrolidin-3-yl]amino]imidazo[4,5-b]pyrazin-2-yl]-5-(trifluoromethyl)phenol (100 mg, 237.85 μmol, 24.7% yield for 2 steps, N/A purity) was obtained as a white solid.

LC-MS (ES⁺, m/z): 421.2 [(M+H)⁺].

¹H NMR (400 MHz, DMSO-d₆) δ=7.99 (s, 1H), 7.22 (s, 1H), 7.13 (s, 1H), 5.29-5.19 (m, 1H), 3.53 (s, 3H), 3.04 (s, 3H), 2.89-2.81 (m, 1H), 2.78-2.70 (m, 1H), 2.59 (br t, J=9.3 Hz, 2H), 2.32 (s, 3H), 2.24-2.19 (m, 1H), 2.17 (s, 3H), 1.84-1.72 (m, 1H).

### Example 61 3-methyl-2-(1-methyl-5-morpholino-1H-imidazo[4,5-b]pyrazin-2-yl)-5-(trifluoromethyl)phenol

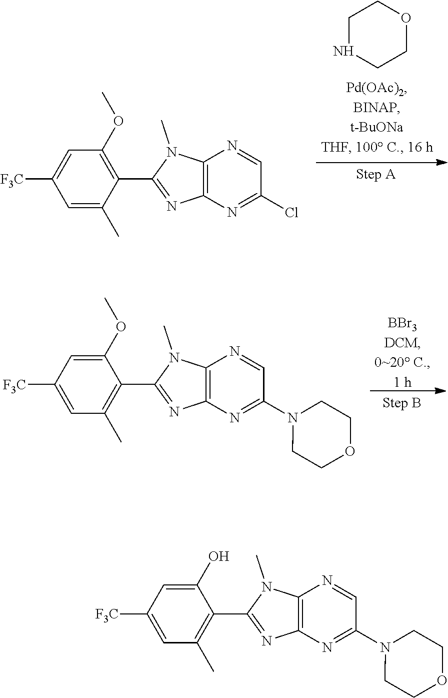

### Step A:

To the solution of 5-chloro-2-[2-methoxy-6-methyl-4-(trifluoromethyl)phenyl]-1-methyl-imidazo[4,5-b]pyrazine (150.00 mg, 420.49 μmol, 1 eq) in THF (15 mL) was added t-BuONa (80.82 mg, 840.97 μmol, 2 eq), Pd(OAc)₂ (9.44 mg, 42.05 μmol, 0.1 eq), BINAP (52.36 mg, 84.10 μmol, 0.2 eq), and morpholine (109.90 mg, 1.26 mmol, 111.01 μL, 3 eq) and the mixture was purged 3 times with N₂, then the reaction mixture was stirred at 100° C. for 16 hr in a sealed tube. LCMS indicated that the reaction was completed. The reaction mixture was poured onto 5 mL H₂O. Then the resulting solution was extracted with EA (5 mL×2). The combined organic layers were washed successively with water (5 mL×2) and brine (5 mL×1), dried over anhydrous Na₂SO₄, filtered, and concentrated to give a residue. The residue was purified by prep-TLC (SiO₂, DCM:MeOH=20:1). 4-[2-[2-Methoxy-6-methyl-4-(trifluoromethyl)phenyl]-1-methyl-imidazo[4,5-b]pyrazin-5-yl]morpholine (100 mg, 245.47 μmol, 58.38% yield, N/A purity) was obtained as a yellow solid.

LC-MS (ES⁺, m/z): 408.1 [(M+H)⁺].

¹H NMR (400 MHz, DMSO-d₆) δ=8.21 (s, 1H), 7.42 (s, 1H), 7.35 (s, 1H), 3.84 (s, 3H), 3.80-3.74 (m, 4H), 3.54-3.49 (m, 7H), 2.19 (s, 3H).

### Step B:

To the solution of 4-[2-[2-methoxy-6-methyl-4-(trifluoromethyl)phenyl]-1-methyl-imidazo[4,5-b]pyrazin-5-yl]morpholine (90 mg, 220.92 μmol, 1 eq) in DCM (1 mL) was added BBr₃ (166.04 mg, 662.76 μmol, 63.86 μL, 3 eq) at 0° C. and the reaction mixture was stirred at 20° C. for 1 hr. LCMS indicated that the reaction was completed. The reaction mixture was poured onto 5 mL H₂O. Then the resulting solution was extracted with EA (5 mL×2). The combined organic layers were washed successively with water (5 mL×2) and brine (5 mL×1), dried over anhydrous Na₂SO₄, filtered, and concentrated to give a residue. The residue was purified by prep-HPLC (column: Phenomenex luna C18 100 mm×40 mm×3 μm; mobile phase: [H₂O (0.2% FA)-ACN]; gradient: 15%-50% B over 8.0 min) to give the title product. 3-Methyl-2-(1-methyl-5-morpholino-imidazo[4,5-b]pyrazin-2-yl)-5-(trifluoromethyl)phenol (56.9 mg, 144.65 μmol, 65.48% yield, N/A purity) was obtained as a yellow solid.

LC-MS (ES⁺, m/z): 394.1 [(M+H)⁺].

¹H NMR (400 MHz, DMSO-d₆) δ=10.72 (br s, 1H), 8.20 (s, 1H), 7.23 (s, 1H), 7.14 (s, 1H), 3.81-3.74 (m, 4H), 3.56 (s, 3H), 3.54-3.47 (m, 4H), 2.17 (s, 3H).

### Example 62 5-chloro-2-(5-((4,4-difluoro-1-methylpyrrolidin-3-yl)amino)-1-methyl-1H-imidazo[4,5-b]pyridin-2-yl)-3-methylphenol HCl Salt

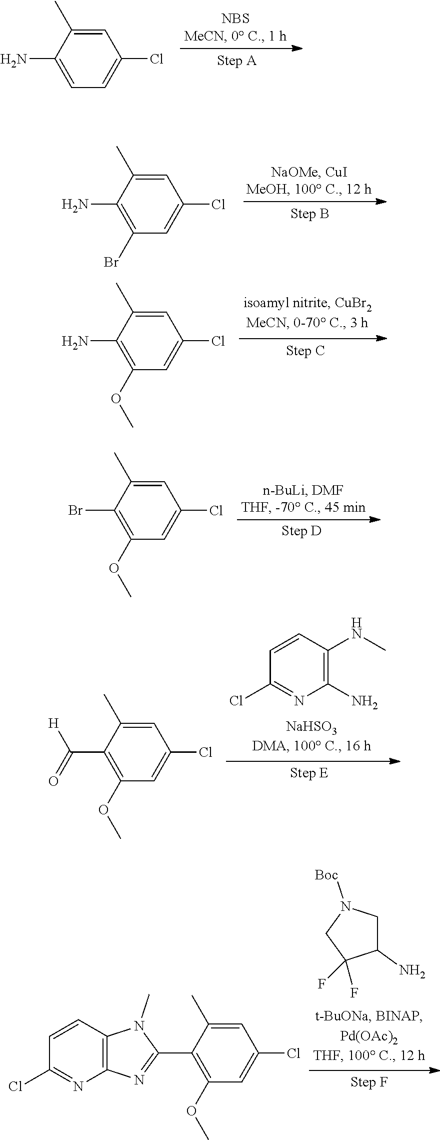

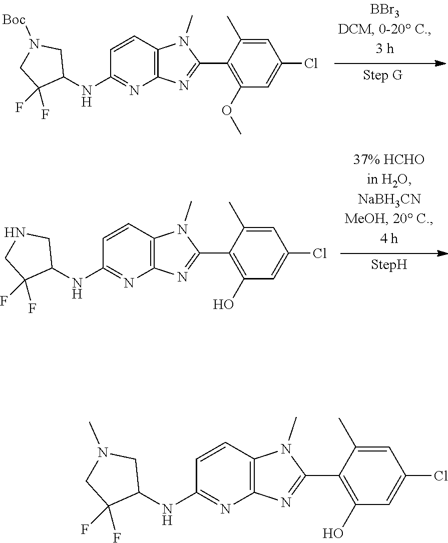 [SMILES: COc1cc(Cl)cc(C)c1-c1nc2nc(NC3CN(C(=O)OC(C)(C)C)CC3(F)F)ccc2n1C ; Cc1cc(Cl)cc(O)c1-c1nc2nc(NC3CNCC3(F)F)ccc2n1C ; Cc1cc(Cl)cc(O)c1-c1nc2nc(NC3CN(C)CC3(F)F)ccc2n1C]

### Step A:

A mixture of 4-chloro-2-methyl-aniline (35 g, 247.18 mmol, 1 eq) in MeCN (350 mL) and NBS (52.79 g, 296.61 mmol, 1.2 eq) at 0° C. was degassed and purged 3 times with N₂, and then the mixture was stirred at 0° C. for 1 hr under N₂ atmosphere. LCMS indicated that the reaction was completed. The reaction solution was concentrated and then poured onto 200 mL H₂O, followed by 200 mL EA. After that, the aqueous phase was separated and extracted with EA (200 mL×2). The combined organic layers were washed successively with brine (200 mL×3), dried over anhydrous Na₂SO₄, filtered, and concentrated to give a residue. The residue was purified by column Chromatography (SiO₂, petroleum ether/ethyl acetate=1/0 to 15/1). 2-Bromo-4-chloro-6-methyl-aniline (35 g, 158.73 mmol, 64.22% yield, N/A purity) was obtained as a brown solid.

LC-MS (ES⁺, m/z): 219.9 [(M+H)⁺].

### Step B:

To a mixture of 2-bromo-4-chloro-6-methyl-aniline (50 g, 226.76 mmol, 1 eq) in MeOH (480 mL) was added NaOMe (40.83 g, 226.76 mmol, 120 mL, 30% purity, 1 eq) and CuI (56.14 g, 294.79 mmol, 1.3 eq), the reaction mixture was degassed and purged 3 times with N₂, then the mixture was stirred at 100° C. for 12 hr under N₂ atmosphere. TLC indicated that the reaction was completed. The reaction mixture was poured onto 300 mL H₂O, followed by 300 mL EA. After that, the aqueous phase was separated and extracted with EA (300 mL×2). The combined organic layers were washed successively with brine (300 mL×3), dried over anhydrous Na₂SO₄, filtered, and concentrated to give a residue. The residue was purified by column chromatography (SiO₂, petroleum ether/ethyl acetate=1/0 to 10/1). 4-Chloro-2-methoxy-6-methyl-aniline (22 g, 128.19 mmol, 56.53% yield, N/A purity) was obtained as a brown solid.

¹H NMR (400 MHz, DMSO-d₆) δ=6.72 (d, J=2.3 Hz, 1H), 6.65 (d, J=2.0 Hz, 1H), 4.53 (s, 2H), 3.77 (s, 3H), 2.06 (s, 3H).

### Step C:

To a mixture of 4-chloro-2-methoxy-6-methyl-aniline (5 g, 29.13 mmol, 1 eq) in MeCN (125 mL) was added isoamyl nitrite (5.12 g, 43.70 mmol, 5.88 mL, 1.5 eq) and the mixture was stirred 5 min and then CuBr₂ (7.03 g, 31.46 mmol, 1.47 mL, 1.08 eq) was added at 0° C. and the mixture was degassed and purged 3 times with N₂, and then the mixture was stirred at 70° C. for 3 hr under N₂ atmosphere. TLC indicated that the reaction was completed. The reaction solution was filtered and then concentrated to give the residue. The residue was purified by column chromatography (SiO₂, petroleum ether/ethyl acetate=1/0). Compound 2-bromo-5-chloro-1-methoxy-3-methyl-benzene (2.9 g, 12.31 mmol, 42.27% yield, N/A purity) was obtained as a white solid.

¹H NMR (400 MHz, chloroform-d) δ=6.89 (d, J=2.1 Hz, 1H), 6.74 (d, J=2.3 Hz, 1H), 3.89 (s, 3H), 2.40 (s, 3H).

### Step D:

To the solution of 2-bromo-5-chloro-1-methoxy-3-methyl-benzene (3.5 g, 14.86 mmol, 1.0 eq) in THF (35 mL) was added n-BuLi (2.5 M, 11.89 mL, 2.0 eq) and the reaction mixture was stirred at −70° C. for 15 min, then morpholine-4-carbaldehyde (5.18 g, 44.59 mmol, 4.50 mL, 3.0 eq) was added. The resulting reaction mixture was stirred at −70° C. for 30 min. TLC (PE/EA=10/1, product Rf=0.50, Color Developing Reagent: UV 254 nm) indicated that the reaction was completed. The reaction mixture was poured onto 50 mL H₂O at 0° C., followed by 10 mL DCM. After that, the aqueous phase was separated and extracted with DCM (30 mL×3). The combined organic layers were washed successively with water (20 mL×2) and brine (20 mL×1), dried over anhydrous Na₂SO₄, filtered, and concentrated to give a residue. The residue was purified by column chromatography (SiO₂, petroleum ether/ethyl acetate=100/0 to 100/1). 4-Chloro-2-methoxy-6-methyl-benzaldehyde (1.2 g, 5.20 mmol, 34.99% yield, 80% purity) was obtained as a white solid.

LC-MS (ES⁺, m/z): 185.1 [(M+H)⁺].

### Step E:

To a mixture of 4-chloro-2-methoxy-6-methyl-benzaldehyde (280.00 mg, 1.52 mmol, 1 eq) in DMA (10 mL) was added 6-chloro-N3-methyl-pyridine-2,3-diamine (215.12 mg, 1.36 mmol, 0.9 eq) and NaHSO₃ (189.39 mg, 1.82 mmol, 127.96 μL, 1.2 eq), and then the mixture was stirred at 100° C. for 16 hr under N₂ atmosphere. LCMS indicated that the reaction was completed. The reaction mixture was poured onto 20 mL H₂O, followed by 20 mL EA. After that, the aqueous phase was separated and extracted with EA (20 mL×2). The combined organic layers were washed successively with brine (20 mL×3), dried over anhydrous Na₂SO₄, filtered, and concentrated to give a residue. The residue was purified by prep-TLC (Petroleum ether:Ethyl acetate=1:1) to give the title product. Compound 5-chloro-2-(4-chloro-2-methoxy-6-methyl-phenyl)-1-methyl-imidazo[4,5-b]pyridine (260 mg, 806.98 μmol, 53.21% yield, N/A purity) was obtained as a white solid.

LC-MS (ES⁺, m/z): 322.1 [(M+H)⁺].

¹H NMR (400 MHz, DMSO-d₆) δ=8.17 (d, J=8.4 Hz, 1H), 7.40 (d, J=8.4 Hz, 1H), 7.16 (dd, J=1.4, 13.2 Hz, 2H), 3.77 (s, 3H), 3.57 (s, 3H), 2.07 (s, 3H).

### Step F:

to a mixture of 5-chloro-2-(4-chloro-2-methoxy-6-methyl-phenyl)-1-methyl-imidazo[4,5-b]pyridine (200 mg, 620.75 μmol, 1 eq) in THF (8 mL) was added tert-butyl 4-amino-3,3-difluoro-pyrrolidine-1-carboxylate (151.75 mg, 682.83 μmol, 1.1 eq), BINAP (77.31 mg, 124.15 μmol, 0.2 eq), Pd(OAc)₂ (13.94 mg, 62.08 μmol, 0.1 eq) and NaOtBu (119.31 mg, 1.24 mmol, 2 eq) and the mixture was purged 3 times with N₂. Then the mixture was stirred at 100° C. for 12 hr. LCMS indicated that the reaction was completed. The reaction mixture was poured onto 20 mL H₂O, followed by 20 mL EA. After that, the aqueous phase was separated and extracted with EA (20 mL×2). The combined organic layers were washed successively with brine (20 mL×3), dried over anhydrous Na₂SO₄, filtered, and concentrated to give a residue. The residue was purified by prep-HPLC (column: Waters Xbridge BEH C18 100 mm×30 mm×100 μm; mobile phase: [A: H₂O (10 mM NH₄HCO₃); B: ACN]; B %: 35.00%-65.00%, 8.00 min) to get the product. tert-Butyl 4-[[2-(4-chloro-2-methoxy-6-methyl-phenyl)-1-methyl-imidazo[4,5-b]pyridin-5-yl]amino]-3,3-difluoro-pyrrolidine-1-carboxylate (70 mg, 137.81 μmol, 22.2% yield) was obtained as white solid.

LC-MS (ES⁺, m/z): 508.1 [(M+H)⁺].

1H NMR (DMSO-d6, 400 MHz): δ (ppm) 7.71-7.81 (m, 1H), 7.06-7.16 (m, 2H), 6.82-6.93 (m, 1H), 6.55-6.67 (m, 1H), 4.97-5.25 (m, 1H), 3.83-3.94 (m, 1H), 3.70-3.81 (m, 5H), 3.41-3.47 (m, 3H), 3.10-3.23 (m, 1H), 2.00-2.14 (m, 3H), 1.40-1.46 (m, 9H)

### Step G:

To the solution of tert-butyl 4-[[2-(4-chloro-2-methoxy-6-methyl-phenyl)-1-methyl-imidazo[4,5-b]pyridin-5-yl]amino]-3,3-difluoro-pyrrolidine-1-carboxylate (50 mg, 98.43 μmol, 1 eq) in DCM (3 mL) was added BBr₃ (123.30 mg, 492.17 μmol, 47.42 μL, 5 eq) at 0° C. and the reaction mixture was stirred at 20° C. for 3 hr. LCMS indicated that the reaction was completed. The reaction mixture was poured onto 10 mL saturated aqueous Na₂CO₃. Then the resulting solution was extracted with DCM (10 mL×4). The combined organic layers were washed successively with water (10 mL×2) and brine (10 mL×1), dried over anhydrous Na₂SO₄, filtered, and concentrated to give a residue. The residue was used for the next step directly without further purification. 5-Chloro-2-[5-[(4,4-difluoropyrrolidin-3-yl)amino]-1-methyl-imidazo[4,5-b]pyridin-2-yl]-3-methyl-phenol (40 mg, crude) was obtained as a white solid. LC-MS (ES⁺, m/z): 394.2 [(M+H)⁺].

### Step H:

To the solution of 5-chloro-2-[5-[(4,4-difluoropyrrolidin-3-yl)amino]-1-methyl-imidazo[4,5-b]pyridin-2-yl]-3-methyl-phenol (35 mg, 88.87 μmol, 1 eq) in MeOH (3 mL) was added the solution of HCHO in H₂O (36.06 mg, 444.37 μmol, 33.08 μL, 37% purity, 5 eq) at 20° C. for 1 hr and NaBH₃CN was added (27.92 mg, 444.37 μmol, 5 eq) and the reaction mixture was stirred at 20° C. for 3 hr. LCMS indicated that the reaction was completed. After completion, the solvent was removed by concentration to give the residue. The residue was purified by prep-HPLC (column: Phenomenex luna C18 100 mm×40 mm×5 μm; mobile phase: [H₂O (0.04% HCl)-ACN]; gradient: 5%-30% B over 8.0 min) to give the title product. 5-Chloro-2-[5-[(4,4-difluoro-1-methyl-pyrrolidin-3-yl)amino]-1-methyl-imidazo[4,5-b]pyridin-2-yl]-3-methyl-phenol (4.85 mg, 11.89 μmol, 12.1% yield for 2 steps, N/A purity) was obtained as a white solid.

LC-MS (ES⁺, m/z): 408.1 [(M+H)⁺].

¹H NMR (400 MHz, METHANOL-d₄) δ=8.12 (d, J=9.1 Hz, 1H), 7.07-7.00 (m, 2H), 6.95 (s, 1H), 5.33 (br d, J=10.1 Hz, 1H), 4.22-4.06 (m, 2H), 4.05-3.93 (m, 1H), 3.84 (s, 3H), 3.58 (br s, 1H), 3.09 (s, 3H), 2.22 (d, J=2.9 Hz, 3H).

¹H NMR (400 MHz, DMSO-d₆) δ=11.36-11.14 (m, 1H), 8.15 (br d, J=8.6 Hz, 1H), 8.04-7.86 (m, 1H), 7.04 (br d, J=3.8 Hz, 2H), 6.96 (br d, J=7.9 Hz, 1H), 5.35-5.07 (m, 1H), 4.06-3.81 (m, 3H), 3.72 (s, 3H), 3.54-3.50 (m, 1H), 2.93-2.86 (m, 3H), 2.16 (d, J=9.8 Hz, 3H).

### Example 63: 1-(2-(2-hydroxy-6-methyl-4-(trifluoromethyl)phenyl)-1-methyl-1H-imidazo[4,5-b]pyrazin-5-yl)piperidin-4-ol

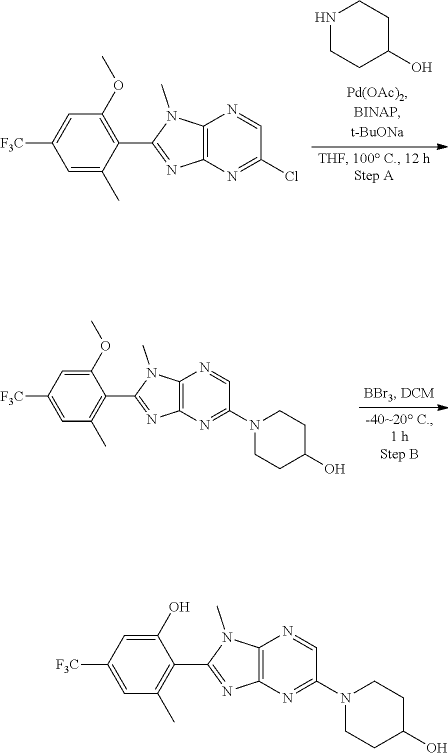 [SMILES: COc1cc(C(F)(F)F)cc(C)c1-c1nc2nc(Cl)cnc2n1C ; OC1CCNCC1 ; COc1cc(C(F)(F)F)cc(C)c1-c1nc2nc(N3CCC(O)CC3)cnc2n1C ; Cc1cc(C(F)(F)F)cc(O)c1-c1nc2nc(N3CCC(O)CC3)cnc2n1C]

### Step A:

To a solution of 5-chloro-2-[2-methoxy-6-methyl-4-(trifluoromethyl)phenyl]-1-methyl-imidazo[4,5-b]pyrazine (200 mg, 560.65 μmol, 1 eq) and piperidin-4-ol (113.42 mg, 1.12 mmol, 2 eq) in THF (3 mL) was added Pd(OAc)₂ (12.59 mg, 56.06 μmol, 0.1 eq) and BINAP (69.82 mg, 112.13 μmol, 0.2 eq) and tBuONa (107.76 mg, 1.12 mmol, 2 eq). The mixture was stirred at 100° C. for 12 h. The reaction mixture was quenched by addition of H₂O 20 mL, and then extracted with EtOAc (10 mL×3). The combined organic layers were washed with brine 20 mL, dried over Na₂SO₄, filtered and concentrated under reduced pressure to give a residue. The residue was purified by prep-TLC (SiO₂, PE:EtOAc=0:1) to give 1-[2-[2-methoxy-6-methyl-4-(trifluoromethyl)phenyl]-1-methyl-imidazo[4,5-b]pyrazin-5-yl]piperidin-4-ol (90 mg, 38%) as a yellow solid.

¹H NMR (400 MHz, DMSO-d₆) δ=8.20 (s, 1H), 7.42 (s, 1H), 7.34 (s, 1H), 4.72 (d, 1H), 4.03 (br d, 2H), 3.84 (s, 3H), 3.77-3.68 (m, 1H), 3.49 (s, 3H), 3.18 (br s, 2H), 2.21-2.17 (m, 3H), 1.89-1.76 (m, 2H), 1.52-1.36 (m, 2H).

MS: 422.1 [(M+H)⁺].

### Step B:

To a solution of 1-[2-[2-methoxy-6-methyl-4-(trifluoromethyl)phenyl]-1-methyl-imidazo[4,5-b]pyrazin-5-yl]piperidin-4-ol (80 mg, 189.84 μmol, 1 eq) in DCM (6 mL) was added BBr₃ (237.79 mg, 949.18 μmol, 91.46 μL, 5 eq) at −40° C. The mixture was stirred at 25° C. for 1 h. The reaction mixture was poured onto 10 mL H₂O and then the pH was adjusted to 7 with saturated NaHCO₃. Then, the resulting solution was extracted with EtOAc (5 mL×2). The combined organic layers were washed successively with water (5 mL×2) and brine (20 ml×1), dried over anhydrous Na₂SO₄, filtered, and concentrated to give a residue. The residue was purified by prep-HPLC column: Waters Xbridge C18 150 mm×50 mm×10 μm; mobile phase: [H₂O (10 mM NH₄HCO₃)-ACN]; gradient: 20%-50% B over 6.0 min. Compound 1-[2-[2-hydroxy-6-methyl-4-(trifluoromethyl)phenyl]-1-methyl-imidazo[4,5-b]pyrazin-5-yl]piperidin-4-ol (20.63 mg, 26%) was obtained as a yellow solid.

¹H NMR (400 MHz, DMSO-d₆) δ=10.68 (br s, 1H), 8.19 (s, 1H), 7.22 (s, 1H), 7.13 (s, 1H), 4.71 (d, 1H), 4.08-4.00 (m, 2H), 3.77-3.69 (m, 1H), 3.54 (s, 3H), 3.18 (ddd, 2H), 2.18 (s, 3H), 1.87-1.80 (m, 2H), 1.49-1.39 (m, 2H).

MS: 408.1 [(M+H)⁺].

### Example 64 5-chloro-2-(6-fluoro-1-methyl-5-morpholino-1H-imidazo[4,5-b]pyridin-2-yl)-3-methylphenol

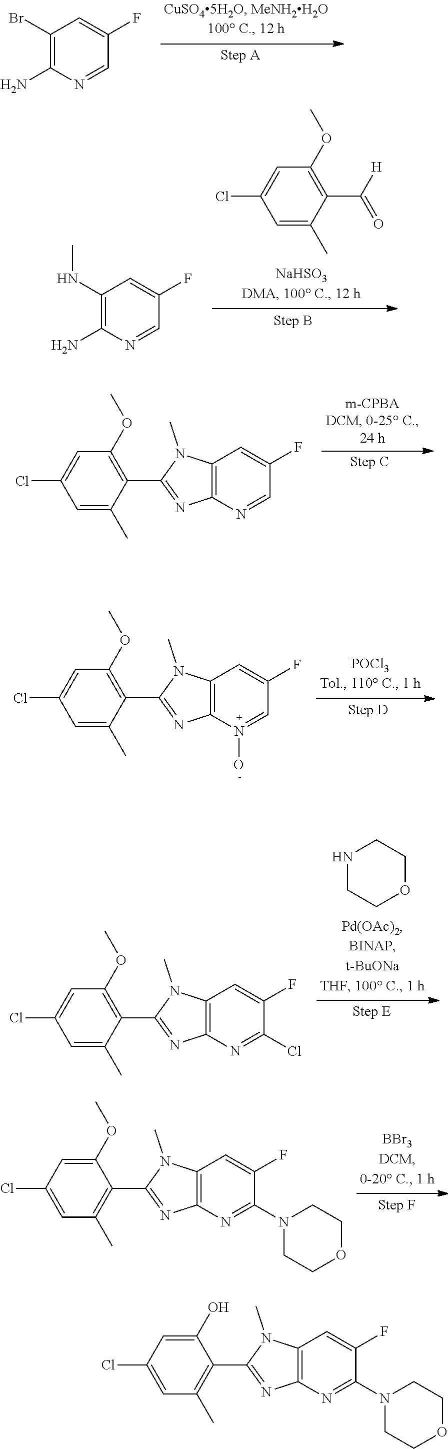

### Step A:

To the solution of 3-bromo-5-fluoropyridin-2-amine (3 g, 15.71 mmol, 1 eq) in MeNH₂ in H₂O (3.00 g, 28.98 mmol, 30% purity) was added CuSO₄·5H₂O (392.17 mg, 1.57 mmol, 0.1 eq) and the resulting reaction mixture was stirred at 100° C. for 12 hr. LCMS indicated that the reaction was completed. The reaction mixture was poured onto 100 mL H₂O, followed by 60 mL EA. After that, the aqueous phase was separated and extracted with EA (60 mL×2). The combined organic layers were washed successively with water (50 mL×2) and brine (50 mL×1), dried over anhydrous Na₂SO₄, filtered, and concentrated to give a residue. The residue was purified by column chromatography (SiO₂, petroleum ether/ethyl acetate=100/1 to 0/1). 5-Fluoro-N3-methyl-pyridine-2,3-diamine (2.9 g, 20.55 mmol, 65.41% yield, N/A purity) was obtained as a gray solid.

LC-MS (ES⁺, m/z): 142.1 [(M+H)⁺].

¹H NMR (400 MHz, DMSO-d₆) δ=7.15 (d, J=2.6 Hz, 1H), 6.42 (dd, J=2.5, 11.0 Hz, 1H), 5.33 (s, 2H), 5.26 (br d, J=3.1 Hz, 1H), 2.69 (d, J=4.9 Hz, 3H)

### Step B:

To the solution of 5-fluoro-N3-methyl-pyridine-2,3-diamine (2.5 g, 17.71 mmol, 1.0 eq) in DMA (25 mL) was added 4-chloro-2-methoxy-6-methyl-benzaldehyde (3.2 g, 17.33 mmol, 9.79e-1.0 eq), and NaHSO₃ (3.69 g, 35.42 mmol, 2.49 mL, 2.0 eq) and the reaction mixture was stirred at 100° C. for 12 hr. LCMS indicated that the reaction was completed. The reaction mixture was poured onto 60 mL H₂O, followed by 30 mL EA. After that, the aqueous phase was separated and extracted with EA (30 mL×2). The combined organic layers were washed successively with water (20 mL×2) and brine (20 mL×1), dried over anhydrous Na₂SO₄, filtered, and concentrated to give a residue. The residue was purified by column chromatography (SiO₂, petroleum ether/ethyl acetate=100/1 to 0/1). 2-(4-Chloro-2-methoxy-6-methyl-phenyl)-6-fluoro-1-methyl-imidazo[4,5-b]pyridine (3.4 g, 11.12 mmol, 62.79% yield, N/A purity) was obtained as a yellow solid.

LC-MS (ES⁺, m/z): 306.0 [(M+H)⁺].

1H NMR (400 MHz, DMSO-d6) δ=8.45-8.40 (m, 1H), 8.12 (dd, J=2.8, 8.9 Hz, 1H), 7.15 (dd, J=1.3, 13.6 Hz, 2H), 3.77 (s, 3H), 3.54 (s, 3H), 2.08 (s, 3H)

### Step C:

To the solution of 2-(4-chloro-2-methoxy-6-methyl-phenyl)-6-fluoro-1-methyl-imidazo[4,5-b]pyridine (3.4 g, 11.12 mmol, 1.0 eq) in DCM (34 mL) was added m-CPBA (4.52 g, 22.24 mmol, 85% purity, 2.0 eq) at 0° C. and the resulting reaction mixture was stirred at 25° C. for 24 hr. LCMS indicated that the reaction was completed. The reaction mixture was poured onto 80 mL H₂O, followed by 30 mL DCM. After that, the aqueous phase was separated and extracted with DCM (30 mL×2). The combined organic layers were washed successively with water (20 mL×2) and brine (20 mL×1), dried over anhydrous Na₂SO₄, filtered, and concentrated to give a residue. The residue was purified by column chromatography (SiO₂, DCM/MeOH=100/1 to 1/1). 2-(4-Chloro-2-methoxy-6-methyl-phenyl)-6-fluoro-1-methyl-4-oxido-imidazo [4,5-b]pyridin-4-ium (3.4 g, 10.57 mmol, 95.03% yield, N/A purity) was obtained as a yellow solid.

LC-MS (ES⁺, m/z): 322.0 [(M+H)⁺].

¹H NMR (400 MHz, DMSO-d₆) δ=8.52 (dd, J=2.0, 5.9 Hz, 1H), 7.87 (dd, J=2.0, 7.8 Hz, 1H), 7.21-7.11 (m, 2H), 3.78 (s, 3H), 3.54 (s, 3H), 2.09 (s, 3H)

### Step D:

To the solution of 2-(4-chloro-2-methoxy-6-methyl-phenyl)-6-fluoro-1-methyl-4-oxido-imidazo[4,5-b]pyridin-4-ium (3.4 g, 10.57 mmol, 1.0 eq) in toluene (34 mL) was added POCl₃ (3.24 g, 21.14 mmol, 1.97 mL, 2.0 eq) and the resulting reaction mixture was stirred at 110° C. for 1 hr. LCMS indicated that the reaction was completed. The reaction mixture was poured onto 50 mL H₂O, followed by 25 mL EA. After that, the aqueous phase was separated and extracted with EA (25 mL×2). The combined organic layers were washed successively with water (20 mL×2) and brine (20 mL×1), dried over anhydrous Na₂SO₄, filtered, and concentrated to give a residue. The residue was purified by column chromatography (SiO₂, petroleum ether/ethyl acetate=100/1 to 0/1). 5-Chloro-2-(4-chloro-2-methoxy-6-methyl-phenyl)-6-fluoro-1-methyl-imidazo[4,5-b] pyridine (60 mg, 176.38 μmol, 1.67% yield, N/A purity) was obtained as a white solid. 5-Chloro-2-(4-chloro-2-methoxy-6-methyl-phenyl)-6-fluoro-1-methyl-imidazo[4,5-b] pyridine (1.1 g, 970.08 μmol, 9.18% yield, 30% purity) was obtained as a white solid.

LC-MS (ES⁺, m/z): 339.9 [(M+H)⁺].

¹H NMR (400 MHz, DMSO-d₆) δ=8.41 (d, J=8.7 Hz, 1H), 7.16 (dd, J=1.5, 14.4 Hz, 2H), 3.77 (s, 3H), 3.56 (s, 3H), 2.07 (s, 3H)

### Step E:

To the solution of 5-chloro-2-(4-chloro-2-methoxy-6-methyl-phenyl)-6-fluoro-1-methyl-imidazo[4,5-b]pyridine (550 mg, 485.04 μmol, 1.0 eq, 30% purity) in THF (5 mL) was added morpholine (46.48 mg, 533.54 μmol, 46.95 μL, 1.1 eq), BINAP (60.40 mg, 97.01 μmol, 0.2 eq), t-BuONa (93.22 mg, 970.08 μmol, 2.0 eq), and Pd(OAc)₂ (10.89 mg, 48.50 μmol, 0.1 eq) and the resulting reaction mixture was stirred at 100° C. for 1 hr. LCMS indicated that the reaction was completed. The reaction mixture was poured onto 50 mL H₂O, followed by 25 mL EA. After that, the aqueous phase was separated and extracted with EA (25 mL×2). The combined organic layers were washed successively with water (20 mL×2) and brine (20 mL×1), dried over anhydrous Na₂SO₄, filtered, and concentrated to give a residue. The residue was purified by column chromatography (SiO₂, petroleum ether/ethyl acetate=100/1 to 0/1). 4-[2-(4-Chloro-2-methoxy-6-methyl-phenyl)-6-fluoro-1-methyl-imidazo[4,5-b]pyridin-5-yl]morpholine (Batch 2 total: 380 mg, 486.13 μmol, 50.11% yield, 50% purity) was obtained as a yellow oil.

LC-MS (ES+, m/z): 391.1 [(M+H)⁺].

¹H NMR (400 MHz, DMSO-d₆) δ=8.03 (d, J=12.3 Hz, 1H), 7.20-7.08 (m, 2H), 3.77-3.76 (m, 4H), 3.75 (s, 3H), 3.47 (s, 3H), 3.30-3.24 (m, 4H), 2.07 (s, 3H)

### Step F:

To the solution of 4-[2-(4-chloro-2-methoxy-6-methyl-phenyl)-6-fluoro-1-methyl-imidazo[4,5-b]pyridin-5-yl]morpholine (380 mg, 486.13 μmol, 1.0 eq) in DCM (3.8 mL), BBr₃ (608.93 mg, 2.43 mmol, 234.20 μL, 5.0 eq) was added at 0° C. and the resulting reaction mixture was stirred at 20° C. for 1 hr. LCMS indicated that the reaction was completed. After completion, the solvent was removed by concentration to give the crude product. The residue was purified by prep-HPLC (Waters Xbridge C18 150 mm×50 mm×10 μm; mobile phase: [H₂O (10 mM NH₄HCO₃)-ACN]; gradient: 35%-55% B over 6.0 min) to give the title product. 5-chloro-2-(6-fluoro-1-methyl-5-morpholino-imidazo[4,5-b]pyridin-2-yl)-3-methyl-phenol (55 mg, 140.66 μmol, 28.94% yield, 96.37% purity) was obtained as a white solid.

LC-MS (ES+, m/z): 377.1 [(M+H)⁺].

¹H NMR (400 MHz, DMSO-d₆) δ=10.35 (br s, 1H), 8.02 (d, J=12.4 Hz, 1H), 6.94 (d, J=1.4 Hz, 1H), 6.87 (d, J=1.8 Hz, 1H), 3.81-3.74 (m, 4H), 3.52 (s, 3H), 3.29-3.25 (m, 4H), 2.05 (s, 3H)

### Example 65 2-(6-(hydroxymethyl)-5-methoxy-1-methyl-1H-imidazo[4,5-b]pyrazin-2-yl)-3-methyl-5-(trifluoromethyl)phenol

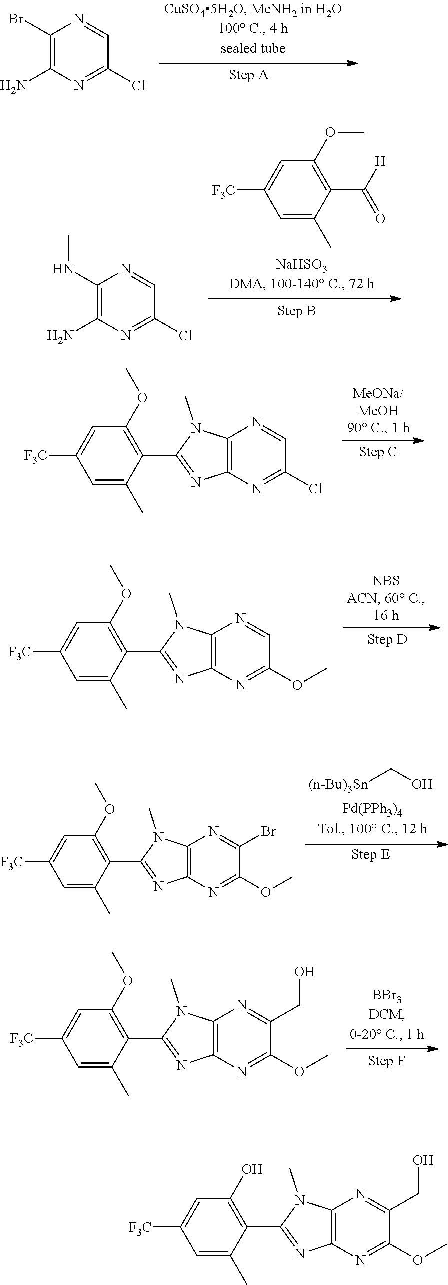

### Step A:

To the solution of 3-bromo-6-chloro-pyrazin-2-amine (3 g, 14.39 mmol, 1 eq) in MeNH₂ in H₂O (6.21 g, 60.00 mmol, 20 mL, 4.17 eq) was added CuSO₄·5H₂O (359.36 mg, 1.44 mmol, 0.1 eq) and the reaction mixture was stirred at 100° C. for 4 hr in a sealed tube under N₂ atmosphere. LCMS indicated that the reaction was completed. The reaction mixture was cooled to room temperature and filtered through Celite. The celite pad was rinsed with DCM (2×200 mL) and concentrated in vacuo to get the residue. The residue was purified by column chromatography (SiO₂, petroleum ether/ethyl acetate=10/1 to 1/1). 5-Chloro-N2-methyl-pyrazine-2,3-diamine (Batch×2 total: 4.5 g, 28.38 mmol, 65.72% yield, N/A purity) was obtained as a yellow solid.

LC-MS (ES⁺, m/z): 159.3 [(M+H)⁺].

¹H NMR (400 MHz, DMSO-d₆) δ=7.23 (s, 1H), 6.41 (br s, 2H), 6.33 (br d, J=4.1 Hz, 1H), 2.81 (d, J=4.6 Hz, 3H).

### Step B:

To the solution of 5-chloro-N2-methyl-pyrazine-2,3-diamine (3 g, 18.92 mmol, 1 eq) in DMA (80 mL) was added NaHSO₃ (2.95 g, 28.38 mmol, 2.00 mL, 1.5 eq) and 2-methoxy-6-methyl-4-(trifluoromethyl)benzaldehyde (4.95 g, 22.70 mmol, 1.2 eq) and the reaction mixture was stirred at 100° C. for 48 hr. Then the mixture was stirred at 140° C. for 24 hr. LCMS indicated that the reaction was completed. The reaction mixture was poured onto 100 mL H₂O. Then the resulting solution was extracted with EA (120 mL×2). The combined organic layers were washed successively with water (100 mL×2) and brine (100 mL×1), dried over anhydrous Na₂SO₄, filtered, and concentrated to give a residue. The residue was purified by column (SiO₂, petroleum ether/ethyl acetate=10:1-1:1) then the residue was purified by prep-HPLC (column: Agela DuraShell C18 250 mm×70 mm×10 μm; mobile phase: [H₂O (10 mM NH₄HCO₃)-ACN]; gradient: 45%-65% B over 20.0 min) to give the title product. 5-Chloro-2-[2-methoxy-6-methyl-4-(trifluoromethyl)phenyl]-1-methyl-imidazo[4,5-b]pyrazine (2.7 g, 7.57 mmol, 40.01% yield) was obtained as a yellow solid.

LC-MS (ES⁺, m/z): 357.0 [(M+H)⁺].

¹H NMR (400 MHz, DMSO-d₆) δ=8.60 (s, 1H), 7.46 (s, 1H), 7.40 (s, 1H), 3.86 (s, 3H), 3.61 (s, 3H), 2.22 (s, 3H).

### Step C:

To 5-chloro-2-[2-methoxy-6-methyl-4-(trifluoromethyl)phenyl]-1-methyl-imidazo[4,5-b]pyrazine (500 mg, 1.40 mmol, 1 eq) was added a 30% sodium methoxide solution in MeOH (204.64 mg, 1.40 mmol, 10 mL, 30%) and the reaction mixture was stirred at 90° C. for 1 h. LCMS indicated that the reaction was completed. The reaction mixture was poured onto 5 mL H₂O. Then the resulting solution was extracted with DCM (10 mL×2). The combined organic layers were washed successively with water (5 mL×2) and brine (5 mL×1), dried over anhydrous Na₂SO₄, filtered, and concentrated to give a residue. The residue was purified by prep-TLC (SiO₂, PE:EA=3:1). 5-Methoxy-2-[2-methoxy-6-methyl-4-(trifluoromethyl)phenyl]-1-methyl-imidazo[4,5-b]pyrazine (400 mg, 1.14 mmol, 81.00% yield, N/A purity) was obtained as a white solid.

LC-MS (ES⁺, m/z): 353.2 [(M+H)⁺].

¹H NMR (400 MHz, DMSO-d₆) δ=8.13 (s, 1H), 7.44 (s, 1H), 7.37 (s, 1H), 3.97 (s, 3H), 3.85 (s, 3H), 3.57 (s, 3H), 2.20 (s, 3H).

### Step D:

To the solution of 5-methoxy-2-[2-methoxy-6-methyl-4-(trifluoromethyl)phenyl]-1-methyl-imidazo[4,5-b]pyrazine (400 mg, 1.14 mmol, 1 eq) in ACN (10 mL) was added NBS (404.14 mg, 2.27 mmol, 2 eq) and the reaction mixture was stirred at 60° C. for 16 hr. LCMS indicated that the reaction was completed. After completion, the solvent was removed by concentration to give the residue. The residue was purified by prep-TLC (SiO₂, petroleum ether/ethyl acetate=1:1). 5-Bromo-6-methoxy-2-[2-methoxy-6-methyl-4-(trifluoromethyl)phenyl]-3-methyl-imidazo[4,5-b]pyrazine (450 mg, 1.04 mmol, 91.92% yield, N/A purity) was obtained as a white solid.

LC-MS (ES⁺, m/z): 431.2 [(M+H)⁺].

¹H NMR (400 MHz, DMSO-d₆) δ=7.44 (s, 1H), 7.37 (s, 1H), 4.04 (s, 3H), 3.85 (s, 3H), 3.55 (s, 3H), 2.20 (s, 3H).

### Step E:

To the solution of 5-bromo-6-methoxy-2-[2-methoxy-6-methyl-4-(trifluoromethyl)phenyl]-3-methyl-imidazo[4,5-b]pyrazine (200 mg, 463.81 μmol, 1 eq) in toluene (10 mL) was added Pd(PPh₃)₄ (53.60 mg, 46.38 μmol, 0.1 eq) and tributylstannylmethanol (595.70 mg, 1.86 mmol, 4 eq), then the reaction mixture was stirred at 100° C. for 12 hr after purging 3 times with N₂. LCMS indicated that the reaction was completed. After completion, the solvent was removed by concentration to give the residue. The residue was purified by prep-TLC (SiO₂, petroleum ether/ethyl acetate=1:1). [5-Methoxy-2-[2-methoxy-6-methyl-4-(trifluoromethyl)phenyl]-1-methyl-imidazo[4,5-b]pyrazin-6-yl]methanol (110 mg, 287.70 μmol, 62.03% yield, N/A purity) was obtained as a white solid.

LC-MS (ES⁺, m/z): 383.3 [(M+H)⁺].

¹H NMR (400 MHz, DMSO-d₆) δ=7.44 (s, 1H), 7.36 (s, 1H), 5.18 (t, J=5.9 Hz, 1H), 4.67 (d, J=6.0 Hz, 2H), 3.99 (s, 3H), 3.84 (s, 3H), 3.57 (s, 3H), 2.20 (s, 3H).

### Step F:

To the solution of [5-methoxy-2-[2-methoxy-6-methyl-4-(trifluoromethyl)phenyl]-1-methyl-imidazo[4,5-b]pyrazin-6-yl]methanol (100 mg, 261.55 μmol, 1 eq) in DCM (3 mL) was added BBr₃ (196.57 mg, 784.65 μmol, 75.60 μL, 3 eq) at 0° C. and the reaction mixture was stirred at 20° C. for 1 hr. LCMS indicated that the reaction was completed. After completion, the solvent was removed by concentration to give the residue. The residue was purified by prep-HPLC (column: Phenomenex luna C18 100 mm×40 mm×3 μm; mobile phase: [H₂O (0.2% FA)-ACN]; gradient: 25%-60% B over 8.0 min) to give the title product. 2-[6-(Hydroxymethyl)-5-methoxy-1-methyl-imidazo[4,5-b]pyrazin-2-yl]-3-methyl-5-(trifluoromethyl)phenol (42.05 mg, 114.17 μmol, 43.65% yield, N/A purity) was obtained as a white solid.

LC-MS (ES⁺, m/z): 369.2 [(M+H)⁺].

¹H NMR (400 MHz, DMSO-d₆) δ=10.72 (s, 1H), 7.24 (s, 1H), 7.14 (s, 1H), 5.17 (br s, 1H), 4.67 (br s, 2H), 4.00 (s, 3H), 3.61 (s, 3H), 2.18 (s, 3H).

### Example 66: 5-chloro-3-methyl-2-(1-methyl-5-morpholino-imidazo[4,5-b]pyrazin-2-yl)phenol

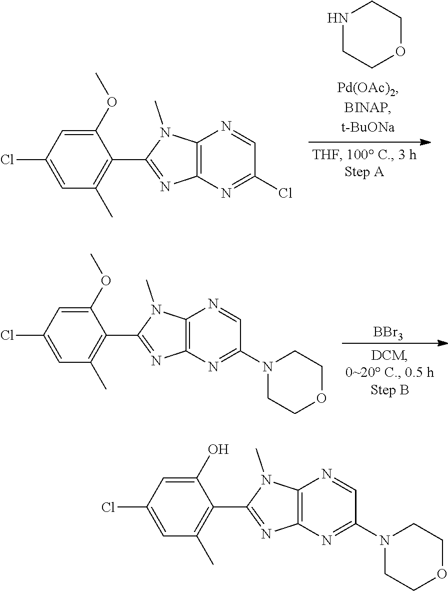

### Step A:

To a solution of 5-chloro-2-(4-chloro-2-methoxy-6-methyl-phenyl)-1-methyl-imidazo[4,5-b]pyrazine (200 mg, 618.86 μmol, 1 eq) and morpholine (64.70 mg, 742.63 μmol, 65.35 μL, 1.2 eq) in THF (3 mL) was added Pd(OAc)₂ (13.89 mg, 61.89 μmol, 0.1 eq) and BINAP (77.07 mg, 123.77 μmol, 0.2 eq) and tBuONa (118.95 mg, 1.24 mmol, 2 eq). The mixture was stirred at 100° C. for 3 hr. The reaction mixture was quenched by addition of H₂O 20 mL, and then extracted with EtOAc (10 mL×3). The combined organic layers were washed with brine 20 mL, dried over Na₂SO₄, filtered and concentrated under reduced pressure to give a residue. The residue was purified by prep-TLC (SiO₂, PE:EtOAc=1:1) to get compound 4-[2-(4-chloro-2-methoxy-6-methyl-phenyl)-1-methyl-imidazo[4,5-b]pyrazin-5-yl]morpholine (100 mg, 43%) as a yellow solid.

LC-MS (ES⁺, m/z): 374.4 [(M+H)⁺].

1H NMR (400 MHz, CDCl3) δ=7.96 (s, 1H), 6.99 (d, 1H), 6.87 (d, 1H), 3.89 (t, 4H), 3.76 (s, 3H), 3.65-3.61 (m, 4H), 3.59 (s, 3H), 2.20 (s, 3H).

MS: 374.4 [(M+H)⁺]

### Step B:

To a solution of 4-[2-(4-chloro-2-methoxy-6-methyl-phenyl)-1-methyl-imidazo[4,5-b]pyrazin-5-yl]morpholine (80 mg, 214.00 μmol, 1 eq) in DCM (2 mL) was added BBr₃ (268.06 mg, 1.07 mmol, 103.10 μL, 5 eq) at 0° C. The mixture was stirred at 25° C. for 0.5 hr. The reaction mixture was poured onto 10 mL H₂O and then the pH was adjusted to 7 with saturated NaHCO₃. Then, the resulting solution was extracted with EtOAc (5 mL×2). The combined organic layers were washed successively with water (5 mL×2) and brine (20 ml×1), dried over anhydrous Na₂SO₄, filtered, and concentrated to give a residue. The residue was purified by prep-HPLC column: Waters Xbridge C18 150 mm×50 mm×10 μm; mobile phase: [H₂O (10 mM NH₄HCO₃)-ACN]; gradient: 20%-50% B over 8.0 min. Compound 4-[2-(4-chloro-2-methoxy-6-methyl-phenyl)-1-methyl-imidazo[4,5-b]pyrazin-5-yl]morpholine (100 mg, 43%) was obtained as a yellow solid.

¹H NMR (400 MHz, DMSO-d₆) δ=10.48 (br s, 1H), 8.18 (s, 1H), 6.95 (s, 1H), 6.89 (d, 1H), 3.76 (br d, 4H), 3.54 (s, 3H), 3.51 (br d, 4H), 2.08 (s, 3H)

MS: 360.1 [(M+H)⁺].

### Example 67 2-[5-(dimethylamino)-6-(hydroxymethyl)-1-methyl-imidazo[4,5-b]pyridin-2-yl]-3-methyl-5-(trifluoromethyl)phenol

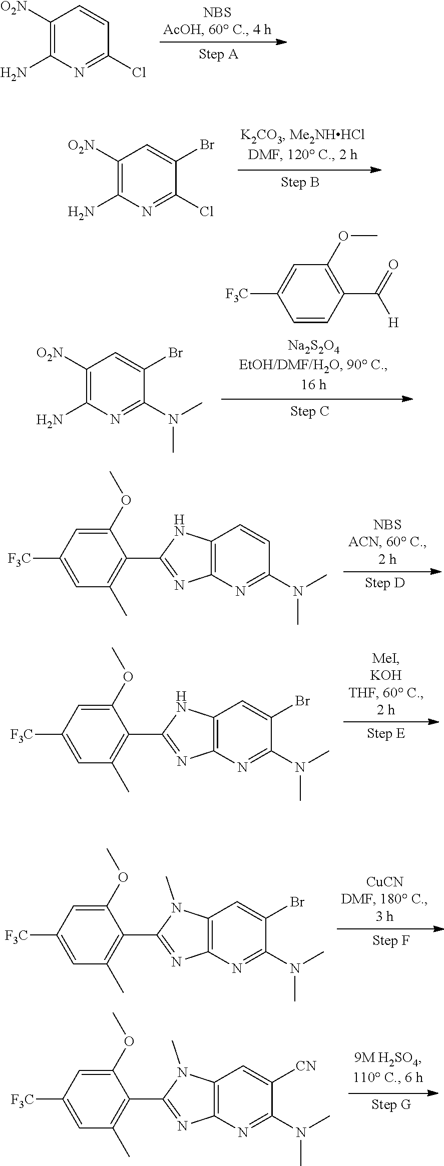

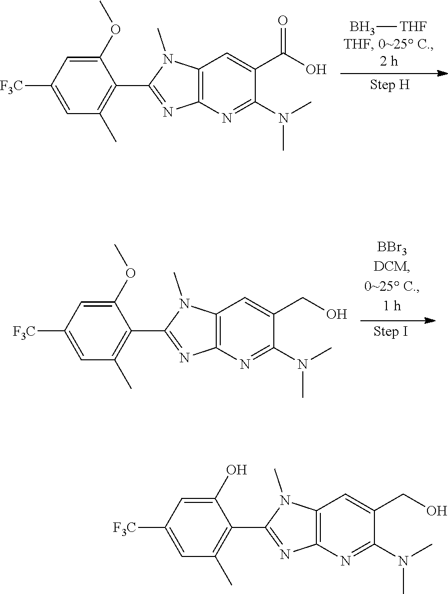

### Step A:

To the solution of 6-chloro-3-nitro-pyridin-2-amine (20 g, 115.24 mmol, 1.0 eq) in AcOH (300 mL) was added NBS (22.56 g, 126.76 mmol, 1.1 eq). The resulting reaction mixture was stirred at 60° C. for 4 h under N₂. LCMS indicated that the reaction was completed. The precipitate was collected by filtration, washed with water (100 mL×2), and concentrated to give a residue. The residue was used in the next step without further purification. 5-Bromo-6-chloro-3-nitro-pyridin-2-amine (27 g, crude) was obtained as a yellow solid.

LC-MS (ES+, m/z): 249.8 [(M−H)+].

1H NMR (400 MHz, DMSO-d6) δ=8.62 (s, 1H), 8.33 (br s, 2H).

### Step B:

To the solution of 5-bromo-6-chloro-3-nitro-pyridin-2-amine (2 g, 7.92 mmol, 1.0 eq) in DMF (20 mL) was added N-methylmethanamine hydrochloride (3.23 g, 39.61 mmol, 5.0 eq), K₂CO₃ (5.47 g, 39.61 mmol, 5.0 eq) in a sealed tube. The resulting reaction mixture was stirred at 120° C. for 2 h. LCMS indicated that the reaction was completed. The reaction mixture was poured onto 200 mL H₂O. Then the resulting solution was extracted with EA (100 mL×2). The combined organic layers were washed successively with water (50 mL×2) and brine (50 mL×1), dried over anhydrous Na₂SO₄, filtered, and concentrated to give a residue. The residue was used in the next step without further purification. 3-Bromo-N2,N2-dimethyl-5-nitro-pyridine-2,6-diamine (Batch×5 total: 10 g, crude) was obtained as a yellow solid.

LC-MS (ES+, m/z): 260.9 [(M+H)+].

1H NMR (400 MHz, DMSO-d6) δ=8.27 (s, 1H), 7.90 (br s, 2H), 3.17 (s, 6H)

### Step C:

To the solution of 2-methoxy-6-methyl-4-(trifluoromethyl)benzaldehyde (919.23 mg, 4.21 mmol, 1.1 eq) in EtOH (100 mL) and DMF (15 mL) was added 3-bromo-N2,N2-dimethyl-5-nitro-pyridine-2,6-diamine (1 g, 3.83 mmol, 1.0 eq), then 1M aqueous Na₂S₂O₄ (sodium dithionite (3.33 g, 19.15 mmol, 4.17 mL, 5.0 eq) in H₂O (19.15 mL) was added, the reaction mixture was stirred at 90° C. for 16 h under N₂. LCMS indicated that the reaction was completed. The reaction mixture was poured onto 50 mL H₂O. Then the resulting solution was extracted with EA (50 mL×2). The combined organic layers were washed successively with water (50 mL×2) and brine (50 mL×1), dried over anhydrous Na₂SO₄, filtered, and concentrated to give a residue. The residue was purified by column chromatography (SiO₂, petroleum ether/ethyl acetate=1/0 to 4/1) to give the title product. 2-[2-Methoxy-6-methyl-4-(trifluoromethyl)phenyl]-N,N-dimethyl-1H-imidazo[4,5-b]pyridin-5-amine (1.2 g, 3.43 mmol, 89.43% yield, N/A purity) was obtained as a yellow oil. (80.2% yield for 3 steps).

LC-MS (ES+, m/z): 351.1 [(M+H)+].

1H NMR (400 MHz, DMSO-d6) δ=12.44 (s, 1H), 7.80 (d, J=8.9 Hz, 1H), 7.33 (s, 1H), 7.25 (s, 1H), 6.59 (d, J=8.9 Hz, 1H), 3.80 (s, 3H), 3.07 (s, 6H), 2.26 (s, 3H).

### Step D:

To the solution of 2-[2-methoxy-6-methyl-4-(trifluoromethyl)phenyl]-N,N-dimethyl-1H-imidazo[4,5-b]pyridin-5-amine (1 g, 2.85 mmol, 1.0 eq) in ACN (10 mL) was added NBS (508.04 mg, 2.85 mmol, 1.0 eq). The resulting reaction mixture was stirred at 60° C. for 2 h under N₂. LCMS indicated that the reaction was completed. The reaction mixture was concentrated to give a residue. The residue was purified by column chromatography (SiO₂, petroleum ether/ethyl acetate=20/0 to 5/1) to give the title product. 6-Bromo-2-[2-methoxy-6-methyl-4-(trifluoromethyl)phenyl]-N,N-dimethyl-1H-imidazo[4,5-b]pyridin-5-amine (750 mg, 1.75 mmol, 61.21% yield, N/A purity) was obtained as a yellow solid.

LC-MS (ES+, m/z): 429.0 [(M+H)+].

1H NMR (400 MHz, DMSO-d6) δ=13.04 (s, 1H), 8.27 (s, 1H), 7.35 (s, 1H), 7.29 (s, 1H), 3.81 (s, 3H), 2.88 (s, 6H), 2.25 (s, 3H).

### Step E:

To the solution of 6-bromo-2-[2-methoxy-6-methyl-4-(trifluoromethyl)phenyl]-N,N-dimethyl-1H-imidazo[4,5-b]pyridin-5-amine (600 mg, 1.40 mmol, 1.0 eq) in THF (6 mL) was added KOH (235.28 mg, 4.19 mmol, 3.0 eq), CH3I (238.09 mg, 1.68 mmol, 104.42 μL, 1.2 eq). The resulting reaction mixture was stirred at 60° C. for 2 h under N₂. LCMS indicated that the reaction was completed. The reaction mixture was poured onto 50 mL H₂O. Then the resulting solution was extracted with EA (50 mL×2). The combined organic layers were washed successively with water (30 mL×2) and brine (30 mL×1), dried over anhydrous Na₂SO₄, filtered, and concentrated to give a residue. The residue was purified by prep-TLC (SiO₂, PE:EA=4:1) to give the title product. 6-Bromo-2-[2-methoxy-6-methyl-4-(trifluoromethyl)phenyl]-N,N,1-trimethyl-imidazo[4,5-b]pyridin-5-amine (300 mg, 676.80 μmol, 48.42% yield, N/A purity) was obtained as a yellow solid.

LC-MS (ES⁺, m/z): 443.0 [(M+H)⁺].

1H NMR (400 MHz, DMSO-d6) δ=8.41 (s, 1H), 7.42 (s, 1H), 7.34 (s, 1H), 3.82 (s, 3H), 3.51 (s, 3H), 2.85 (s, 6H), 2.16 (s, 3H).

### Step F:

The solution of 6-bromo-2-[2-methoxy-6-methyl-4-(trifluoromethyl)phenyl]-N,N,1-trimethyl-imidazo[4,5-b]pyridin-5-amine (270 mg, 609.12 μmol, 1.0 eq) in DMF (8 mL) was added CuCN (272.77 mg, 3.05 mmol, 665.28 μL, 5.0 eq). The resulting reaction mixture was stirred at 180° C. for 3 h in a microwave device. LCMS indicated that the reaction was completed. The reaction mixture was poured onto 100 mL H₂O. Then the resulting solution was extracted with EA (50 mL×2). The combined organic layers were washed successively with water (50 mL×2) and brine (50 mL×1), dried over anhydrous Na₂SO₄, filtered, and concentrated to give a residue. The crude product was used directly. 5-(Dimethylamino)-2-[2-methoxy-6-methyl-4-(trifluoromethyl)phenyl]-1-methyl-imidazo[4,5-b]pyridine-6-carbonitrile (0.2 g, crude) was obtained as a yellow solid.

LC-MS (ES+, m/z): 390.3 [(M+H)+].

### Step G:

To 5-(dimethylamino)-2-[2-methoxy-6-methyl-4-(trifluoromethyl)phenyl]-1-methyl-imidazo[4,5-b]pyridine-6-carbonitrile (160 mg, 410.92 μmol, 1.0 eq) was added a solution of H₂SO₄ (1.5 mL) and H₂O (1.5 mL). The resulting reaction mixture was stirred at 110° C. for 6 h. LCMS indicated that the reaction was completed. The reaction mixture was poured into 30 mL ice-water carefully and the pH was adjusted to pH=7 with NaHCO₃ solid. Then the resulting solution was extracted with EA (50 mL×2), dried over anhydrous Na₂SO₄, filtered, and concentrated to give a residue. The residue was purified by prep-TLC (SiO₂, DCM:MeOH=15:1) to give the title product. 5-(Dimethylamino)-2-[2-methoxy-6-methyl-4-(trifluoromethyl)phenyl]-1-methyl-imidazo[4,5-b]pyridine-6-carboxylic acid (140 mg, 342.82 μmol, 83.43% yield, N/A purity) was obtained as a yellow oil.

LC-MS (ES+, m/z): 409.1 [(M+H)+].

### Step H:

To the solution of 5-(dimethylamino)-2-[2-methoxy-6-methyl-4-(trifluoromethyl)phenyl]-1-methyl-imidazo[4,5-b]pyridine-6-carboxylic acid (60 mg, 146.92 μmol, 1.0 eq) in THF (1 mL) was added BH₃·THF (1 M, 1.03 mL, 7.0 eq) at 0° C. The resulting reaction mixture was stirred at 25° C. for 2 h under N₂. LCMS indicated that the reaction was completed. The reaction mixture was poured onto 50 mL H₂O. Then the resulting solution was extracted with EA (30 mL×2). The combined organic layers were washed successively with water (30 mL×2) and brine (30 mL×1), dried over anhydrous Na₂SO₄, filtered, and concentrated to give a residue. The residue was purified by prep-TLC (SiO₂, DCM:MeOH=10:1) to give the title product. [5-(Dimethylamino)-2-[2-methoxy-6-methyl-4-(trifluoromethyl)phenyl]-1-methyl-imidazo[4,5-b]pyridin-6-yl]methanol (50 mg, 126.78 μmol, 86.29% yield, N/A purity) was obtained as a yellow oil.

LC-MS (ES+, m/z): 395.1 [(M+H)+].

1H NMR (400 MHz, DMSO-d6) δ=8.21 (s, 1H), 7.45 (s, 1H), 7.38 (s, 1H), 5.49-5.46 (t, J=5.3 Hz, 1H), 4.66-4.64 (d, J=5.3 Hz, 2H), 3.83 (s, 3H), 3.61 (s, 3H), 2.87 (s, 6H), 2.12 (s, 3H).

### Step I:

The solution of [5-(dimethylamino)-2-[2-methoxy-6-methyl-4-(trifluoromethyl)phenyl]-1-methyl-imidazo[4,5-b]pyridin-6-yl]methanol (10 mg, 25.36 μmol, 1.0 eq) in DCM (1 mL) was added BBr₃ (19.06 mg, 76.07 μmol, 7.33 μL, 3.0 eq) at 0° C. The resulting reaction mixture was stirred at 25° C. for 1 h. LCMS indicated that the reaction was completed. The reaction mixture was concentrated applying a stream of N₂. The residue was purified by prep-HPLC (basic condition, column: Waters Xbridge C18 150 mm×50 mm×10 μm; mobile phase: [H₂O (0.05% NH₃ H₂O+10 mM NH₄HCO₃)-ACN]; gradient: 25%-55% B over 8.0 min) to give the title product. 2-[5-(Dimethylamino)-6-(hydroxymethyl)-1-methyl-imidazo[4,5-b]pyridin-2-yl]-3-methyl-5-(trifluoromethyl)phenol (4.15 mg, 10.91 μmol, 43.03% yield) was obtained as a white solid.

LC-MS (ES+, m/z): 381.1 [(M+H)+].

1H NMR (400 MHz, DMSO-d6) δ=10.57 (br s, 1H), 8.00 (s, 1H), 7.22 (s, 1H), 7.12 (s, 1H), 5.32-5.29 (t, J=5.4 Hz, 1H), 4.66-4.65 (d, J=5.1 Hz, 2H), 3.55 (s, 3H), 2.76 (s, 6H), 2.15 (s, 3H).

### Example 68: 3-methyl-2-(1-methyl-5-(4-methylpiperazin-1-yl)-1H-imidazo[4,5-b]pyrazin-2-yl)-5-(trifluoromethyl)phenol formate Salt

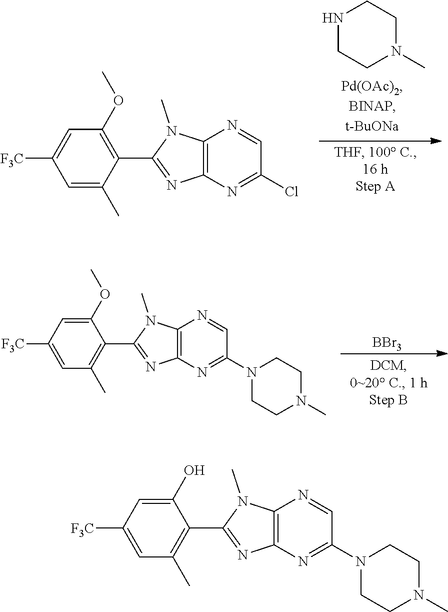

### Step A:

To a solution of 5-chloro-2-[2-methoxy-6-methyl-4-(trifluoromethyl)phenyl]-1-methyl-imidazo[4,5-b]pyrazine (200.00 mg, 560.65 μmol, 1 eq) in THF (2 mL) was added t-BuONa (107.76 mg, 1.12 mmol, 2 eq), Pd(OAc)₂ (12.59 mg, 56.06 μmol, 0.1 eq), BINAP (69.82 mg, 112.13 μmol, 0.2 eq), and 1-methylpiperazine (112.31 mg, 1.12 mmol, 124.38 μL, 2 eq) and the reaction mixture was stirred at 100° C. for 16 h in a sealed tube. After completion, the solvent was removed by concentration to give the residue. The residue was purified by prep-TLC (SiO₂, DCM/MeOH=10:1) to give 2-[2-methoxy-6-methyl-4-(trifluoromethyl)phenyl]-1-methyl-5-(4-methylpiperazin-1-yl)imidazo[4,5-b]pyrazine (50 mg, 21%) as a yellow solid.

¹H NMR (400 MHz, DMSO-d₆) δ=8.30 (s, 1H), 7.43 (s, 1H), 7.36 (s, 1H), 3.84 (s, 3H), 3.78-3.58 (m, 1H), 3.52 (s, 3H), 3.49-3.37 (m, 2H), 3.24-2.94 (m, 3H), 2.81-2.69 (m, 2H), 2.19 (s, 3H), 1.24 (s, 3H).

MS: 421.3 [(M+H)⁺].

### Step B:

To a solution of 2-[2-methoxy-6-methyl-4-(trifluoromethyl)phenyl]-1-methyl-5-(4-methylpiperazin-1-yl)imidazo[4,5-b]pyrazine (40 mg, 95.14 μmol, 1 eq) in DCM (2 mL) was added BBr₃ (71.50 mg, 285.42 μmol, 27.50 μL, 3 eq) at 0° C. and the reaction mixture was stirred at 20° C. for 1 h. After completion, the solvent was removed by concentration to give the residue. The residue was purified by prep-HPLC (column: Phenomenex luna C18 100 mm×40 mm×3 μm; mobile phase: [H₂O (0.2% FA)-ACN]; gradient: 10%-40% B over 8.0 min) to give 3-methyl-2-[1-methyl-5-(4-methylpiperazin-1-yl)imidazo[4,5-b]pyrazin-2-yl]-5-(trifluoromethyl)phenol (22.7 mg, 57%) as a white solid.

LC-MS (ES⁺, m/z): 407.1 [(M+H)⁺].

¹H NMR (400 MHz, DMSO-d₆) δ=10.70 (br s, 1H), 8.19 (s, 1H), 7.23 (s, 1H), 7.13 (s, 1H), 3.59-3.55 (m, 4H), 3.55 (s, 3H), 2.53-2.52 (m, 4H), 2.25 (s, 3H), 2.17 (s, 3H).

MS: 407.1 [(M+H)⁺].

### Example 69: 5-chloro-3-methyl-2-(1-methyl-5-(4-methylpiperazin-1-yl)-1H-imidazo[4,5-b]pyrazin-2-yl)phenol

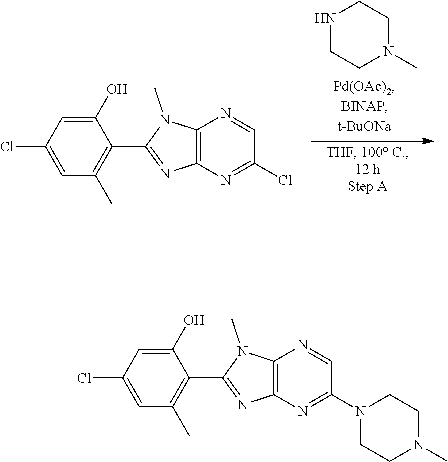

### Step A:

To a solution of 5-chloro-2-(5-chloro-1-methyl-imidazo[4,5-b]pyrazin-2-yl)-3-methyl-phenol (100 mg, 323.47 μmol, 1 eq) and 1-methylpiperazine (38.88 mg, 388.16 μmol, 43.06 μL, 1.2 eq) in THF (3 mL) was added Pd(OAc)₂ (7.26 mg, 32.35 μmol, 0.1 eq) and BINAP (40.28 mg, 64.69 μmol, 0.2 eq) and tBuONa (62.17 mg, 646.93 μmol, 2 eq). The mixture was stirred at 100° C. for 12 hr. The reaction mixture was quenched by addition of H₂O 20 mL, and then extracted with EtOAc (10 mL×3). The combined organic layers were washed with brine 20 mL, dried over Na₂SO₄, filtered and concentrated under reduced pressure to give a residue. The residue was purified by prep-HPLC column: Waters Xbridge C18 150 mm×50 mm×10 μm; mobile phase: [H₂O (10 mM NH₄HCO₃)-ACN]; gradient: 20%-60% B over 8.0 min. Compound 5-chloro-3-methyl-2-[1-methyl-5-(4-methylpiperazin-1-yl)imidazo[4,5-b]pyrazin-2-yl]phenol (33.21 mg, 85.24 μmol, 26.35% yield, 95.70% purity) was obtained as a white solid.

¹H NMR (400 MHz, DMSO-d₆) δ=10.63-10.23 (m, 1H), 8.16 (s, 1H), 6.95 (s, 1H), 6.88 (d, 1H), 3.56-3.52 (m, 7H), 2.47-2.44 (m, 4H), 2.23 (s, 3H), 2.08 (s, 3H).

MS: 373.0 [(M+H)⁺]

### Example 70 5-chloro-2-[6-(hydroxymethyl)-5-methoxy-1-methyl-imidazo[4,5-b]pyridin-2-yl]-3-methyl-Phenol

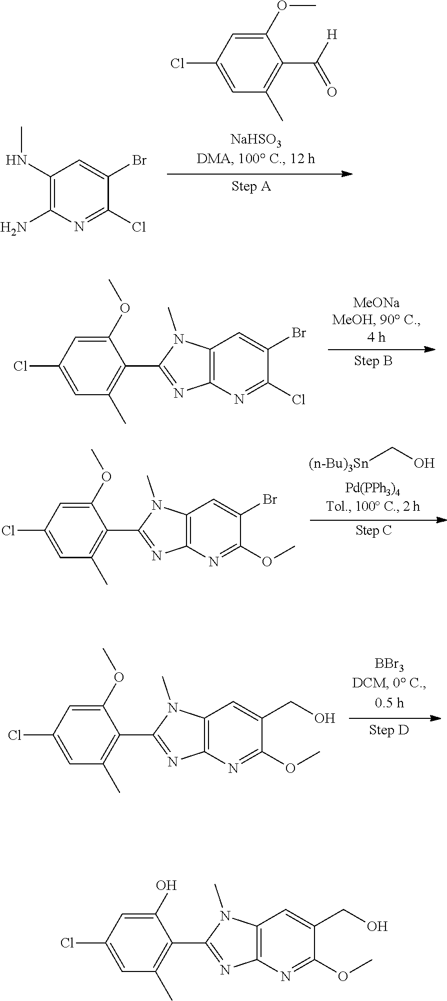

### Step A:

To a solution of 5-bromo-6-chloro-N3-methyl-pyridine-2,3-diamine (500 mg, 1.48 mmol, 1 eq) in DMA (8 mL) were added 4-chloro-2-methoxy-6-methyl-benzaldehyde (273.22 mg, 1.48 mmol, 1 eq) and NaHSO₃ (308.01 mg, 2.96 mmol, 208.11 μL, 2 eq). The mixture was stirred at 100° C. for 12 hr. LCMS indicated that the reaction was completed. The reaction mixture was quenched by addition of H₂O 40 mL, and then the mixture was extracted with EA (20 mL×3). The combined organic layers were washed with brine 50 mL×3, dried over Na₂SO₄, filtered and concentrated under reduced pressure to give a residue. The residue was purified by column chromatography (SiO₂, petroleum ether/ethyl acetate=1/0 to 12/88) and by prep-HPLC column: Waters Xbridge BEH C18 100 mm×30 mm×10 μm; mobile phase: [H₂O (10 mM NH₄HCO₃)-ACN]; gradient: 45%-75% B over 8.0 min to give the title product. 6-Bromo-5-chloro-2-(4-chloro-2-methoxy-6-methyl-phenyl)-1-methyl-imidazo[4,5-b]pyridine (410 mg, 1.02 mmol, 69.07% yield, N/A purity) was obtained as a white solid.

LC-MS (ES+, m/z): 399.9 [(M+H)+].

1H NMR (400 MHz, DMSO-d6) δ=8.69 (s, 1H), 7.16 (br d, J=13.6 Hz, 2H), 3.77 (s, 3H), 3.56 (s, 3H), 2.07 (s, 3H)

### Step B:

To a solution of 6-bromo-5-chloro-2-(4-chloro-2-methoxy-6-methyl-phenyl)-1-methyl-imidazo[4,5-b]pyridine (300 mg, 747.97 μmol, 1 eq) in MeOH (4 mL) was added MeONa (40.41 mg, 747.97 μmol, 4 mL, 1 eq). The mixture was stirred at 90° C. for 4 hr. LCMS indicated that the reaction was completed. The reaction mixture was quenched by addition of H₂O 30 mL, and then extracted with EA (10 mL×3). The combined organic layers were washed with brine 30 mL, dried over Na₂SO₄, filtered and concentrated under reduced pressure to give a residue. The residue was purified by prep-TLC (SiO₂, PE:EA=1:1). Compound 6-bromo-2-(4-chloro-2-methoxy-6-methyl-phenyl)-5-methoxy-1-methyl-imidazo[4,5-b]pyridine (250 mg, 630.25 μmol, 84.26% yield, N/A purity) was obtained as a yellow solid.

LC-MS (ES⁺, m/z): 396.0 [(M+H)⁺].

### Step C:

To a solution of 6-bromo-2-(4-chloro-2-methoxy-6-methyl-phenyl)-5-methoxy-1-methyl-imidazo[4,5-b]pyridine (80 mg, 201.68 μmol, 1 eq) in toluene (3 mL) was added tri-butylstannylmethanol (259.03 mg, 806.72 μmol, 4 eq) and Pd(PPh₃)₄ (23.31 mg, 20.17 μmol, 0.1 eq). The mixture was stirred at 100° C. for 2 hr. LCMS indicated that the reaction was completed. The reaction mixture was quenched by addition of H₂O 10 mL, and then extracted with EA (5 mL×3). The combined organic layers were washed with brine 10 mL, dried over Na₂SO₄, filtered and concentrated under reduced pressure to give a residue. The residue was purified by prep-TLC (SiO₂, PE:EA=3:1). [2-(4-Chloro-2-methoxy-6-methyl-phenyl)-5-methoxy-1-methyl-imidazo[4,5-b]pyridin-6-yl]methanol (80 mg, 184.02 μmol, 91.24% yield, 80% purity) was obtained as a white solid.

LC-MS (ES⁺, m/z): 348.3 [(M+H)⁺].

¹H NMR (400 MHz, DMSO-d₆) δ=7.97 (s, 1H), 7.14 (d, J=1.5 Hz, 1H), 7.12 (s, 1H), 5.23 (t, J=5.4 Hz, 1H), 4.58 (d, J=5.5 Hz, 2H), 3.92 (s, 3H), 3.76 (s, 3H), 3.51 (s, 3H), 2.06 (s, 3H)

### Step D:

To a solution of [2-(4-chloro-2-methoxy-6-methyl-phenyl)-5-methoxy-1-methyl-imidazo[4,5-b]pyridin-6-yl]methanol (70 mg, 201.27 μmol, 1 eq) in DCM (2 mL) was added BBr₃ (252.11 mg, 1.01 mmol, 96.97 μL, 5 eq) at 0° C. The mixture was stirred at 0° C. for 0.5 hr. LCMS indicated that the reaction was completed. The reaction mixture was poured onto 10 mL H₂O and then the pH was adjusted to 7 with saturated NaHCO₃. Then the resulting solution was extracted with EA (5 mL×2). The combined organic layers were washed successively with water (5 mL×2) and brine (20 mL×1), dried over anhydrous Na₂SO₄, filtered, and concentrated to give a residue. The residue was purified by prep-HPLC column: Waters Xbridge C18 150 mm×50 mm×10 μm; mobile phase: [H₂O (10 mM NH₄HCO₃)-ACN]; gradient: 20%-50% B over 8.0 min. Compound 5-chloro-2-[6-(hydroxymethyl)-5-methoxy-1-methyl-imidazo[4,5-b]pyridin-2-yl]-3-methyl-phenol (10.04 mg, 29.54 μmol, 14.68% yield, 98.19% purity) was obtained as a white solid.

LC-MS (ES⁺, m/z): 334.1 [(M+H)⁺].

¹H NMR (400 MHz, DMSO-d₆) δ=10.32 (br s, 1H), 7.96 (s, 1H), 6.94 (d, J=1.4 Hz, 1H), 6.87 (d, J=1.9 Hz, 1H), 5.25 (s, 1H), 4.58 (d, J=5.1 Hz, 2H), 3.92 (s, 3H), 3.55 (s, 3H), 2.05 (s, 3H)

### Example 71: 2-(5-(3-(dimethylamino)azetidin-1-yl)-1-methyl-1H-imidazo[4,5-b]pyrazin-2-yl)-3-methyl-5-(trifluoromethyl)phenol formate Salt

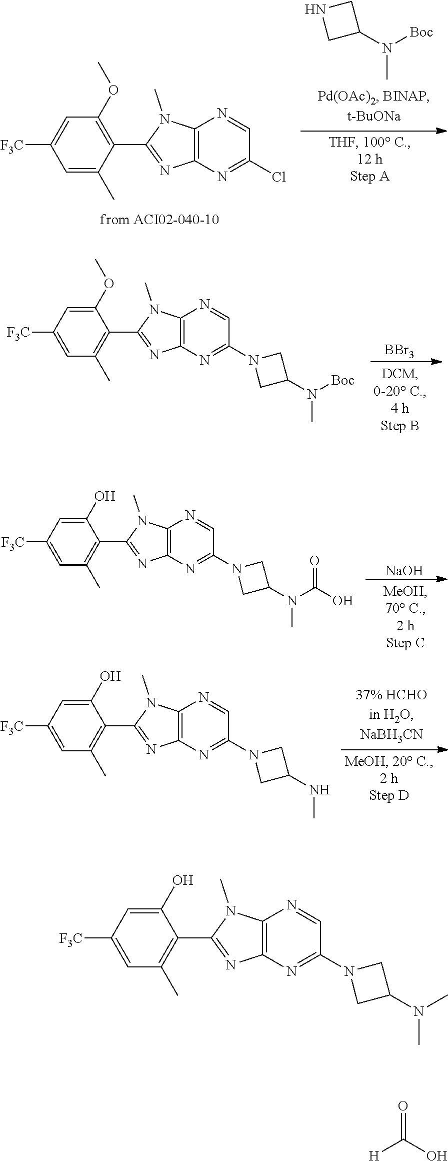

### Step A:

To a solution of 5-chloro-2-[2-methoxy-6-methyl-4-(trifluoromethyl)phenyl]-1-methyl-imidazo[4,5-b]pyrazine (300.00 mg, 840.97 μmol, 1 eq) in THF (10 mL) was added sodium 2-methylpropan-2-olate (161.64 mg, 1.68 mmol, 2 eq), Pd(OAc)₂ (18.88 mg, 84.10 μmol, 0.1 eq), BINAP (104.73 mg, 168.19 μmol, 0.2 eq), and tert-butyl N-(azetidin-3-yl)-N-methyl-carbamate (313.26 mg, 1.68 mmol, 2 eq) and the mixture was purged 3 times with N₂. Then the reaction mixture was stirred at 100° C. for 24 h in a sealed tube. After completion of the reaction, the solvent was removed by concentration to give the residue. The residue was purified by prep-TLC (SiO₂, petroleum ether/ethyl acetate=1:1) to give tert-butyl N-[1-[2-[2-methoxy-6-methyl-4-(trifluoromethyl)phenyl]-1-methyl-imidazo[4,5-b]pyrazin-5-yl]azetidin-3-yl]-N-meth yl-carbamate (200 mg, 47%) as a yellow solid.

¹H NMR (400 MHz, DMSO-d₆) δ=7.75 (s, 1H), 7.42 (s, 1H), 7.35 (s, 1H), 4.25 (dt, 2H), 4.11-4.05 (m, 2H), 4.04-4.00 (m, 1H), 3.85-3.80 (m, 3H), 3.51 (s, 3H), 2.89 (s, 3H), 2.18 (s, 3H), 1.41 (s, 9H).

MS: 507.2 [(M+H)⁺].

### Step B:

To a solution of tert-butyl N-[1-[2-[2-methoxy-6-methyl-4-(trifluoromethyl)phenyl]-1-methyl-imidazo[4,5-b]pyrazin-5-yl]azetidin-3-yl]-N-methyl-carbamate (200 mg, 394.85 μmol, 1 eq) in DCM (2 mL) was added BBr₃ (296.75 mg, 1.18 mmol, 114.14 μL, 3 eq) at 0° C. and the reaction mixture was stirred at 20° C. for 4 h. After completion, the solvent was removed by concentration to give the residue. The residue was purified by prep-TLC (SiO₂, DCM/MeOH=10:1) to give [1-[2-[2-hydroxy-6-methyl-4-(trifluoromethyl)phenyl]-1-methyl-imidazo[4,5-b]pyrazin-5-yl]azetidin-3-yl]-methyl-carbamic acid (170 mg, 59%) as a yellow solid. LC-MS (ES⁺, m/z): 437.2 [(M+H)⁺].

¹H NMR (400 MHz, DMSO-d₆) δ=11.12-9.90 (m, 1H), 7.83 (s, 1H), 7.22 (s, 1H), 7.14-7.11 (m, 1H), 4.35 (br t, J=7.8 Hz, 1H), 4.17-4.06 (m, 1H), 3.99 (td, J=4.4, 8.6 Hz, 1H), 3.64 (br d, J=2.0 Hz, 2H), 3.52 (s, 3H), 2.84 (s, 3H), 2.17 (s, 3H).

MS: 437.2 [(M+H)⁺].

### Step C:

To the solution of [1-[2-[2-hydroxy-6-methyl-4-(trifluoromethyl)phenyl]-1-methyl-imidazo[4,5-b]pyrazin-5-yl]azetidin-3-yl]-methyl-carbamic acid (150.00 mg, 343.73 μmol, 1 eq) in MeOH (3 mL) was added NaOH (27.50 mg, 687.46 μmol, 2 eq) and the reaction mixture was stirred at 70° C. for 2 h. After completion, the solvent was removed by concentration to give a residue. The residue was purified by prep-TLC (SiO₂, DCM:MeOH=10:1) to give 3-methyl-2-[1-methyl-5-[3-(methylamino)azetidin-1-yl]imidazo[4,5-b]pyrazin-2-yl]-5-(trifluoromethyl)phenol (20 mg, 15%) as a yellow solid.

¹H NMR (400 MHz, DMSO-d₆) δ=7.71 (s, 1H), 7.24 (s, 1H), 7.15 (s, 1H), 4.20 (br d, 2H), 3.81-3.71 (m, 2H), 3.71-3.63 (m, 1H), 3.60-3.53 (m, 3H), 3.53-3.38 (m, 1H), 2.30-2.26 (m, 3H), 2.17 (s, 3H).

MS: 393.2 [(M+H)⁺].

### Step D:

To a solution of 3-methyl-2-[1-methyl-5-[3-(methylamino)azetidin-1-yl]imidazo[4,5-b]pyrazin-2-yl]-5-(trifluoromethyl)phenol (15 mg, 38.23 μmol, 1 eq) in MeOH (1 mL) was added HCHO in H₂O (15.51 mg, 191.14 μmol, 14.23 μL, 37% purity, 5 eq) for 1 h and then NaBH₃CN (12.01 mg, 191.14 μmol, 5 eq) was added at 20° C. for 1 h. After completion, the solvent was removed by concentration to give the residue. The residue was purified by prep-HPLC (column: Phenomenex luna C18 100 mm×40 mm×3 μm; mobile phase: [H₂O (0.2% FA)-ACN]; gradient: 10%-40% B over 8.0 min) to give 2-[5-[3-(dimethylamino)azetidin-1-yl]-1-methyl-imidazo[4,5-b]pyrazin-2-yl]-3-methyl-5-(trifluoromethyl)phenol (3.02 mg, 19%) as a white solid.

¹H NMR (400 MHz, DMSO-d₆) δ=10.86-10.51 (m, 1H), 7.71 (s, 1H), 7.22 (s, 1H), 7.13 (s, 1H), 4.10 (br s, 2H), 3.86-3.78 (m, 2H), 3.54 (s, 3H), 3.25 (br d, 1H), 2.17 (s, 3H), 2.13 (s, 6H).

MS: 407.2 [(M+H)⁺].

### Example 72 (R)-2-(6-methoxy-1-methyl-5-((1-methylpyrrolidin-3-yl)amino)-1H-imidazo[4,5-b]pyrazin-2-yl)-3-methyl-5-(trifluoromethyl)phenol formate Salt

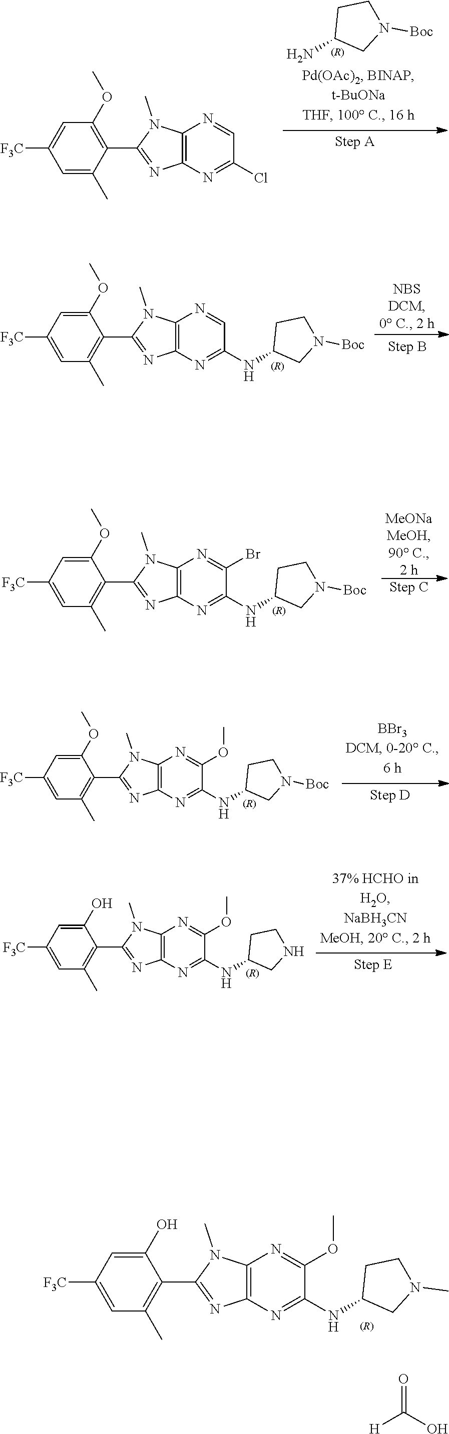

### Step A:

To the solution of 5-chloro-2-[2-methoxy-6-methyl-4-(trifluoromethyl)phenyl]-1-methyl-imidazo[4,5-b]pyrazine (600.00 mg, 1.68 mmol, 1 eq) in THF (15 mL) was added sodium 2-methylpropan-2-olate (323.28 mg, 3.36 mmol, 2 eq), Pd(OAc)₂ (37.76 mg, 168.19 μmol, 0.1 eq), BINAP (209.46 mg, 336.39 μmol, 0.2 eq), and tert-butyl (3R)-3-aminopyrrolidine-1-carboxylate (626.53 mg, 3.36 mmol, 570.61 μL, 2 eq) and the mixture was purged 3 times with N₂. Then the reaction mixture was stirred at 100° C. for 16 hr in a sealed tube. LCMS indicated that the reaction was completed. After completion, the solvent was removed by concentration to give the residue. The residue was purified by prep-TLC (SiO₂, petroleum ether/ethyl acetate=1:1). tert-Butyl (3R)-3-[[2-[2-methoxy-6-methyl-4-(trifluoromethyl)phenyl]-1-methyl-imidazo[4,5-b]pyrazin-5-yl]amino]pyrrolidine-1-carboxylate (500 mg, 987.13 μmol, 58.69% yield) was obtained as a yellow solid.

LC-MS (ES⁺, m/z): 507.1 [(M+H)⁺].

¹H NMR (400 MHz, DMSO-d₆) δ=7.80 (s, 1H), 7.41 (s, 1H), 7.34 (s, 1H), 7.23-7.15 (m, 1H), 4.42-4.27 (m, 1H), 3.83 (s, 3H), 3.68-3.55 (m, 1H), 3.47 (s, 3H), 3.17 (d, J=5.3 Hz, 1H), 3.18-3.15 (m, 2H), 2.23-2.18 (m, 3H), 2.17-2.09 (m, 1H), 1.95-1.81 (m, 1H), 1.45-1.35 (m, 9H).

### Step B:

To the solution of tert-butyl (3R)-3-[[2-[2-methoxy-6-methyl-4-(trifluoromethyl)phenyl]-1-methyl-imidazo[4,5-b]pyrazin-5-yl]amino]pyrrolidine-1-carboxylate (500 mg, 987.13 μmol, 1 eq) in DCM (10 mL) was added NBS (439.23 mg, 2.47 mmol, 2.5 eq) and the reaction mixture was stirred at 0° C. for 2 hr. LCMS indicated that the reaction was completed. After completion, the solvent was removed by concentration to give the residue. The residue was purified by prep-TLC (SiO₂, petroleum ether/ethyl acetate=1:1). tert-Butyl (3R)-3-[[5-bromo-2-[2-methoxy-6-methyl-4-(trifluoromethyl)phenyl]-3-methyl-imidazo[4,5-b]pyrazin-6-yl]amino]pyrrolidine-1-carboxylate (400 mg, 683.28 μmol, 69.22% yield, N/A purity) was obtained as a yellow solid.

LC-MS (ES⁺, m/z): 585.3 [(M+H)⁺].

¹H NMR (400 MHz, DMSO-d₆) δ=7.42 (s, 1H), 7.35 (s, 1H), 6.36 (br d, J=6.8 Hz, 1H), 4.56-4.38 (m, 1H), 3.84 (s, 3H), 3.75-3.61 (m, 1H), 3.47 (s, 3H), 3.30-3.14 (m, 3H), 2.25-2.16 (m, 4H), 2.12-1.97 (m, 1H), 1.41 (br s, 9H).

### Step C:

To the solution of tert-butyl (3R)-3-[[5-bromo-2-[2-methoxy-6-methyl-4-(trifluoromethyl)phenyl]-3-methyl-imidazo[4,5-b]pyrazin-6-yl]amino]pyrrolidine-1-carboxylate (400.00 mg, 683.28 μmol, 1 eq) in MeOH (8 mL) was added NaOMe (99.76 mg, 683.28 μmol, 3 mL, 37% purity) and the reaction mixture was stirred at 90° C. for 2 hr. LCMS indicated that the reaction was completed. After completion, the solvent was removed by concentration to give the residue. The residue was purified by prep-TLC (SiO₂, petroleum ether/ethyl acetate=1:1). tert-Butyl (3R)-3-[[5-methoxy-2-[2-methoxy-6-methyl-4-(trifluoromethyl)phenyl]-3-methyl-imidazo[4,5-b]pyrazin-6-yl]amino]pyrrolidine-1-carboxylate (300 mg, 559.13 μmol, 81.83% yield, N/A purity) was obtained as a yellow solid.

LC-MS (ES⁺, m/z): 537.4 [(M+H)⁺].

¹H NMR (400 MHz, DMSO-d₆) δ=7.39 (s, 1H), 7.31 (s, 1H), 6.50 (br s, 1H), 4.52-4.36 (m, 1H), 4.05-4.03 (m, 3H), 3.83 (s, 3H), 3.71-3.57 (m, 1H), 3.44 (s, 3H), 3.41 (br s, 1H), 3.31-3.14 (m, 2H), 2.18 (s, 3H), 2.16-2.00 (m, 2H), 1.40 (br d, J=5.1 Hz, 9H).

### Step D:

To the solution of tert-butyl (3R)-3-[[5-methoxy-2-[2-methoxy-6-methyl-4-(trifluoromethyl)phenyl]-3-methyl-imidazo[4,5-b]pyrazin-6-yl]amino]pyrrolidine-1-carboxylate (300 mg, 559.13 μmol, 1 eq) in DCM (7 mL) was added BBr₃ (490.26 mg, 1.96 mmol, 188.56 μL, 3.5 eq) at 0° C. and the reaction mixture was stirred at 20° C. for 6 hr. LCMS indicated that the reaction was completed. The reaction mixture was poured onto 8 mL saturated NaHCO₃. Then the resulting solution was extracted with DCM (10 mL×4). The combined organic layers were washed successively with water (10 mL×2) and brine (10 mL×1), dried over anhydrous Na₂SO₄, filtered, and concentrated to give a residue. The residue was used for the next step directly without further purification. 2-[5-Methoxy-3-methyl-6-[[(3R)-pyrrolidin-3-yl]amino]imidazo[4,5-b]pyrazin-2-yl]-3-methyl-5-(trifluoromethyl)phenol (170 mg, crude) was obtained as a yellow solid.

LC-MS (ES⁺, m/z): 423.3 [(M+H)⁺].

### Step E:

To the solution of 2-[5-methoxy-3-methyl-6-[[(3R)-pyrrolidin-3-yl]amino]imidazo[4,5-b]pyrazin-2-yl]-3-methyl-5-(trifluoromethyl)phenol (170 mg, 402.46 μmol, 1 eq) in MeOH (5 mL) was added HCHO in H₂O (163.30 mg, 2.01 mmol, 149.82 μL, 37% purity, 5 eq). The reaction mixture was stirred at 20° C. for 1 h, followed by the addition of NaBH₃CN (126.46 mg, 2.01 mmol, 5 eq). The reaction mixture was stirred at 20° C. for 1 h. The reaction mixture was poured onto 25 mL saturated Na₂CO₃. Then the resulting solution was extracted with DCM (25 mL×4). The combined organic layers were washed successively with water (10 mL×2) and brine (10 mL×1), dried over anhydrous Na₂SO₄, filtered, and concentrated to give a residue. The residue was purified by prep-HPLC (column: Phenomenex Luna C18 100 mm×30 mm×3 μm; mobile phase: [H₂O (0.2% FA)-ACN]; gradient: 1%-35% B over 8.0 min) to give the title product. 2-[5-Methoxy-3-methyl-6-[[(3R)-1-methylpyrrolidin-3-yl]amino]imidazo[4,5-b]pyrazin-2-yl]-3-methyl-5-(trifluoromethyl)phenol (61.4 mg, 140.69 μmol, 25.2% yield for 2 steps, N/A purity) was obtained as a white solid.

LC-MS (ES⁺, m/z): 437.2 [(M+H)⁺].

¹H NMR (400 MHz, DMSO-d₆) δ=7.18 (s, 1H), 7.12 (s, 1H), 6.22 (d, J=6.9 Hz, 1H), 4.47 (td, J=2.0, 6.7 Hz, 1H), 4.03 (s, 3H), 3.48 (s, 3H), 2.91 (dd, J=6.9, 9.6 Hz, 1H), 2.82-2.74 (m, 1H), 2.69-2.55 (m, 2H), 2.36 (s, 3H), 2.27 (td, J=5.3, 8.2 Hz, 1H), 2.16 (s, 3H), 1.84 (br d, J=3.1 Hz, 1H).

¹H NMR (400 MHz, chloroform-d) δ=7.20 (s, 1H), 7.06 (s, 1H), 5.77 (br s, 1H), 4.72 (br d, J=7.6 Hz, 1H), 4.08-4.01 (m, 3H), 3.61 (s, 3H), 3.44-3.32 (m, 2H), 3.00-2.91 (m, 1H), 2.72-2.62 (m, 1H), 2.59 (s, 3H), 2.55-2.44 (m, 1H), 2.27 (s, 3H), 2.12-1.99 (m, 1H).

MS: 413.2 [(M+H)⁺]

### Example 73: 5-chloro-3-methyl-2-(5-morpholino-1H-imidazo[4,5-b]pyrazin-2-yl)phenol

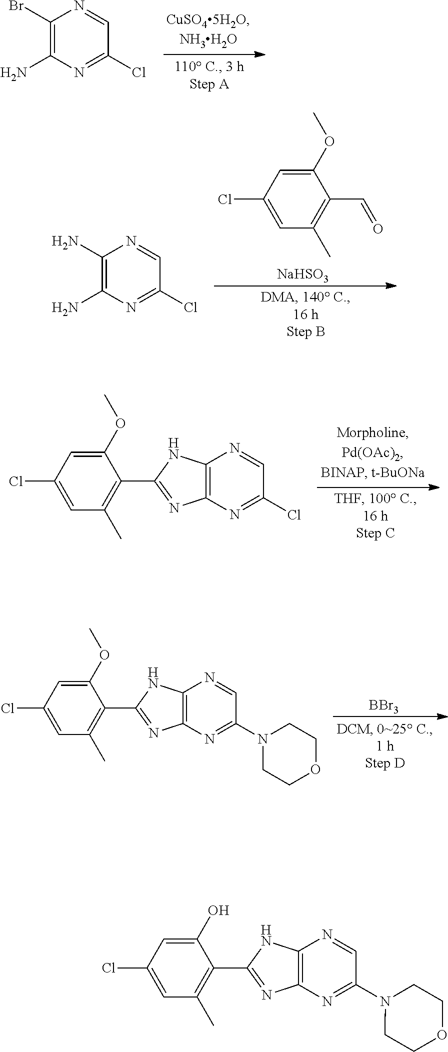

### Step A:

To the solution of 3-bromo-6-chloro-pyrazin-2-amine (3.5 g, 16.79 mmol, 1.0 eq) in NH₃·H₂O (20 mL) was added CuSO₄SO₄·5 H₂O (419.25 mg, 1.68 mmol, 0.1 eq). The reaction mixture was stirred at 110° C. for 3 h in a sealed tube. After filtration via celite pad, the celite pad was washed with DCM 100 mL, the filtrate was concentrated under reduced pressure to give a residue. The residue was purified by column chromatography (SiO₂, petroleum ether/ethyl acetate=8/1 to 1/2) to give 5-chloropyrazine-2,3-diamine (2 g, 82%) as a white solid.

1H NMR (400 MHz, DMSO-d6) δ=7.13 (s, 1H), 6.39 (s, 2H), 6.07 (s, 2H).

MS: 144.9 [(M+H)⁺]

### Step B:

To the solution of 5-chloropyrazine-2,3-diamine (0.5 g, 3.46 mmol, 1.0 eq) and 4-chloro-2-methoxy-6-methyl-benzaldehyde (702.40 mg, 3.80 mmol, 1.1 eq) in DMA (8 mL) was added NaHSO₃ (539.87 mg, 5.19 mmol, 364.78 μL, 1.5 eq), the reaction mixture was stirred at 140° C. for 16 h under N₂. The reaction mixture was poured onto 50 mL H₂O. Then, the resulting solution was extracted with EtOAc (50 mL×2). The combined organic layers were washed successively with water (50 mL×2) and brine (50 mL×1), dried over anhydrous Na₂SO₄, filtered, and concentrated to give a residue. The residue was purified by column chromatography (SiO₂, petroleum ether/ethyl acetate=8/1 to 1/1) to give 5-chloro-2-(4-chloro-2-methoxy-6-methyl-phenyl)-1H-imidazo[4,5-b]pyrazine (0.5 g, 47%) as a yellow solid.

1H NMR (400 MHz, DMSO-d6) δ=13.82-13.75 (m, 1H), 8.56-8.49 (m, 1H), 7.18-6.90 (m, 2H), 3.79 (s, 3H), 2.23 (s, 3H).

MS: 309.0 [(M+H)⁺]

### Step C:

To a solution of 5-chloro-2-(4-chloro-2-methoxy-6-methyl-phenyl)-1H-imidazo[4,5-b]pyrazine (300 mg, 970.40 μmol, 1.0 eq) and morpholine (253.62 mg, 2.91 mmol, 256.19 μL, 3.0 eq) in THF (5 mL) was added t-BuONa (186.52 mg, 1.94 mmol, 2.0 eq), BINAP (120.85 mg, 194.08 μmol, 0.2 eq) and Pd(OAc)2 (21.79 mg, 97.04 μmol, 0.1 eq). The reaction mixture was stirred at 100° C. for 16 h in a sealed tube. The reaction mixture was poured onto 50 mL H₂O. Then, the resulting solution was extracted with EtOAc (50 mL×2). The combined organic layers were washed successively with water (50 mL×2) and brine (50 mL×1), dried over anhydrous Na₂SO₄, filtered, and concentrated to give a residue. The residue was purified by prep-TLC(SiO₂, DCM:MeOH=20:1) to give 4-[2-(4-chloro-2-methoxy-6-methyl-phenyl)-1H-imidazo[4,5-b]pyrazin-5-yl]morpholine (0.15 g, 43%) as a yellow solid.

1H NMR (400 MHz, DMSO-d6) δ=13.02-12.82 (m, 1H), 8.20-8.12 (m, 1H), 7.12-7.10 (m, 1H), 7.08-7.07 (m, 1H), 3.78-3.75 (m, 7H), 3.54-3.48 (m, 4H), 2.20 (s, 3H).

MS: 360.1 [(M+H)⁺]

### Step D:

To a solution of 4-[2-(4-chloro-2-methoxy-6-methyl-phenyl)-1H-imidazo[4,5-b]pyrazin-5-yl]morpholine (60 mg, 166.75 μmol, 1.0 eq) in DCM (3 mL) was added BBr₃ (125.33 mg, 500.26 μmol, 48.20 μL, 3.0 eq) at 0° C. The resulting reaction mixture was stirred at 25° C. for 1 h. The reaction mixture was filtered, concentrated by N₂ to give a residue. The residue was purified by prep-HPLC (FA condition, column: Phenomenex Luna C18 100 mm×30 mm×5 μm; mobile phase: [H₂O (0.2% FA)-ACN]; gradient: 20%-50% B over 8.0 min) to give 5-chloro-3-methyl-2-(5-morpholino-1H-imidazo[4,5-b]pyrazin-2-yl)phenol (16.44 mg, 27%) as a yellow solid.

¹H NMR (400 MHz, DMSO-d₆) δ=8.17 (s, 1H), 6.90 (s, 1H), 6.87 (d, 1H), 3.77-3.75 (m, 4H), 3.53-3.50 (m, 4H), 2.29 (s, 3H).

MS: 346.0 [(M+H)⁺]

### Example 74: (R)-5-fluoro-3-methyl-2-(1-methyl-5-(methyl(1-methylpyrrolidin-3-yl)amino)-1H-imidazo[4,5-b]pyrazin-2-yl)phenol formate Salt

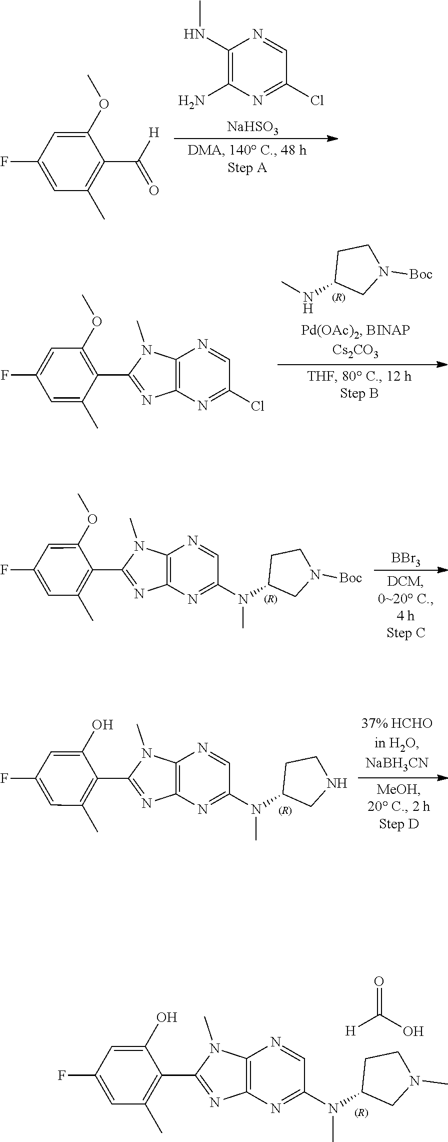

### Step A:

To a solution of 5-chloro-N2-methyl-pyrazine-2,3-diamine (300 mg, 1.89 mmol, 1 eq) in DMA (3 mL) was added NaHSO₃ (393.70 mg, 3.78 mmol, 266.01 μL, 2 eq) and 4-fluoro-2-methoxy-6-methyl-benzaldehyde (318.11 mg, 1.89 mmol, 1 eq). The mixture was stirred at 140° C. for 48 h. The reaction mixture was partitioned between DCM 30 mL and H₂O 30 mL. The solution was extracted with DCM (20 mL×3) and the organic phase was separated, washed with brine 50 mL, dried over Na₂SO₄, filtered and concentrated under reduced pressure to give a residue. The residue was purified by prep-TLC (SiO₂, PE:EtOAc=3:1, TM Rf=0.51). 5-Chloro-2-(4-fluoro-2-methoxy-6-methyl-phenyl)-1-methyl-imidazo[4,5-b]pyrazine (300 mg, 90%) was obtained as light yellow solid.

MS: 307.1 [(M+H)⁺].

### Step B:

To the solution of 5-chloro-2-(4-fluoro-2-methoxy-6-methyl-phenyl)-1-methyl-imidazo[4,5-b]pyrazine (150 mg, 489.04 μmol, 1 eq) in THF (8 mL) was added tert-butyl (3R)-3-(methylamino)pyrrolidine-1-carboxylate (195.89 mg, 978.08 μmol, 2 eq), Pd(OAc)₂ (10.98 mg, 48.90 μmol, 0.1 eq), BINAP (60.90 mg, 97.81 μmol, 0.2 eq), and Cs₂CO₃ (478.02 mg, 1.47 mmol, 3 eq) and the reaction mixture was stirred at 80° C. for 12 h in a sealed tube. After completion, the solvent was removed by concentration to give the crude product. The residue was purified by prep-TLC (SiO₂, DCM:MeOH=20:1). tert-Butyl (3R)-3-[[2-(4-fluoro-2-methoxy-6-methyl-phenyl)-1-methyl-imidazo[4,5-b]pyrazin-5-yl]-methyl-amino]pyrrolidine-1-carboxylate (100 mg, 43%) was obtained as a yellow oil.

¹H NMR (400 MHz, DMSO-d₆) δ=8.04 (s, 1H), 7.03-6.95 (m, 1H), 6.89 (dd, 1H), 5.15 (br s, 1H), 3.76 (s, 3H), 3.66-3.51 (m, 2H), 3.48 (s, 3H), 3.29-3.19 (m, 2H), 2.99 (s, 3H), 2.10 (s, 3H), 1.99 (s, 2H), 1.41 (br s, 9H).

MS: 471.3 [(M+H)⁺].

### Step C:

To a solution of tert-butyl (3R)-3-[[2-(4-fluoro-2-methoxy-6-methyl-phenyl)-1-methyl-imidazo[4,5-b]pyrazin-5-yl]-methyl-amino]pyrrolidine-1-carboxylate (100 mg, 212.52 μmol, 1 eq) in DCM (3 mL) was added BBr₃ (266.21 mg, 1.06 mmol, 102.39 μL, 5 eq) at 0° C. and the reaction mixture was stirred at 20° C. for 4 h. The reaction mixture was poured onto 5 mL sat. NaHCO₃ and then adjusted the pH to 7. Then, the resulting solution was extracted with DCM (10 mL×4). The combined organic layers were washed successively with water (10 mL×2) and brine (10 mL×1), dried over anhydrous Na₂SO₄, filtered, and concentrated to give a residue. The residue was used for the next step directly without further purification.

MS: 357.2 [(M+H)⁺].

### Step D:

To the solution of 5-fluoro-3-methyl-2-[1-methyl-5-[methyl-[(3R)-pyrrolidin-3-yl]amino]imidazo[4,5-b]pyrazin-2-yl]phenol (100 mg, 280.59 μmol, 1 eq) in MeOH (3 mL) was added 37% HCHO in H₂O (113.85 mg, 1.40 mmol, 104.45 μL, 37% purity, 5 eq). The reaction mixture was stirred at 20° C. for 1 h, then NaBH₃CN (88.16 mg, 1.40 mmol, 5 eq) was added, and the reaction mixture was stirred at 20° C. for 2 h. The reaction mixture was poured onto 5 mL saturated NaHCO₃ and then the pH was adjusted to 7. Then, the resulting solution was extracted with DCM (10 mL×4). The combined organic layers were washed successively with water (10 mL×2) and brine (10 mL×1), dried over anhydrous Na₂SO₄, filtered, and concentrated to give a residue. The residue was purified by prep-HPLC (column: Phenomenex Luna C18 100 mm×30 mm×5 μm; mobile phase: [H₂O (0.2% FA)-ACN]; gradient: 10%-40% B over 8.0 min) to give 5-fluoro-3-methyl-2-[1-methyl-5-[methyl-[(3R)-1-methylpyrrolidin-3-yl]amino]imidazo[4,5-b]pyrazin-2-yl]phenol formate salt (14 mg, 18% over two steps) as a white solid.

¹H NMR (400 MHz, DMSO-d₆) δ=10.91-10.12 (m, 1H), 7.96 (s, 1H), 6.72 (dd, 1H), 6.64 (dd, 1H), 5.24 (br s, 1H), 3.51 (s, 3H), 3.03 (s, 3H), 2.86 (br s, 1H), 2.74 (br s, 1H), 2.65-2.55 (m, 1H), 2.39-2.34 (m, 1H), 2.34-2.31 (m, 3H), 2.26-2.13 (m, 1H), 2.09 (s, 3H), 1.78 (br s, 1H).

MS: 371.3 [(M+H)⁺].

### Example 75: 1-[2-[2-hydroxy-6-methyl-4-(trifluoromethyl)phenyl]-1-methyl-imidazo[4,5-b]pyrazin-5-yl]-4-methyl-piperidin-4-ol

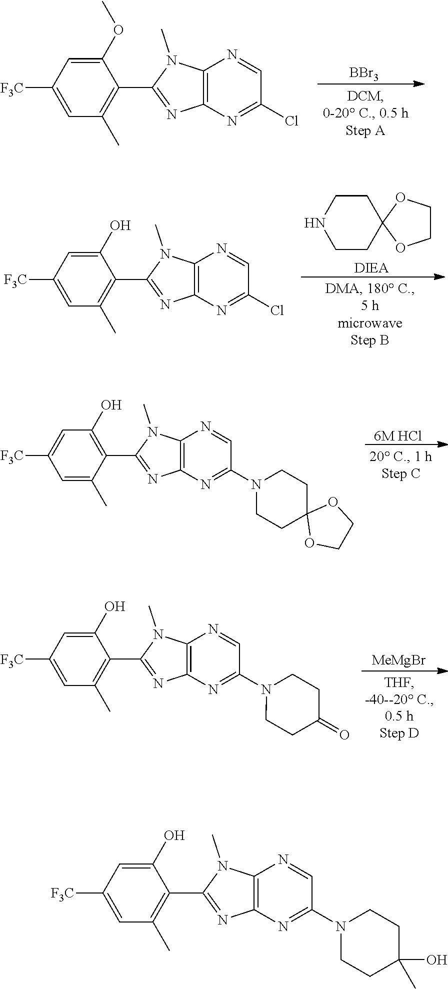

### Step A:

To a solution of 5-chloro-2-[2-methoxy-6-methyl-4-(trifluoromethyl)phenyl]-1-methyl-imidazo[4,5-b]pyrazine (500 mg, 1.40 mmol, 1 eq) in DCM (7 mL) was added BBr₃ (1.76 g, 7.01 mmol, 675.27 μL, 5 eq) at 0° C. The mixture was stirred at 20° C. for 0.5 h. The reaction mixture was poured onto 10 mL H₂O and then the pH was adjusted to 7 with saturated NaHCO₃. Then, the resulting solution was extracted with EtOAc (10 mL×2). The combined organic layers were washed successively with water (10 mL×2) and brine (20 mL×1), dried over anhydrous Na₂SO₄, and concentrated to give a residue. The residue was purified by column chromatography (SiO₂, petroleum ether/ethyl acetate=1/0 to 80/20). 2-(5-Chloro-1-methyl-imidazo[4,5-b]pyrazin-2-yl)-3-methyl-5-(trifluoromethyl)phenol (400 mg, 83%) was obtained as a yellow solid.

1H NMR (400 MHz, DMSO-d6) δ=10.89 (br s, 1H), 8.59 (s, 1H), 7.27 (s, 1H), 7.16 (s, 1H), 3.65 (s, 3H), 2.20 (s, 3H)

MS: 343.2 [(M+H)⁺]

### Step B:

To a solution of 2-(5-chloro-1-methyl-imidazo[4,5-b]pyrazin-2-yl)-3-methyl-5-(trifluoromethyl)phenol (200 mg, 583.60 μmol, 1 eq) and 1,4-dioxa-8-azaspiro[4.5]decane (1.68 g, 11.70 mmol, 1.5 mL, 20.05 eq) in DMA (1.5 mL) was added DIEA (377.12 mg, 2.92 mmol, 508.25 μL, 5 eq). The mixture was stirred at 180° C. for 5 h in a microwave device. The reaction mixture was quenched by addition of H₂O 20 mL, and then extracted with EtOAc (10 mL×3). The combined organic layers were washed with brine 20 mL, dried over Na₂SO₄, filtered and concentrated under reduced pressure to give a residue. The residue was purified by prep-TLC (SiO₂, PE:EtOAc=0:1). 2-[5-(1,4-Dioxa-8-azaspiro[4.5]decan-8-yl)-1-methyl-imidazo[4,5-b]pyrazin-2-yl]-3-methyl-5-(trifluoromethyl)phenol (120 mg, 46% yield) was obtained as a yellow solid.

MS: 450.1 [(M+H)⁺]

### Step C:

To 2-[5-(1,4-dioxa-8-azaspiro[4.5]decan-8-yl)-1-methyl-imidazo[4,5-b]pyrazin-2-yl]-3-methyl-5-(trifluoromethyl)phenol (120 mg, 267.01 μmol, 1 eq) was added an aqueous solution of 6 M HCl (2 mL). The mixture was stirred at 25° C. for 1 h. The reaction mixture was poured onto 20 mL H₂O and then the pH was adjusted to 7 with saturated NaHCO₃. Then, the resulting solution was extracted with EtOAc (10 mL×2). The combined organic layers were washed successively with water (20 mL×2) and brine (20 ml×1), dried over anhydrous Na₂SO₄, and concentrated to give a residue. The residue was used for the next step directly without further purification.

MS: 406.1 [(M+H)⁺]

### Step D:

To a solution of 1-[2-[2-hydroxy-6-methyl-4-(trifluoromethyl)phenyl]-1-methyl-imidazo[4,5-b]pyrazin-5-yl]piperidin-4-one (100 mg, 172.68 μmol, 1 eq) in THF (2 mL) was added MeMgBr (3 M, 1.5 mL, 26.06 eq) at −40° C. The mixture was stirred at −40 to −20° C. for 1 h. The reaction mixture was quenched by addition of H₂O (10 mL), and then extracted with EtOAc (4 mL×3). The combined organic layers were washed with brine 10 mL, dried over Na₂SO₄, filtered and concentrated under reduced pressure to give a residue. The residue was purified by prep-TLC (SiO₂, PE:EtOAc=0:1), then by prep-HPLC column: Waters Xbridge Prep OBD C18 150 mm×40 mm×10 μm; mobile phase: [H₂O (10 mM NH₄HCO₃)-ACN]; gradient: 30%-60% B over 6.0 min. 1-[2-[2-Hydroxy-6-methyl-4-(trifluoromethyl)phenyl]-1-methyl-imidazo[4,5-b]pyrazin-5-yl]-4-methyl-piperidin-4-ol (17.29 mg, 24% over two steps) was obtained as an off-white solid.

¹H NMR (400 MHz, DMSO-d₆) δ=10.67 (br s, 1H), 8.18 (s, 1H), 7.22 (s, 1H), 7.13 (s, 1H), 4.38 (s, 1H), 3.89 (td, 2H), 3.54 (s, 3H), 3.45-3.39 (m, 2H), 2.18 (s, 3H), 1.60-1.53 (m, 4H), 1.17 (s, 3H).

MS: 422.2 [(M+H)⁺]

### Example 76: racemate 5-chloro-3-methyl-2-[1-methyl-5-(6-methyl-3,3a,4,5,7,7a-hexahydro-2H-pyrrolo[2,3-c]pyridin-1-yl)imidazo[4,5-b]pyrazin-2-yl]phenol

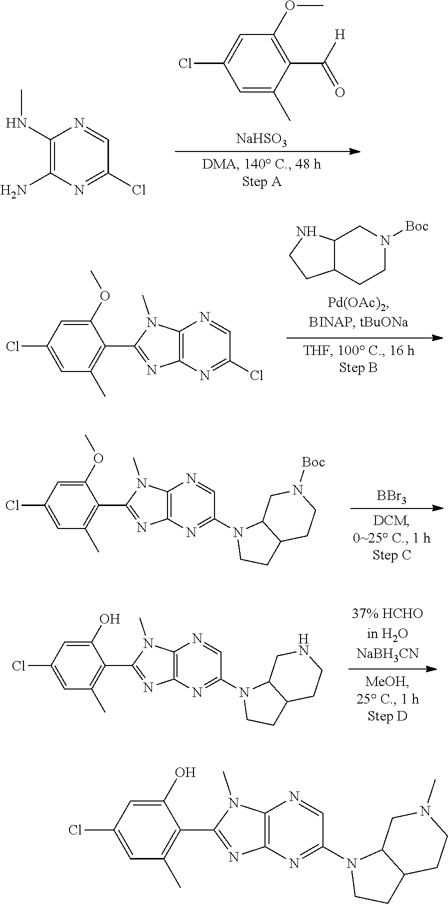

### Step A:

To a solution of 5-chloro-N2-methyl-pyrazine-2,3-diamine (601.30 mg, 3.79 mmol, 1.0 eq) and 4-chloro-2-methoxy-6-methyl-benzaldehyde (700 mg, 3.79 mmol, 1.0 eq) in DMA (20 mL) was added NaHSO₃ (789.11 mg, 7.58 mmol, 533.18 μL, 2.0 eq), the reaction mixture was stirred at 140° C. for 48 h under N₂. The reaction mixture was poured onto 80 mL H₂O. Then, the resulting solution was extracted with EtOAc (50 mL×2). The combined organic layers were washed successively with water (50 mL×2) and brine (50 mL×1), dried over anhydrous Na₂SO₄, filtered, and concentrated to give a residue. The residue was triturated with 50 mL PE:EtOAc=3:1 and filtered to afford a solid. 5-chloro-2-(4-chloro-2-methoxy-6-methyl-phenyl)-1-methyl-imidazo[4,5-b]pyrazine (0.8 g, 65%) was obtained as a yellow solid.

1H NMR (400 MHz, DMSO-d6) δ=8.58 (s, 1H), 7.21 (s, 1H), 7.17 (s, 1H), 3.79 (s, 3H), 3.59 (s, 3H), 2.13 (s, 3H).

MS: 323.1 [(M+H)₁]

### Step B:

To the solution of 5-chloro-2-(4-chloro-2-methoxy-6-methyl-phenyl)-1-methyl-imidazo[4,5-b]pyrazine (400 mg, 1.24 mmol, 1.0 eq) and tert-butyl 1,2,3,3a,4,5,7,7a-octahydropyrrolo[2,3-c]pyridine-6-carboxylate (560.22 mg, 2.48 mmol, 2.0 eq) in THF (10 mL) was added t-BuONa (237.89 mg, 2.48 mmol, 2.0 eq), BINAP (154.14 mg, 247.54 μmol, 0.2 eq), and Pd(OAc)₂ (27.79 mg, 123.77 μmol, 0.1 eq), the reaction mixture was stirred at 100° C. for 16 h under N₂. The reaction mixture was poured onto 80 mL H₂O. Then, the resulting solution was extracted with EtOAc (50 mL×2). The combined organic layers were washed successively with water (50 mL×2) and brine (50 mL×1), dried over anhydrous Na₂SO₄, filtered, and concentrated to give a residue. The residue was purified by column chromatography (SiO₂, petroleum ether/ethyl acetate=6/1 to 1/1) to give tert-butyl 1-[2-(4-chloro-2-methoxy-6-methyl-phenyl)-1-methyl-imidazo[4,5-b]pyrazin-5-yl]-3,3a,4,5,7,7a-hexahydro-2H-pyrrolo[2,3-c]pyridine-6-carboxylat e (0.27 g, 34% yield) as a yellow solid.

MS: 513.2 [(M+H)⁺]

### Step C:

To a solution of tert-butyl 1-[2-(4-chloro-2-methoxy-6-methyl-phenyl)-1-methyl-imidazo[4,5-b]pyrazin-5-yl]-3,3a,4,5,7,7a-hexahydro-2H-pyrrolo[2,3-c]pyridine-6-carboxylate (250 mg, 389.84 μmol, 1.0 eq) in DCM (8 mL) was added BBr₃ (488.32 mg, 1.95 mmol, 187.82 μL, 5.0 eq) at 0° C. The resulting reaction mixture was stirred at 25° C. for 1 h. The reaction mixture was quenched with 50 mL of saturated aqueous NaHCO₃. The resulting solution was extracted with EtOAc (30 mL×2). The combined organic layers were washed successively with water (30 mL×2) and brine (30 mL×1), dried over anhydrous Na₂SO₄, filtered, and concentrated to give a residue. The residue was purified by prep-TLC (SiO₂, DCM:MeOH=10:1) to give 2-[5-(2,3,3a,4,5,6,7,7a-octahydropyrrolo[2,3-c]pyridin-1-yl)-1-methyl-imidazo[4,5-b]pyrazin-2-yl]-5-chloro-3-methyl-phenol (120 mg, 77%) as a yellow solid.

MS: 399.1 [(M+H)⁺]

### Step D:

To a solution of 2-[5-(2,3,3a,4,5,6,7,7a-octahydropyrrolo[2,3-c]pyridin-1-yl)-1-methyl-imidazo[4,5-b]pyrazin-2-yl]-5-chloro-3-methyl-phenol (0.1 g, 250.70 μmol, 1.0 eq) in MeOH (2 mL) was added 37% HCHO (101.72 mg, 1.25 mmol, 93.32 μL, 5.0 eq) in H₂O, the reaction mixture was stirred at 25° C. for 0.5 h. Then, NaBH₃CN (78.77 mg, 1.25 mmol, 5.0 eq) was added and the reaction mixture was stirred at 25° C. for 0.5 h under N₂. The reaction mixture was poured onto 80 mL saturated aqueous NaHCO₃. Then, the resulting solution was extracted with EtOAc (50 mL×2). The combined organic layers were washed successively with water (50 mL×2) and brine (50 mL×1), dried over anhydrous Na₂SO₄, filtered, and concentrated to give a residue. The residue was purified by prep-HPLC (neutral condition, column: Waters Xbridge Prep OBD C18 150 mm×40 mm×10 μm; mobile phase: [H₂O (10 mM NH₄HCO₃)-ACN]; gradient: 25%-55% B over 8.0 min) to give 5-chloro-3-methyl-2-[1-methyl-5-(6-methyl-3,3a,4,5,7,7a-hexahydro-2H-pyrrolo[2,3-c]pyridin-1-yl)imidazo[4,5-b]pyrazin-2-yl]phenol (49.37 mg, 47% yield) as a white solid.

¹H NMR (400 MHz, DMSO-d₆) δ=10.43 (br s, 1H), 7.73 (s, 1H), 6.95 (d, 1H), 6.89 (d, 1H), 4.20 (br d, 1H), 3.59-3.56 (m, 1H), 3.50 (s, 3H), 3.42-3.37 (m, 1H), 3.08 (br s, 1H), 2.40-2.31 (m, 1H), 2.17 (br s, 3H), 2.13-2.05 (m, 5H), 2.05-2.01 (m, 1H), 2.00-1.88 (m, 2H), 1.77 (br d, 1H), 1.69 (br d, 1H).

MS: 413.2 [(M+H)⁺]

### Examples 77A and 77B: enantiopure 5-chloro-3-methyl-2-[1-methyl-5-(6-methyl-3,3a,4,5,7,7a-hexahydro-2H-pyrrolo[2,3-c]pyridin-1-yl)imidazo[4,5-b]pyrazin-2-yl]phenol

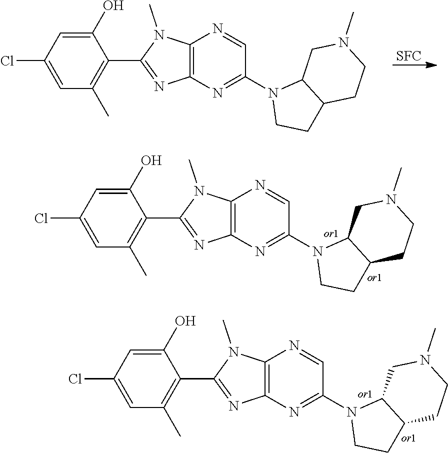

Racemate 5-chloro-3-methyl-2-[1-methyl-5-(6-methyl-3,3a,4,5,7,7a-hexahydro-2H-pyrrolo[2,3-c]pyridin-1-yl)imidazo[4,5-b]pyrazin-2-yl]phenol was separated by SFC (column: DAICEL CHIRALPAK AD (250 mm×30 mm×10 μm); mobile phase: [CO₂—EtOH (0.1% NH₃ H₂O)]; B %: 25%, isocratic elution mode) to give enantiopure 5-chloro-3-methyl-2-[1-methyl-5-[rel-(3aS,7aR)-6-methyl-3,3a,4,5,7,7a-hexahydro-2H-pyrrolo[2,3-c]pyridin-1-yl]imidazo[4,5-b]pyrazin-2-yl]phenol example 77A as a white solid (first eluting peak).

¹H NMR (400 MHz, DMSO-d₆) δ=10.49 (br s, 1H), 7.73 (s, 1H), 6.92 (d, 1H), 6.87 (d, 1H), 4.19 (br d, 1H), 3.56-3.50 (m, 4H), 3.39-3.37 (m, 1H), 3.08 (br s, 1H), 2.35-2.34 (m, 1H), 2.16 (br s, 3H), 2.08-2.04 (m, 5H), 2.02-1.98 (m, 1H), 1.95-1.92 (m, 2H), 1.77-1.75 (br d, 1H), 1.70-1.67 (br d, 1H).

MS: 413.2 [(M+H)⁺];

and enantiopure 5-chloro-3-methyl-2-[1-methyl-5-[rel-(3aS,7aR)-6-methyl-3,3a,4,5,7,7a-hexahydro-2H-pyrrolo[2,3-c]pyridin-1-yl]imidazo[4,5-b]pyrazin-2-yl]phenol example 77B as a white solid (second eluting peak).

¹H NMR (400 MHz, DMSO-d₆) δ=10.48 (br s, 1H), 7.72 (s, 1H), 6.91 (d, 1H), 6.86 (d, 1H), 4.20 (br d, 1H), 3.58-3.54 (m, 1H), 3.50 (s, 3H), 3.42-3.38 (m, 1H), 3.08 (br s, 1H), 2.37-2.32 (m, 1H), 2.16 (br s, 3H), 2.13-2.04 (m, 5H), 2.00-1.98 (m, 1H), 1.96-1.89 (m, 2H), 1.77-1.75 (br d, 1H), 1.71-1.68 (br d, 1H).

MS: 413.2 [(M+H)⁺]

### Example 78: 1-[2-(4-chloro-2-hydroxy-6-methyl-phenyl)-1-methyl-imidazo[4,5-b]pyrazine-5-yl]piperidin-4-ol

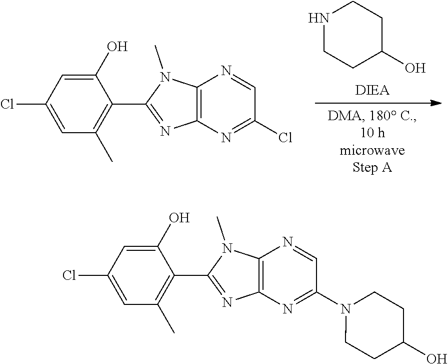 [SMILES: Cc1cc(Cl)cc(O)c1-c1nc2nc(Cl)cnc2n1C ; OC1CCNCC1 ; Cc1cc(Cl)cc(O)c1-c1nc2nc(N3CCC(O)CC3)cnc2n1C]

### Step A:

To a solution of 5-chloro-2-(5-chloro-1-methyl-imidazo[4,5-b]pyrazin-2-yl)-3-methyl-phenol (60 mg, 194.08 μmol, 1 eq) in DMA (1 mL) was added piperidin-4-ol (392.61 mg, 3.88 mmol, 20 eq), and DIEA (125.42 mg, 970.40 μmol, 169.03 μL, 5 eq) and the resulting reaction mixture was stirred at 180° C. for 10 h in a microwave device. The reaction mixture was purified by prep-HPLC (column: Waters Xbridge Prep OBD C18 150 mm×40 mm×10 μm; mobile phase: [H₂O (10 mM NH₄HCO₃)-ACN]; gradient: 20%-50% B over 6.0 min) to give 1-[2-(4-chloro-2-hydroxy-6-methyl-phenyl)-1-methyl-imidazo[4,5-b]pyrazine-5-yl]piperidin-4-ol (33.81 mg, 45%) as a yellow solid.

¹H NMR (400 MHz, DMSO-d₆) δ=10.42 (br s, 1H), 8.16 (s, 1H), 6.94 (s, 1H), 6.88 (s, 1H), 4.71 (br d, 1H), 4.04 (br d, 2H), 3.72 (br s, 1H), 3.52 (s, 3H), 3.17 (br t, 2H), 2.09 (s, 3H), 1.89-1.75 (m, 2H), 1.43 (br d, 2H).

### Example 79: 1: 3-methyl-2-(1-methyl-5-morpholino-imidazo[4,5-b]pyridin-2-yl)-5-(trifluoromethyl)phenol

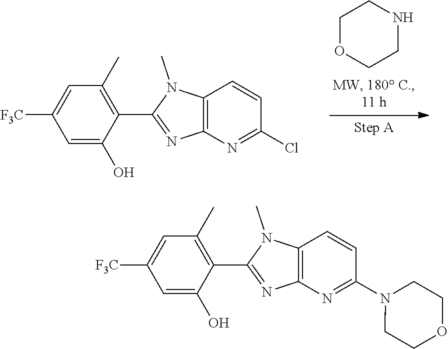 [SMILES: Cc1cc(C(F)(F)F)cc(O)c1-c1nc2nc(Cl)ccc2n1C ; C1COCCN1 ; Cc1cc(C(F)(F)F)cc(O)c1-c1nc2nc(N3CCOCC3)ccc2n1C]

### Step A:

In a microwave vial, 2-(5-chloro-1-methyl-imidazo[4,5-b]pyridin-2-yl)-3-methyl-5-(trifluoromethyl)-phenol (52 mg, 0.148 mmol) was suspended in morpholine (3.0 mL, 34.4 mmol). The vial was irradiated in the microwave device at 150° C. for 11 h. The mixture was cooled, diluted with water and extracted three times with dichloromethane. The combined organic layers were dried over Na₂SO₄, filtered and concentrated under reduced pressure. The crude product was purified by flash chromatography (Silica 10 g column, 40-100% ethyl acetate in heptane) to afford 3-methyl-2-(1-methyl-5-morpholino-imidazo[4,5-b]pyridin-2-yl)-5-(trifluoromethyl)phenol as a white solid. 1H NMR (80 MHz, DMSO-d6) δ 10.57 (s, 1H), 7.89 (d, 1H), 7.25-7.07 (m, 2H), 6.90 (d, 1H), 3.85-3.64 (m, 4H), 3.57-3.36 (m, 7H), 2.14 (s, 3H).

MS: 393.13 [(M+H)⁺]

### Example 80: 1-[2-(4-chloro-2-hydroxy-6-methyl-phenyl)-1-methyl-imidazo[4,5-b]pyrazin-5-yl]-4-methyl-piperidin-4-ol

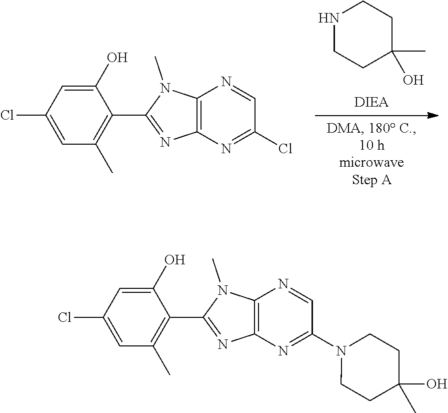

### Step A:

To a solution of 5-chloro-2-(5-chloro-1-methyl-imidazo[4,5-b]pyrazin-2-yl)-3-methyl-phenol (60 mg, 194.08 μmol, 1 eq) in DMA (1 mL) was added 4-methylpiperidin-4-ol (447.06 mg, 3.88 mmol, 20 eq), and DIEA (125.42 mg, 970.40 μmol, 169.03 μL, 5 eq) and the resulting reaction mixture was stirred at 180° C. for 10 h in microwave device. The reaction mixture was purified by prep-HPLC (column: Phenomenex Luna C18 100 mm×30 mm×5 μm; mobile phase: [H₂O (0.2% FA)-ACN]; gradient: 10%-40% B over 8.0 min) to give 1-[2-(4-chloro-2-hydroxy-6-methyl-phenyl)-1-methyl-imidazo[4,5-b]pyrazin-5-yl]-4-methyl-piperidin-4-ol (40.46 mg, 53%) as a yellow solid.

¹H NMR (400 MHz, DMSO-d₆) δ=10.43 (br s, 1H), 8.15 (s, 1H), 6.94 (s, 1H), 6.88 (s, 1H), 4.36 (s, 1H), 3.95-3.81 (m, 2H), 3.51 (s, 3H), 3.45-3.34 (m, 2H), 2.09 (s, 3H), 1.55 (br t, 4H), 1.16 (s, 3H).

MS: 388.1 [(M+H)⁺]

### Example 81: 5-chloro-3-methyl-2-[1-methyl-5-[methyl-[(3R)-1-methylpyrrolidin-3-yl]amino]imidazo-[4,5-b]pyrazin-2-yl]phenol

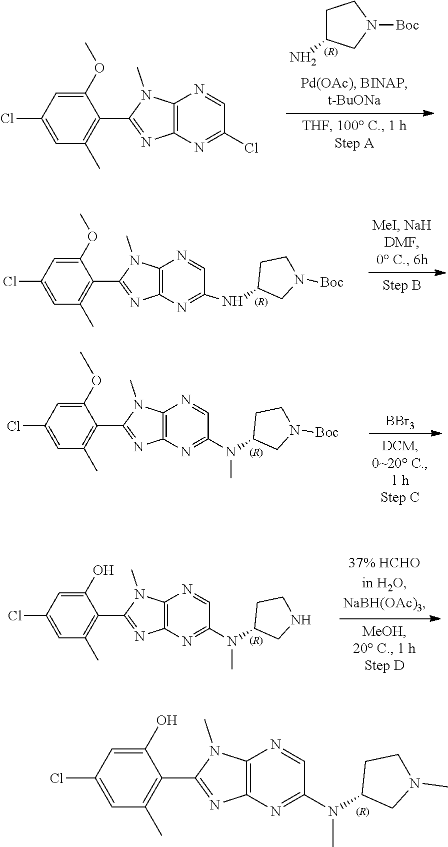

### Step A:

To a solution of 5-chloro-2-(4-chloro-2-methoxy-6-methyl-phenyl)-1-methyl-imidazo[4,5-b]pyrazine (500 mg, 1.55 mmol, 1 eq) in THF (5 mL) was added tert-butyl (3R)-3-aminopyrrolidine-1-carboxylate (288.16 mg, 1.55 mmol, 262.44 μL, 1 eq), BINAP (192.67 mg, 309.43 μmol, 0.2 eq), t-BuONa (297.37 mg, 3.09 mmol, 2 eq) and Pd(OAc)₂ (34.73 mg, 154.71 μmol, 0.1 eq) and the resulting reaction mixture was stirred at 100° C. for 1 h. The reaction mixture was poured onto 50 mL H₂O, followed by 25 mL EtOAc. After that, the aqueous phase was separated and extracted with EtOAc (20 mL×2). The combined organic layers were washed successively with water (20 mL×2) and brine (20 ml×1), dried over anhydrous Na₂SO₄, filtered, and concentrated to give a residue. The residue was purified by column chromatography (SiO₂, petroleum ether/ethyl acetate=100/1 to 0/1) to give tert-butyl (3R)-3-[[2-(4-chloro-2-methoxy-6-methyl-phenyl)-1-methyl-imidazo[4,5-b]pyrazin-5-yl]amino]pyrrolidine-1-carboxylate (630 mg, 52% yield) as a yellow solid.

¹H NMR (400 MHz, DMSO-d₆) δ=7.78 (s, 1H), 7.20-7.19 (m, 1H), 7.21-7.15 (m, 1H), 7.15 (s, 1H), 4.43-4.27 (m, 1H), 3.77 (s, 3H), 3.68-3.59 (m, 1H), 3.45 (s, 3H), 3.42-3.35 (m, 2H), 3.26-3.10 (m, 2H), 2.10 (d, J=1.8 Hz, 3H), 1.93-1.82 (m, 1H), 1.44-1.35 (m, 9H).

MS: 473.2 [(M+H)⁺]

### Step B:

To a solution of tert-butyl (3R)-3-[[2-(4-chloro-2-methoxy-6-methyl-phenyl)-1-methyl-imidazo[4,5-b]pyrazin-5-yl]amino]pyrrolidine-1-carboxylate (500 mg, 1.06 mmol, 1 eq) in DMF (3 mL) was added NaH (42.28 mg, 1.06 mmol, 60% purity, 1 eq) at 0° C. for 0.5 h, then Mel (150.05 mg, 1.06 mmol, 65.81 μL, 1 eq) was added at 0° C. and the resulting reaction mixture was stirred at 0° C. for 5.5 h. The reaction mixture was poured onto 20 mL saturated NH₄Cl, followed by 10 mL EtOAc. After that, the aqueous phase was separated and extracted with EtOAc (20 mL×2). The combined organic layers were washed successively with water (20 mL×2) and brine (20 ml×1), dried over anhydrous Na₂SO₄, filtered, and concentrated to give a residue. The residue was purified by column chromatography (SiO₂, petroleum ether/ethyl acetate=100/1 to 0/1) to deliver tert-butyl (3R)-3-[[2-(4-chloro-2-methoxy-6-methyl-phenyl)-1-methyl-imidazo[4,5-b]pyrazin-5-yl]-methyl-amino] pyrrolidine-1-carboxylate (310 mg, 60.%) as a yellow oil.

¹H NMR (400 MHz, DMSO-d6) δ=8.05 (s, 1H), 7.19-7.14 (m, 1H), 7.14-7.09 (m, 1H), 5.15 (br d, J=6.9 Hz, 1H), 3.77 (s, 3H), 3.59-3.52 (m, 1H), 3.48 (s, 3H), 3.46-3.40 (m, 1H), 3.30-3.12 (m, 2H), 3.03-2.97 (m, 3H), 2.10 (s, 3H), 2.07 (br s, 2H), 1.41 (br s, 9H).

MS: 487.4 [(M+H)⁺]

### Step C:

To a solution of tert-butyl (3R)-3-[[2-(4-chloro-2-methoxy-6-methyl-phenyl)-1-methyl-imidazo[4,5-b]pyrazin-5-yl]-methyl-amino]pyrrolidine-1-carboxylate (250 mg, 513.35 μmol, 1 eq) in DCM (3 mL) was added BBr₃ (643.04 mg, 2.57 mmol, 247.32 μL, 5 eq) at 0° C. and the resulting reaction mixture was stirred at 20° C. for 1 h. The reaction mixture was poured onto 20 mL H₂O and adjusted the pH to 7 with saturated NaHCO₃. Then, the resulting solution was extracted with EtOAc (20 mL×2). The combined organic layers were washed successively with water (20 mL×2) and brine (20 ml×1), dried over anhydrous Na₂SO₄, filtered, and concentrated to give a residue. The residue was used for the next step directly without further purification (yellow oil).

MS: 373.3 [(M+H)⁺]

### Step D:

To a solution of 5-chloro-3-methyl-2-[1-methyl-5-[methyl-[(3R)-pyrrolidin-3-yl]amino]imidazo[4,5-b]pyrazin-2-yl]phenol (70 mg, 187.74 μmol, 1 eq) in MeOH (1 mL) was added HCHO (11.27 mg, 375.49 μmol, 10.34 μL, 37% purity, 2 eq) in H₂O, and NaBH(OAc)₃ (59.69 mg, 281.61 μmol, 1.5 eq) and the resulting reaction mixture was stirred at 20° C. for 1 h. The reaction mixture was poured onto 20 mL H₂O and the pH was adjusted to 7 with saturated NaHCO₃. Then, the resulting solution was extracted with EtOAc (10 mL×2). The combined organic layers were washed successively with water (20 mL×2) and brine (20 ml×1), dried over anhydrous Na₂SO₄, filtered, and concentrated to give a residue. The residue was purified by prep-HPLC (column: Phenomenex Luna C18 100 mm×30 mm×5 μm; mobile phase: [H₂O (0.2% FA)-ACN]; gradient: 1%-30% B over 8.0 min) to give 5-chloro-3-methyl-2-[1-methyl-5-[methyl-[(3R)-1-methylpyrrolidin-3-yl]amino]imidazo[4,5-b]pyrazin-2-yl]phenol (25.3 mg, 35%) as an off-white solid.

¹H NMR (400 MHz, DMSO-d₆) δ=10.75-9.83 (m, 1H), 7.96 (s, 1H), 6.95 (d, 1H), 6.89 (d, 1H), 5.32-5.11 (m, 1H), 3.51 (s, 3H), 3.03 (s, 3H), 2.82 (dt, 1H), 2.71 (br d, 1H), 2.58-2.52 (m, 1H), 2.30 (s, 3H), 2.28-2.23 (m, 1H), 2.22-2.12 (m, 1H), 2.08 (s, 3H), 1.75 (br d, 1H).

MS: 387.1 [(M+H)⁺].

### Example 82 2-(2-((2-hydroxyethyl)amino)-1-methyl-1H-imidazo[4,5-b]pyridin-5-yl)-5-(trifluoro-methyl)phenol

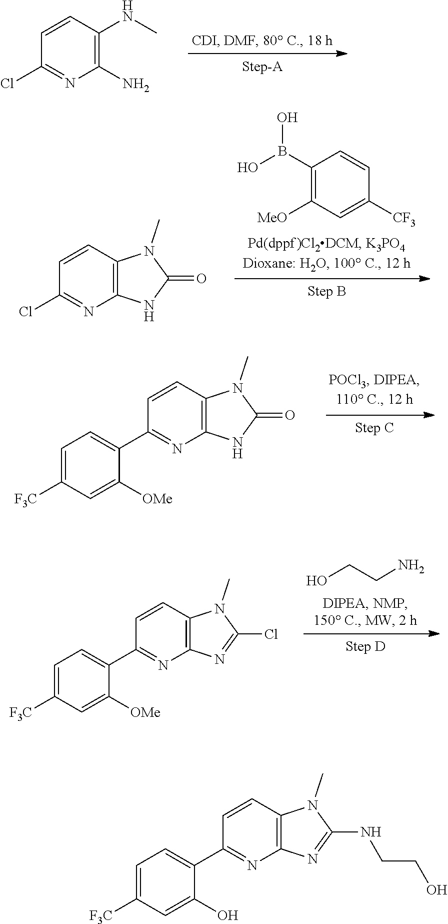

### Step A:

To a stirred solution of 6-chloro-N3-methylpyridine-2,3-diamine (4.0 g, 25.471 mmol) in DMF (40 mL) was added 1,1′-carbonyldiimidazole (8.26 g, 50.942 mmol) at RT and the mixture was heated to 80° C. for 18 h. The reaction mixture was cooled at rt then diluted with ice cold water (100 mL) and stirred for 5 min. The solid was filtered, washed with water (200 mL) and dried under vacuum to afford 5-chloro-1-methyl-1,3-dihydro-2H-imidazo[4,5-b]pyridin-2-one (3.0 g, 64.15%) as a brown solid. ¹H NMR (400 MHz DMSO-D6): δ=11.77 (m, 1H), 7.47-7.49 (d, 1H), 7.09-7.11 (d, 1H), 3.3 (d, 3H).

MS: 184.0 [(M+H)⁺].

### Step B:

To a stirred solution of 5-chloro-1-methyl-1,3-dihydro-2H-imidazo[4,5-b]pyridin-2-one (500 mg, 2.723 mmol) in 1,4-dioxane (10 mL) and water (4 mL) were added (2-methoxy-4-(trifluoromethyl)phenyl)boronic acid (718.810 mg, 3.268 mmol), and tripotassium phosphate (1156.186 mg, 5.447 mmol) at RT, the mixture was purged with nitrogen for 5 min, then Pd(dppf)Cl₂·DCM (111.201 mg, 0.136 mmol) was added at RT. The resulting mixture was stirred for 12 h at 110° C. in a sealed tube. The reaction mixture was cooled to RT, diluted with H₂O (30 mL) and stirred for 10 min and extracted with EtOAc (2×80 mL). The combined organic layers were washed with brine solution (50 mL), dried over anhydrous sodium sulfate, filtered and concentrated under reduced pressure to get a crude residue. The crude product was purified by column chromatography by using 100-200 mesh silica, 80% EtOAc in hexane. The pure fractions were evaporated to dryness to get the desired compound 5-(2-methoxy-4-(trifluoromethyl)phenyl)-1-methyl-1,3-dihydro-2H-imidazo[4,5-b]pyridin-2-one (0.3 g, 34%).

MS: 324.2 [(M+H)⁺].

### Step C:

To a stirred solution of 5-(2-methoxy-4-(trifluoromethyl)phenyl)-1-methyl-1,3-dihydro-2H-imidazo[4,5-b]pyridin-2-one (300 mg, 0.928 mmol) in POCl₃ (8 mL) was added N,N-diisopropylethylamine (119.944 mg, 0.928 mmol) at 0° C., the resulting mixture was stirred for 12 h at 110° C. in a sealed tube. The reaction mixture was cooled to RT, diluted with H₂O (20 mL) and basified with saturated NaHCO₃ solution (100 mL), stirred for 10 min and extracted with EtOAc (2×200 mL). The combined organic layers were washed with brine solution (100 mL), dried over anhydrous sodium sulfate, filtered and concentrated under reduced pressure. The resulted crude product was purified by column chromatography using 100-200 mesh silica, 50% EtOAc in hexane. Pure fractions were evaporated to get 2-chloro-5-(2-methoxy-4-(trifluoromethyl)phenyl)-1-methyl-1H-imidazo[4,5-b]pyridine (0.2 g, 62%).

### Step D:

To a stirred solution of 2-chloro-5-(2-methoxy-4-(trifluoromethyl)phenyl)-1-methyl-1H-imidazo[4,5-b]pyridine (200 mg, 0.585 mmol) in NMP (8 mL) were added 2-aminoethan-1-ol (71.502 mg, 1.171 mmol) and DIPEA (151.294 mg, 1.171 mmol) at RT. The resulting mixture was stirred for 2 h at 150° C., under microwave conditions. The reaction mixture was diluted with water (50 mL) and extracted with EtOAc (2×150 mL). The combined organic layers were separated, washed with brine solution (100 mL), dried over anhydrous sodium sulfate, filtered and concentrated under reduced pressure to get a crude product. The crude compound was purified by column chromatography using silica gel (100-200 mesh) and eluted with MeOH/DCM as a gradient. The pure fractions were collected and concentrated under reduced pressure to afford a 8:2 mixture of 2-((5-(2-methoxy-4-(trifluoromethyl)phenyl)-1-methyl-1H-imidazo[4,5-b]pyridin-2-yl)amino)ethan-1-ol and 2-(2-((2-hydroxyethyl)amino)-1-methyl-1H-imidazo[4,5-b]pyridin-5-yl)-5-(trifluoromethyl)phenol. This mixture fraction was further purified by Prep-HPLC and both of the compounds were separated (Prep HPLC method: column: YMC TRIART 100 mm 3 U buffer A: 10 mM ABC BUFFER B: ACN solvent: ACN-water-THF; mobile phase conditions (% of B): 0/20, 2/30, 12/50, 14/50, 14.10/100, 18/100, 18.10/40, 21/30; Flow: 14 mL/min ANL-MCL-PREP-017. The title compound (fraction 2) was collected and lyophilized to afford 2-(2-((2-hydroxyethyl)amino)-1-methyl-1H-imidazo[4,5-b]pyridin-5-yl)-5-(trifluoromethyl)phenol (12 mg, 7%).

¹H NMR (400 MHz DMSO-D6): δ=15.26 (s, 1H), 8.16-8.18 (d, 1H), 7.80-7.82 (d, 1H), 7.73-7.75 (d, 1H), 7.51 (bs, 1H), 7.15 (bs, 2H), 4.84 (t, 1H), 3.63 (m, 2H), 3.58 (s, 3H), 3.51 (m, 2H)

MS: 353.2 [(M+H)⁺].

### Example 83: (R)-2-(1-methyl-2-((1-methylpiperidin-3-yl)amino)-1H-imidazo[4,5-b]pyridin-5-yl)-5-(trifluoromethyl)phenol

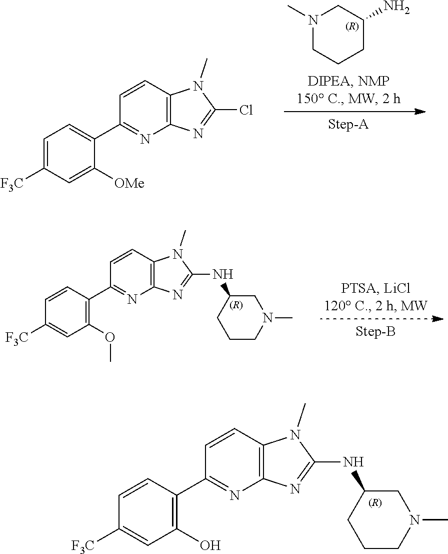

### Step-A:

To a stirred solution of 2-chloro-5-(2-methoxy-4-(trifluoromethyl)phenyl)-1-methyl-1H-imidazo[4,5-b]pyridine (400 mg, 1.171 mmol) in NMP (8 mL) were added (R)-1-methylpiperidin-3-amine (267.336 mg, 2.341 mmol) and DIPEA (453.883 mg, 3.512 mmol) at RT. The resulting mixture was stirred for 2 h at 150° C. under microwave conditions. The reaction mixture was diluted with water (50 mL) and extracted with EtOAc (2×140 mL). The combined organic layers were separated, washed with brine solution (100 mL), dried over anhydrous sodium sulfate, filtered and concentrated under reduced pressure to get a crude product. The crude product was purified by column chromatography using silica gel (100-200 mesh) and eluted with 10% MeOH/DCM as a gradient. The product was eluted at 5% MeOH/DCM. The pure fractions were collected and concentrated under reduced pressure to get compound (R)-5-(2-methoxy-4-(trifluoromethyl)phenyl)-1-methyl-N-(1-methylpiperidin-3-yl)-1H-imidazo[4,5-b]pyridin-2-amine (150 mg, 31%).

MS: 420.2 [(M+H)⁺].

### Step-B:

To a stirred solution of (R)-5-(2-methoxy-4-(trifluoromethyl)phenyl)-1-methyl-N-(1-methylpiperidin-3-yl)-1H-imidazo[4,5-b]pyridin-2-amine (200 mg, 0.477 mmol) in dimethylformamide (8 mL) were added p-toluenesulfonic acid monohydrate (453.496 mg, 2.384 mmol) and lithium chloride (101.037 mg, 2.384 mmol) and the mixture was stirred at 120° C. for 2 h, under microwave conditions. The reaction mixture was diluted with water (100 mL) and extracted with EtOAc (2×300 mL). The combined organic layer were separated, washed with brine solution (100 mL), dried over anhydrous sodium sulfate, filtered and concentrated under reduced pressure. The resultant crude compound was purified by Prep HPLC purification. (mobile phase A: 10 mM ammonium bicarbonate; mobile phase B: ACN; column: XBRIDGE C18 (25 mm×150 mm, 10 μm) gradient (% of B) 0 min (35), 11 min (58), 11.05 min (98), 14 min (98), 14.05 min (35), 17 min (35) water; flow: 25 mL/min).

¹H NMR (400 MHz, DMSO) δ 15.24 (s, 1H), 8.18 (d, 1H), 7.82 (d, 1H), 7.75 (d, 1H), 7.15 (d, 3H), 3.98 (q, 1H), 3.58 (s, 3H), 2.96 (d, 1H), 2.74-2.62 (m, 1H), 2.20 (s, 3H), 1.99-1.82 (m, 3H), 1.78-1.69 (m, 1H), 1.57 (q, 1H), 1.37 (qd, 1H).

MS: 406.3 [(M+H)⁺].

### Example 84: 1-(2-(2-hydroxy-6-methyl-4-(trifluoromethyl)phenyl)-1-methyl-1H-imidazo[4,5-b]pyrazin-5-yl)-3-methylazetidin-3-ol

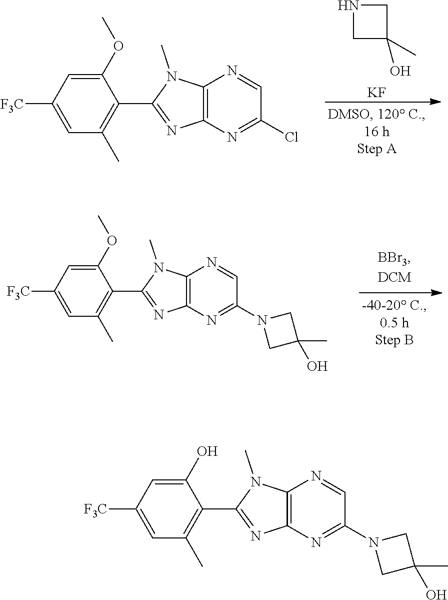

### Step A:

To the solution of 5-chloro-2-[2-methoxy-6-methyl-4-(trifluoromethyl)phenyl]-1-methyl-imidazo[4,5-b]pyrazine (200 mg, 560.65 μmol, 1 eq) in DMSO (5 mL) were added potassium fluoride (KF) (97.72 mg, 1.68 mmol, 39.40 μL, 3 eq), and 3-methylazetidin-3-ol (488.44 mg, 5.61 mmol, 10 eq) and the reaction mixture was stirred at 120° C. for 16 hr. LCMS indicated that the reaction was completed. The reaction mixture was poured onto 15 mL H₂O. Then the resulting solution was extracted with EA (15 mL×4). The combined organic layers were washed successively with water (15 mL×2) and brine (15 mL×1), dried over anhydrous Na₂SO₄, filtered, and concentrated to give a residue. The residue was purified by prep-TLC (SiO₂, petroleum ether/ethyl acetate=1:1). 1-[2-[2-Methoxy-6-methyl-4-(trifluoromethyl)phenyl]-1-methyl-imidazo[4,5-b]pyrazin-5-yl]-3-methyl-azetidin-3-ol (200 mg, 490.93 μmol, 87.56% yield) was obtained as a yellow oil.

MS: 408.0 [(M+H)⁺].

### Step B:

To the solution of 1-[2-[2-methoxy-6-methyl-4-(trifluoromethyl)phenyl]-1-methyl-imidazo[4,5-b]pyrazin-5-yl]-3-methyl-azetidin-3-ol (100 mg, 245.47 μmol, 1 eq) in DCM (2 mL) was added BBr₃ (184.48 mg, 736.40 μmol, 70.96 μL, 3 eq) at −40° C. and the reaction mixture was stirred at 20° C. for 0.5 hr. LCMS indicated that the reaction was completed. The reaction mixture was poured onto 5 mL H₂O at 0° C. Then the resulting solution was extracted with DCM (5 mL×4). The combined organic layers were washed successively with water (5 mL×2) and brine (5 mL×1), dried over anhydrous Na₂SO₄, filtered, and concentrated to give a residue. The residue was purified by prep-HPLC (column: Waters Xbridge Prep OBD C18 150 mm×40 mm×10 μm; mobile phase: [H₂O (10 mM NH₄HCO₃NH₄HCO₃)-ACN]; gradient: 20%-50% B over 8.0 min) to give the title product. 1-[2-[2-hydroxy-6-methyl-4-(trifluoromethyl)phenyl]-1-methyl-imidazo[4,5-b]pyrazin-5-yl]-3-methyl-azetidin-3-ol (8 mg, 20.16 μmol, 8.21% yield, 99.11% purity) as an off-white solid.

LC-MS (ES+, m/z): 394.1 [(M+H)+].

1H NMR (400 MHz, DMSO-d6) δ=10.72 (br s, 1H), 7.71 (s, 1H), 7.21 (s, 1H), 7.13 (s, 1H), 5.74-5.47 (m, 1H), 4.01-3.92 (m, 2H), 3.91-3.80 (m, 2H), 3.54 (s, 3H), 2.17 (s, 3H), 1.48 (s, 3H).

MS: 394.1 [(M+H)⁺].

### Examples 85A and 85B

### rel-(3R)-1-[2-(4-chloro-2-hydroxy-6-methyl-phenyl)-1-methyl-imidazo[4,5-b]pyrazin-5-yl]pyrrolidin-3-ol and rel-(3S)-1-[2-(4-chloro-2-hydroxy-6-methyl-phenyl)-1-methyl-imidazo[4,5-b]pyrazin-5-yl]pyrrolidin-3-ol

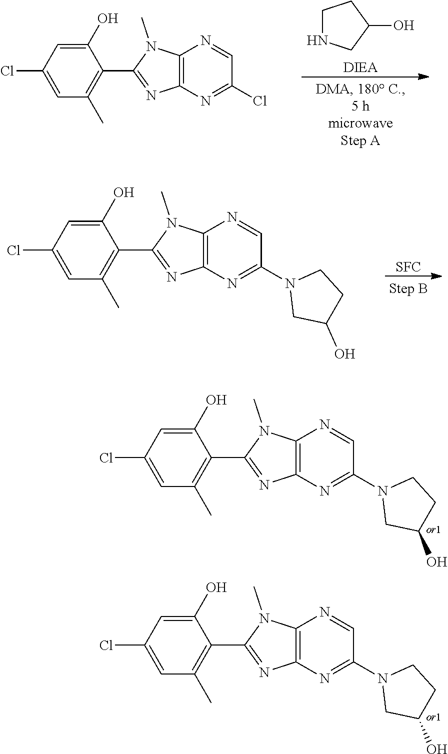

### Step A:

To the solution of 5-chloro-2-(5-chloro-1-methyl-imidazo[4,5-b]pyrazin-2-yl)-3-methyl-phenol (100 mg, 323.47 μmol, 1 eq) in DMA (2 mL) was added DIEA (209.02 mg, 1.62 mmol, 281.70 μL, 5 eq), and pyrrolidin-3-ol (563.61 mg, 6.47 mmol, 523.80 μL, 20 eq) and the reaction mixture was stirred at 180° C. for 5 hr in a microwave device. LCMS indicated that the reaction was completed. After completion, the reaction mixture was purified by prep-HPLC (column: Waters Xbridge Prep OBD C18 150 mm×40 mm×10 μm; mobile phase: [H₂O (10 mM NH₄HCO₃)-ACN]; gradient: 15%-50% B over 8.0 min) to give the title product. 1-[2-(4-chloro-2-hydroxy-6-methyl-phenyl)-1-methyl-imidazo[4,5-b]pyrazin-5-yl]pyrrolidin-3-ol (70 mg, 194.55 μmol, 60.14% yield) as a yellow solid.

MS: 360.1 [(M+H)⁺].

### Step B:

1-[2-(4-Chloro-2-hydroxy-6-methyl-phenyl)-1-methyl-imidazo[4,5-b]pyrazin-5-yl]pyrrolidin-3-ol was further separated by SFC (column: DAICEL CHIRALPAK AD (250 mm×30 mm, 10 μm); mobile phase: [CO₂-MeOH (0.1% NH3H₂O)]; B %: 24%, isocratic elution mode) to give the title product. rel-(3R)-1-[2-(4-chloro-2-hydroxy-6-methyl-phenyl)-1-methyl-imidazo[4,5-b]pyrazin-5-yl]pyrrolidin-3-ol example 85A (15.20 mg, 40.87 μmol, 24.51% yield, 96.74% purity, first eluting peak) as an off-white solid.

¹H NMR (400 MHz, DMSO-d6) δ=10.44 (br s, 1H), 7.74 (s, 1H), 6.93 (s, 1H), 6.88 (d, 1H), 5.00 (br s, 1H), 4.44 (br s, 1H), 3.63-3.53 (m, 3H), 3.51 (s, 3H), 3.43-3.39 (m, 1H), 2.08 (s, 3H), 2.06-2.01 (m, 1H), 1.99-1.90 (m, 1H).

¹H NMR (400 MHz, DMSO-d6+D2O) δ=7.71 (s, 1H), 6.90 (s, 1H), 6.85 (d, 1H), 4.42 (br d, 1H), 3.58-3.49 (m, 3H), 3.47 (s, 3H), 3.41-3.33 (m, 1H), 2.09-2.04 (m, 1H), 2.03 (s, 3H), 1.96-1.90 (m, 1H).

MS: 360.1 [(M+H)⁺].

and rel-(3S)-1-[2-(4-chloro-2-hydroxy-6-methyl-phenyl)-1-methyl-imidazo[4,5-b]pyrazin-5-yl]pyrrolidin-3-ol example 85B (23.75 mg, 64.99 μmol, 38.97% yield, 98.46% purity, second eluting peak) as an off-white solid.

¹H NMR (400 MHz, DMSO-d6) δ=10.34 (br s, 1H), 7.74 (s, 1H), 6.93 (s, 1H), 6.88 (d, 1H), 5.00 (br d, 1H), 4.44 (br s, 1H), 3.55 (br d, 3H), 3.51 (s, 3H), 3.43-3.37 (m, 1H), 2.08 (s, 3H), 2.07-2.01 (m, 1H), 1.98-1.89 (m, 1H).

¹H NMR (400 MHz, DMSO-d6+D2O) δ=7.72 (s, 1H), 6.91 (s, 1H), 6.86 (d, J=1.5 Hz, 1H), 4.42 (br d, J=2.1 Hz, 1H), 3.59-3.50 (m, 3H), 3.49 (s, 3H), 3.42-3.34 (m, 1H), 2.11-2.06 (m, 1H), 2.05-2.02 (m, 3H), 1.94 (br d, J=6.3 Hz, 1H).

MS: 360.1 [(M+H)⁺].

### Examples 86A⁺86B rel-(3R)-1-[2-(4-chloro-2-hydroxy-6-methyl-phenyl)-1-methyl-imidazo[4,5-b]pyrazin-5-yl]-3-methyl-pyrrolidin-3-ol and rel-(3S)-1-[2-(4-chloro-2-hydroxy-6-methyl-phenyl)-1-methyl-imidazo[4,5-b]pyrazin-5-yl]-3-methyl-pyrrolidin-3-ol

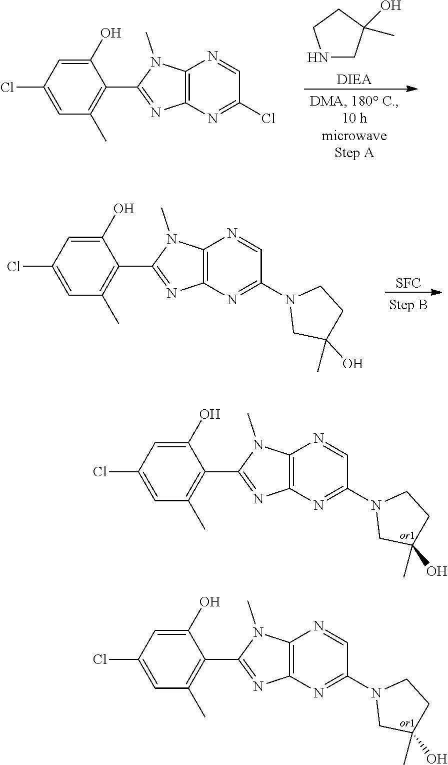

### Step A:

To a solution of 5-chloro-2-(5-chloro-1-methyl-imidazo[4,5-b]pyrazin-2-yl)-3-methyl-phenol (70 mg, 226.43 μmol, 1 eq) in DMA (2 mL) was added DIEA (146.32 mg, 1.13 mmol, 197.20 μL, 5 eq) and 3-methylpyrrolidin-3-ol (458.05 mg, 4.53 mmol, 20 eq). The mixture was stirred at 180° C. for 10 h in a microwave device. LCMS indicated that the reaction was completed. The reaction mixture was quenched by addition of H₂O 10 mL, and then extracted with EA (10 mL×3). The combined organic layers were washed with brine 10 mL, dried over Na₂SO₄, filtered and concentrated under reduced pressure to give a residue. The residue was purified by prep-HPLC (column: Waters Xbridge Prep OBD C18 150 mm×40 mm×10 μm; mobile phase: [H₂O (10 mM NH₄HCO₃)-ACN]; gradient: 20%-50% B over 6.0 min) to give the compound 1-[2-(4-chloro-2-hydroxy-6-methyl-phenyl)-1-methyl-imidazo[4,5-b]pyrazin-5-yl]-3-methyl-pyrrolidin-3-ol (40 mg, 107.00 μmol, 47.26% yield) as a white solid.

MS: 374.2 [(M+H)⁺].

### Step B:

1-[2-(4-Chloro-2-hydroxy-6-methyl-phenyl)-1-methyl-imidazo[4,5-b]pyrazin-5-yl]-3-methyl-pyrrolidin-3-ol was further separated by SFC (column: DAICEL CHIRALPAK AD (250 mm×30 mm, 10 μm); mobile phase: [CO₂-EtOH (0.1% NH₃H₂O)]; B %: 30%, isocratic elution mode). rel-(3R)-1-[2-(4-chloro-2-hydroxy-6-methyl-phenyl)-1-methyl-imidazo[4,5-b]pyrazin-5-yl]-3-methyl-pyrrolidin-3-ol example 86A (9.11 mg, 24.21 μmol, 22.63% yield, 99.36% purity. The first eluting peak) was obtained as a white solid.

¹H NMR (400 MHz, DMSO-d6) δ=10.49 (br s, 1H), 7.70 (s, 1H), 6.87 (br d, 2H), 4.83 (s, 1H), 3.58 (br d), 3.51 (s, 3H), 3.49-3.39 (m, 2H), 2.07 (s, 3H), 2.01-1.89 (m, 2H), 1.38 (s, 3H)

MS: 374.1 [(M+H)⁺].

And rel-(3S)-1-[2-(4-chloro-2-hydroxy-6-methyl-phenyl)-1-methyl-imidazo[4,5-b]pyrazin-5-yl]-3-methyl-pyrrolidin-3-ol example 86B (7.43 mg, 19.69 μmol, 18.40% yield, 99.07% purity. The second eluting peak) was obtained as a white solid.

¹H NMR (400 MHz, DMSO-d6) δ=10.50 (s, 1H), 7.70 (s, 1H), 6.87 (br d, J=19.3 Hz, 2H), 4.83 (s, 1H), 3.60-3.55 (m, 2H), 3.51 (s, 3H), 3.46 (br s, 2H), 2.07 (s, 3H), 2.00-1.89 (m, 2H), 1.38 (s, 3H)

MS: 374.1 [(M+H)⁺].

### Example 87 5-chloro-3-fluoro-2-[1-methyl-5-[(3R)-1-methylpyrrolidin-3-yl]oxy-imidazo[4,5-b]pyrazin-2-yl]phenol

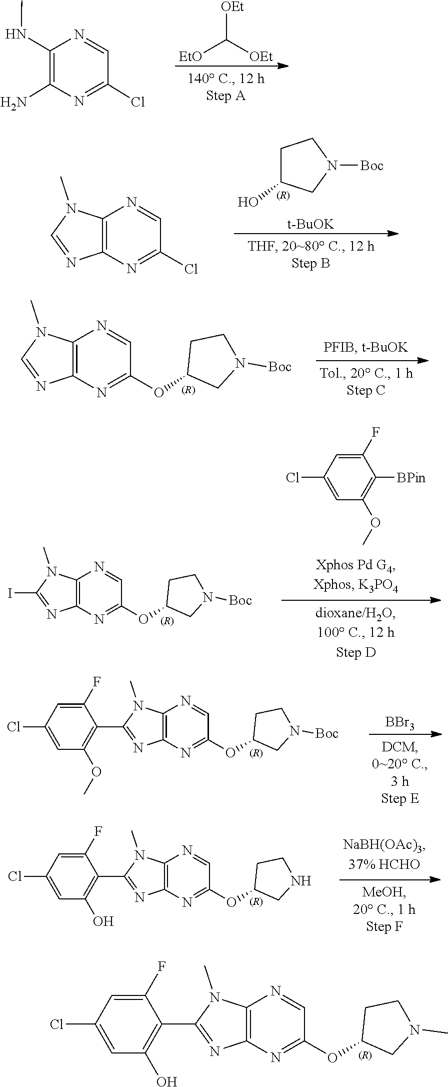

### Step A:

A solution of 5-chloro-N2-methyl-pyrazine-2,3-diamine (4 g, 25.22 mmol, 1.0 eq) in diethoxymethoxyethane (35.64 g, 240.49 mmol, 40.00 mL, 9.53 eq) was stirred at 140° C. for 12 hr. LCMS indicated that the reaction was completed. The reaction mixture was poured onto 80 mL H₂O, followed by 30 mL EA. The aqueous phase was separated and extracted with EA (20 mL×3). The combined organic layers were washed successively with water (20 mL×2) and brine (20 mL×1), dried over anhydrous Na₂SO₄, filtered, and concentrated to give a residue. The crude product was triturated with PE:EA=3:1 at 20° C. for 12 h. 5-Chloro-1-methyl-imidazo[4,5-b]pyrazine (3.2 g, 18.98 mmol, 75.26% yield) was obtained as a yellow solid.

1H NMR (400 MHz, DMSO-d6) δ=8.82 (s, 1H), 8.54 (s, 1H), 3.88 (s, 3H)

MS: 196.1 [(M+H)⁺].

### Step B:

To the solution of tert-butyl (3R)-3-hydroxypyrrolidine-1-carboxylate (4.00 g, 21.35 mmol, 1.2 eq) in THF (60 mL) was added t-BuOK (3.99 g, 35.59 mmol, 2.0 eq) and the reaction mixture was stirred at 20° C. for 30 min, then 5-chloro-1-methyl-imidazo[4,5-b]pyrazine (3 g, 17.80 mmol, 1.0 eq) was added. The resulting reaction mixture was stirred at 80° C. for 11.5 h. LCMS indicated that the reaction was completed. The reaction mixture was poured onto 50 mL H₂O, followed by 10 mL EA. After that, the aqueous phase was separated and extracted with EA (20 mL×3). The combined organic layers were washed successively with water (20 mL×2) and brine (20 mL×1), dried over anhydrous Na₂SO₄, filtered, and concentrated to give a residue. The residue was purified by column chromatography (SiO₂, petroleum ether/ethyl acetate=100/1 to 1/1). tert-Butyl (3R)-3-(1-methylimidazo[4,5-b]pyrazin-5-yl)oxypyrrolidine-1-carboxylate (1.2 g, 3.76 mmol, 21.12% yield) was obtained as a yellow solid.

¹H NMR (400 MHz, DMSO-d6) δ=8.55 (s, 1H), 8.07 (s, 1H), 5.52 (br s, 1H), 3.85 (s, 3H), 3.71-3.60 (m, 1H), 3.45 (br dd, 2H), 3.37 (br s, 1H), 2.22 (br dd, 1H), 2.13 (br s, 1H), 1.40 (br d, 9H)

MS: 320.2 [(M+H)⁺].

### Step C:

To the solution of tert-butyl (3R)-3-(1-methylimidazo[4,5-b]pyrazin-5-yl)oxypyrrolidine-1-carboxylate (1.2 g, 3.76 mmol, 1.0 eq) in toluene (12 mL) was added t-BuOK (505.97 mg, 4.51 mmol, 1.2 eq) and 1,2,3,4,5-pentafluoro-6-iodo-benzene (1.22 g, 4.13 mmol, 1.1 eq). The reaction mixture was stirred at 20° C. for 1 hr. LCMS indicated that the reaction was completed. The reaction solution was filtered to remove t-BuOK and then concentrated to give the crude product. The crude product was purified by prep-TLC (SiO₂, PE:EA=1:1). tertBbutyl (3R)-3-(2-iodo-1-methyl-imidazo[4,5-b]pyrazin-5-yl)oxypyrrolidine-1-carboxylate (0.9 g, 2.02 mmol, 53.79% yield) was obtained as a yellow solid.

¹H NMR (400 MHz, DMSO-d6) δ=8.01 (s, 1H), 5.49 (br s, 1H), 3.76 (s, 3H), 3.69-3.58 (m, 1H), 3.45 (br d, 2H), 3.40-3.35 (m, 1H), 2.26-2.17 (m, 1H), 2.12 (br s, 1H), 1.39 (br d, 9H)

MS: 390.1 (M−tBu+H)⁺].

### Step D:

To the solution of tert-butyl (3R)-3-(2-iodo-1-methyl-imidazo[4,5-b]pyrazin-5-yl)oxypyrrolidine-1-carboxylate (0.4 g, 898.36 μmol, 1.0 eq) in the mixed solvent of dioxane (4 mL) and H₂O (1 mL) was successively added 2-(4-chloro-2-fluoro-6-methoxy-phenyl)-4,4,5,5-tetramethyl-1,3,2-dioxaborolane (471.92 mg, 988.20 μmol, 1.1 eq), XPhos Pd G4 (77.30 mg, 89.84 μmol, 0.1 eq) and K₃PO₄ (381.38 mg, 1.80 mmol, 2.0 eq), then the mixture was degassed 3 times with N₂. The resulting reaction mixture was stirred at 100° C. for 12 hr under N₂. LCMS indicated that the reaction was completed. The reaction mixture was poured onto 20 mL saturated EDTA, followed by 10 mL EA. The solution was stirred at 20° C. for 0.5 h. After that, the aqueous phase was separated and extracted with EA (20 mL×2). The combined organic layers were washed successively with water (20 mL×2) and brine (20 mL×1), dried over anhydrous Na₂SO₄, filtered, and concentrated to give a residue. The residue was purified by prep-TLC (SiO₂, PE:EA=1:1). tert-Butyl (3R)-3-[2-(4-chloro-2-fluoro-6-methoxy-phenyl)-1-methyl-imidazo[4,5-b]pyrazin-5-yl]oxypyrrolidine-1-carboxylate (0.42 g, 439.41 μmol, 24.46% yield, 50% purity) was obtained as a yellow oil.

MS: 478.2 [(M+H)⁺].

### Step E:

To the solution of tert-butyl (3R)-3-[2-(4-chloro-2-fluoro-6-methoxy-phenyl)-1-methyl-imidazo[4,5-b]pyrazin-5-yl]oxypyrrolidine-1-carboxylate (0.3 g, 313.86 μmol, 1.0 eq, 50% purity) in DCM (5 mL) was added BBr₃ (393.14 mg, 1.57 mmol, 151.21 μL, 5.0 eq) at 0° C. The resulting reaction mixture was stirred at 20° C. for 3 hr. LCMS indicated that the reaction was completed. After completion of the reaction, the solvent was removed by concentration to give the crude product. The residue was purified by prep-HPLC (column: Phenomenex Luna C18 100 mm×30 mm×5 μm; mobile phase: [H₂O (0.2% FA)-ACN]; gradient: 1%-30% B over 8.0 min) to give the title product. 5-Chloro-3-fluoro-2-[1-methyl-5-[(3R)-pyrrolidin-3-yl]oxy-imidazo[4,5-b]pyrazin-2-yl]phenol (50 mg, 137.45 μmol, 43.79% yield) was obtained as a yellow solid.

1H NMR (400 MHz, DMSO-d6) δ=8.03 (s, 1H), 6.78 (br d, 1H), 6.74 (s, 1H), 5.55-5.51 (m, 1H), 3.66 (s, 3H), 3.37-3.35 (m, 1H), 3.15 (br d, 3H), 2.25-2.18 (m, 1H), 2.07 (br s, 1H)

MS: 363.9 [(M+H)⁺].

### Step F:

To the solution of 5-chloro-3-fluoro-2-[1-methyl-5-[(3R)-pyrrolidin-3-yl]oxy-imidazo[4,5-b]pyrazin-2-yl]phenol (50 mg, 137.45 μmol, 1.0 eq) in MeOH (1 mL) was added HCHO (22.31 mg, 274.90 μmol, 20.47 μL, 37% purity, 2.0 eq) and the reaction mixture was stirred at 20° C. for 10 min, then NaBH(OAc)₃ (43.70 mg, 206.17 μmol, 1.5 eq) was added. The resulting reaction mixture was stirred at 20° C. for 50 min. LCMS indicated that the reaction was completed. The reaction mixture was poured onto 10 mL H₂O and then the pH was adjusted to 8-9 with solid NaHCO₃. The resulting solution was extracted with EA (10 mL×3). The combined organic layers were washed successively with water (20 mL×2) and brine (20 mL×1), dried over anhydrous Na₂SO₄, filtered, and concentrated to give a residue. The residue was purified by prep-HPLC (column: Waters Xbridge Prep OBD C18 150 mm×40 mm×10 μm; mobile phase: [H₂O (10 mM NH₄HCO₃)-ACN]; gradient: 5%-55% B over 8.0 min) to give the title product 5-chloro-3-fluoro-2-[1-methyl-5-[(3R)-1-methylpyrrolidin-3-yl]oxy-imidazo[4,5-b]pyrazin-2-yl]phenol (32 mg, 83.21 μmol, 60.54% yield, 98.24% purity) as a white solid.

¹H NMR (400 MHz, DMSO-d6) δ=11.31 (s, 1H), 8.08 (s, 1H), 7.06 (dd, 1H), 6.90 (s, 1H), 5.46-5.37 (m, 1H), 3.66 (s, 3H), 2.86 (dd, 1H), 2.78-2.72 (m, 2H), 2.44-2.34 (m, 2H), 2.30 (s, 3H), 1.94-1.84 (m, 1H)

MS: 378.1 [(M+H)⁺].

### Biological Assay Description

### 1.1 NLRP3 Inhibition Assay

The following assays were used to determine the inhibitory activity of test compounds on the NLRP3 inflammasome pathway using common stimuli Nigericin (Invivogen) or monosodium urate crystals (MSU) (Invivogen).

### Cell Culture

Human monocyte-like cells were cultured in RPMI-1640 Glutamax medium supplemented with 10% heat inactivated FBS (Fetal Bovine Serum) and 50 U/mL penicillin-streptomycin (Life Technologies).

### NLRP3 Inflammasome Pathway Activation Assay

Human monocyte-like cells were seeded at 25000 per well in a 384-well plate and were differentiated overnight into macrophages with 10 ng/mL PMA (Phorbol Myristate Acetate). The following day, medium containing 10 ng/mL LPS (Lipopolysaccharide) was added. After 3 hours of LPS priming, concentrations of test compound in the range from 100 μM to 380 μM were added 30 min prior to NLRP3 inflammasome pathway stimulation with Nigericin 3.75 μM or MSU 200 μg/mL for 3 h.

### Measurement of IL-1β

For IL-1p quantification, supernatants were analyzed using HTRF kit according to the manufacturer's instructions (Cisbio 62HIL1BPEH). Briefly, in a 384-well ProxiPlate™ microplate, 8 μl of sample was mixed, with 5 μl of Anti-IL1β Cryptate antibody (40×) and Anti-IL1β XL antibody (40×). Then, incubated overnight at RT. Reading was done using an EnVision Reader (PerkinElmer).

IC₅₀ (concentration corresponding to 50% inhibition) were determined using GraphPad Prism 9.

The following example compounds were measured:

TABLE 7

| | IC₅₀ | IC₅₀ |
|---|---|---|
| | Human | Human |
| | monocyte- | monocyte-like |
| | like cells | cells |
| Examples | MSU | Nigericin |
| 1 | ++ | ++ |
| 2 | ++ | ++ |
| 3 | ++ | ++ |
| 4 | ++ | ++ |
| 5 | ++ | nd |
| 6 | + | nd |
| 7 | ++ | ++ |
| 8 | +++ | +++ |
| 9 | +++ | ++ |
| 10A | ++ | nd |
| 10 | +++ | nd |
| 10B | +++ | nd |
| 11 | ++ | nd |
| 12 | ++ | nd |
| 13 | ++ | nd |
| 14 | ++ | nd |
| 15 | ++ | nd |
| 16 | ++ | nd |
| 17 | ++ | nd |
| 18A | +++ | +++ |
| 18B | ++ | nd |
| 19 | +++ | +++ |
| 20 | ++ | + |
| 21 | ++ | nd |
| 22A | ++ | nd |
| 22B | ++ | nd |
| 23 | +++ | ++ |
| 24 | ++ | +++ |
| 25 | +++ | ++ |
| 26 | +++ | ++ |
| 27 | +++ | +++ |
| 28 | +++ | +++ |
| 29 | +++ | ++ |
| 30 | ++ | nd |
| 31 | ++ | nd |
| 32 | +++ | ++ |
| 33 | ++ | ++ |
| 34 | ++ | nd |
| 35 | ++ | nd |
| 36 | ++ | nd |
| 37A | ++ | nd |
| 37B | ++ | nd |
| 38 | ++ | nd |
| 39 | ++ | nd |
| 40 | +++ | +++ |
| 41 | ++ | nd |
| 42 | ++ | nd |
| 43 | +++ | nd |
| 44 | ++ | nd |
| 45 | ++ | nd |
| 46 | +++ | nd |
| 47 | +++ | nd |
| 48 | ++ | nd |
| 49 | ++ | nd |
| 50 | ++ | nd |
| 51 | +++ | +++ |
| 52 | +++ | nd |
| 53 | +++ | nd |
| 54 | ++ | nd |
| 55 | ++ | nd |
| 56 | +++ | nd |
| 57 | ++ | nd |
| 58 | +++ | nd |
| 59 | +++ | nd |
| 60 | +++ | nd |
| 61 | +++ | nd |
| 62 | ++ | nd |
| 63 | +++ | nd |
| 64 | ++ | nd |
| 65 | +++ | nd |
| 66 | +++ | nd |
| 67 | +++ | nd |
| 68 | ++ | nd |
| 69 | ++ | nd |
| 70 | +++ | nd |
| 71 | ++ | nd |
| 72 | +++ | nd |
| 73 | ++ | nd |
| 74 | +++ | nd |
| 75 | ++ | nd |
| 76 | +++ | nd |
| 77A | +++ | nd |
| 77B | +++ | nd |
| 78 | +++ | nd |
| 79 | +++ | nd |
| 80 | +++ | nd |
| 81 | +++ | nd |
| 84 | ++ | nd |
| 85A | +++ | nd |
| 85B | +++ | nd |
| 86A | +++ | nd |
| 86B | +++ | nd |
| 87 | +++ | nd |

Legend:
+++ IC₅₀ < 100 nM;
++ IC₅₀ 100 nM < x < 1 μM;
+ IC₅₀ > 1 μM,
nd = not determined.

The tested compounds showed inhibition of IL-1beta release in human monocyte-like cells: using MSU or Nigericin as activators, see Table 7.

The same NLRP3 inhibition assays were used to determine the inhibitory activity of representative example test compounds of formulas II′/II and formula III on the NLRP3 inflammasome pathway using common stimuli Nigericin (Invivogen) or monosodium urate crystals (MSU) (Invivogen).

The following example compounds were measured:

TABLE 8

| | IC₅₀ | IC₅₀ |
|---|---|---|
| | Human | Human |
| | monocyte- | monocyte-like |
| | like cells | cells |
| Examples | MSU | Nigericin |
| 1 | ++ | ++ |
| 82 | − | nd |
| 2 | ++ | ++ |
| 83 | − | nd |

Legend:
+++ IC₅₀ < 100 nM;
++ IC₅₀ 100 nM < x < 500 nM;
+ IC₅₀ 500 nM < x < 1.5 μM ;
− IC₅₀ >1.5 μM,
nd = not determined.

As seen from Table 8, representative test compounds of formula II′/II of the invention showed significantly increased inhibition of IL-1beta release in human monocyte-like cells compared to corresponding compounds of formula III.

### 1.2 Inhibition of IL-1β in an LPS/ATP Induced Acute Peritonitis Mouse Model

1.2.1 The efficacy of Example compounds 53 and 18 in an LPS/ATP-induced acute peritonitis was evaluated: LPS (Lipopolysaccharide) and ATP (Adenosine triphosphate) were administered to BALB/c mice by intraperitoneal (i.p.) injection to elicit an acute inflammatory response in the peritoneal cavity.

### Method

Mice (BALB/c mice, aged 6-8 w, females) were challenged with 50 μg/kg LPS by intraperitoneal (i.p.) injection. 120 min after LPS administration, ATP was delivered by i.p. injection at 50 mM in 200 μL. Test compounds (Example compounds 53 and 18 were each formulated at a dosing volume of 10 mL/kg in 0.5% carboxymethylcellulose sodium salt (CMC-Na) with 0.2% tween 80 in water. Test compounds were dosed 30 min before LPS injection. 30 min post ATP injection, animals were sacrificed by CO₂. For the peritoneal wash (PW) collection 3 mL cold phosphate-buffered saline (PBS)/heparin (25 U per mL) was injected in the peritoneal cavity using a 25 G needle and the abdominal area was gently massaged for 2 min. Lavage fluid was collected, and supernatant was harvested after centrifugation (300×g, for 10 min at 4° C.). Supernatants were recovered and snap frozen on dry ice.

Cytometric Bead Array (CBA) Assay for IL-1β detection was performed on the peritoneal wash supernatant as follows: supernatants or cytokine standards were incubated with the capture beads mixture for 1 hour at room temperature. Dilution factor in peritoneal lavage: for TNF and IL-1β, samples were diluted 2-fold; for IL-6, samples were diluted 20-fold. The capture beads were then incubated with the phycoerythrin (PE) Detection Reagents mixture containing PE-conjugated anti-mouse IL-1β, TNF, and IL-6 for 1 hour at room temperature. After washing in Wash Buffer containing 1×PBS, blocking protein, and detergent, the beads were re-suspended and analyzed by flow cytometry.

The cytokine analysis raw data were analyzed using FACP. Data was exported and plotted using GraphPad Prism 9.5.1 (733). Data were expressed as the Mean±SD. One-way ANOVA was used to compare significance among groups.

### Results

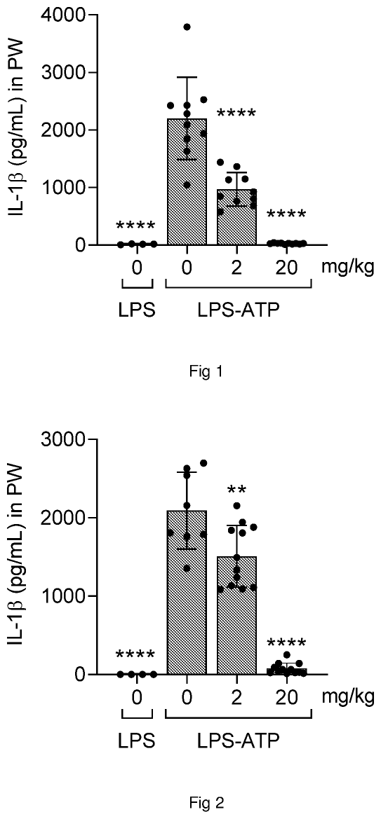

Mice challenged with LPS and ATP showed increased IL-1β levels in peritoneal wash (PW), indicating successful model induction. As seen from FIG. 1, example compound 53 significantly reduced IL-1β release in PW in a dose response manner in an LPS-ATP acute peritonitis model (n=10, One-way ANOVA with Dunnett's multiplicity adjustment. Comparisons against group: LPS-ATP, 0 mg/kg. ****p<0.0001). As seen from FIG. 2, example compound 18 significantly reduced IL-1p release in PW in a dose response manner in an LPS-ATP acute peritonitis model (n=8-12, One-way ANOVA with Dunnett's multiplicity adjustment. Comparisons against group: LPS-ATP, 0 mg/kg. ****p<0.0001, **p<0.005).

Results demonstrate the efficacy of compounds of the invention in reducing IL-1p release in vivo.

1. A compound having the formula (I′) 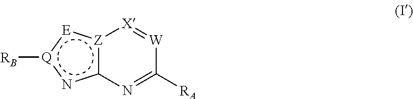
or a stereoisomer, a racemic mixture, a tautomer, a polymorph, a pharmaceutically acceptable salt, a prodrug, a hydrate, or a solvate thereof;
wherein
X′ is selected from CH or N;
W is selected from N, CH or CR_{c};
Q is selected from N and C;
E is selected from NRₐ and CRₐ;
Z is selected from N and C;
wherein at least one of Q and Z is C, and/or E is CRₐ;
R_{c} is selected from the group consisting of —C₁-C₄alkyl, —O—C₁-C₄alkyl, —C₁-C₄alkyl-OH, -halo or —C₁-C₄alkyl-Hal;
Rₐ is selected from the group consisting of —H and —C₁-C₃alkyl;
R_{A} and R_{B} are each selected from 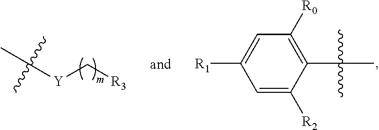
wherein one of R_{A} and R_{B} is 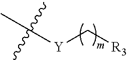
and the other of R_{A} and R_{B} is 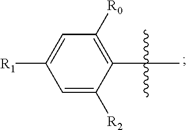
R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH, NR_{d}, O or is a bond;
R_{d} is selected from the group consisting of —C₁-C₄alkyl, —C₁-C₄alkyl-OH, or —C₁-C₄alkyl-Hal;
R₃ is selected from the group consisting of
4-, 5- or 6-membered heterocycloalkyl containing one or two heteroatoms, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH; —NR₅R₆ and halo; or
8-, 9- or 10-membered bicyclic fused heterocycloalkyl containing one to three heteroatoms wherein said heteroatom(s) is/are independently selected from N and O, optionally substituted at any available position with one or two substituents independently selected from the group consisting of C₁-C₄alkyl, haloC₁-C₄alkyl, hydroxyC₁-C₄alkyl, —OH, —NR₅R₆ and halo; or
C₃-C₆cycloalkyl optionally substituted with one or two substituents independently selected from the group consisting of C₁-C₄alkyl, haloC₁-C₄alkyl, hydroxyC₁-C₄alkyl, —OH and halo; or
C₁-C₆alkyl optionally substituted with one or two substituents independently selected from the group consisting of —OH, halo, haloC₁-C₄alkyl, C₁-C₄alkoxy and —NR₅R₆;
R₅ and R₆ are independently selected from H and C₁-C₃alkyl; and
m is 0, 1 or 2.

2. A compound according to claim 1 having the formula (II′) 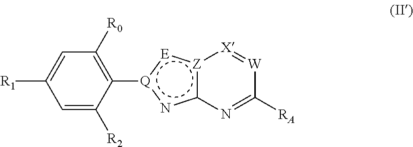
or a stereoisomer, a racemic mixture, a tautomer, a polymorph, a pharmaceutically acceptable salt, a prodrug, a hydrate, or a solvate thereof;
wherein
X′ is selected from CH or N;
W is selected from N, CH or CR_{c};
Q is selected from N and C;
E is selected from NRₐ and CRₐ;
Z is selected from N and C;
wherein at least one of Q and Z is C, and/or E is CRₐ;
R_{c} is selected from the group consisting of C₁-C₄alkyl, —O—C₁-C₄alkyl, C₁-C₄alkyl-OH, halo or haloC₁-C₄alkyl;
R_{d} is selected from the group consisting of —C₁-C₄alkyl, —C₁-C₄alkyl-OH, or —C₁-C₄alkyl-Hal;
Rₐ is selected from the group consisting of —H and —C₁-C₃alkyl;
R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
R_{A} is 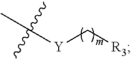
Y is selected from NH, NR_{d}, O or is a bond;
R₃ is selected from the group consisting of
4-, 5- or 6-membered heterocycloalkyl containing one or two heteroatoms, preferably one heteroatom, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH; —NR₅R₆ and halo; or
8-, 9- or 10-membered bicyclic fused heterocycloalkyl containing one to three heteroatoms wherein said heteroatom(s) is/are independently selected from N and O, optionally substituted at any available position with one or two substituents independently selected from the group consisting of C₁-C₄alkyl, haloC₁-C₄alkyl, hydroxyC₁-C₄alkyl, —OH, —NR₅R₆ and halo; or
C₃-C₆cycloalkyl optionally substituted with one or two substituents independently selected from the group consisting of C₁-C₄alkyl, haloC₁-C₄alkyl, hydroxyC₁-C₄alkyl, —OH and halo; or
C₁-C₆alkyl optionally substituted with one or two substituents independently selected from the group consisting of —OH, halo, haloC₁-C₄alkyl, C₁-C₄alkoxy and —NR₅R₆;
R₅ and R₆ are independently selected from H and C₁-C₃alkyl; and
m is 0, 1 or 2.

3. A compound according to claim 1 having the formula (I) 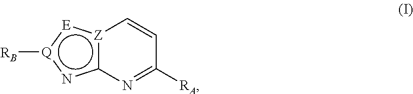
or a stereoisomer, a racemic mixture, a tautomer, a polymorph, a pharmaceutically acceptable salt, a prodrug, a hydrate, or a solvate thereof;
wherein
Q is selected from N and C;
E is selected from NRₐ and CRₐ;
Z is selected from N and C;
wherein at least one of Q and Z is C, and/or E is CRₐ; and
Rₐ is selected from the group consisting of —H and —C₁-C₃alkyl;
R_{A} and R_{B} are each selected from 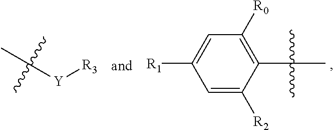
wherein one of R_{A} and R_{B} is 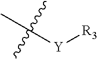
and the other of R_{A} and R_{B} is 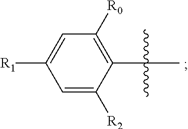
R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH and O; and
R₃ is selected from the group consisting of
4-, 5- or 6-membered heterocycloalkyl containing one or two heteroatoms, preferably one heteroatom, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH and halo;
C₃-C₆cycloalkyl optionally substituted with one or two substituents independently selected from the group consisting of C₁-C₄alkyl, haloC₁-C₄alkyl, hydroxyC₁-C₄alkyl, —OH and halo; and
C₁-C₆alkyl optionally substituted with one or two substituents independently selected from the group consisting of —OH, halo, haloC₁-C₄alkyl, C₁-C₄alkoxy and —NR₅R₆ wherein R₅ and R₆ are independently selected from H and C₁-C₃alkyl.

4. A compound according to any one of claims 1 to 3 having the formula (II) 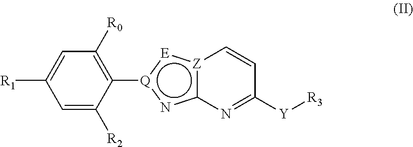
or a stereoisomer, a racemic mixture, a tautomer, a polymorph, a pharmaceutically acceptable salt, a prodrug, a hydrate, or a solvate thereof;
wherein
Q is selected from N and C;
E is selected from NRₐ and CRₐ;
Z is selected from N and C;
wherein at least one of Q and Z is C, and/or E is CRₐ;
Rₐ is selected from the group consisting of —H and —C₁-C₃alkyl;
R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH and O; and
R₃ is selected from the group consisting of
4-, 5- or 6-membered heterocycloalkyl containing one or two heteroatoms, preferably one heteroatom, wherein said heteroatom(s) is/are independently selected from N and O, optionally substituted with one or two substituents independently selected from the group consisting of C₁-C₄alkyl, haloC₁-C₄alkyl, hydroxyC₁-C₄alkyl, —OH and halo;
C₃-C₆cycloalkyl optionally substituted with one or two substituents independently selected from the group consisting of C₁-C₄alkyl, haloC₁-C₄alkyl, hydroxyC₁-C₄alkyl, —OH and halo; and
C₁-C₄alkyl optionally substituted with one or two substituents independently selected from the group consisting of —OH, halo, haloC₁-C₄alkyl, C₁-C₄alkoxy and —NR₅R₆ wherein R₅ and R₆ are independently selected from H and C₁-C₃alkyl.

5. The compound according to claim 1 or 3 having the formula (III) 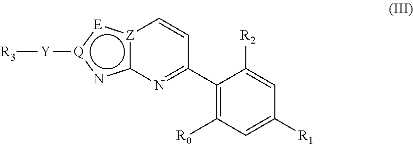
or a stereoisomer, a racemic mixture, a tautomer, a polymorph, a pharmaceutically acceptable salt, a prodrug, a hydrate, or a solvate thereof;
wherein Q, E, Z, R₀, R₁, R₂, R₃, and Y are as defined in claim 1 or 3.

6. The compound according to claim 2 having the formula (II′a) 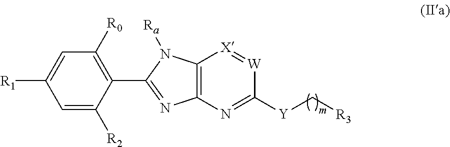
or a stereoisomer, a racemic mixture, a tautomer, a polymorph, a pharmaceutically acceptable salt, a prodrug, a hydrate, or a solvate thereof;
wherein X′, W, R₀, R₁, R₂, R₃, Rₐ, m and Y are as defined in claim 1.

7. The compound according to claim 2 having the formula (II′b) 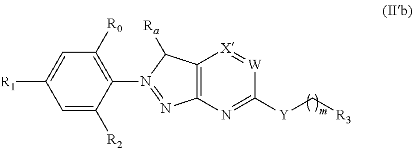
or a stereoisomer, a racemic mixture, a tautomer, a polymorph, a pharmaceutically acceptable salt, a prodrug, a hydrate, or a solvate thereof;
wherein X′, W, R₀, R₁, R₂, R₃, Rₐ, m and Y are as defined in claim 1.

8. The compound according to any one of claims 2 to 4 having the formula (IIa) 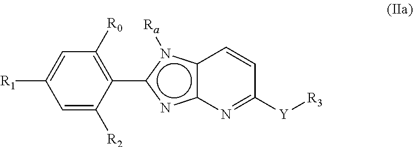
or a stereoisomer, a racemic mixture, a tautomer, a polymorph, a pharmaceutically acceptable salt, a prodrug, a hydrate, or a solvate thereof;
wherein R₀, R₁, R₂, R₃, Rₐ and Y are as defined in any one of claims 2 to 4.

9. The compound according to any one of claims 2 to 4 having the formula (IIb) 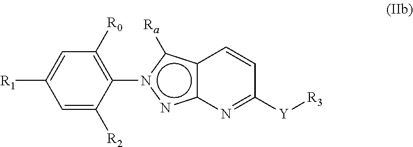
or a stereoisomer, a racemic mixture, a tautomer, a polymorph, a pharmaceutically acceptable salt, a prodrug, a hydrate, or a solvate thereof;
wherein R₀, R₁, R₂, R₃, Rₐ and Y are as defined in any one of claims 2 to 4.

10. The compound according to claim 2 having the formula (IV) 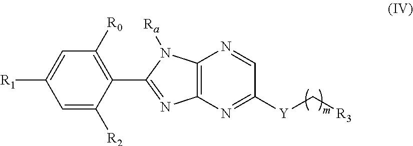
or a stereoisomer, a racemic mixture, a tautomer, a polymorph, a pharmaceutically acceptable salt, a prodrug, a hydrate, or a solvate thereof;
wherein R₀, R₁, R₂, R₃, Rₐ, m and Y are as defined in claim 2.

11. The compound according to claim 2 having the formula (V) 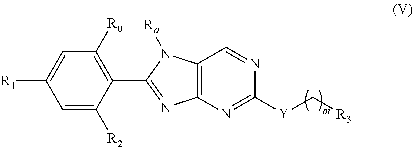
or a stereoisomer, a racemic mixture, a tautomer, a polymorph, a pharmaceutically acceptable salt, a prodrug, a hydrate, or a solvate thereof;
wherein R₀, R₁, R₂, R₃, Rₐ, m and Y are as defined in claim 2.

12. The compound according to any one of the preceding claims, wherein
R₃ is selected from the group consisting of
4-, 5- or 6-membered heterocycloalkyl containing one heteroatom, wherein said heteroatom is N or O, optionally substituted with one or two substituents independently selected from the group consisting of C₁-C₄alkyl;
C₃-C₆cycloalkyl optionally substituted with one or two substituents independently selected from the group consisting of C₁-C₄alkyl, —OH and halo;
hydroxyC₁-C₆alkyl; and
C₁-C₆alkyl optionally substituted with —NR₅R₆ wherein R₅ and R₆ are independently selected from H and C₁-C₃alkyl.

13. The compound according to any one of the preceding claims, wherein R₃ is selected from the group consisting of 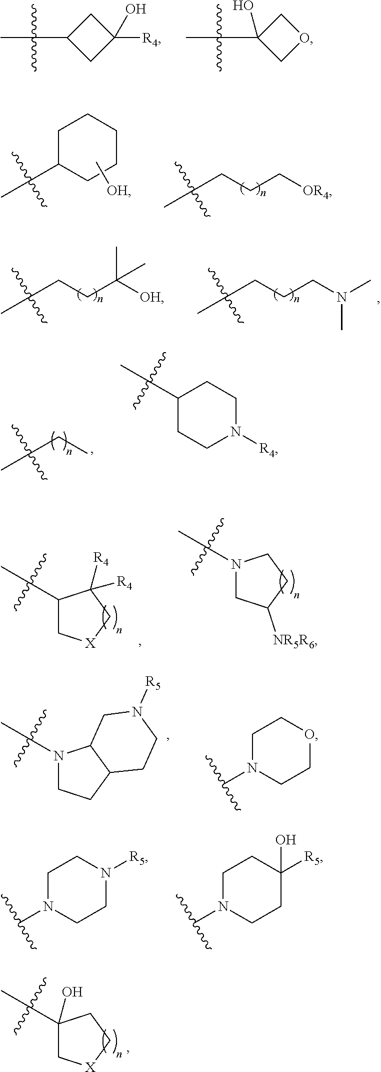
Me, and CF₃;
wherein
X is selected from O and NR₄;
R₄ is independently selected from H, halo or C₁-C₃alkyl;
R₅ is independently selected from H or C₁-C₃alkyl, preferably H or Me;
R₆ is selected from the group consisting of C₁-C₄alkyl; and
n is selected from 0, 1 or 2.

14. The compound according to any one of claims 1 to 10, wherein R₃ is selected from the group consisting of 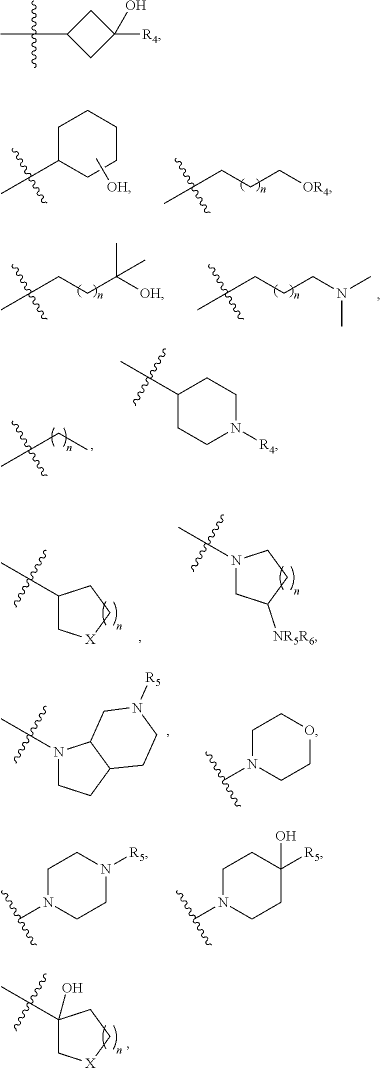
Me, and CF₃;
wherein
X is selected from O and NR₄;
R₄ is independently selected from H, halo or C₁-C₃alkyl;
R₅ is independently selected from —H or -Me;
R₆ is selected from the group consisting of C₁-C₄alkyl; and
n is selected from 0, 1 or 2.

15. The compound according to claim 11, wherein R₃ is selected from the group consisting of 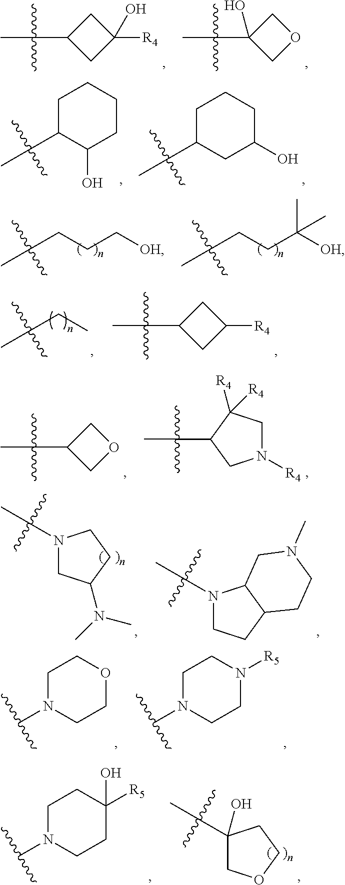
Me, and CF₃;
wherein
R₄ is independently selected from H, F, or C₁-C₃alkyl; and
R₅ is methyl; and
n is selected from 0, 1 or 2.

16. The compound according to any one of claims 1 to 10, wherein R₃ is selected from the group consisting of 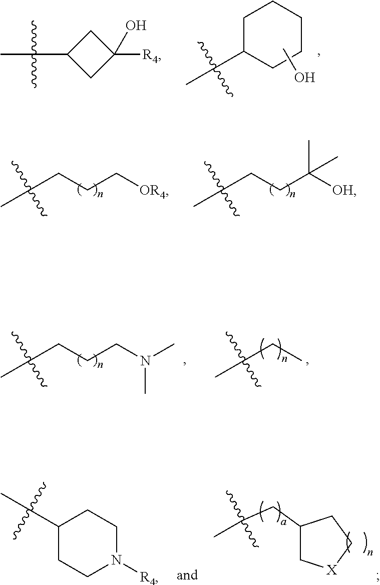
wherein
X is selected from O and NR₄;
R₄ is independently selected from —H or —C₁-C₃alkyl;
a is selected from 0 and 1; and
n is selected from 0, 1 or 2.

17. The compound according to claim 16, wherein R₃ is selected from the group consisting of 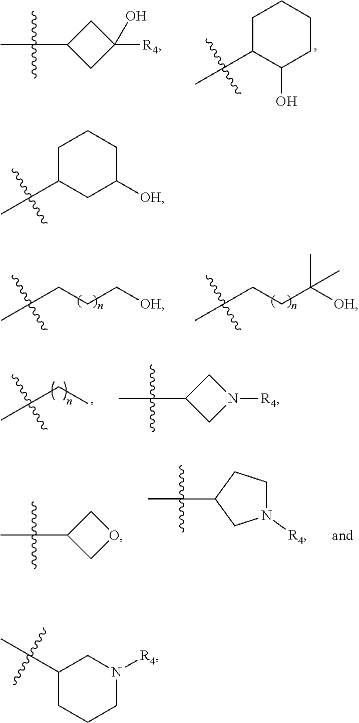
wherein R₄ is independently selected from —H or —C₁-C₃alkyl; and
n is selected from 0, 1 or 2.

18. The compound according to any one of the preceding claims, wherein
R₀ is —H or —CH₃;
R₁ is —CF₃For -halo, preferably —Cl; and
R₂ is —OH.

19. The compound according to any of the preceding claims, which is selected from 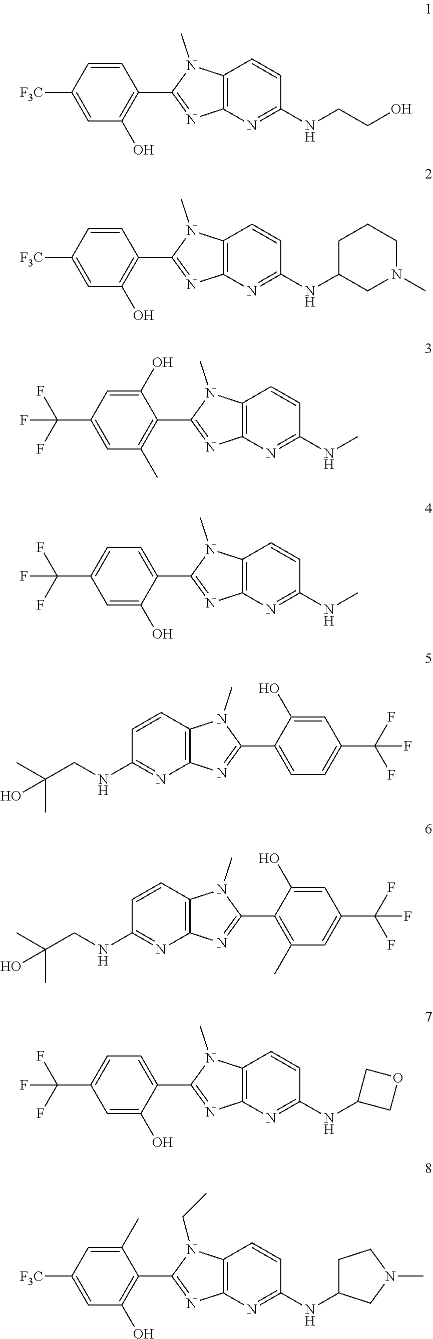 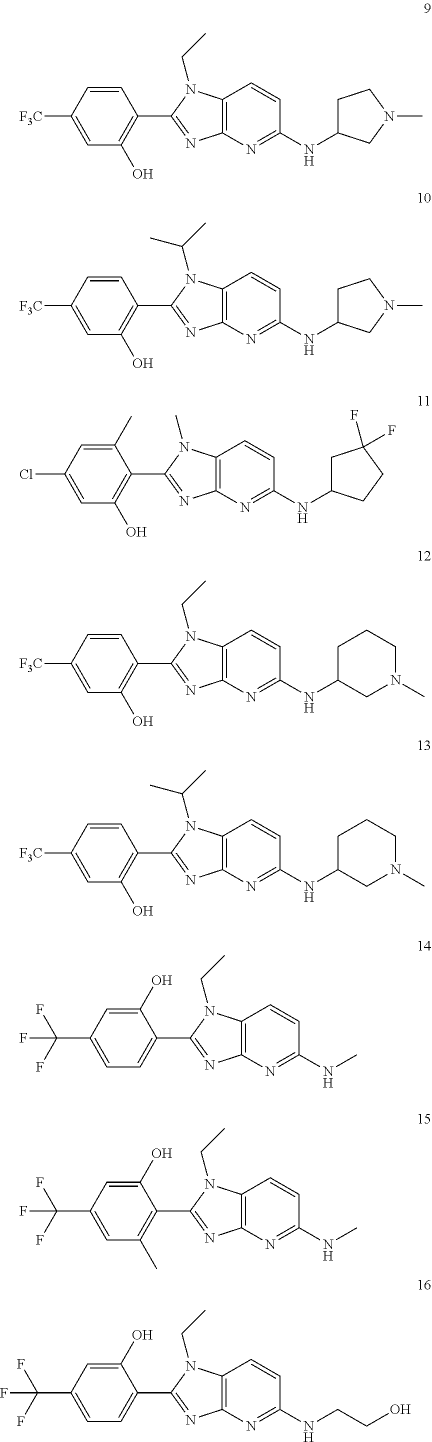 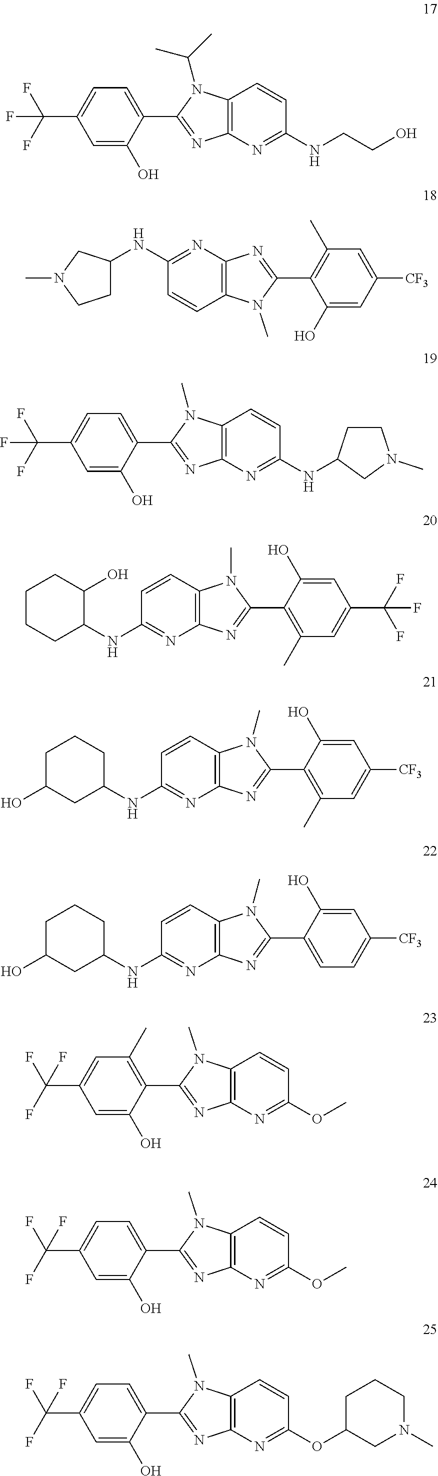 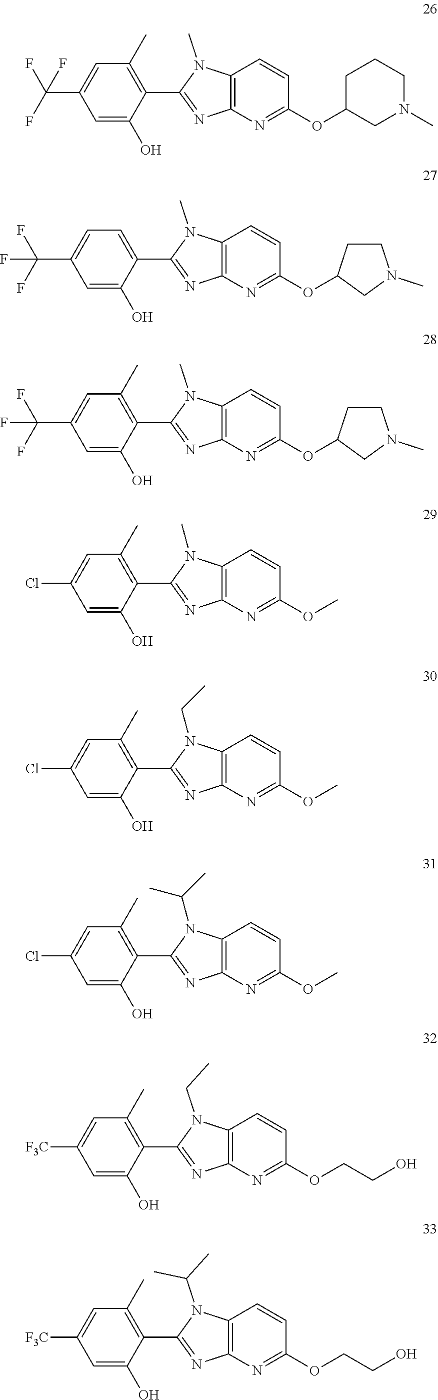 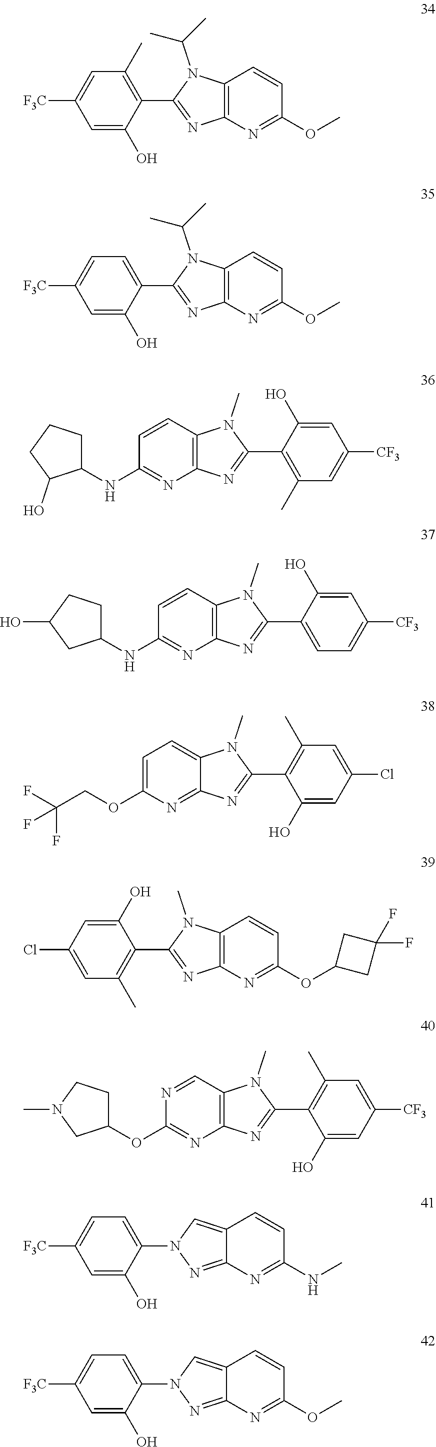 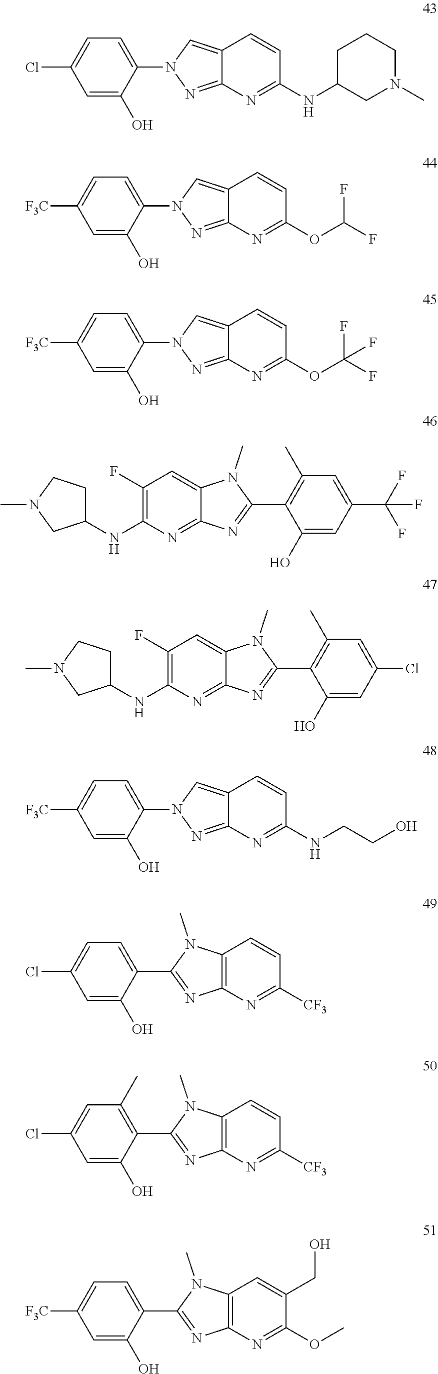 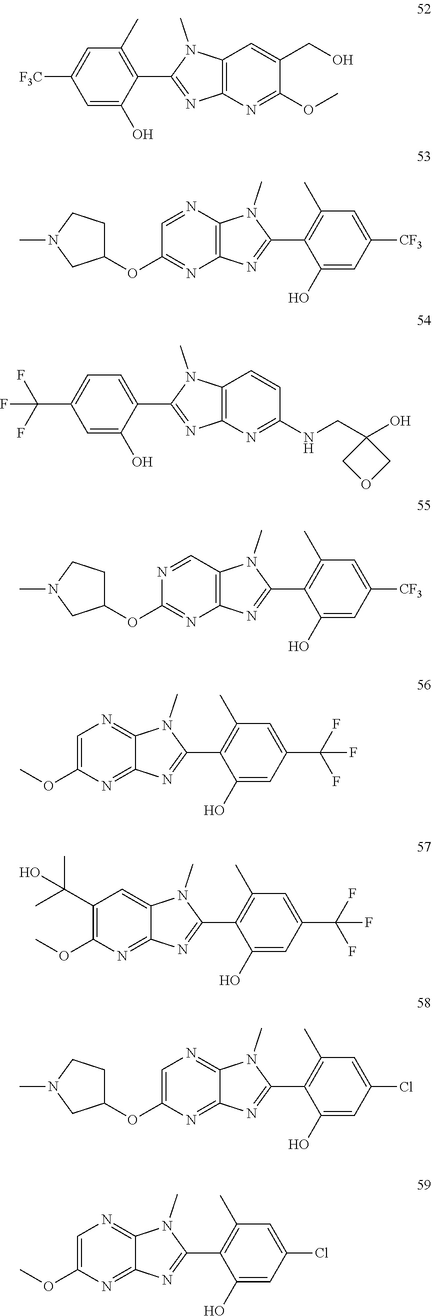 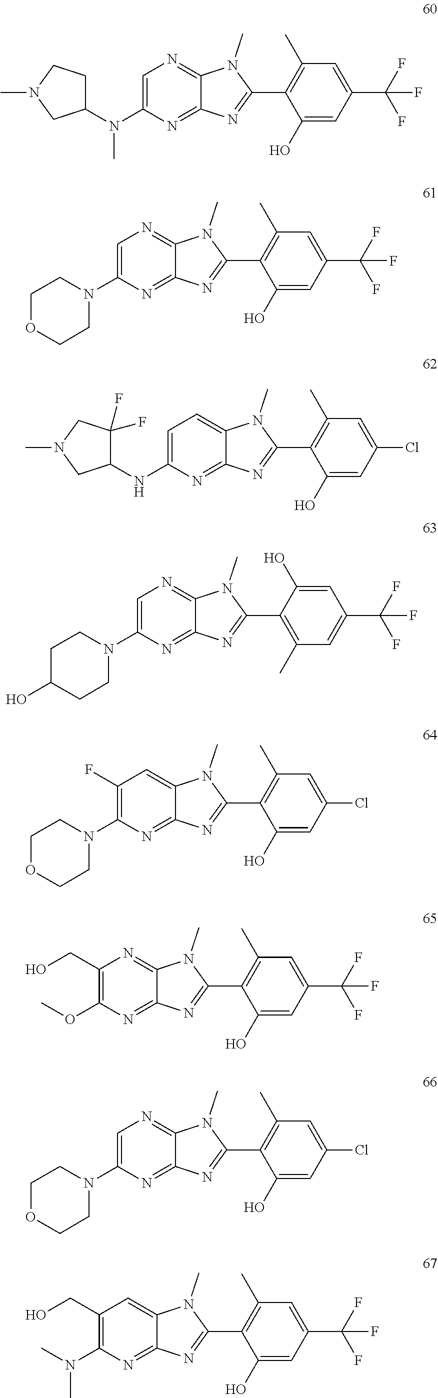 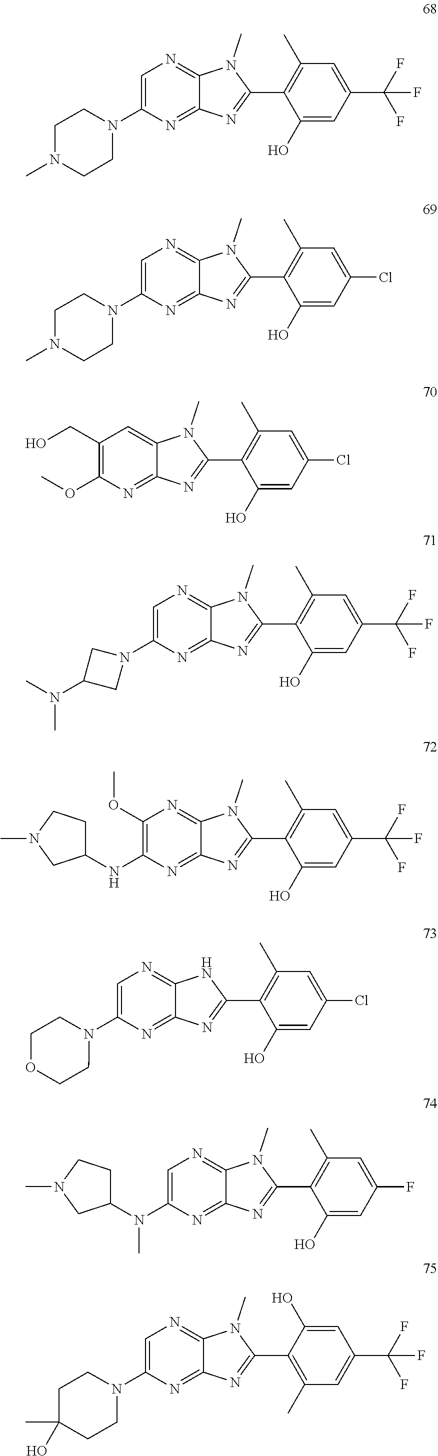 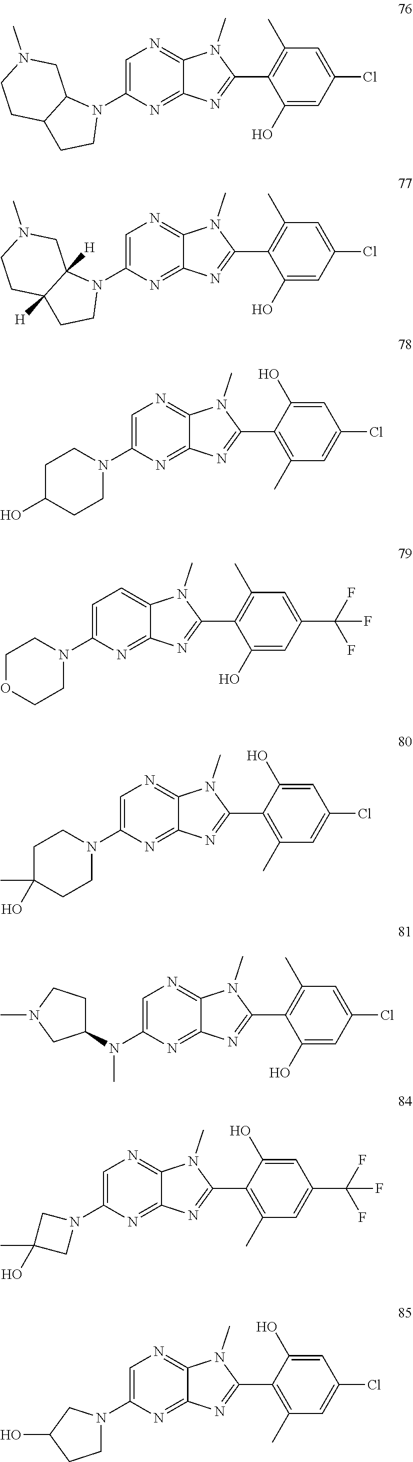 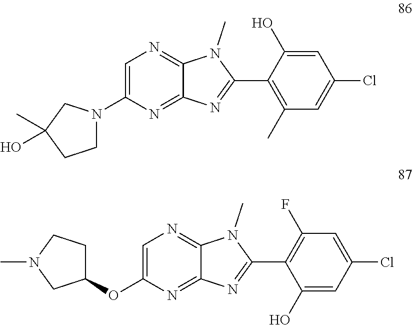
or a stereoisomer, a racemic mixture, a tautomer, a polymorph, a pharmaceutically acceptable salt, a prodrug, a hydrate, or a solvate thereof.

20. A pharmaceutical composition comprising a compound as defined in any one of claims 1 to 19, or a stereoisomer, a racemic mixture, a tautomer, a polymorph, a pharmaceutically acceptable salt, a prodrug, a hydrate, or a solvate thereof, and optionally comprising at least one pharmaceutically acceptable carrier, diluent, adjuvant or excipient.

21. The compound according to any one of claims 1 to 19, or a stereoisomer, a racemic mixture, a tautomer, a polymorph, a pharmaceutically acceptable salt, a prodrug, a hydrate, or a solvate thereof, for use as a medicament.

22. The compound according to any one of claims 1 to 19, or a stereoisomer, a racemic mixture, a tautomer, a polymorph, a pharmaceutically acceptable salt, a prodrug, a hydrate, or a solvate thereof, for use in the treatment, alleviation or prevention of a disease, or a disorder or an abnormality which is responsive to the modulation of a component of the NLRP3 inflammasome pathway and/or which is responsive to the modulation of IL-1 beta and/or IL-18 levels.

23. The compound for use according to claim 22, wherein the modulation is the reduction and/or inhibition of IL-1 beta.

24. The compound for use according to claim 22, wherein the component of the inflammasome pathway is NLRP3 inflammasome.

25. The compound for use according to claim 22 or 24, wherein the activation of NLRP3 inflammasome pathway is inhibited.

26. The compound for use according to any one of claims 22 to 25, wherein the disease, the disorder or the abnormality is selected from Alzheimer's disease, Parkinson's disease, amyotrophic lateral sclerosis, demyelination, viral encephalitis, epilepsy, stroke, atherosclerosis, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease (IBD) (including Crohn's disease, ulcerative colitis), hepatitis, nonalcoholic fatty liver disease, nonalcoholic steatohepatitis, hypertension, myocardial infarction, heart failure, coronary artery disease, oxalate-induced nephropathy, graft-versus host disease, type 1 diabetes, type 2 diabetes, Edema (DME), Geographic Atrophy (GA), rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, skin contact hypersensitivity, sunburn, psoriasis, hidradenitis suppurativa (HS), epidermolysis bullosa, acne, eczema, alopecia areata, actinic keratosis, hyperostosis, osteitis syndrome (SAPHO), vitiligo, atopic dermatitis, cutaneous lupus, multiple sclerosis (MS), psoriasis, Behcet's disease, Sjogren's syndrome, Schnitzler syndrome, chronic obstructive pulmonary disorder (COPD), asthma, steroid-resistant asthma, Coronavirus-associated inflammatory pathologies including Coronavirus-associated respiratory distress syndrome (CARDS), asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, acute kidney disease, chronic kidney disease, lupus nephritis, diabetic nephropathy, alcoholic liver disease, skin contact hypersensitivity, sunburn, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, primary biliary cholangitis, antiphospholipid syndrome, refractory celiac disease, pancreatitis, autoimmune pancreatitis, mucocutaneous lymph node syndrome, lung cancer metastasis, pancreatic cancers, gastric cancers, myelodisplastic syndrome, leukemia; polymyositis, colitis, helminth infection, bacterial infection, abdominal aortic aneurism, wound healing, migraine, depression, psychological stress, pain, neuropathic pain, pericarditis including Dressler's syndrome, ischaemia reperfusion injury, frontotemporal dementia, HIV-associated neurocognitive disorder, traumatic spinal cord injury, traumatic brain injury, inflammatory pain, chronic pain, neuropathic pain, metastatic cancer-induced bone pain, chemotherapy induced peripheral neuropathy and migraine; ankylosing spondylitis and cytokine release syndrome; preferably the disorder is selected from Alzheimer's disease, Parkinson's disease, amyotrophic lateral sclerosis, demyelination, multiple sclerosis, encephalomyelitis, leukoencephalopathy, viral encephalitis, epilepsy, stroke, traumatic brain injury, spinal cord injury, atherosclerosis, asthma, allergic inflammation, cryopyrin-associated periodic syndromes (CAPS), gout, inflammatory bowel disease (IBD), non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, rheumatoid arthritis, myelodysplastic syndrome, anti-neutrophil cytoplasmic antibody-associated vasculitis (AAV), acute kidney disease, chronic kidney disease, lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, Hereditary Recurrent Fevers (HRFs), acne, atopic dermatitis, hidradenitis suppurativa (HS), and amyloidosis (including AL amyloidosis, AA amyloidosis, ATTR amyloidosis, hereditary amyloidoses (including apolipoprotein A-I (AApoAI), apolipoprotein A-II (AApoAII), gelsolin (AGel), fibrinogen (AFib), and lysozyme (ALys)), Beta-2 microglobulin amyloidosis, iAPP amyloidosis).

27. The compound for use according to claim 26, wherein the disease, the disorder or the abnormality is selected from Alzheimer's disease, Parkinson's disease and multiple sclerosis.

28. The compound for use according to claim 26, wherein the disease, the disorder or the abnormality is selected from cryopyrin-associated periodic syndromes (CAPS), non-alcoholic fatty liver disease (NAFLD), non-alcoholic steatohepatitis (NASH), chronic kidney disease and gout.

29. The compound for use according to claim 26, wherein the disease, the disorder or the abnormality is a skin disease, disorder, or abnormality selected from hidradenitis suppurativa (HS), dermatitis, psoriasis, skin contact hypersensitivity, acne, periodic fever syndrome (HIDS), Sweet's syndrome, eczema, skin lesions, burn, wound, wound healing, trauma, sunburn, actinic keratosis, deficiency of interleukin 1 receptor (DIRA) antagonist, epidermolysis bullosa, vitiligo, atopic dermatitis, cutaneous lupus, and alopecia areata.

30. The compound for use according to claim 29, wherein the disease, the disorder or the abnormality is hidradenitis suppurativa (HS).

31. Use of a compound according to any one of claims 1 to 19, or a stereoisomer, a racemic mixture, a tautomer, a polymorph, a pharmaceutically acceptable salt, a prodrug, a hydrate, or a solvate thereof, as an analytical reference or an in vitro screening tool.
